(19) United States
(12) Patent Application Publication
(10) Pub. No.: US 2025/0066505 A1
(43) Pub. Date: Feb. 27, 2025

(54) DIAGNOSTIC METHODS USING ANTI-MUC1* ANTIBODIES

(71) Applicant: Minerva Biotechnologies Corporation, Waltham, MA (US)

(72) Inventor: Cynthia Bamdad, Boston, MA (US)

(21) Appl. No.: 18/935,308

(22) Filed: Nov. 1, 2024

Related U.S. Application Data
(62) Division of application No. 18/612,767, filed on Mar. 21, 2024.
Application No. 18/612,767 is a continuation of application No. 16/975,625, filed on Aug. 25, 2020, now Pat. No. 11,976,132.
Application No. 16/975,625 is a continuation-in-part of application No. PCT/US2018/062569, filed on Nov. 27, 2018.
(60) Provisional application No. 62/791,661, filed on Jan. 11, 2019.
(60) Provisional application No. 62/640,697, filed on Mar. 9, 2018.
(60) Provisional application No. 62/635,378, filed on Feb. 26, 2018.

(51) Int. Cl. C07K 16/30; A61K 35/17; A61K 39/00; A61K 47/68; G01N 33/574

(52) U.S. Cl. CPC C07K 16/3092; A61K 35/17; A61K 39/4611; A61K 39/4631; A61K 39/46447; A61K 47/6851; G01N 33/57492; C07K 2317/34

(57) ABSTRACT

The present application discloses a method of determining suitability of treating a patient suffering from cancer or metastasis of cancer characterized by aberrant expression of MUC1, with a MUC1* targeting therapeutic.

### CROSS-REFERENCE TO RELATED APPLICATIONS

This application is a divisional application of U.S. patent application Ser. No. 18/612,767, filed on Mar. 21, 2024, which is a continuation application of U.S. patent application Ser. No. 16/975,625 filed Aug. 25, 2020, now U.S. Pat. No. 11,976,132, which is the U.S. national phase of International Application No. PCT/US2019/019566 filed Feb. 26, 2019 which claims priority to U.S. Provisional Patent Application No. 62/791,661 filed Jan. 11, 2019, U.S. Provisional Patent Application No. 62/640,697 filed Mar. 9, 2018, and U.S. Provisional Patent Application No. 62/635,378 filed Feb. 26, 2018, the entire contents of each of which are hereby incorporated by reference. International Application No. PCT/US2019/019566 s a continuation-in-part of International Application No. PCT/US2018/062569 filed Nov. 27, 2018, the entire contents of which are hereby incorporated by reference.

### DESCRIPTION OF THE XML FILE SUBMITTED ELECTRONICALLY

The instant application contains a Sequence Listing which has been submitted electronically in XML format and is hereby incorporated by reference in its entirety. Said XML copy, created on Mar. 20, 2024, is named 56699-739_401SL.xml and is 522,295 bytes in size.

### BACKGROUND OF THE INVENTION

The present invention is directed to a method of diagnosing cancer and determining suitability of treating a patient suffering from cancer or metastasis of cancer characterized by aberrant expression of MUC1, with a MUC1* targeting therapeutic, comprising contacting cells or tissue of a patient diagnosed with or suspected of having cancer, with an antibody that binds to a form of MUC1 that is devoid of the tandem repeat domain, wherein the presence of specific binding of the antibody to the cleaved or truncated form of MUC1, and wherein such binding is in an abnormal pattern, indicates that a MUC1* targeting therapeutic is suitable to be used to treat the patient.

Here, we define MUC1* as a transmembrane cleavage product of MUC1 that functions as a growth factor receptor and is devoid of the tandem repeat sequences. However, MUC1 can be cleaved by different enzymes, which cleave at different sites. Which cleavage enzyme clips MUC1 may be tissue specific or patient specific. The conformation of the extra cellular domain of MUC1* may change depending on which cleavage enzyme cleaves it. Anti-MUC1* antibodies may bind to the extra cellular domain of the transmembrane receptor that remains after cleavage.

In one aspect, the antibody may bind to a peptide of Primary Sequence of MUC1 Growth Factor (PSMGFR), PSMGFR N-10, PSMGFR C-10, or may bind to PSMGFR N-10 but not to PSMGFR C-10, or may bind to PSMGFR C-10 but not to PSMGFR N-10, or may bind to the PSMGFR N+20 peptides such as N+20/C-22, N+20/C-41, or N+20/C-27 peptide, or a N+9/C-9 peptide. The antibody may bind to a peptide having a sequence that is extended N-terminally beyond the PSMGFR sequence. The antibody may bind to a peptide of sequence N+20-PSMGFR or N+9-PSMGFR. In one aspect of the invention, diagnostic assays employing anti-MUC1* antibodies or fragments thereof are used to screen patients to determine their potential benefit from a MUC1* targeting therapeutic. In one aspect of the invention, the antibody used in the diagnostic and the antibody or fragment thereof that is incorporated into the therapeutic are derived from the same antibody. The species of the diagnostic antibody and the therapeutic antibody do not need to be the same.

In one example, (i) a suspect cellular or tissue specimen, which may be a biopsy, from a patient diagnosed with cancer or suspected of developing cancer is contacted with an anti-MUC1* antibody; (ii) a normal cellular or tissue specimen from the patient or from a healthy donor is contacted with the same anti-MUC1* antibody, which may be an archived reference specimen; (iii) antibody binding is detected; (iv) the extent and pattern of antibody binding to the suspect specimen is compared to that of the normal specimen; (v) a determination that the suspect specimen overexpresses MUC1*, or expresses MUC1* in a uniform pattern as opposed to expression that is restricted to the apical border, indicates that the patient is suffering from a MUC1* positive cancer; (vi) a therapeutic agent that incorporates an anti-MUC1* antibody, or fragment thereof, is administered to the patient.

In another aspect of the invention, anti-MUC1* antibodies can be attached to an imaging agent for use in a patient as a whole body diagnostic to determine if the patient has a MUC1* positive tumor or, depending on the specific antibody used, if the patient would benefit from a therapeutic comprising all or a fragment of the antibody that is attached to the imaging agent. The species of the diagnostic antibody and the therapeutic antibody do not need to be the same. Antibodies generated in camelid species are particularly useful for in vivo diagnostic assays because camelids generated small monovalent antibodies that have a short half-life in humans.

In another aspect of the invention, anti-MUC1* antibodies, which may be attached to an imaging agent are used intra-surgically to detect or mark cancerous tissues so they can be excised during the surgery.

In another aspect of the invention, anti-MUC1* antibodies or fragments thereof that bind to a peptide having some or all of the sequence of the PSMGFR peptide are used for the diagnosis and/or treatment of breast cancers.

In another aspect of the invention, anti-MUC1* antibodies or fragments thereof that bind to a peptide having some or all of the sequence of the PSMGFR peptide, extended at the N-terminus by as many as 20 amino acids are used for the diagnosis and/or treatment of pancreatic cancers.

In another aspect of the invention, anti-MUC1* antibodies or fragments thereof that bind to a peptide having some or all of the sequence of the PSMGFR peptide, extended at the N-terminus by as many as 20 amino acids are used for the diagnosis and/or treatment of esophageal cancers.

In another aspect of the invention, anti-MUC1* antibodies or fragments thereof that bind to a peptide having some or all of the sequence of the PSMGFR peptide, extended at the N-terminus by as many as 20 amino acids are used for the diagnosis and/or treatment of prostate cancers.

In one aspect, the MUC1* targeting therapeutic may be a cancer immunotherapy. The MUC1* targeting therapeutic may be a CAR T, a BiTE, an ADC (antibody drug conjugate), a bispecific antibody or an antibody mimic.

The MUC1* targeting therapeutic may be an antibody that binds to a cleaved form of MUC1 wherein the cleaved form is the extra cellular domain of the transmembrane receptor that remains after cleavage. The antibody may bind to a peptide known as Primary Sequence of MUC1 Growth Factor (PSMGFR) or to a peptide that is N-terminally extended for up to 20 amino acids beyond the PSMGFR sequence. The antibody used in the therapeutic may be derived from the antibody used in the diagnostic assay, but need not be generated in the same species animal.

The inventive method may be an in vitro assay. The assay may be carried out on a tissue specimen, bodily fluid sample, or a blood sample.

In another aspect, the assay may be an in vivo assay. An imaging agent may be attached to the antibody.

In another aspect, the invention may comprise a second antibody, and the steps may comprise determining the ratio of the amount of a first antibody to a second antibody. The first antibody may bind to an extra cellular domain of the transmembrane receptor that remains after cleavage and the second antibody may bind to a portion of the MUC1 extra cellular domain that is N-terminal of the cleavage site, such as the tandem repeat sequences.

In another aspect, in reference to all of the above methods, the non-human, human or humanized anti-MUC1* antibody or antibody fragment or antibody-like protein may specifically bind to
(i) PSMGFR region of MUC1;
(ii) PSMGFR peptide as set forth in SEQ ID NO:4;
(iii) PSMGFR N+20/C-22, a peptide having amino acid sequence of SNIKFRPGSVVVQLTLAFREGTINVHDVETQFNQYKTEAASRY (SEQ ID NO:5);
(iv) PSMGFR N+12/C-22, a peptide having amino acid sequence of SVVVQLTLAFREGTINVHDVETQFNQYKTEAASRY (SEQ ID NO:6);
(v) PSMGFR N+9/C-30, a peptide having amino acid sequence of VQLTLAFREGTINVHDVETQFNQY (SEQ ID NO:7);
(vi) PSMGFR N+20/C-41, a peptide having amino acid sequence of SNIKFRPGSVVVQLTLAFREGTIN (SEQ ID NO:8)
(vii) PSMGFR N+20/C-27, a peptide having amino acid sequence of SNIKFRPGSVVVQLTLAFREGTINVHDVETQFNQYKTE (SEQ ID NO:9); or
(viii) PSMGFR N+9/C-9, a peptide having amino acid sequence of VQLTLAFREGTINVHDVETQFNQYKTEAASRYNLTISDVSVSDVP (SEQ ID NO:10).

The antibody that binds to the extra cellular domain of the transmembrane receptor that remains after cleavage may be SDIX SRY polyclonal antibody, MNC2 monoclonal antibody, MNE6 monoclonal antibody, or monoclonal antibodies 1E4, 29H1, 31A1, 32C1, and 45C11 reactive with PSMGFR N+20/C-27; 17H6, 39H5, 3C5, 8A9 reactive with PSMGFR N+9/C-9; 18G12, 20A10, 25E6, 28F9, and 18B4 reactive with PSMGFR, as well as MNC2 and MNE6, which are also reactive with PSMGFR. These antibodies may be human, humanized, mouse, camelid, llama, alpaca, camel, rabbit, goat, hamster or other non-human species.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1A-1D shows photographs of adjacent serial sections of breast cancer tissue arrays and graphical representations of the pathologist scores, according to Allred scoring system. Pathologist score is 0-3, where 0 showed no staining and 3 is the greatest staining. The graphs are also color coded, where a pathologist score zero is black, 1 is yellow, 2 is orange, and 3 is red; tissues that scored zero when probed with an antibody that recognizes full-length MUC1 but positive when probed with an antibody that recognizes MUC1* were colored green; and missing or uninterpretable tissues were scored −1. FIG. 1A shows photographs of the breast cancer tissue arrays after they were stained with VU4H5, which is an antibody that binds to the tandem repeat domains of full-length MUC1. FIG. 1B shows graphs of the pathologist scores for the tissues pictured in FIG. 1A. FIG. 1C shows photographs of the breast cancer tissue arrays after they were stained with MNC2, which is an antibody that binds to an epitope within the PSMGFR region of MUC1*. FIG. 1D shows graphs of the pathologist scores for the tissues pictured in FIG. 1C.

FIG. 2A-2B shows pie chart graphs of the pathologist scores of the arrays shown in FIG. 1A and FIG. 1C. FIG. 2A shows that the antibody that binds to tandem repeats of full-length MUC1 misses 30% of breast cancers. FIG. 2B shows that the anti-MUC1* antibody MNC2 recognizes 95% of breast cancers. Anti-MUC1-full-length only binds strongly to 10% of the breast tumors, while anti-MUC1* antibody MNC2 binds strongly to about 50% of breast tumors.

FIG. 3A-3B shows pie chart graphs of the pathologist scores and a photograph of breast cancer array BR1141 after staining with anti-MUC1* antibody huMNC2-scFv-Fc.

FIG. 4A-4C shows photographs, at two different magnifications, of individual breast cancer specimens from breast cancer array BR1141 after staining with anti-MUC1* antibody huMNC2-scFv-Fc. The position in the array, cancer sub-type, tumor grade, TNM (Tumor stage, Node involvement, and Metastasis) and pathologist score are indicated in figures. Standard immunohistochemistry methods were used. Antibody concentration was titered using the highest concentration at which the antibody showed expected staining of normal tissues without staining stroma. The antibody was conjugated to a biotin through its Fc region, to avoid false positive due to anti-human secondary antibodies staining host antibodies as well as B cell follicles. FIG. 4A shows the specimen at position A7 which was negative for huMNC2 reactive cells. FIG. 4B shows the specimen at position A9 which is a Grade 2 cancer, with lymph node involvement that scored +1 for huMNC2 reactivity. FIG. 4C shows the specimen at position B10 which is a larger Grade 2 tumor, with lymph node involvement that scored +2 for huMNC2 reactivity.

FIG. 5A-5B shows photographs, at two different magnifications, of individual breast cancer specimens from breast cancer array BR1141 after staining with anti-MUC1* antibody huMNC2-scFv-Fc. FIG. 5A shows the specimen at position D7 which is a Grade 2 cancer, without lymph node involvement that scored +3 for huMNC2 reactivity. FIG. 5B shows the specimen at position F6 which is a Grade 2 tumor, with lymph node involvement that scored +4 for huMNC2 reactivity.

FIG. 6A-6B shows pie chart graphs of the pathologist scores and a photograph of ovarian cancer array BC1115a after staining with anti-MUC1* antibody huMNC2-scFv-Fc.

FIG. 7A-7C shows magnified photographs of different cancer sub-types after staining with anti-MUC1* antibody huMNC2-scFv-Fc. FIG. 7A shows a photograph of a Grade 2 breast tumor that pathologist scored +4. FIG. 7B shows a photograph of a Grade 2 ovarian tumor that pathologist scored +3. FIG. 7C shows a photograph of a Grade 3 pancreatic tumor that pathologist scored +3. IHC studies of over 1,000 tumor specimens showed that huMNC2-scFv recognized 95% of Breast Cancers (90% triple negative), 83% Ovarian, 78% Pancreatic & 71% Lung Cancers.

FIG. 8A-8D shows magnified photographs of different cancer sub-types after staining with anti-MUC1* antibody huMNC2-scFv-Fc. FIG. 8A shows a photograph of a Grade 2 breast tumor that pathologist scored +2. FIG. 8B shows a photograph of a Grade 3 ovarian tumor that pathologist scored +3. FIG. 8C shows a photograph of a Grade 3 pancreatic tumor, with lymph node involvement that pathologist scored +3. FIG. 8D shows a photograph of a lung cancer that pathologist scored +3.

FIG. 9A-9I shows magnified photographs of various normal tissues after staining with anti-MUC1* antibody huMNC2-scFv-Fc. Conditions and concentrations used were identical to those used for studying cancerous tissues. FIG. 9A shows normal adrenal gland tissue. FIG. 9B shows normal brain tissue. FIG. 9C shows normal breast tissue. FIG. 9D shows normal stomach tissue. FIG. 9E shows normal heart tissue. FIG. 9F shows normal kidney tissue. FIG. 9G shows normal testis tissue. FIG. 9H shows normal intestine tissue. FIG. 9I shows normal liver tissue.

FIG. 10A-10F shows photographs of normal kidney tissues after staining with anti-MUC1* antibody huMNC2-scFv-Fc. Conditions and concentrations used were identical to those used for studying cancerous tissues. FIG. 10A shows normal kidney tissue with huMNC2 reactivity limited to the apical border, which is normal expression. FIG. 10B is the same tissue at greater magnification. FIG. 10C shows another example of normal kidney tissue with undetectable huMNC2 reactivity. FIG. 10D is the same tissue at greater magnification. FIG. 10E shows another example of normal kidney tissue with huMNC2 reactivity limited to the apical border, which is normal expression. FIG. 10F is the same tissue at greater magnification. Further studies showed that less than 10% of normal kidney tissue showed huMNC2 reactivity at distal collecting tubules wherein such reactivity was strictly limited to the apical border, which is a normal expression pattern.

FIG. 11A-11B shows pie chart graphs of the pathologist scores and a photograph of esophageal cancer array BC001113 after staining with anti-MUC1* antibody huMNC2-scFv-Fc.

FIG. 12A-12F shows photographs, at two different magnifications, of individual esophageal cancer specimens from esophageal cancer array BC001113, after staining with anti-MUC1* antibody huMNC2-scFv-Fc. The position in the array, cancer sub-type, tumor grade, and pathologist score are indicated in figures. FIG. 12A shows the specimen at position A4 which was negative for huMNC2 reactive cells. FIG. 12B shows the same specimen at greater magnification. FIG. 12C shows the specimen at position D2 which the pathologist scored as trace reactivity to huMNC2. FIG. 12D shows the same specimen at greater magnification. FIG. 12E shows the specimen at position B8 which the pathologist scored as +1 reactivity to huMNC2. FIG. 12F shows the same specimen at greater magnification.

FIG. 13A-13D shows photographs, at two different magnifications, of individual esophageal cancer specimens from esophageal cancer array BC001113, after staining with anti-MUC1* antibody huMNC2-scFv-Fc. The position in the array, cancer sub-type, tumor grade, and pathologist score are indicated in figures. FIG. 13A shows the specimen at position D6, a Grade 4 tumor, which the pathologist scored +2. FIG. 13B shows the same specimen at greater magnification. FIG. 13C shows the specimen at position D5, a Grade 3 tumor, which the pathologist scored +3. FIG. 12D shows the same specimen at greater magnification.

FIG. 14A-14B shows pie chart graphs of the pathologist scores and a photograph of pancreatic cancer array PA805b after staining with anti-MUC1* antibody huMNC2-scFv-Fc.

FIG. 15A-15D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 15A shows the specimen at position F3, a Grade 3 tumor, which the pathologist scored +3. FIG. 15B shows the same specimen at greater magnification. FIG. 15C shows the specimen at position B1, a Grade 1 tumor, which the pathologist scored +2. FIG. 15D shows the same specimen at greater magnification.

FIG. 16A-16D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 16A shows the specimen at position A2, a Grade 1 tumor, which the pathologist scored +2. FIG. 16B shows the same specimen at greater magnification. FIG. 16C shows the specimen at position C3, a Grade 2 tumor, which the pathologist scored +2. FIG. 16D shows the same specimen at greater magnification.

FIG. 17A-17D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 17A shows the specimen at position C6, a Grade 2 tumor, which the pathologist scored +2. FIG. 17B shows the same specimen at greater magnification. FIG. 17C shows the specimen at position D1, a larger Grade 3 tumor, with lymph node involvement that the pathologist scored +3. FIG. 17D shows the same specimen at greater magnification.

FIG. 18A-18D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 18A shows the specimen at position E2, a Grade 1 tumor, which the pathologist scored +2. FIG. 18B shows the same specimen at greater magnification. FIG. 18C shows the specimen at position E10, a smaller Grade 3 tumor, with lymph node involvement that the pathologist scored +3. FIG. 18D shows the same specimen at greater magnification.

FIG. 19 shows a photograph of pancreatic cancer array PA805b that was stained with the secondary antibody alone, as a control.

FIG. 20A-20B shows photographs of pancreatic cancer array PA805b that were stained with an anti-MUC1* monoclonal antibody or an anti-MUC1* polyclonal antibody. FIG. 20A shows a photograph of the pancreatic cancer array that was stained with anti-MUC1* monoclonal antibody huMNC2-scFv. FIG. 20B shows a photograph of the pancreatic cancer array that was stained with anti-MUC1* polyclonal antibody SDIX. Both polyclonal and monoclonal antibodies were generated by immunizing the animals with the PSMGFR peptide. The circled specimens are indicated because they show different staining when probed with the monoclonal versus the polyclonal antibody. The numbers beneath each specimen indicate the pathologist score, when probed with huMNC2-scFv, followed by a slash mark, then the tumor grade.

FIGS. 21A-21D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 22A-22D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 23A-23D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 24A-24D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 25A-25D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 26A-26D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 27A-27D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 28A-28D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 29A-29D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 30A-30D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 31A-31D show photographs of individual tumor tissue specimens from the pancreatic cancer array, comparing the staining intensity and pattern of staining when the patient sample is probed with monoclonal antibody MNC2 or polyclonal antibody SDIX, wherein both antibodies bind to the PSMGFR peptide. In the figures, (A) is stained with MNC2, (B) is the same tissue but at greater magnification, (C) is stained with SDIX, and (D) is that same tissue but shown at greater magnification.

FIGS. 32A-32D show photographs of individual tissue specimens from the pancreatic cancer array, but the specimens that are shown are normal pancreatic tissues. The staining intensity and pattern of staining of monoclonal antibody MNC2 is compared to that of polyclonal antibody SDIX. In the figures, (A) is stained with MNC2, (B) is the same tissue but at greater magnification, (C) is stained with SDIX, and (D) is that same tissue but shown at greater magnification.

FIGS. 33A-33D show photographs of individual tissue specimens from the pancreatic cancer array, but the specimens that are shown are normal pancreatic tissues. The staining intensity and pattern of staining of monoclonal antibody MNC2 is compared to that of polyclonal antibody SDIX. In the figures, (A) is stained with MNC2, (B) is the same tissue but at greater magnification, (C) is stained with SDIX, and (D) is that same tissue but shown at greater magnification.

FIGS. 34A-34D show photographs of individual tissue specimens from the pancreatic cancer array, but the specimens that are shown are normal pancreatic tissues. The staining intensity and pattern of staining of monoclonal antibody MNC2 is compared to that of polyclonal antibody SDIX. In the figures, (A) is stained with MNC2, (B) is the same tissue but at greater magnification, (C) is stained with SDIX, and (D) is that same tissue but shown at greater magnification.

FIG. 35A-35B shows cartoons of MUC1* expression on cancer cells and on normal hematopoietic stem cells. FIG. 35A depicts MUC1* on a cancer cell being probed by anti-MUC1* monoclonal antibody, MNC2. FIG. 35B depicts MUC1* on normal hematopoietic stem cells being probed by anti-MUC1* monoclonal antibody, MNC3. Both antibodies were generated by immunizing the animal with a PSMGFR peptide. MNC2 does not bind to normal hematopoietic stem cells but MNC3 does.

FIG. 36A-36B shows FACS analysis of human hematopoietic stem cells stained with anti-PSMGFR antibodies. FIG. 36A shows a graph of FACS results showing that the SDIX polyclonal antibody and the MNC3 monoclonal antibody recognize nearly 100% of the hematopoietic stem cells but MNC2 monoclonal antibody does not bind to them. FIG. 36B shows an overlay of the FACs scans, which shows that MNC2 binding is no different than the control antibody, while MNC3 produces a clear shift in the cell populations. All three antibodies were generated by immunizing with the PSMGFR peptide.

FIG. 37 shows a graph of FACS analysis of cells that express 90% full-length MUC1 after addition of a catalytic domain of cleavage enzyme MMP9, then probing with anti-full-length MUC1 antibody VU4H5 or anti-MUC1* antibody MNC2.

FIG. 38A-38C lists new anti-MUC1* monoclonal antibodies that were generated by immunizing animals with one of three different peptides derived from the MUC1* extra cellular domain sequence. FIG. 38A lists monoclonal antibodies that were generated when animals were immunized with the PSMGFR peptide. FIG. 38B lists monoclonal antibodies that were generated when animals were immunized with the PSMGFR N+20/C-27 peptide. FIG. 38C lists monoclonal antibodies that were generated when animals were immunized with the PSMGFR N+9/C-9 peptide. The −1 or −2 designation refers to sister clones from the same well. Concentrations of stock antibody solutions are given.

FIG. 39 shows a graph of an ELISA experiment testing the ability of monoclonal antibodies, generated by immunizing with the PSMGFR peptide, to bind to other peptides derived from the sequence of the MUC1* extra cellular domain. All monoclonal antibodies were first selected based on their ability to bind to the immunizing peptide. To further elucidate the epitope within that peptide to which the antibody binds, antibodies were tested for their ability to bind to the PSMGFR peptide, the N-10 peptide or the C-10 peptide.

FIG. 40A-40B shows graphs of FACS analysis of the new PSMGFR anti-MUC1* monoclonal antibodies binding to T47D breast cancer cells. FIG. 40A shows the Mean Fluorescence Intensity. FIG. 40B shows the percent of cells that stained positive with the respective antibody.

FIG. 41 shows a graph of an ELISA experiment testing the ability of monoclonal antibodies, generated by immunizing with the PSMGFR N+20/C-27 peptide, to bind to other peptides derived from the sequence of the MUC1* extra cellular domain. All monoclonal antibodies were first selected based on their ability to bind to the immunizing peptide. To further elucidate the epitope within that peptide to which the antibody binds, antibodies were tested for their ability to bind to the PSMGFR peptide, the N-10 peptide or the C-10 peptide.

FIG. 42A-42B shows graphs of FACS analysis of the new PSMGFR N+20/C-27 anti-MUC1* monoclonal antibodies binding to T47D breast cancer cells. FIG. 42A shows the Mean Fluorescence Intensity. FIG. 42B shows the percent of cells that stained positive with the respective antibody.

FIG. 43 shows a graph of an ELISA experiment testing the ability of monoclonal antibodies, generated by immunizing with the PSMGFR N+9/C-9 peptide, to bind to other peptides derived from the sequence of the MUC1* extra cellular domain. All monoclonal antibodies were first selected based on their ability to bind to the immunizing peptide. To further elucidate the epitope within that peptide to which the antibody binds, antibodies were tested for their ability to bind to the PSMGFR peptide, the N-10 peptide or the C-10 peptide.

FIG. 44A-44B shows graphs of FACS analysis of the new PSMGFR N+9/C-9 anti-MUC1* monoclonal antibodies binding to T47D breast cancer cells. FIG. 44A shows the Mean Fluorescence Intensity. FIG. 44B shows the percent of cells that stained positive with the respective antibody.

FIG. 45A-45C shows photographs of adjacent serial sections from a pancreatic cancer array, which was stained by standard IHC methods with various anti-MUC1* antibodies. FIG. 45A shows the array stained with the SDIX polyclonal anti-MUC1* antibody, wherein the immunogen for the antibody was the PSMGFR peptide. FIG. 45B shows the array stained with the 18B4 monoclonal anti-MUC1* antibody, wherein the immunogen for the antibody was the PSMGFR peptide. FIG. 45C shows the array stained with the 1E4 monoclonal anti-MUC1* antibody, wherein the immunogen for the antibody was the PSMGFR N+20/C-27 peptide.

FIG. 46A-46F shows photographs of individual specimens from the pancreatic cancer array shown in FIG. 45. FIG. 46A shows a specimen stained with the SDIX polyclonal antibody. FIG. 46B shows the same tissue specimen at a greater magnification. FIG. 46C shows the adjacent tissue section stained with the 18B4 monoclonal antibody. FIG. 46D shows the same tissue specimen at a greater magnification. FIG. 46E shows the adjacent tissue section stained with the 1E4 monoclonal antibody. FIG. 46F shows the same tissue specimen at a greater magnification.

FIG. 47A-47D shows photographs of individual specimens from the pancreatic cancer array shown in FIG. 45. FIG. 47A shows a specimen stained with the SDIX polyclonal antibody. FIG. 47B shows the same tissue specimen at a greater magnification. FIG. 47C shows the adjacent tissue section stained with the 18B4 monoclonal antibody. FIG. 47D shows the same tissue specimen at a greater magnification.

FIG. 48A-48D shows photographs of individual specimens from the pancreatic cancer array shown in FIG. 45. FIG. 48A shows a specimen stained with the SDIX polyclonal antibody. FIG. 48B shows the same tissue specimen at a greater magnification. FIG. 48C shows the adjacent tissue section stained with the 18B4 monoclonal antibody. FIG. 48D shows the same tissue specimen at a greater magnification.

FIG. 49A-49D shows photographs of individual specimens from the pancreatic cancer array shown in FIG. 45. FIG. 49A shows a specimen stained with the SDIX polyclonal antibody. FIG. 49B shows the same tissue specimen at a greater magnification. FIG. 49C shows the adjacent tissue section stained with the 1E4 monoclonal antibody. FIG. 49D shows the same tissue specimen at a greater magnification. Comparing FIG. 49A to FIG. 49C, it is clear that the monoclonal antibody generated by immunizing with the PSMGFR N+20/C-27 peptide recognizes a different cell population within the tumor than that recognized by the polyclonal antibody, SDIX, that was generated by immunizing with the PSMGFR peptide.

FIG. 50A-50D shows photographs of individual specimens from the pancreatic cancer array shown in FIG. 45. FIG. 50A shows a specimen stained with the SDIX polyclonal antibody. FIG. 50B shows the same tissue specimen at a greater magnification. FIG. 50C shows the adjacent tissue section stained with the 1E4 monoclonal antibody. FIG. 50D shows the same tissue specimen at a greater magnification. Comparing FIG. 50A to FIG. 50C, it is clear that the monoclonal antibody generated by immunizing with the PSMGFR N+20/C-27 peptide recognizes a different cell population within the tumor than that recognized by the polyclonal antibody, SDIX, that was generated by immunizing with the PSMGFR peptide.

FIG. 51A-51C shows photographs of adjacent serial sections from a pancreatic cancer array, which was stained by standard IHC methods with various anti-MUC1* antibodies. FIG. 51A shows the array stained with the SDIX polyclonal anti-MUC1* antibody, wherein the immunogen for the antibody was the PSMGFR peptide. FIG. 51B shows the array stained with the 20A10 monoclonal anti-MUC1* antibody, wherein the immunogen for the antibody was the PSMGFR peptide. FIG. 51C shows the array stained with the 29H1 monoclonal anti-MUC1* antibody, wherein the immunogen for the antibody was the PSMGFR N+20/C-27 peptide.

FIG. 52A-52D shows photographs of adjacent serial sections from a pancreatic cancer array, which was stained by standard IHC methods with various anti-MUC1* antibodies. FIG. 52A shows the array stained with the 17H6 monoclonal anti-MUC1* antibody, wherein the immunogen for the antibody was the PSMGFR N+9/C-9 peptide. FIG. 52B shows the array stained with the 32C1 monoclonal anti-MUC1* antibody, wherein the immunogen for the antibody was the PSMGFR N+20/C-27 peptide. FIG. 52C shows the array stained with the 45C11 monoclonal anti-MUC1* antibody, wherein the immunogen for the antibody was the PSMGFR N+20/C-27 peptide. FIG. 52D shows the array stained with the 31A1 monoclonal anti-MUC1* antibody, wherein the immunogen for the antibody was the PSMGFR N+20/C-27 peptide.

FIG. 53A-53F shows photographs and graphical representations of pathologist staining scores of adjacent serial sections from a pancreatic cancer array, which was stained by standard IHC methods with either antibodies that recognize full-length MUC1 or an antibody that only recognizes MUC1*. FIG. 53A shows the pancreatic cancer array stained with antibody 5E5, which is an antibody that binds to a trapped O-linked glycan in the tandem repeat domain of full-length MUC1. FIG. 53B shows the pathologist's score for each specimen in the array. FIG. 53C shows the pancreatic cancer array stained with anti-MUC1* antibody 29H1, which is an antibody that binds to the PSMGFR N+20/C-27 peptide of MUC1*. FIG. 53D shows the pathologist's score for each specimen in the array. FIG. 53E shows the pancreatic cancer array stained with antibody VU4H5, which is an antibody that binds to an epitope in the tandem repeat domain of full-length MUC1. FIG. 53F shows the pathologist's score for each specimen in the array. As can be seen if the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1, and that the antibodies that bind to the PSMGFR N+20/C-27 peptide recognize epitopes that are prevalent on pancreatic cancers.

FIG. 54A-54C shows photographs of adjacent serial sections from an esophageal cancer array, which was stained by standard IHC methods with various anti-MUC1* antibodies. FIG. 54A shows the array stained with the SDIX polyclonal anti-MUC1* antibody, wherein the immunogen for the antibody was the PSMGFR peptide. FIG. 54B shows the array stained with the 20A10 monoclonal anti-MUC1* antibody, wherein the immunogen for the antibody was the PSMGFR peptide. FIG. 54C shows the array stained with the 29H1 monoclonal anti-MUC1* antibody, wherein the immunogen for the antibody was the PSMGFR N+20/C-27 peptide. FIG. 54D shows the array stained with the 31A1 monoclonal anti-MUC1* antibody, wherein the immunogen for the antibody was the PSMGFR N+20/C-27 peptide. This figure shows that antibodies SDIX and 20A10 that both bind to the PSMGFR peptide recognize the same tumor tissue specimens, albeit to differing degrees, while antibodies that bind to the PSMGFR N+20/C-27 peptide bind to more esophageal tumor specimens as well as most of those recognized by the anti-PSMGFR antibodies. These results are consistent with the idea that antibodies that bind to the PSMGFR N+20/C-27 peptide are general more specific for esophageal cancers than antibodies that bind to the PSMGFR peptide, but that certain patients may have an esophageal cancer that is better recognized by an anti-MUC1* antibody that binds to the PSMGFR peptide.

FIG. 55A-55C shows photographs of adjacent serial sections from an esophageal cancer array, which was stained by standard IHC methods with various anti-MUC1* antibodies. FIG. 55A shows the array stained with the SDIX polyclonal anti-MUC1* antibody, wherein the immunogen for the antibody was the PSMGFR peptide. FIG. 55B shows the array stained with the 17H6 monoclonal anti-MUC1* antibody, wherein the antibody binds to the PSMGFR N+9/C-9 peptide.

FIG. 55C shows the array stained with the MNC2 monoclonal anti-MUC1* antibody, wherein the antibody binds to the PSMGFR peptide. FIG. 55D shows the array stained with the 45C11 monoclonal anti-MUC1* antibody, wherein the antibody binds to the PSMGFR N+20/C-27 peptide. These results are consistent with the idea that on most esophageal cancers, MUC1 is cleaved by an enzyme that exposes a cryptic epitope that is N-terminal to the PSMGFR sequence.

FIG. 56A-56F shows photographs and graphical representations of pathologist staining scores of adjacent serial sections from a esophageal cancer array, which was stained by standard IHC methods with either antibodies that recognize full-length MUC1 or an antibody that only recognizes MUC1*. FIG. 56A shows the esophageal cancer array stained with antibody 5E5, which is an antibody that binds to a trapped O-linked glycan in the tandem repeat domain of full-length MUC1. FIG. 56B shows the pathologist's score for each specimen in the array. FIG. 56C shows the esophageal cancer array stained with anti-MUC1* antibody 29H1, which is an antibody that binds to the PSMGFR N+20/C-27 peptide of MUC1*. FIG. 56D shows the pathologist's score for each specimen in the array. FIG. 56E shows the esophageal cancer array stained with antibody VU4H5, which is an antibody that binds to an epitope in the tandem repeat domain of full-length MUC1. FIG. 56F shows the pathologist's score for each specimen in the array. As can be seen if the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1, and that the antibodies that bind to the PSMGFR N+20/C-27 peptide recognize epitopes that are prevalent on esophageal cancers.

FIG. 57A-57G shows photographs of the prostate cancer array, which was stained with either antibody 5E5 or VU4H5, which both recognize full-length MUC1 or 29H1 that only recognizes MUC1* and binds to the PSMGFR N+20/C-27 peptide. FIG. 57A shows the esophageal cancer array stained with antibody 5E5. FIG. 57B shows the esophageal cancer array stained with antibody 29H1. FIG. 57B shows the esophageal cancer array stained with antibody 29H1. FIG. 57C shows the esophageal cancer array stained with antibody VU4H5. FIG. 57D shows the esophageal cancer array stained with the secondary antibody only, as a control. FIG. 57E shows the tissue marked by red box in FIG. 57A at greater magnification, wherein staining was done with 5E5. FIG. 57F shows the tissue marked by red box in FIG. 57B at greater magnification, wherein staining was done with 29H1. FIG. 57G shows the tissue marked by red box in FIG. 57C at greater magnification, wherein staining was done with VU4H5. The dashed red boxes indicate just one patient's specimen of many esophageal tumor specimens that stain negative for antibodies that recognize full-length MUC1, but highly positive when probed with anti-MUC1* antibodies, and particularly those antibodies that bind to the PSMGFR N+20/C-27 peptide.

FIG. 58A-58C shows photographs of adjacent serial sections from a prostate cancer array, which was stained by standard IHC methods with various anti-MUC1* antibodies. FIG. 58A shows the array stained with the SDIX polyclonal anti-MUC1* antibody, wherein the immunogen for the antibody was the PSMGFR peptide. FIG. 58B shows the array stained with the 18B4 monoclonal anti-MUC1* antibody, wherein the antibody binds to the PSMGFR peptide. FIG. 58C shows the array stained with the 1E4 monoclonal anti-MUC1* antibody, wherein the antibody binds to the PSMGFR N+20/C-27 peptide.

FIG. 59A-59E shows photographs of adjacent serial sections from a prostate cancer array, which was stained by standard IHC methods with various anti-MUC1* antibodies. FIG. 59A shows the array stained with the MNC2 monoclonal antibody that binds to the PSMGFR peptide but not the C-10 peptide. FIG. 59B shows the array stained with the 18B4 antibody that binds to the PSMGFR peptide. FIG. 59C shows the array stained with the 32C1 antibody that binds to the PSMGFR N+20/C-27 peptide. FIG. 59D shows the array stained with the SDIX polyclonal anti-MUC1* antibody, wherein the immunogen for the antibody was the PSMGFR peptide. FIG. 59E shows the array stained with the 31A1 monoclonal anti-MUC1* antibody that binds to the PSMGFR N+20/C-27 peptide.

FIG. 60A-60F shows photographs and graphical representations of pathologist staining scores of adjacent serial sections from a prostate cancer array, which was stained by standard IHC methods with either antibodies that recognize full-length MUC1 or an antibody that only recognizes MUC1*. FIG. 60A shows the prostate cancer array stained with antibody 5E5, which is an antibody that binds to a trapped O-linked glycan in the tandem repeat domain of full-length MUC1. FIG. 60B shows the pathologist's score for each specimen in the array. FIG. 60C shows the prostate cancer array stained with anti-MUC1* antibody 29H1, which is an antibody that binds to the PSMGFR N+20/C-27 peptide of MUC1*. FIG. 60D shows the pathologist's score for each specimen in the array. FIG. 60E shows the prostate cancer array stained with antibody VU4H5, which is an antibody that binds to an epitope in the tandem repeat domain of full-length MUC1. FIG. 60F shows the pathologist's score for each specimen in the array. As can be seen if the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1, and that the antibodies that bind to the PSMGFR N+20/C-27 peptide recognize epitopes that are prevalent on prostate cancers.

FIG. 61A-61G shows photographs of the prostate cancer array, which was stained with either antibody 5E5 or VU4H5, which both recognize full-length MUC1 or 29H1 that only recognizes MUC1* and binds to the PSMGFR N+20/C-27 peptide. FIG. 61A shows the prostate cancer array stained with antibody 5E5. FIG. 61B shows the prostate cancer array stained with antibody 29H1. FIG. 61B shows the prostate cancer array stained with antibody 29H1. FIG. 61C shows the prostate cancer array stained with antibody VU4H5. FIG. 61D shows the prostate cancer array stained with the secondary antibody only, as a control. FIG. 61E shows the tissue marked by red box in FIG. 61A at greater magnification, wherein staining was done with 5E5.

FIG. 61F shows the tissue marked by red box in FIG. 61B at greater magnification, wherein staining was done with 29H1. FIG. 61G shows the tissue marked by red box in FIG. 61C at greater magnification, wherein staining was done with VU4H5. The dashed red boxes indicate just one patient's specimen of many prostate tumor specimens that stain negative for antibodies that recognize full-length MUC1, but highly positive when probed with anti-MUC1* antibodies, and particularly those antibodies that bind to the PSMGFR N+20/C-27 peptide.

FIG. 62A-62B shows photographs of adjacent serial sections of breast cancer array BR1141 that were stained with two different anti-MUC1* monoclonal antibodies. FIG. 62A shows a photograph of the breast cancer array that was stained with anti-MUC1* monoclonal antibody MNC2. FIG. 62B shows a photograph of the breast cancer array that was stained with anti-MUC1* monoclonal antibody 20A10. Both monoclonal antibodies bind to the PSMGFR peptide, the N-10 peptide but not to the C10 peptide.

FIG. 63A-63B shows photographs of adjacent serial sections of breast cancer array BR1141 that were stained with two different anti-MUC1* monoclonal antibodies. FIG. 63A shows a photograph of the breast cancer array that was stained with anti-MUC1* monoclonal antibody MNC2. FIG. 63B shows a photograph of the breast cancer array that was stained with anti-MUC1* monoclonal antibody 25E6. Both monoclonal antibodies bind to the PSMGFR peptide and to the N-10 peptide. Whereas MNC2 cannot bind to the C-10 peptide, 25E6 shows some low level of binding to the C-10 peptide, indicating that they bind to different epitopes.

FIG. 64A-64B shows photographs of adjacent serial sections of breast cancer array BR1141 that were stained with two different anti-MUC1* monoclonal antibodies. FIG. 64A shows a photograph of the breast cancer array that was stained with anti-MUC1* monoclonal antibody MNC2. FIG. 64B shows a photograph of the breast cancer array that was stained with anti-MUC1* monoclonal antibody 18B4. Both monoclonal antibodies bind to the PSMGFR peptide. However, unlike MNC2, 18B4 cannot bind to the N-10 epitope, indicating that they bind to different epitopes and that 18B4 may require the 10 N-terminal amino acids of the PSMGFR peptide for binding.

FIG. 65A-65B shows photographs of adjacent serial sections of breast cancer array BR1141 that were stained with two different anti-MUC1* monoclonal antibodies. FIG. 65A shows a photograph of the breast cancer array that was stained with anti-MUC1* monoclonal antibody MNC2. FIG. 65B shows a photograph of the breast cancer array that was stained with anti-MUC1* monoclonal antibody 18G12. Both monoclonal antibodies bind to the PSMGFR peptide. However, unlike MNC2, 18G12 binds to the C-10 epitope to some degree, indicating they bind to different epitope within PSMGFR peptide.

FIG. 66A-66B shows photographs of adjacent serial sections of breast cancer array BR1141 that were stained with two different anti-MUC1* monoclonal antibodies. FIG. 66A shows a photograph of the breast cancer array that was stained with anti-MUC1* monoclonal antibody MNC2. FIG. 66B shows a photograph of the breast cancer array that was stained with anti-MUC1* monoclonal antibody 8A9. Monoclonal antibody MNC2 binds to the PSMGFR peptide, whereas 8A9 binds to the PSMGFR N+9/C-9 peptide. The peptides to which they bind, combined with the very different staining patterns indicates that they bind to different MUC1* epitopes.

FIG. 67A-67B shows photographs of adjacent serial sections of breast cancer array BR1141 that were stained with two different anti-MUC1* monoclonal antibodies. FIG. 67A shows a photograph of the breast cancer array that was stained with anti-MUC1* monoclonal antibody MNC2. FIG. 67B shows a photograph of the breast cancer array that was stained with anti-MUC1* monoclonal antibody 28F9. Both monoclonal antibodies bind to the PSMGFR peptide.

FIG. 68A-68B shows photographs of adjacent serial sections of breast cancer array BR1141 that were stained with two different anti-MUC1* monoclonal antibodies. FIG. 68A shows a photograph of the breast cancer array that was stained with anti-MUC1* monoclonal antibody MNC2. FIG. 68B shows a photograph of the breast cancer array that was stained with anti-MUC1* monoclonal antibody 17H6. Monoclonal antibody MNC2 binds to the PSMGFR peptide, whereas 17H6 binds to the PSMGFR N+9/C-9 peptide. The peptides to which they bind, combined with the very different staining patterns indicates that they bind to different MUC1* epitopes.

FIG. 69A-69B shows photographs of adjacent serial sections of breast cancer array BR1141 that were stained with two different anti-MUC1* monoclonal antibodies. FIG. 69A shows a photograph of the breast cancer array that was stained with anti-MUC1* monoclonal antibody MNC2. FIG. 69B shows a photograph of the breast cancer array that was stained with anti-MUC1* monoclonal antibody 3C5. Monoclonal antibody MNC2 binds to the PSMGFR peptide, whereas 3C5 binds to the PSMGFR N+9/C-9 peptide. The peptides to which they bind, combined with the very different staining patterns indicates that they bind to different MUC1* epitopes.

FIG. 70A-70G shows photographs of adjacent serial sections of breast cancer array BR1007 that were stained with four different anti-MUC1* monoclonal antibodies. FIG. 70A shows a photograph of the breast cancer array that was stained with anti-MUC1* monoclonal antibody 20A10, which binds to the PSMGFR peptide. FIG. 70B shows a photograph of the breast cancer array that was stained with anti-MUC1* monoclonal antibody 29H1, which binds to the PSMGFR N+20/C-27 peptide. FIG. 70C shows a photograph of the breast cancer array that was stained with anti-MUC1* monoclonal antibody 45C11, which binds to the PSMGFR N+20/C-27 peptide. FIG. 70D shows a photograph of the breast cancer array that was stained with anti-MUC1* monoclonal antibody 32C1, which binds to the PSMGFR N+20/C-27 peptide.

FIG. 70E shows a photograph of the breast cancer array that was stained with anti-MUC1* monoclonal antibody 18B4, which binds to the PSMGFR peptide. FIG. 70F shows a photograph of the breast cancer array that was stained with anti-MUC1* monoclonal antibody 31A1, which binds to the PSMGFR N+20/C-27 peptide. FIG. 70G shows a photograph of the breast cancer array that was stained with anti-MUC1* monoclonal antibody 17H6, which binds to the PSMGFR N+9/C-9 peptide.

FIG. 71A-71F shows photographs and graphical representations of pathologist staining scores of adjacent serial sections from a breast cancer array, which was stained by standard IHC methods with either antibodies that recognize full-length MUC1 or an antibody that only recognizes MUC1*. FIG. 71A shows the breast cancer array stained with antibody 5E5, which is an antibody that binds to a trapped O-linked glycan in the tandem repeat domain of full-length MUC1. FIG. 71B shows the pathologist's score for each specimen in the array. FIG. 71C shows the breast cancer array stained with anti-MUC1* antibody 29H1, which is an antibody that binds to the PSMGFR N+20/C-27 peptide of MUC1*. FIG. 71D shows the pathologist's score for each specimen in the array. FIG. 71E shows the breast cancer array stained with antibody VU4H5, which is an antibody that binds to an epitope in the tandem repeat domain of full-length MUC1. FIG. 71F shows the pathologist's score for each specimen in the array. As can be seen if the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1.

FIG. 72A-72F shows photographs of adjacent serial sections of breast cancer tissue array BR1141 that have been stained with various anti-MUC1* monoclonal antibodies, wherein all antibodies bind to the PSMGFR peptide. FIG. 72A shows breast cancer specimens that were stained with MNC2. FIG. 72B shows breast cancer specimens that were stained with 20A10. FIG. 72C shows breast cancer specimens that were stained with 25E6. FIG. 72D shows breast cancer specimens that were stained with 28F9. FIG. 72E shows breast cancer specimens that were stained with 18G12. FIG. 72F shows breast cancer specimens that were stained with 18B4. All these antibodies bind to the PSMGFR peptide and roughly produce the same staining pattern of this breast cancer array. However, there are some differences in how these antibodies recognize individual specimens within the array, which could represent MUC1 to MUC1* cleavage by different enzymes. Referring to FIG. 39, MNC2 and 20A10 bind to the N-10 peptide but not to the C-10 peptide, indicating the 10 membrane proximal amino acids are important for their binding. Antibodies 18B4, 18G12 and 25E6 show some binding to the C-10 peptide and 28F9 shows even more binding to C-10 peptide. Notably, 18B4 does not bind to the N-10 peptide, indicating that it binds to an epitope that is more N-terminal within PSMGFR than the others. Red circles indicate specimens of interest for comparison.

FIG. 73A-73F shows photographs of adjacent serial sections of breast cancer tissue array BR1141 that have been stained with various anti-MUC1* monoclonal antibodies, wherein antibodies that bind to the PSMGFR N+9/C-9 peptide are compared to MNC2 and its humanized single chain form, huMNC2-scFv-Fc, which both bind to PSMGFR, N-10 but not to C-10 peptides. FIG. 73A shows breast cancer specimens that were stained with MNC2. FIG. 73B shows breast cancer specimens that were stained with 8A9. FIG. 73C shows breast cancer specimens that were stained with 17H6. FIG. 73D shows breast cancer specimens that were stained with huMNC2-scFv-Fc. FIG. 73E shows breast cancer specimen that was stained with 3C5. FIG. 73F shows breast cancer specimens that were stained with 39H5. Referring now to the patient specimens that are marked by red circles, it is plain to see that antibodies that bind to the PSMGFR N+9/C-9 peptide recognize a population of breast cancer cells that MNC2 anti-PSMGFR antibodies miss or bind weakly to.

In addition to monoclonal antibodies MNC2, MNE6, MNC3, MNC8, and 18B4, 18G12, 20A10, 25E6, 1E4, 29H1, 31A1, 32C1, 45C11, 3C5, 8A9, 17H6, and 39H5 disclosed in the present application, other monoclonal antibody sequences are recited in SEQ ID NOS: 237-349 that are made from inoculation with the PSMGFR peptide.

### DETAILED DESCRIPTION OF THE PREFERRED EMBODIMENTS

In the present application, “a” and “an” are used to refer to both single and a plurality of objects.

As used herein, occasionally, in short hand, a polypeptide is indicated as being “transduced or transfected” into a cell. In these occurrences, it is understood that the nucleic acid encoding the polypeptide sequence is transduced or transfected into the cell, as it is an impossibility that a polypeptide could be transduced or transfected into a cell.

As used herein, occasionally when referring to number of cells injected into an animal or otherwise contextually wherein the number of cells is referred to, “M” refers to millions, and “K” refers to thousands.

As used herein, interchangeable designations for various monoclonal antibodies are used, such as, “MN-C2”, which is interchangeable with “C2”, “Min-C2” and “MNC2”; “MN-E6”, which is interchangeable with “E6”, “Min-E6” and “MNE6”; “MN-C3”, which is interchangeable with “C3”, “Min-C3” and “MNC3”; and “MN-C8”, which is interchangeable with “C8”, “Min-C8” and “MNC8”.

As used herein, “h” or “hu” placed before an antibody construct is short-hand for human or humanized.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human Fabs that are then screened for binding to peptide epitopes from target proteins. The selected Fab regions can then be engineered into a scaffold or framework so that they resemble antibodies.

As used herein, “PSMGFR” is abbreviation for Primary Sequence of the MUC1 Growth Factor Receptor which is identified by SEQ ID NO:4, and thus is not to be confused with a six amino acid sequence. “PSMGFR peptide” or “PSMGFR region” refers to a peptide or region that incorporates the Primary Sequence of the MUC1 Growth Factor Receptor (SEQ ID NO: 4).

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:4). In this regard, the “N-number” as in “N-10 PSMGFR”, “N-15 PSMGFR”, or “N-20 PSMGFR” refers to the number of amino acid residues that have been deleted at the N-terminal end of PSMGFR, likewise “N+10 PSMGFR”, “N+15 PSMGFR”, or “N+20 PSMGFR” refers to the number of amino acid residues that have been added at the N-terminal end of PSMGFR. Likewise “C-number” as in “C-10 PSMGFR”, “C-15 PSMGFR”, or “C-20 PSMGFR” refers to the number of amino acid residues that have been deleted at the C-terminal end of PSMGFR, and “C+10 PSMGFR”, “C+15 PSMGFR”, or “C+20 PSMGFR” refers to the number of amino acid residues that have been added at the C-terminal end of PSMGFR. Moreover, combinations are possible, such as, “N+20/C-27 PSMGFR”, “PSMGFR N+20/C-27” or “N+20/C-27” which refer to the same peptide, in which the N terminus of PSMGFR includes 20 additional amino acids of MUC1 peptide, and is deleted 27 amino acids at the C-terminus of PSMGFR.

As used herein, when it is desired to refer to a genus of PSMGFR peptides, they are referred to as “PSMGFR group”. For example, “N+20 PSMGFR group” refers to peptides that have additional 20 amino acids at the N-terminus, without regard to how the C-terminus is modified, whether amino acids have been deleted, or added and so on.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:4)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

Other clipped amino acid sequences may include SNIKFRPGSVVVQLTLAFREGTINVHDVETQFNQYKTEAASRY (SEQ ID NO:5); or SVVVQLTLAFREGTINVHDVETQFNQYKTEAASRY (SEQ ID NO:6).

As used herein “sequence identity” means homology in sequence of a particular polypeptide or nucleic acid to a reference sequence of nucleic acid or amino acid such that the function of the homologous peptide is the same as the reference peptide or nucleic acid. Such homology can be so close with the reference peptide such that at times the two sequences may be 90%, 95% or 98% identical yet possess the same function in binding or other biological activities.

As used herein, “MUC1 positive” cell refers to a cell that expresses a gene for MUC1, MUC1-Y or MUC1-Z or other MUC1 variant.

As used herein, “MUC1 negative” cell refers to a cell that does not express a gene for MUC1.

As used herein, “MUC1* positive” cell refers to a cell that expresses a gene for MUC1, wherein that gene's expressed protein is a transmembrane protein that is devoid of tandem repeats, which may be a consequence of post-translational modification, cleavage, alternative splicing, or transfecting or transducing a cell with a MUC1 protein that is devoid of tandem repeats.

As used herein, “MUC1* negative” cell refers to a cell that may or may not express a gene for MUC1 but does not express a MUC1 transmembrane protein that is devoid of tandem repeats.

As used herein, “MUC1 positive” cancer cell refers to a cancer cell that overexpresses the gene for MUC1, expresses MUC1 in an aberrant pattern, wherein its expression is not restricted to the apical border and/or expresses a MUC1 that is devoid of tandem repeats.

As used herein, “MUC1 negative” cancer cell refers to a cancer cell that may or may not express a gene for MUC1 but does not overexpress MUC1 or does not overexpress a MUC1 transmembrane protein that is devoid of tandem repeats.

As used herein, “MUC1* positive” cancer cell refers to a cancer cell that overexpresses a MUC1 transmembrane protein that is devoid of tandem repeats.

As used herein, “MUC1* negative” cancer cell refers to a cancer cell that may or may not express a gene for MUC1 but does not overexpress a MUC1 transmembrane protein that is devoid of tandem repeats.

The present invention involves, generally, diagnostic assays related to cancers that are characterized by the aberrant expression of a class of cell surface receptors characterized by interchain binding regions or increased cleavage of extra cellular domain in cancerous tissues. One such set of cancers are those characterized by the aberrant expression of mucin family proteins, such as MUC1, MUC2, MUC3, MUC4, up to and including MUC16. Much of the description of the invention herein is directed to cells and tissues that aberrantly express MUC1, as an example of the larger class of proteins involved in cancers which have extra cellular domains that are increasingly cleaved in cancers and/or have an inter-chain binding region (IBR). It is to be understood that in these instances the description is to be considered exemplary, and that the principles of the invention apply to other transmembrane proteins that function by a similar mechanism. With the disclosure herein, those of ordinary skill in the art will readily be able to identify other transmembrane proteins that function by this or a similar mechanism, and to apply the invention to those cancers characterized by aberrant expression of receptors. The invention is based on a novel mechanism involving transmembrane proteins that have regions of their extra cellular domain that self-aggregate and/or are increasingly cleaved, exemplified by MUC1, which was elucidated by the inventors.

MUC1 comprises several regions termed herein as follows. From the C-terminus inside the cell to the N-terminus outside the cell, the MUC1 protein is comprised of the following domains: 1) cytoplasmic tail; 2) transmembrane section; 3) MGFR; 4) IBR (interchain binding region) 5) UR (unique region); and 6) the tandem repeats.

One aspect of our previous invention featured the discovery that a specific region of the MUC1 receptor, i.e., the IBR, binds strongly to identical regions of other MUC1 molecules. That is, the MUC1 receptor has the ability to aggregate (i.e. self-aggregate) with other MUC1 receptors via the IBR of the respective receptors. A gold nanoparticle experiment was performed that showed that the IBR aggregates with itself which can occlude the binding of ligands to MUC1 or its cleavage product MUC1*. The boundary between the IBR and MGFR varies depending on where MUC1 is cleaved, which is determined by which cleavage enzyme cleaves it.

This self-aggregation may contribute to MUC1 receptor clustering, observed in healthy cells. The discovery that the IBR portion of the MUC1 receptor self-aggregates is consistent with the following mechanistic model for which the inventors present supporting evidence. (1) receptor clustering is associated with the healthy state because the aggregated IBR portions block access of ligands, such as growth factors, modifying enzymes and the like to the neighboring extracellular portions of the MUC1 receptor that act as the functional receptor; clustering also blocks access of intracellular tails to intracellular modifying enzymes and signaling ligands; (2) when the MUC1 receptor is cleaved at a position that releases some or all of the self-aggregating portions, the critical force that keeps the receptors clustered is lost and receptors are free to migrate within the cell membrane or interact with modifying enzymes, secreted ligands such as activating ligands or growth factors or other cell surface receptors. These interactions involve a new, inductive multimerization state, such as dimerization, that triggers a cell proliferation signaling cascade.

Cleavage of MUC1 releases the bulk of the extra cellular domain, including the tandem repeat domain and leaves a transmembrane protein with a truncated extra cellular domain comprising at least the PSMGFR region. Cleavage and release of the bulk of the tandem repeat domain, exposes binding sites of ligands that bind to and dimerize the truncated extra cellular domain, leading to activation of growth and survival pathways. We call the MUC1 cleavage product “MUC1*”.

MUC1* is a growth factor receptor that is activated by ligand induced dimerization of its truncated extra cellular domain. Bivalent antibodies that bind to PSMGFR peptide, which is the 45 amino acid sequence of the membrane proximal portion of MUC1 dimerize MUC1* and stimulate growth. The anti-PSMGFR antibody stimulated growth of T47D MUC1 positive cancer cells in a concentration dependent manner. In a similar experiment, a concentration of the anti-PSMGFR antibody, identified to maximize cancer cell proliferation, was added to a first group of T47D tumor cells, grown as described above. The same amount of the anti-PSMGFR antibody was added to a set of control cells, K293 cells. The addition of the anti-PSMGFR antibody to MUC1 tumor cells (T47D) enhanced proliferation by 180% 24 hours, but had no effect on the control cells.

Ligands that dimerize the extra cellular domain of MUC1* induce growth and survival of cells. Ligands of MUC1* that we identified are NME1, NME2, NME6, NME7-AB and alternative splice variant NME7-X1.

MUC1* is the growth factor receptor that drives the growth of cancer cells, whereas full-length MUC1 does not. Therefore, detection of an amount of MUC1* that is above normal levels is an indicator of cancer and the higher the amount of MUC1*, the worse the cancer. Cleavage of MUC1 may occur at more than one site, depending on which cleavage enzyme the tumor expresses. Cleavage of MUC1 releases the portion of the extracellular domain that contains the tandem repeats and could, depending on cleavage site, contain portions of the unique region or portions of the IBR. The amount of MUC1 that has been cleaved can be inferred by measuring the amount of full-length MUC1 that remains on cells or tissues. This can be accomplished by contacting the cells or tissues with an antibody that binds to the tandem repeats, or the unique region or the IBR. An antibody that binds to the tandem repeat domain is an antibody that is able to bind to a peptide having the sequence PDTRPAPGSTAPPAHGVTSA (SEQ ID NO:235). Commonly used antibodies that bind to the tandem repeat domain include but are not limited to VU4H5 (Santa Cruz Biotechnology, Dallas Texas Cat. No. SC-7313), HMPV, 5E5 (Sorensen et al., Glycobiology, Vol. 16, no. 2, pp. 96-107, 2006), PR81, and LDQ10. In these cases, it is most effective to measure an amount of full-length MUC1 compared to an amount of MUC1* expressed on the same cells or tissues. The ratio of MUC1*:MUC1 full-length is an indicator of cancer and cancer aggressiveness, wherein the more MUC1*, the more aggressive the cancer. Detection of an amount of MUC1* or the ratio of MUC1* to MUC1 full-length can also be used to determine the suitability of a cancer treatment where the therapeutic drug targets MUC1* or MUC1. Similarly, the effectiveness of such a therapy can be evaluated by detecting an amount of MUC1* or the ratio of MUC1* to MUC1 full-length before and after treatment, wherein a reduction in the amount of MUC1* expressed or a shift in the ratio of MUC1* to MUC1 full-length would be an indicator of efficacy.

There may be alternative splice isoforms of MUC1 that do not contain an IBR or tandem repeats. For example, MUC1-Y or MUC1-X. These alternative splice isoforms still have an extra cellular domain that is comprised of the sequence of the PSMGFR peptide, as this is the portion that interacts with growth factors to promote cancer and survival. Therefore, detection of an amount of MUC1* expressed by cells or tissues would still be a valid indicator of cancer and cancer aggressiveness.

The dominant MUC1 species on breast cancer tissue is the transmembrane cleavage product MUC1* not full-length MUC1. Breast tumor micro arrays were probed with either VU4H5 or MNC2. VU4H5 is a monoclonal antibody that only binds to full-length MUC1 because it recognizes an epitope (PDTRPAPGSTAPPAHGVTSA (SEQ ID NO:235) in the tandem repeat domain of full-length MUC1. This epitope is repeated hundreds of times within the tandem repeat domain of full-length MUC1. Therefore, antibody VU4H5 should give a stronger signal that an antibody that binds to a single epitope on the molecule. MNC2 is a monoclonal antibody that we produced by immunizing animals with the PSMGFR peptide (SEQ ID NO: 4). Transfection experiments show that MNC2 does not bind to full-length MUC1. MNC2 binds to a cryptic epitope that is exposed after MUC1 is cleaved to a form of MUC1* that comprises at least the first 35 membrane proximal amino acids of the MUC1* extra cellular domain, as it binds to the PSMGFR peptide (45 amino acids), the N-10 peptide (35 amino acids) but not to the C-10 peptide, indicating that its cognate epitope is encompassed at least in part within the 10 membrane proximal amino acids of the MUC1* extra cellular domain. Importantly, MNC2 competitively inhibits the binding of MUC1* activating growth factors NME1 and NME7-AB.

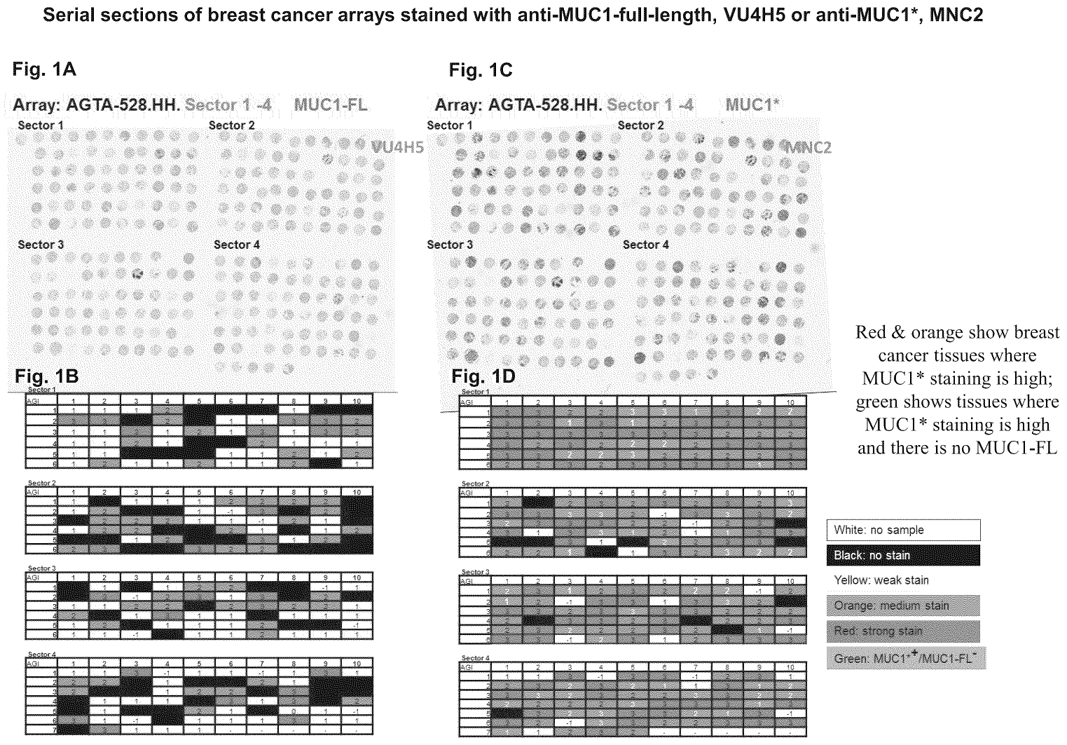

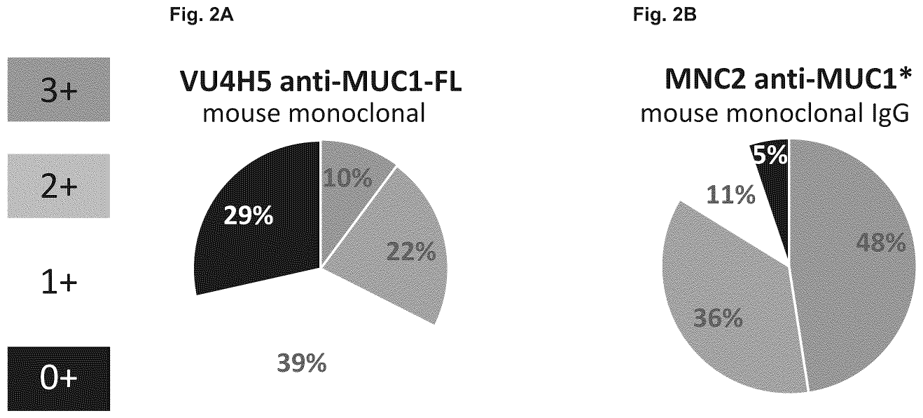

FIG. 1A-1D shows photographs of adjacent serial sections of breast cancer tissue arrays and graphical representations of the pathologist scores, according to Allred scoring system. Pathologist score is 0-3, where 0 showed no staining and 3 is the greatest staining. The graphs are also color coded, where a pathologist score zero is black, 1 is yellow, 2 is orange, and 3 is red; tissues that scored zero when probed with an antibody that recognizes full-length MUC1 but positive when probed with an antibody that recognizes MUC1* were colored green; and missing or uninterpretable tissues were scored −1. FIG. 1A shows photographs of the breast cancer tissue arrays after they were stained with VU4H5, which is an antibody that binds to the tandem repeat domains of full-length MUC1. FIG. 1B shows graphs of the pathologist scores for the tissues pictured in FIG. 1A. FIG. 1C shows photographs of the breast cancer tissue arrays after they were stained with MNC2, which is an antibody that binds to an epitope within the PSMGFR region of MUC1*. FIG. 1D shows graphs of the pathologist scores for the tissues pictured in FIG. 1C. FIG. 2A-2B shows pie chart graphs of the pathologist scores of the arrays shown in FIG. 1A and FIG. 1C. FIG. 2A shows that the antibody that binds to tandem repeats of full-length MUC1 misses 30% of breast cancers. FIG. 2B shows that the anti-MUC1* antibody MNC2 recognizes 95% of breast cancers. Anti-MUC1-full-length only binds strongly to 10% of the breast tumors, while anti-MUC1* antibody MNC2 binds strongly to about 50% of breast tumors. Together these data demonstrate that MUC1*, not full-length MUC1, is the predominant MUC1 species on cancerous tissues. Anti-MUC1* antibodies would detect or diagnose nearly all breast cancers, whereas antibodies that bind to full-length MUC1 would fail to detect about 30% of breast cancers. Further, because MUC1* is a growth factor receptor driving cancer growth, the degree of anti-MUC1* staining of a tissue or cellular specimen would be proportional to the degree or stage of cancer, whereas the expression of full-length MUC1 appears to be inversely proportional to the stage of cancer.

A wide range of cancer cells and tumor specimens were probed with anti-MUC1* antibody MNC2. MNC2 was used to detect MUC1* positive cancers in a wide range of assays, including fluorescence activated cell sorting (FACS), immunofluorescence (IF), immunohistochemistry (IHC). FACS and IF are generally used to study a cell line which is a single immortalized cell that has been propagated in a lab for decades. After decades of propagation in unnatural growth solutions, these cell lines likely show little resemblance to even a single cell within the patient's original tumor and in no way represent the tumor of a recently diagnosed patient seeking treatment. For these reasons, we analyzed thousands of tumor micro arrays, wherein each dot within the array is tumor specimen from a single patient's biopsy. In most cases, the biopsies are from recently diagnosed patients, but the accompanying anonymized patient data gives the age of the patient, cancer sub-type and cancer stage or grade. In some cases we analyzed tissue micro arrays wherein the breast cancers were all HER2+, or all ER+/PR+. In other cases. We analyzed tumor micro arrays that compared the original biopsy specimen to a later metastasis. In these studies, the recognition of tumors by MNC2 was also compared to staining using anti-full-length-MUC1 antibody VU4H5 or a new antibody 5E5 that binds to a trapped O-linked glycan in the tandem repeat domain of full-length MUC1. MNC2 and other anti-MUC1* antibodies consistently recognized tumor tissue better than VU4H5 or 5E5. Normal tissues and normal tissue micro arrays were also extensively studied to determine binding of MNC2 or its humanized singly chain form huMNC2-scFv or huMNC2-scFv-Fc to normal tissues. On normal tissues, expression of MNC2 reactive MUC1* was restricted to the apical border of ducts and glands in a small percentage of only a few tissues. In all cases, MNC2 reactive MUC1* was expressed to a much higher degree in cancerous tissues than in normal tissues and expressed over 50-100% of the cancerous tissues compared to expression of 0.2%-5% of the normal tissue that did express MNC2 reactive MUC1*.

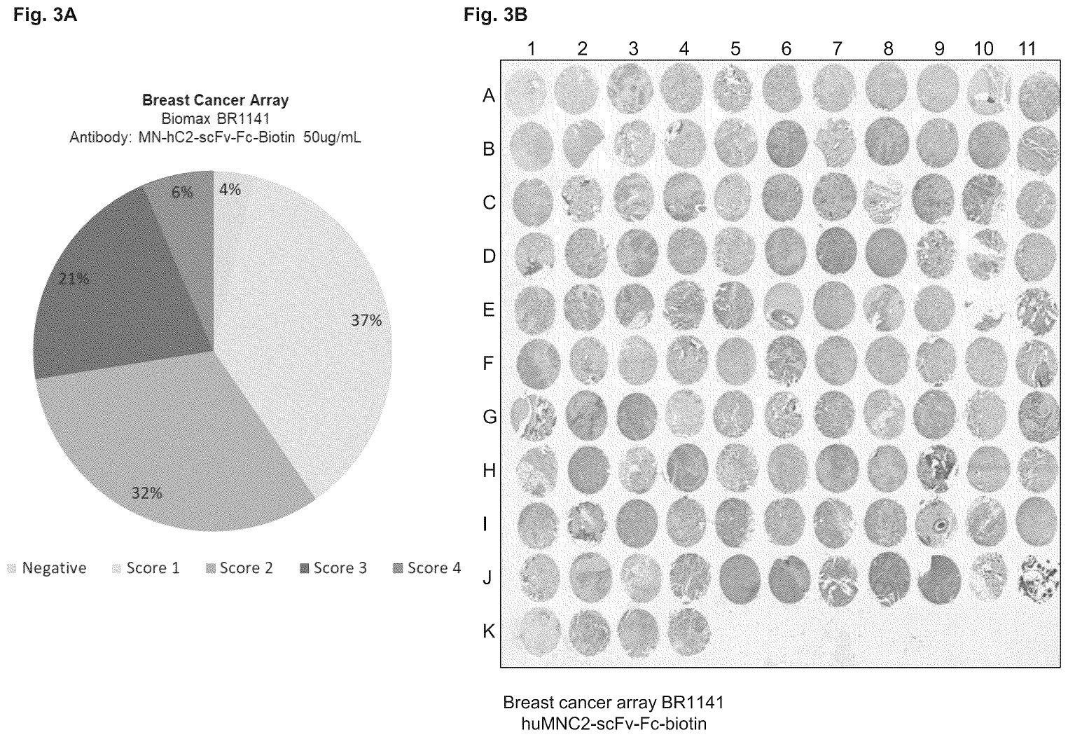

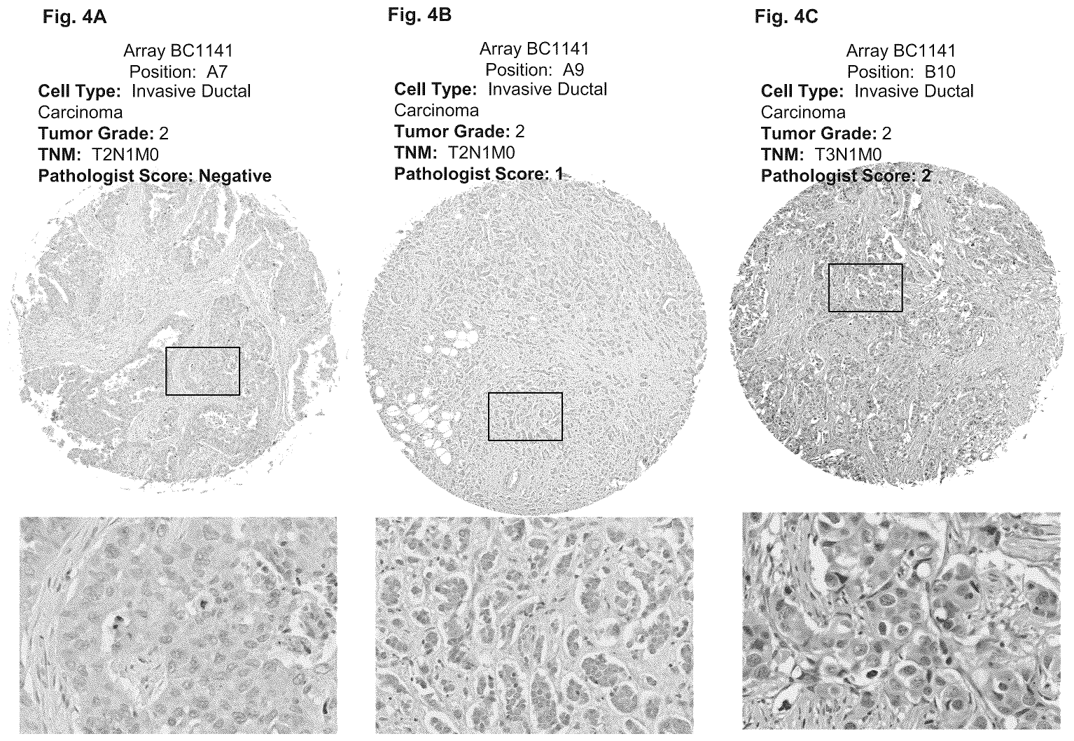

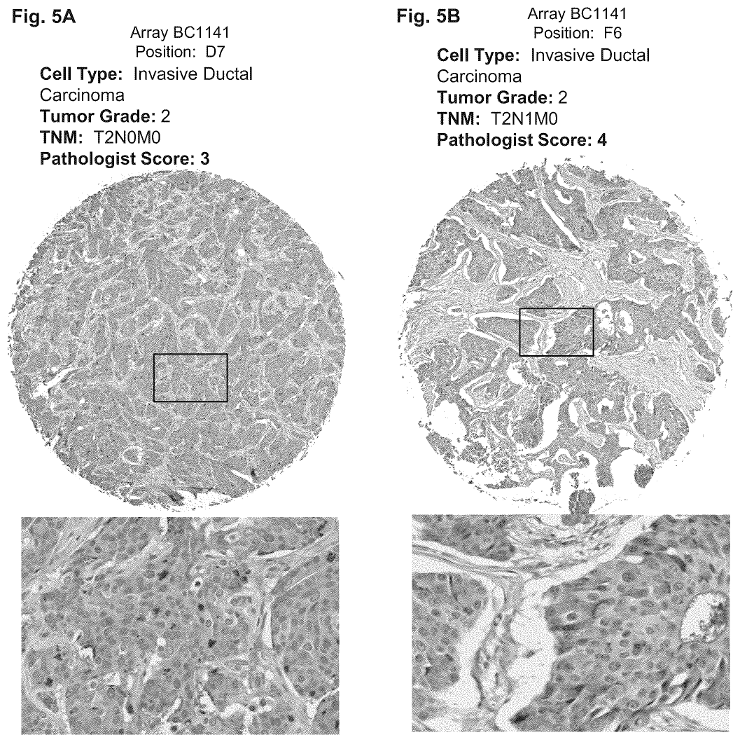

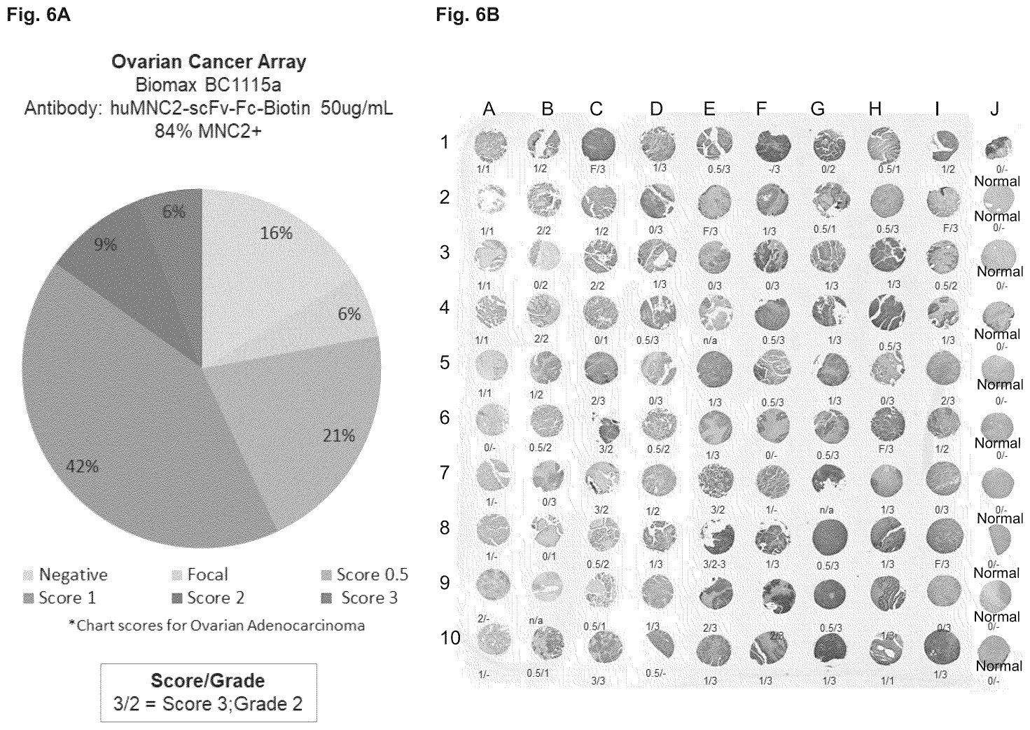

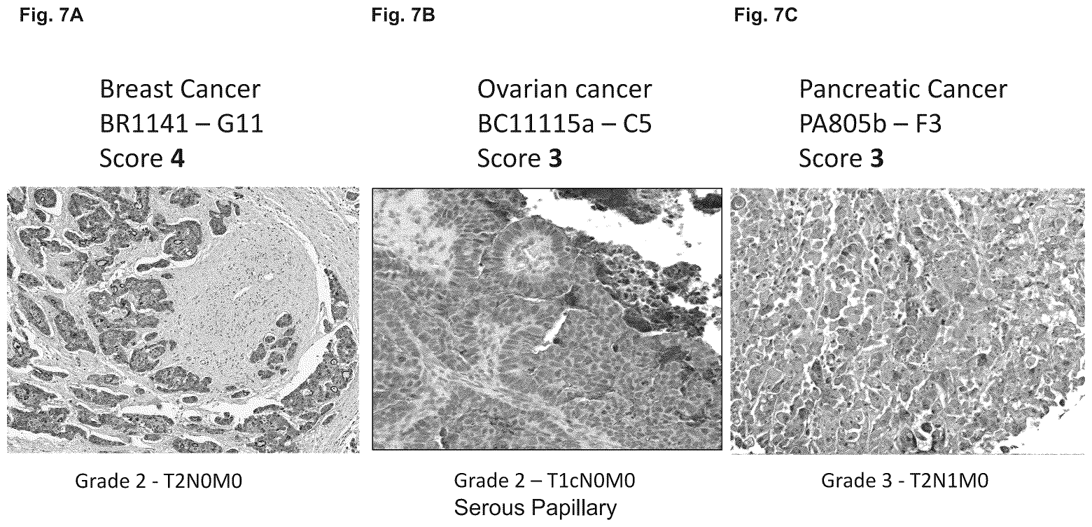

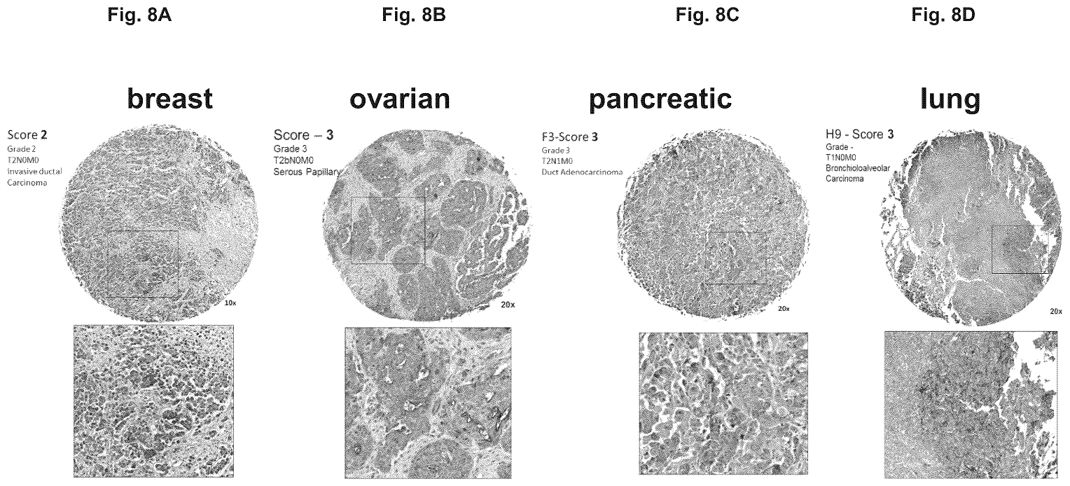

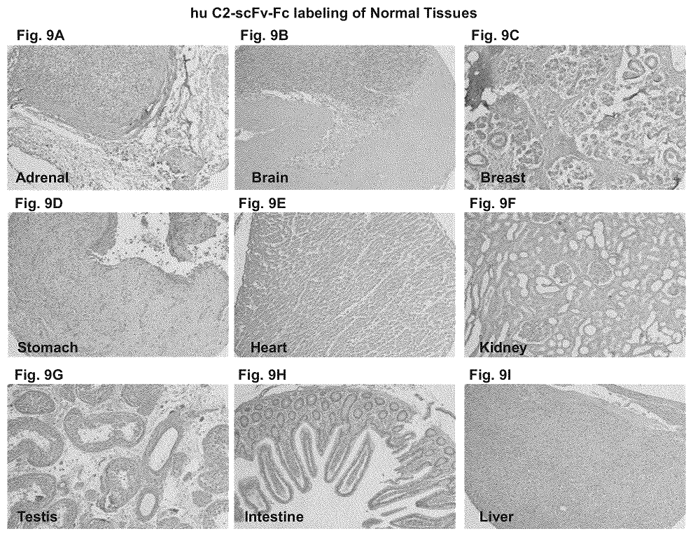

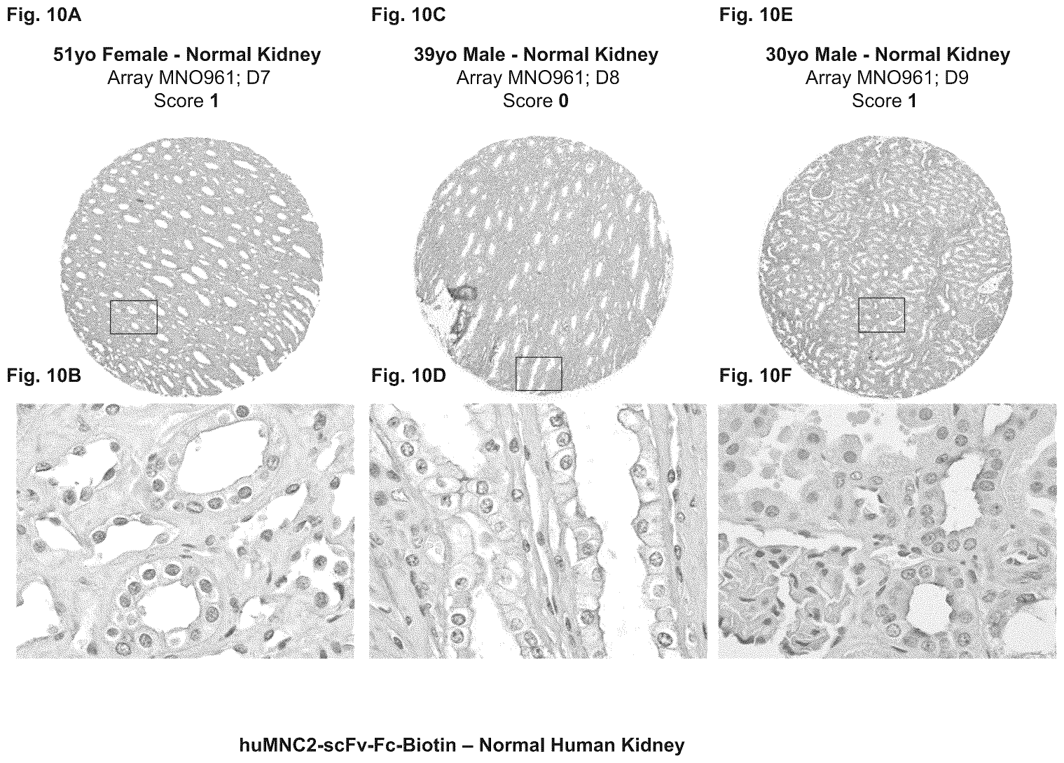

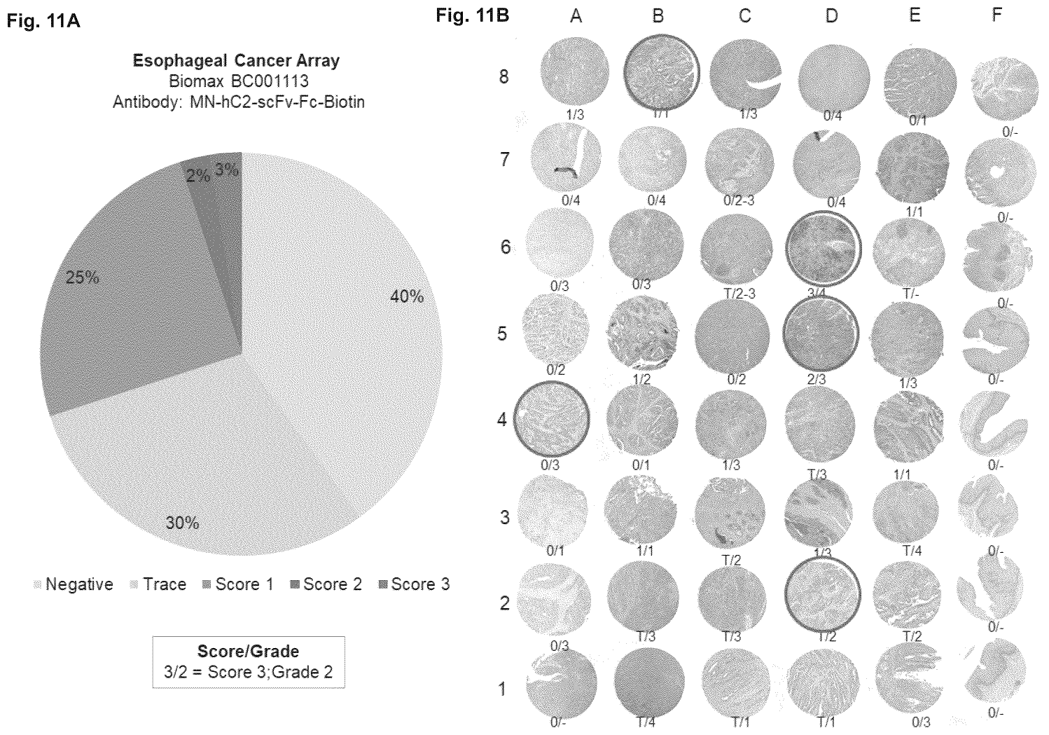

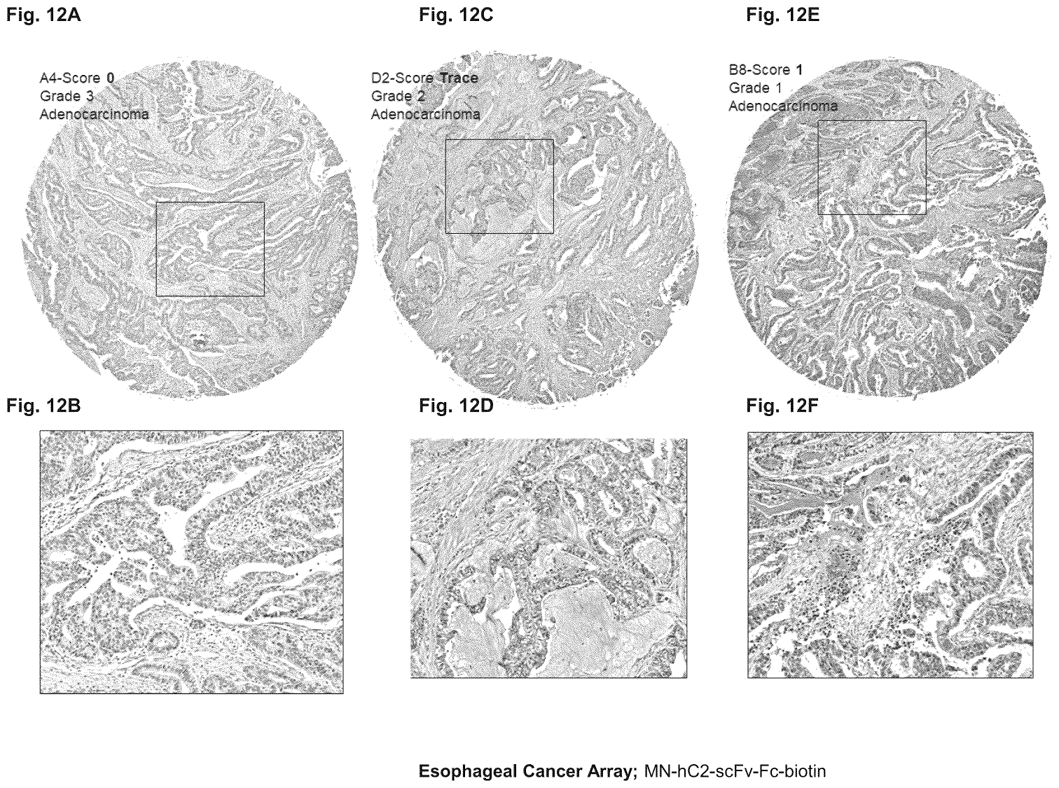

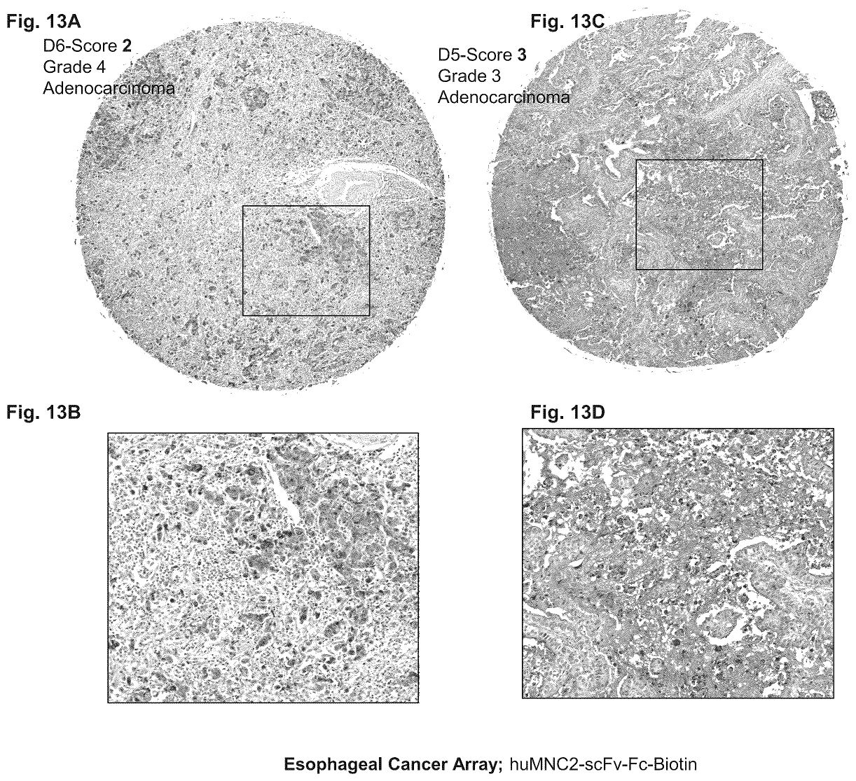

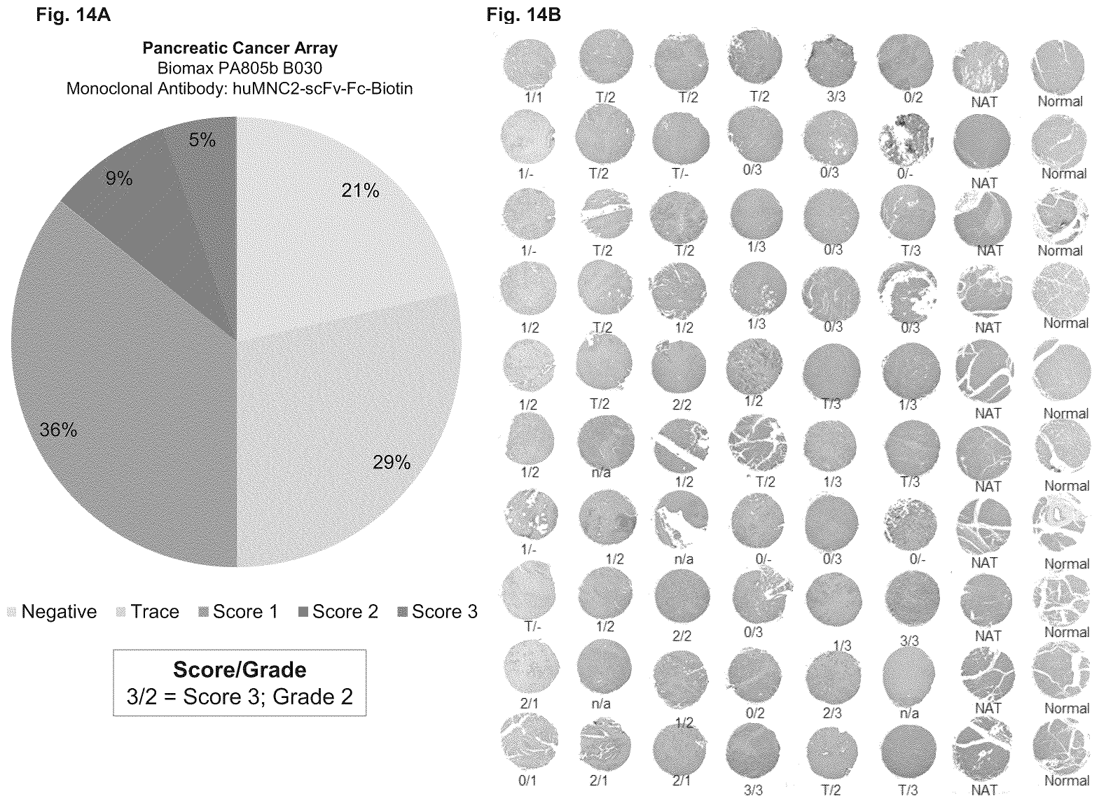

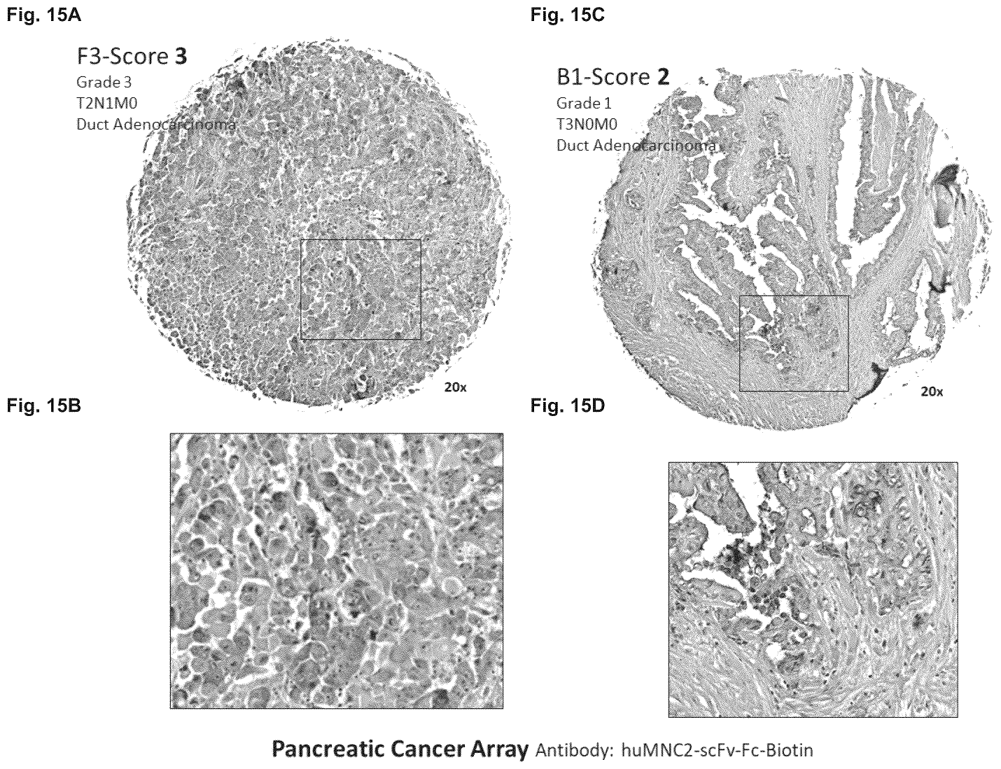

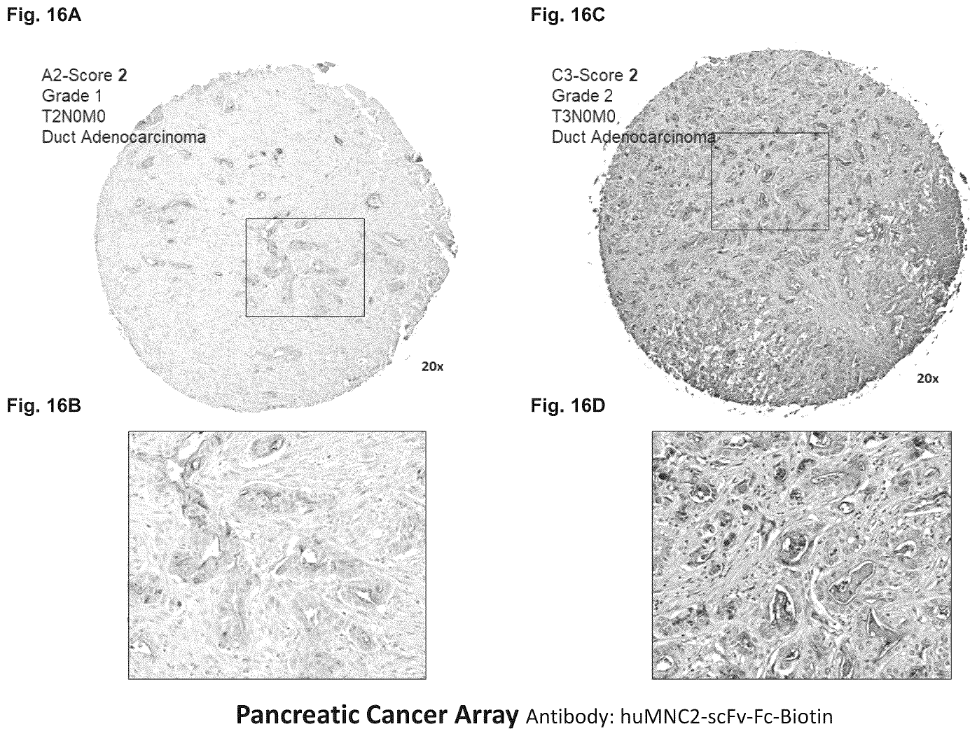

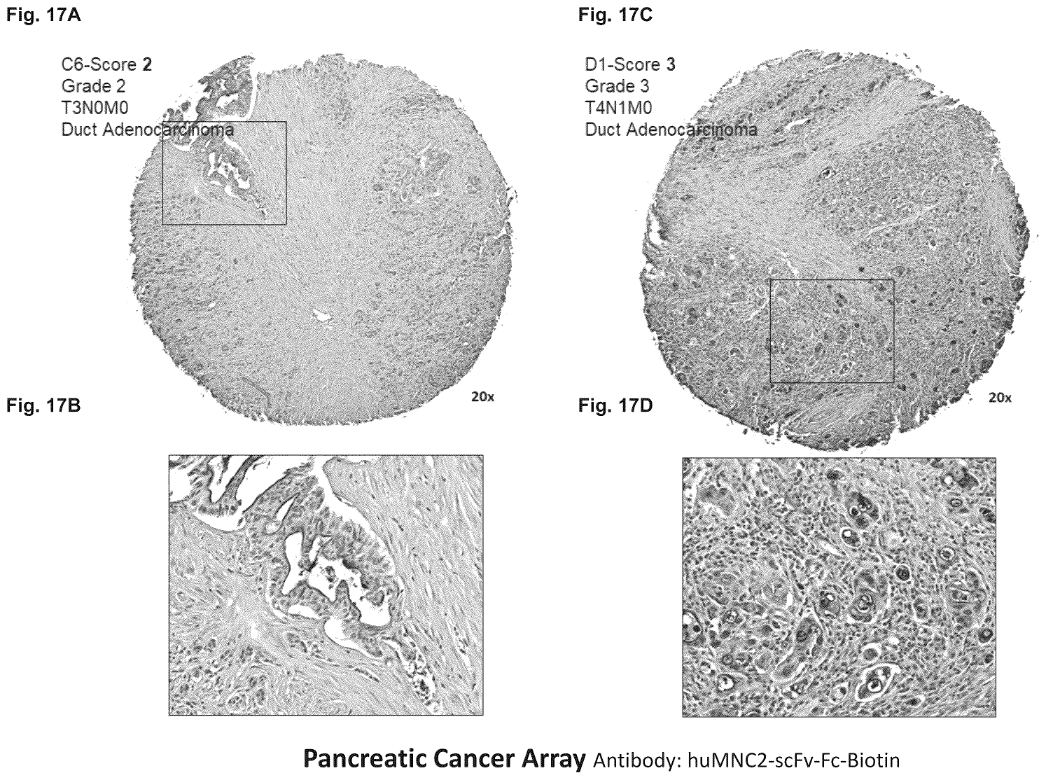

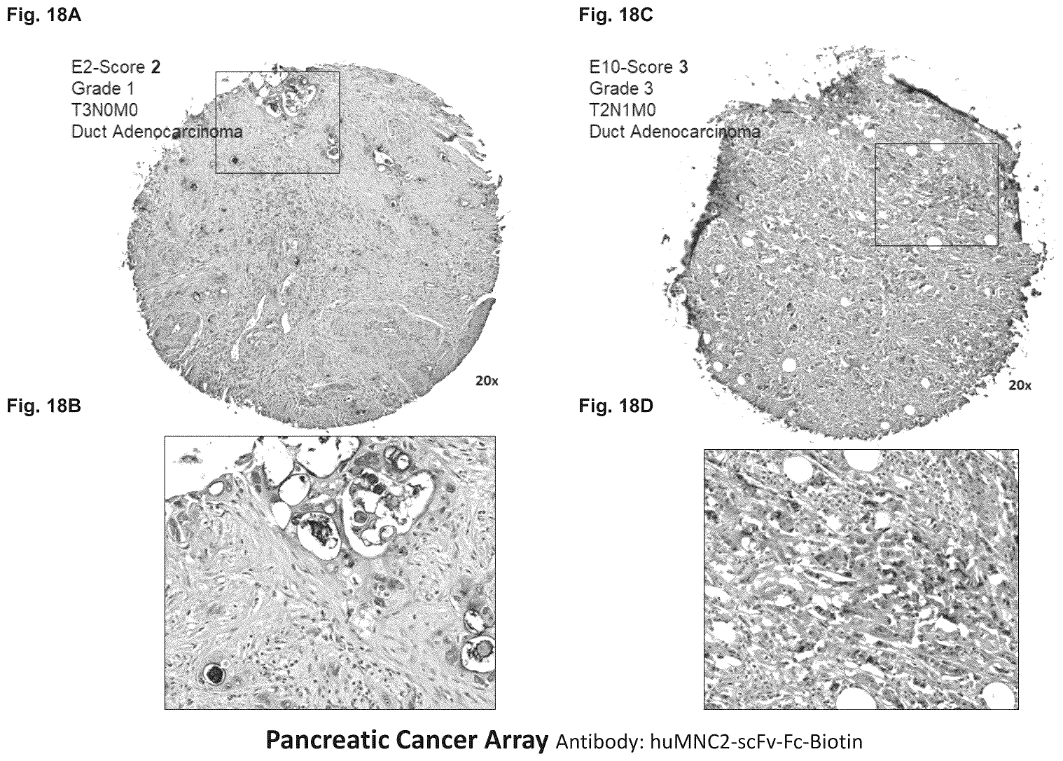

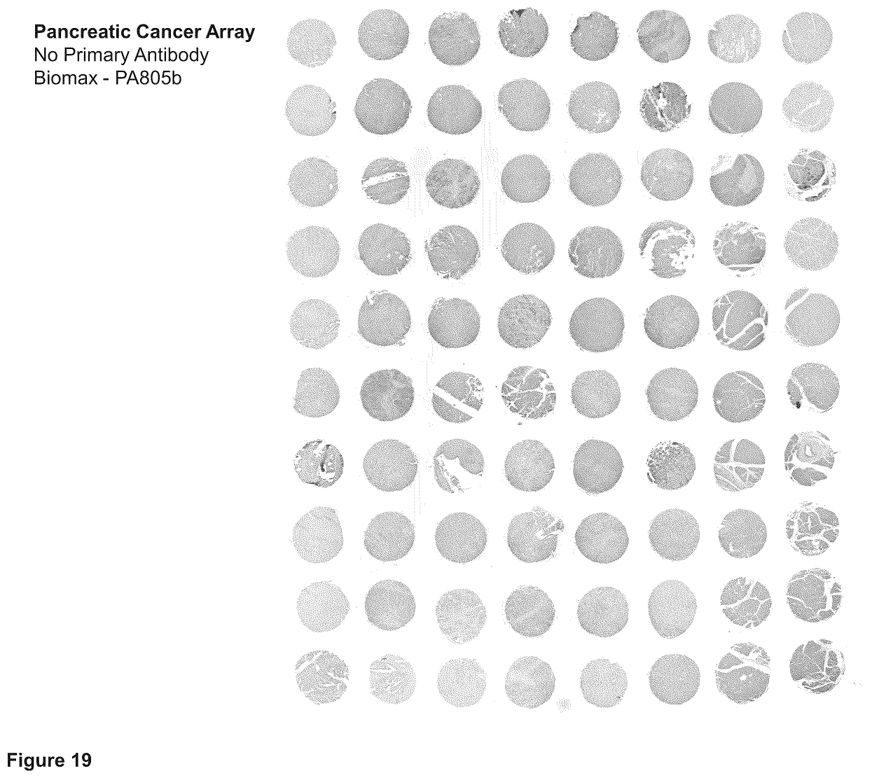

FIG. 3 to FIG. 19 show that monoclonal anti-MUC1* antibody MNC2 binds to high percentages of breast, ovarian, pancreatic, lung and esophageal tumors, while having very little if any binding to normal tissues. FIG. 3A-3B shows pie chart graphs of the pathologist scores and a photograph of breast cancer array BR1141 after staining with anti-MUC1* antibody huMNC2-scFv-Fc. FIG. 4A-4C shows photographs, at two different magnifications, of individual breast cancer specimens from breast cancer array BR1141 after staining with anti-MUC1* antibody huMNC2-scFv-Fc. The position in the array, cancer sub-type, tumor grade, TNM (Tumor stage, Node involvement, and Metastasis) and pathologist score are indicated in figures. Standard immunohistochemistry methods were used. Antibody concentration was titered using the highest concentration at which the antibody showed expected staining of normal tissues without staining stroma. The antibody was conjugated to a biotin through its Fc region, to avoid false positive due to anti-human secondary antibodies staining host antibodies as well as B cell follicles. FIG. 4A shows the specimen at position A7 which was negative for huMNC2 reactive cells. FIG. 4B shows the specimen at position A9 which is a Grade 2 cancer, with lymph node involvement that scored +1 for huMNC2 reactivity. FIG. 4C shows the specimen at position B10 which is a larger Grade 2 tumor, with lymph node involvement that scored +2 for huMNC2 reactivity. FIG. 5A-5B shows photographs, at two different magnifications, of individual breast cancer specimens from breast cancer array BR1141 after staining with anti-MUC1* antibody huMNC2-scFv-Fc. FIG. 5A shows the specimen at position D7 which is a Grade 2 cancer, without lymph node involvement that scored +3 for huMNC2 reactivity. FIG. 5B shows the specimen at position F6 which is a Grade 2 tumor, with lymph node involvement that scored +4 for huMNC2 reactivity. FIG. 6A-6B shows pie chart graphs of the pathologist scores and a photograph of ovarian cancer array BC1115a after staining with anti-MUC1* antibody huMNC2-scFv-Fc. FIG. 7A-7C shows magnified photographs of different cancer sub-types after staining with anti-MUC1* antibody huMNC2-scFv-Fc. FIG. 7A shows a photograph of a Grade 2 breast tumor that pathologist scored +4. FIG. 7B shows a photograph of a Grade 2 ovarian tumor that pathologist scored +3. FIG. 7C shows a photograph of a Grade 3 pancreatic tumor that pathologist scored +3. IHC studies of over 1,000 tumor specimens showed that huMNC2-scFv recognized 95% of Breast Cancers (90% triple negative), 83% Ovarian, 78% Pancreatic and 71% Lung Cancers. FIG. 8A-8D shows magnified photographs of different cancer sub-types after staining with anti-MUC1* antibody huMNC2-scFv-Fc. FIG. 8A shows a photograph of a Grade 2 breast tumor that pathologist scored +2. FIG. 8B shows a photograph of a Grade 3 ovarian tumor that pathologist scored +3. FIG. 8C shows a photograph of a Grade 3 pancreatic tumor, with lymph node involvement that pathologist scored +3. FIG. 8D shows a photograph of a lung cancer that pathologist scored +3. FIG. 9A-9I shows magnified photographs of various normal tissues after staining with anti-MUC1* antibody huMNC2-scFv-Fc. Conditions and concentrations used were identical to those used for studying cancerous tissues. FIG. 9A shows normal adrenal gland tissue. FIG. 9B shows normal brain tissue. FIG. 9C shows normal breast tissue. FIG. 9D shows normal stomach tissue. FIG. 9E shows normal heart tissue. FIG. 9F shows normal kidney tissue. FIG. 9G shows normal testis tissue. FIG. 9H shows normal intestine tissue. FIG. 9I shows normal liver tissue. FIG. 10A-10F shows photographs of normal kidney tissues after staining with anti-MUC1* antibody huMNC2-scFv-Fc. Conditions and concentrations used were identical to those used for studying cancerous tissues. FIG. 10A shows normal kidney tissue with huMNC2 reactivity limited to the apical border, which is normal expression. FIG. 10B is the same tissue at greater magnification. FIG. 10C shows another example of normal kidney tissue with undetectable huMNC2 reactivity. FIG. 10D is the same tissue at greater magnification. FIG. 10E shows another example of normal kidney tissue with huMNC2 reactivity limited to the apical border, which is normal expression. FIG. 10F is the same tissue at greater magnification. Further studies showed that less than 10% of normal kidney tissue showed huMNC2 reactivity at distal collecting tubules wherein such reactivity was strictly limited to the apical border, which is a normal expression pattern. FIG. 11A-11B shows pie chart graphs of the pathologist scores and a photograph of esophageal cancer array BC001113 after staining with anti-MUC1* antibody huMNC2-scFv-Fc. FIG. 12A-12F shows photographs, at two different magnifications, of individual esophageal cancer specimens from esophageal cancer array BC001113, after staining with anti-MUC1* antibody huMNC2-scFv-Fc. The position in the array, cancer sub-type, tumor grade, and pathologist score are indicated in figures. FIG. 12A shows the specimen at position A4 which was negative for huMNC2 reactive cells. FIG. 12B shows the same specimen at greater magnification. FIG. 12C shows the specimen at position D2 which the pathologist scored as trace reactivity to huMNC2. FIG. 12D shows the same specimen at greater magnification. FIG. 12E shows the specimen at position B8 which the pathologist scored as +1 reactivity to huMNC2. FIG. 12F shows the same specimen at greater magnification. FIG. 13A-13D shows photographs, at two different magnifications, of individual esophageal cancer specimens from esophageal cancer array BC001113, after staining with anti-MUC1* antibody huMNC2-scFv-Fc. The position in the array, cancer sub-type, tumor grade, and pathologist score are indicated in figures. FIG. 13A shows the specimen at position D6, a Grade 4 tumor, which the pathologist scored +2. FIG. 13B shows the same specimen at greater magnification. FIG. 13C shows the specimen at position D5, a Grade 3 tumor, which the pathologist scored +3. FIG. 12D shows the same specimen at greater magnification. FIG. 14A-14B shows pie chart graphs of the pathologist scores and a photograph of pancreatic cancer array PA805b after staining with anti-MUC1* antibody huMNC2-scFv-Fc. FIG. 15A-15D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 15A shows the specimen at position F3, a Grade 3 tumor, which the pathologist scored +3. FIG. 15B shows the same specimen at greater magnification. FIG. 15C shows the specimen at position B1, a Grade 1 tumor, which the pathologist scored +2. FIG. 15D shows the same specimen at greater magnification. FIG. 16A-16D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 16A shows the specimen at position A2, a Grade 1 tumor, which the pathologist scored +2. FIG. 16B shows the same specimen at greater magnification. FIG. 16C shows the specimen at position C3, a Grade 2 tumor, which the pathologist scored +2. FIG. 16D shows the same specimen at greater magnification. FIG. 17A-17D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 17A shows the specimen at position C6, a Grade 2 tumor, which the pathologist scored +2. FIG. 17B shows the same specimen at greater magnification. FIG. 17C shows the specimen at position D1, a larger Grade 3 tumor, with lymph node involvement that the pathologist scored +3. FIG. 17D shows the same specimen at greater magnification. FIG. 18A-18D shows photographs, at two different magnifications, of individual pancreatic cancer specimens from pancreatic cancer array PA805b, after staining with anti-MUC1* antibody huMNC2-scFv-Fc. The position in the array, cancer sub-type, tumor grade, and pathologist score are indicated in figures. FIG. 18A shows the specimen at position E2, a Grade 1 tumor, which the pathologist scored +2. FIG. 18B shows the same specimen at greater magnification. FIG. 18C shows the specimen at position E10, a smaller Grade 3 tumor, with lymph node involvement that the pathologist scored +3. FIG. 18D shows the same specimen at greater magnification. FIG. 19 shows a photograph of pancreatic cancer array PA805b that was stained with the secondary antibody alone, as a control.

Although MNC2 recognized about 95% of breast tumors across all breast cancer sub-types, we noticed that some cancer sub-types did not express as much MNC2 reactive MUC1* as breast cancers. In particular, pancreatic, esophageal and prostate cancers expressed lower levels of MNC2 reactive MUC1*. Pancreatic cancer arrays showed that 78% of the tumors were MNC2 reactive but the strength of staining, which is proportional to the tumor's expression levels, was relative weak. The pie chart of FIG. 14A shows that 65% of the pancreatic tumors scored +1 or +2, only 5% scored +3 and none scored +4. The pie chart of FIG. 3A shows that more than half of the breast tumors scored +2 to +3, 6% were +4 and only 4% were negative for MNC2 MUC1* reactivity. Both arrays were stained with the same MNC2 anti-MUC1* antibody and scored by the same board-certified pathologist. We reasoned that the difference between MNC2 staining of MUC1* in breast cancer and pancreatic cancer could be due to differences in cleavage enzymes that cleave MUC1 to MUC1* at different positions that induce conformational or linear changes in the MUC1* extra cellular domain. To investigate, we stained the same pancreatic cancer array with the anti-MUC1* polyclonal antibody SDIX. Although both MNC2 and SDIX were generated by immunizing animals with the PSMGFR peptide, they showed different binding characteristics to tumor tissue. In general, SDIX recognized more pancreatic tissues and stained more robustly than MNC2, although there were cases where MNC2 recognized a tumor that SDIX did not.

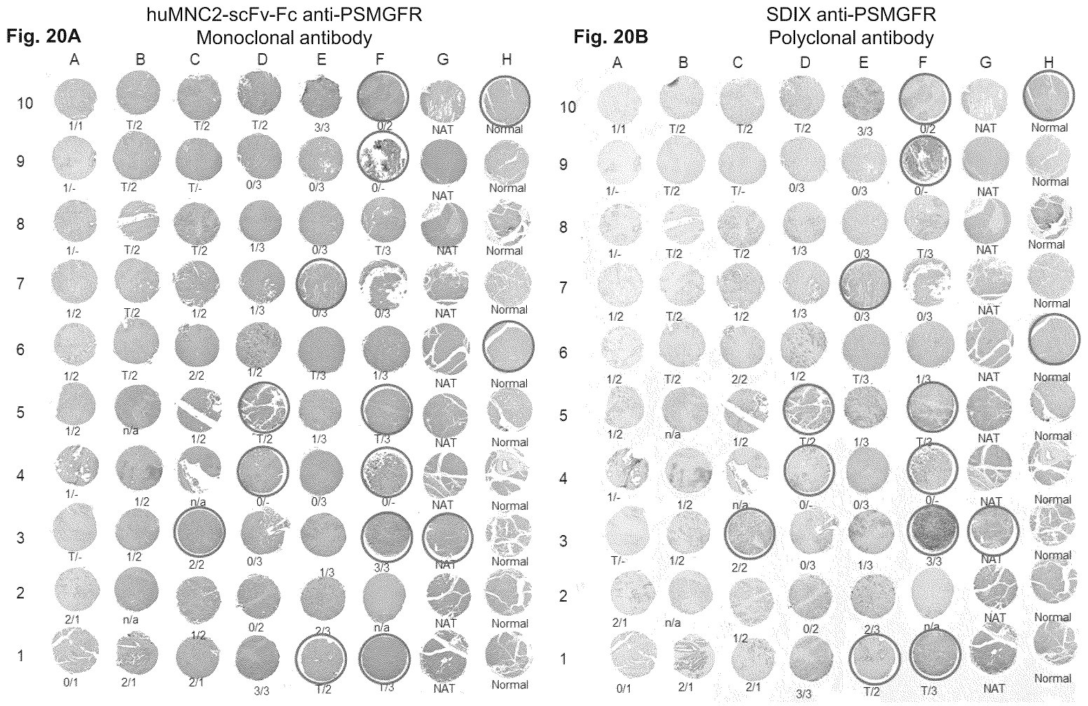

On cancerous tissues, MUC1* is expressed over most of the tissue and is characteristic of cancer, all anatomical barriers have broken down in cancerous tissues. In contrast, on normal tissues, expression of MUC1* is restricted to the apical border of ducts and glands. Expression of MNC2 reactive MUC1* is even further restricted. For example, FIG. 6B shows a photograph of an ovarian cancer micro array. However, Column J is made up of normal ovarian tissues. As can be seen, there is no expression of MNC2 reactive MUC1*. Normal kidney does express some MNC2 reactive MUC1*. As can be seen in FIG. 10A-10F, normal MUC1* expression is weak and restricted to the apical border of about 10% of the distal collecting tubules of normal kidney. Normal pancreas expresses MUC1* that is again tightly restricted to the apical border of acinar cells (FIG. 20). Those skilled in the art can readily identify cancerous tissues and can differentiate between MUC1* expression on normal tissue and on cancerous tissues. In general, MUC1* is grossly overexpressed on cancerous tissues and its expression is not restricted to an apical pattern of expression.

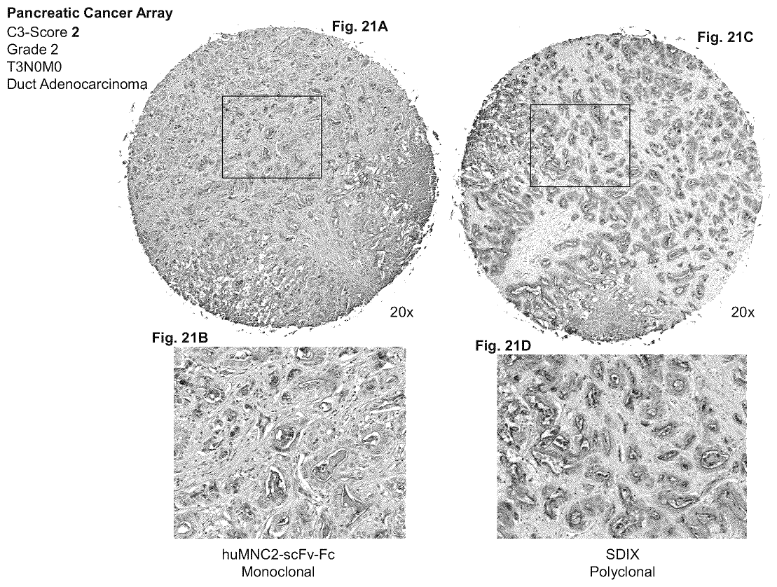

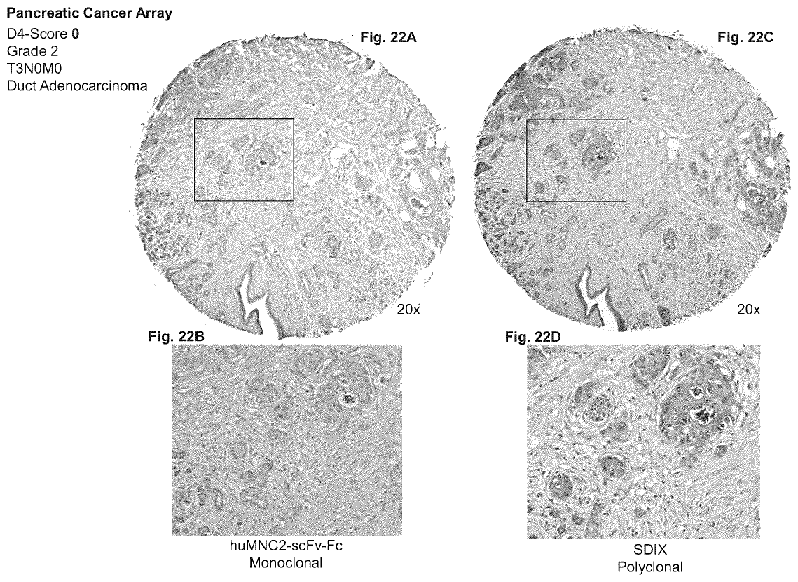

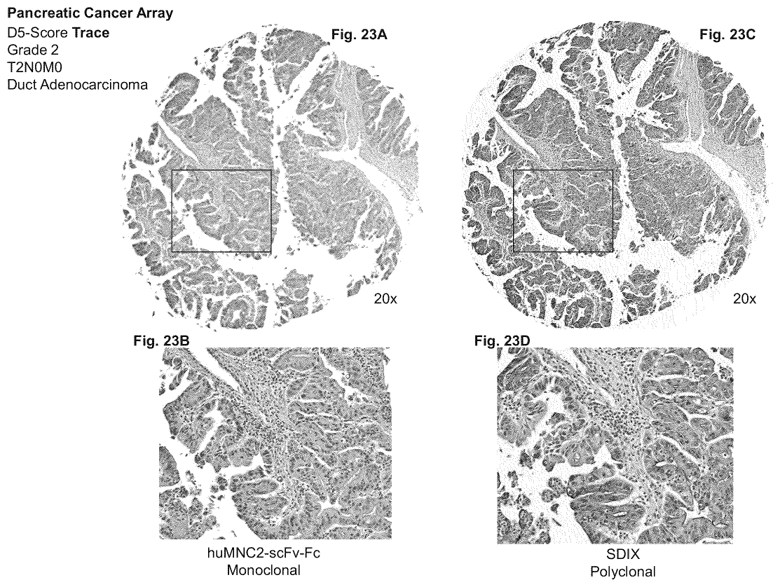

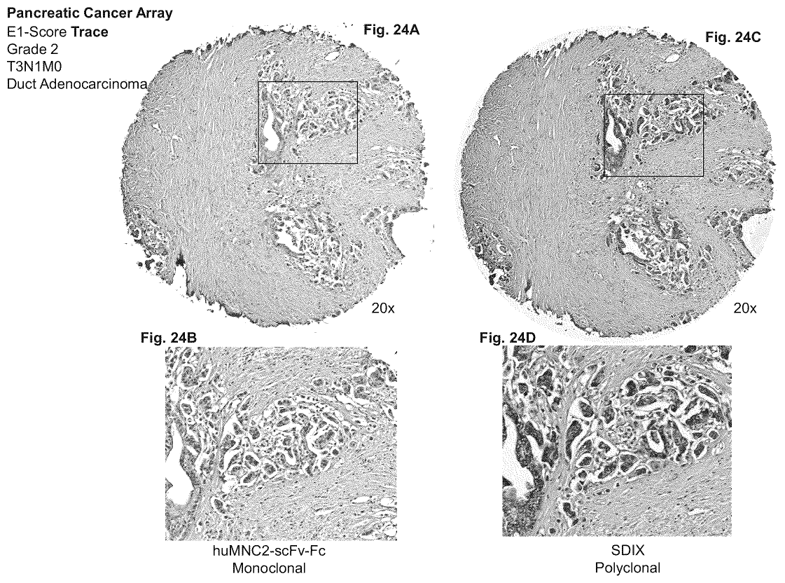

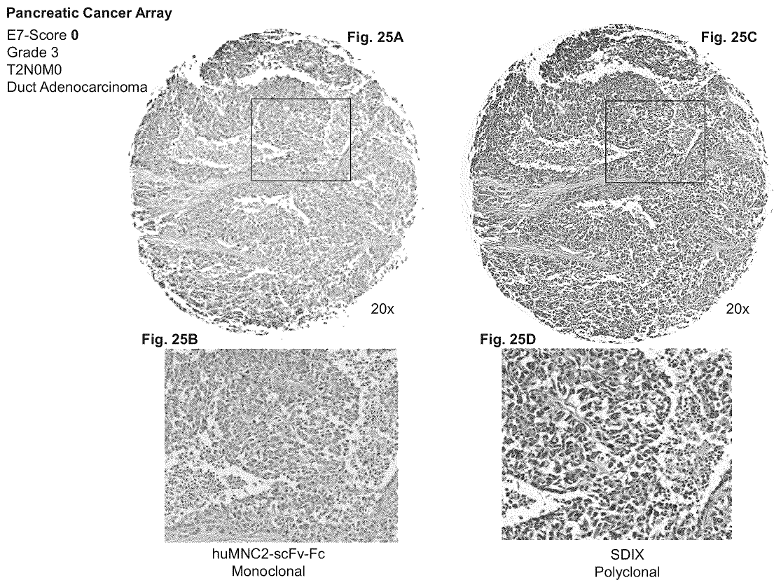

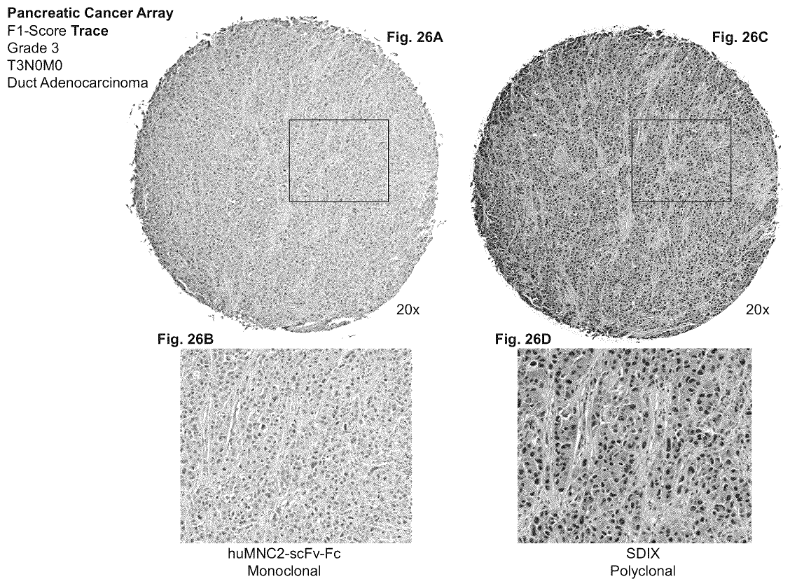

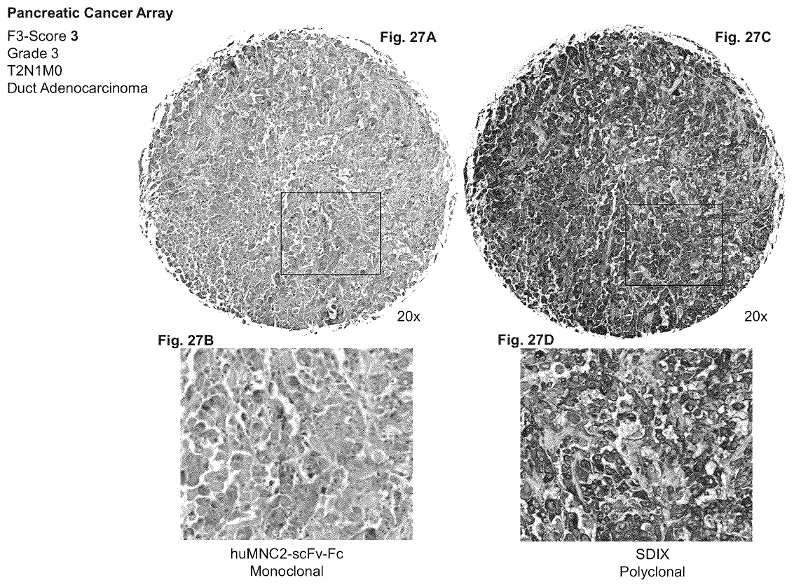

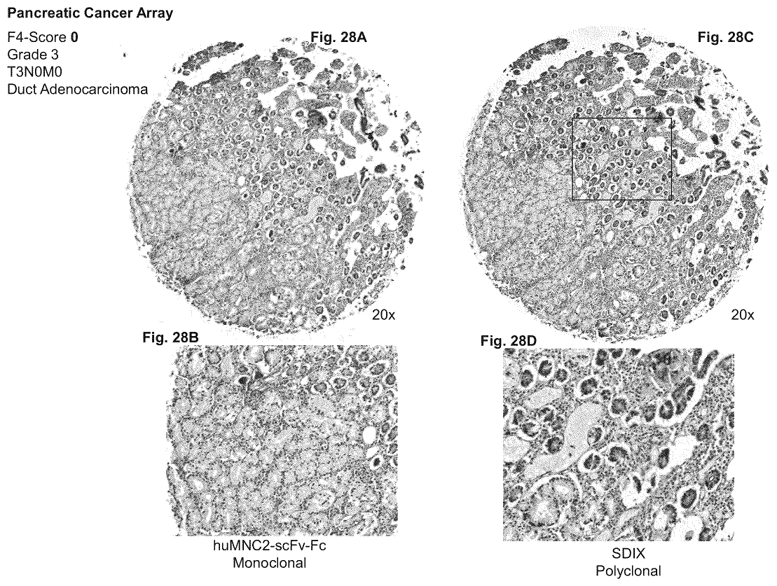

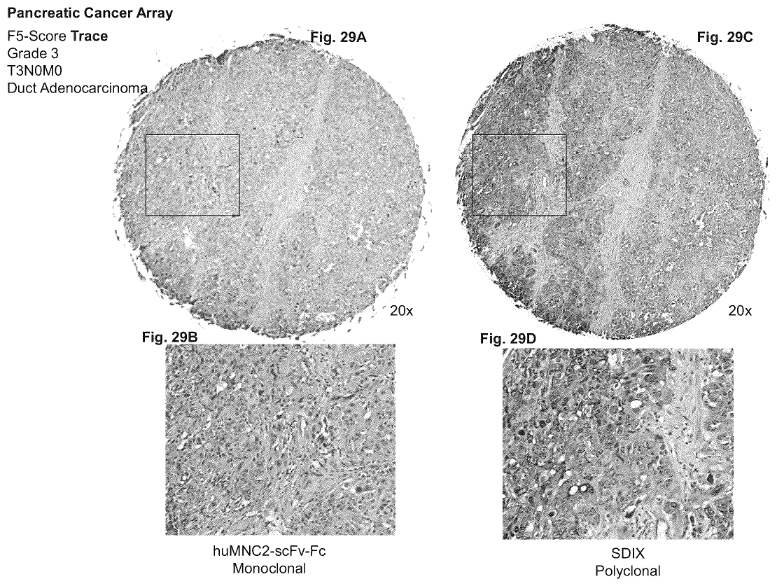

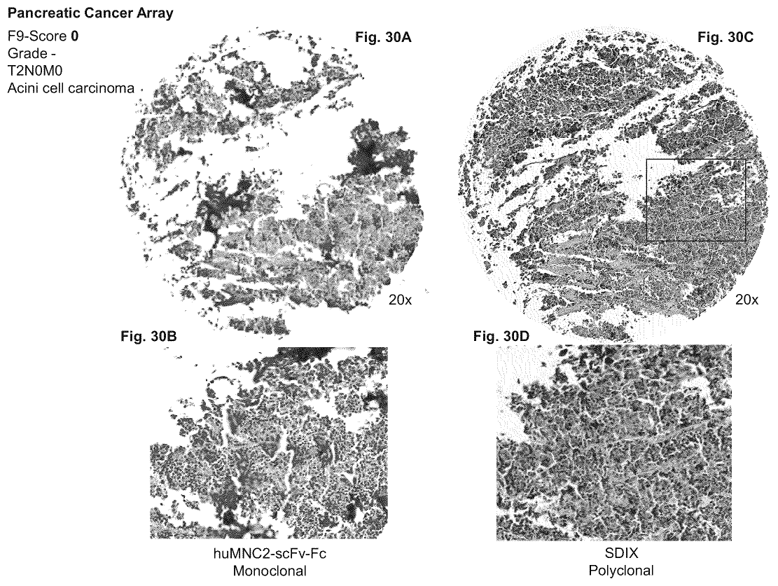

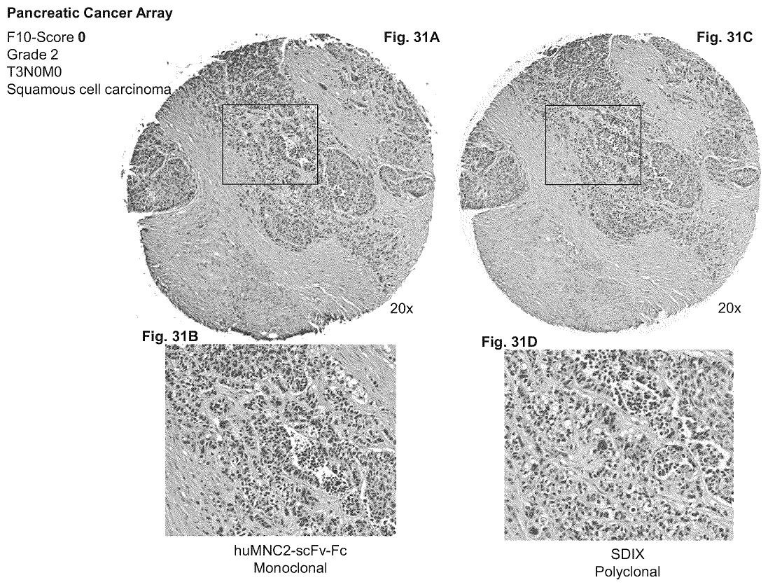

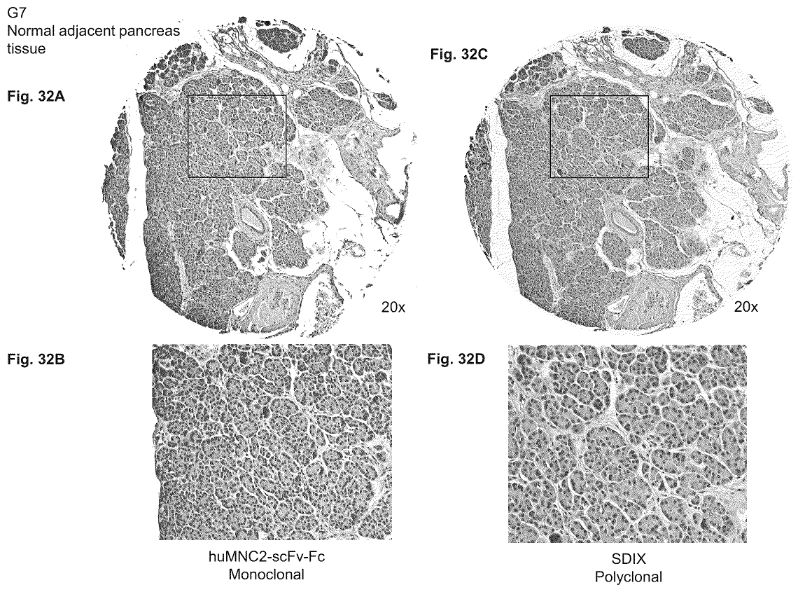

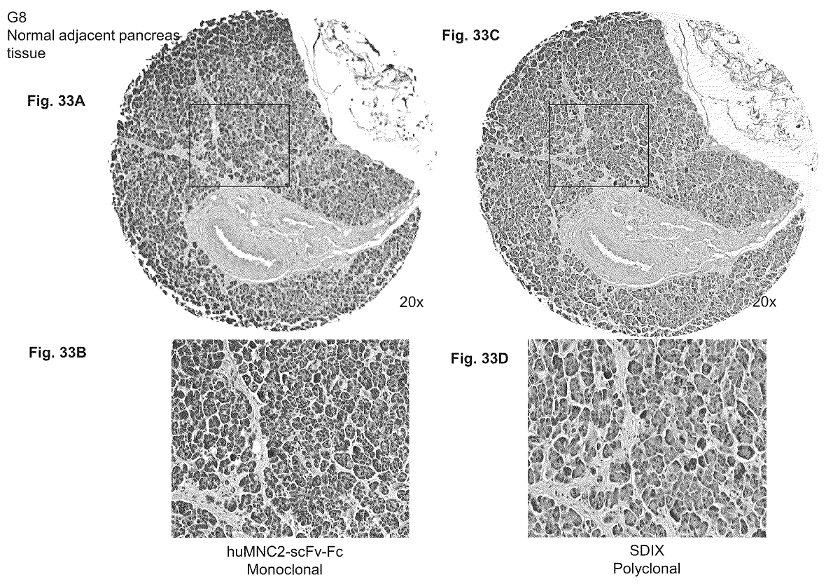

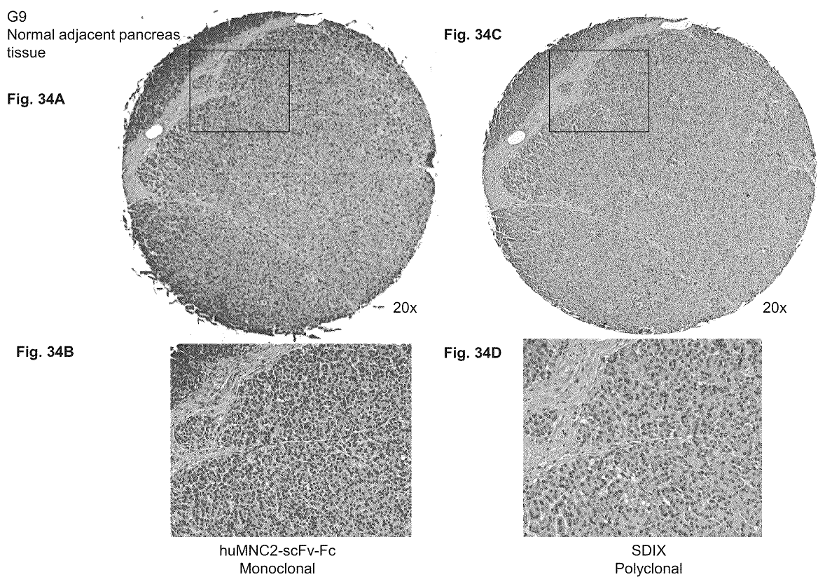

In this FIG. 20 through FIG. 34, we show that a series of pancreatic tumors showed no or minimal staining with monoclonal antibody MNC2, but staining the same tissue with the SDIX polyclonal antibody produced robust staining. Both MNC2 and SDIX were generated by immunizing animal with the same peptide: PSMGFR. However, MNC2 only recognizes a subset of those recognized by SDIX. These results strongly argue that MNC2 recognizes an epitope that is only created in a subset of the tumor. The data suggest that the MNC2 reactive subset of MUC1* can be cancer sub-type specific or patient specific, likely due to cleavage by different cleavage enzymes.

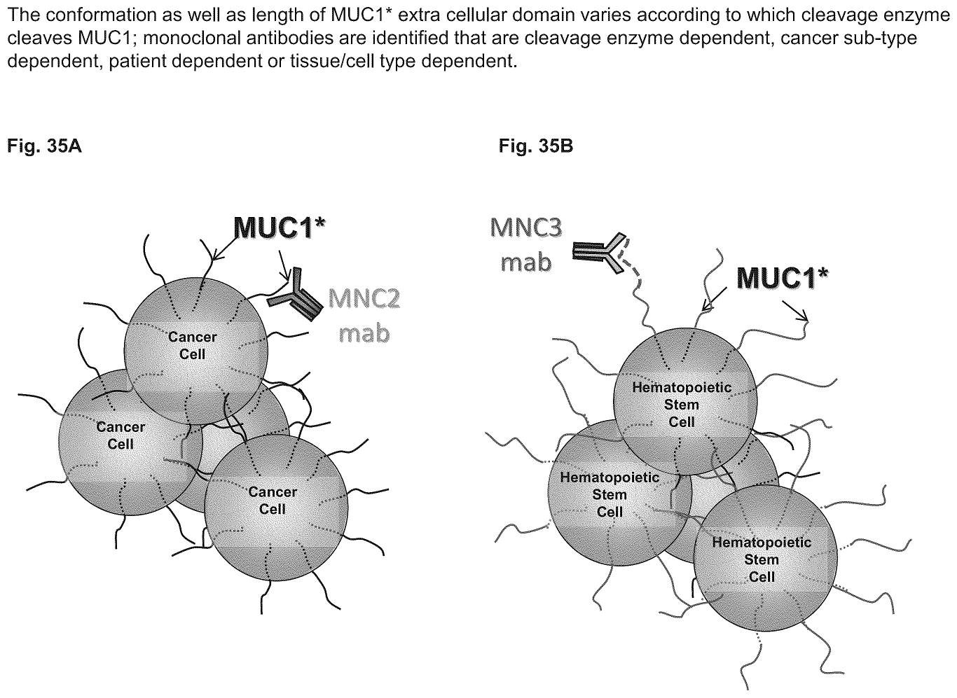

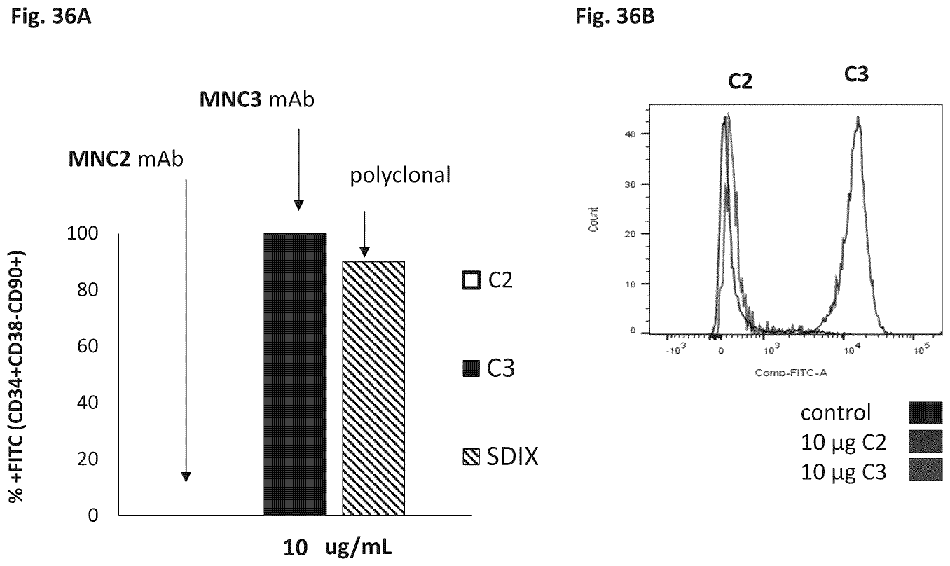

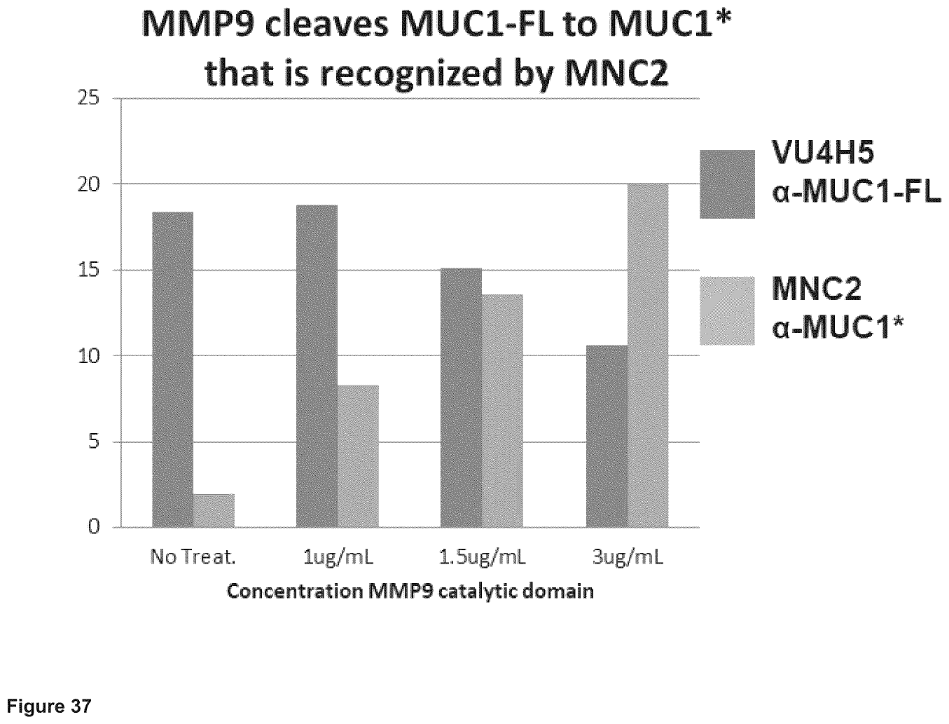

The hypothesis that anti-MUC1* antibody specificity is dependent on the cleavage enzyme that cleaves MUC1 to a MUC1* is supported by data shown in FIG. 35 through FIG. 37. MNC2, MNC3 and SDIX were all generated by immunizing an animal with the PSMGFR peptide. However, monoclonal antibody MNC3 recognizes nearly 100% of hematopoietic stem cells, as does the polyclonal antibody SDIX, while monoclonal antibody MNC2 does not. Conversely, MNC2 binds to nearly 95% of breast tumors, but MNC3 does not. Importantly, we demonstrated that MNC2 recognizes MUC1* after MUC1 is cleaved by cleavage enzyme MMP9, which is overexpressed in most breast cancers, but not in hematopoietic stem cells. Expression of MMP9 is predictor of poor prognosis for most solid tumor cancers (Yousef et al. BMC Cancer 2014, 14:609; Mehner et al, Oncotarget, Vol. 5, No. 9, pp 2736-2749, 2014; Radisky et al., Front Biosci (Landmark Ed).; 20:1144-1163, 2015; Gong et al., Journal of Surgical Oncology 2000; 73:95-99; Latinovic et al., Arch Oncol 2013; 21 (3-4): 109-14; Sillanpaa et al., Gynecologic Oncology 104 (2007) 296-303).

In order to generate new anti-MUC1* monoclonal antibodies that were capable of recognizing a wide range of MUC1*'s that can be cancer sub-type specific, patient specific or to better address tumor heterogeneity, we immunized animals with one of the following peptides derived from the sequence of the MUC1* extra cellular domain:

(i) PSMGFR
peptide
(SEQ ID NO: 4)
GTINVHDVETQFNQYKTEAASRYNLTISDVSVSDVPFPFSAQSGA;
(ii) PSMGFR N + 20/C − 27
(SEQ ID NO: 9)
SNIKFRPGSVVVQLTLAFREGTINVHDVETQFNQYKTE;
or
(iii) PSMGFR N + 9/C − 9
(SEQ ID NO: 10)
VQLTLAFREGTINVHDVETQFNQYKTEAASRYNLTISDVSVSDVP

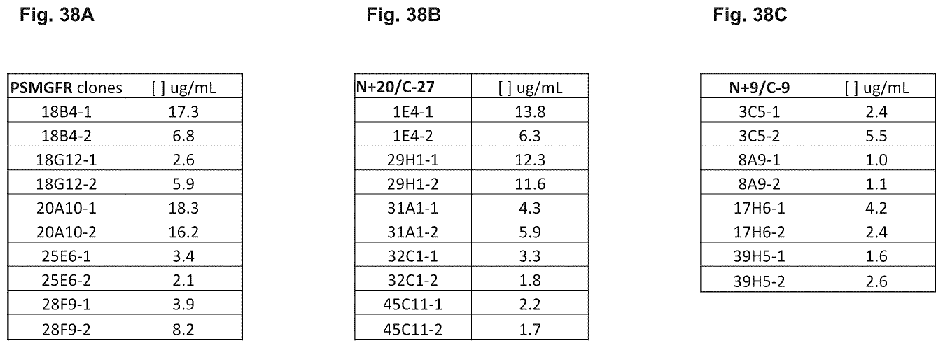

Antibody clones were isolated and a subset from each immunization was selected, first based on their ability to bind to the immunizing peptide, then secondly for their ability to recognize cancerous tissues above normal tissues. FIG. 38A to FIG. 38C shows tables of the selected antibodies, organized according to immunizing peptide. In the tables, designation of −1 or −2 indicates that these are sister clones, which after sequencing showed these were in fact the same antibody. Throughout the rest of the disclosure, antibodies are referred to without the −1 or −2 designation.

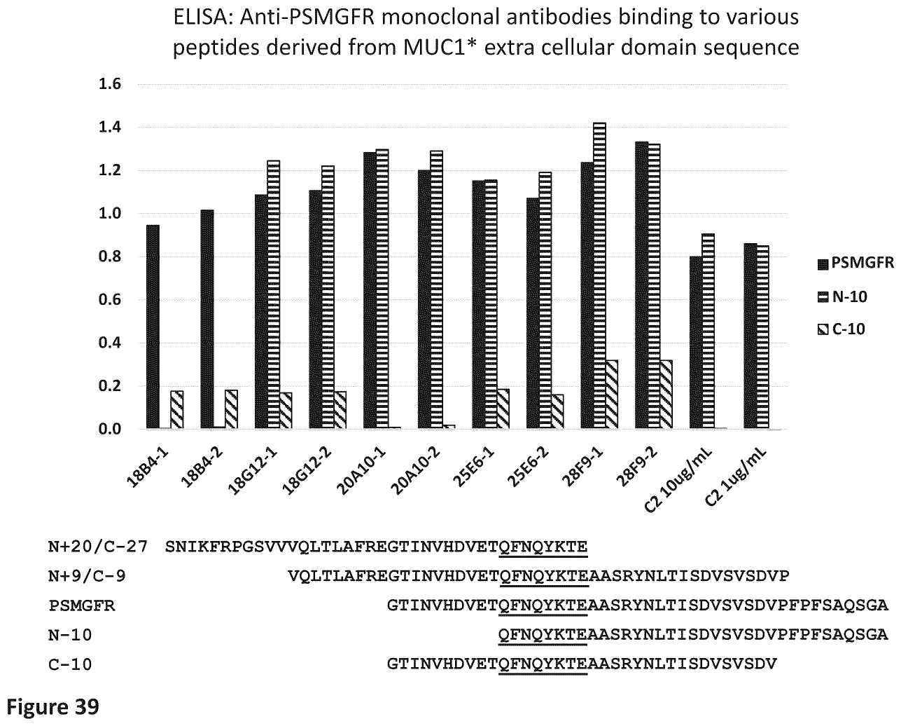

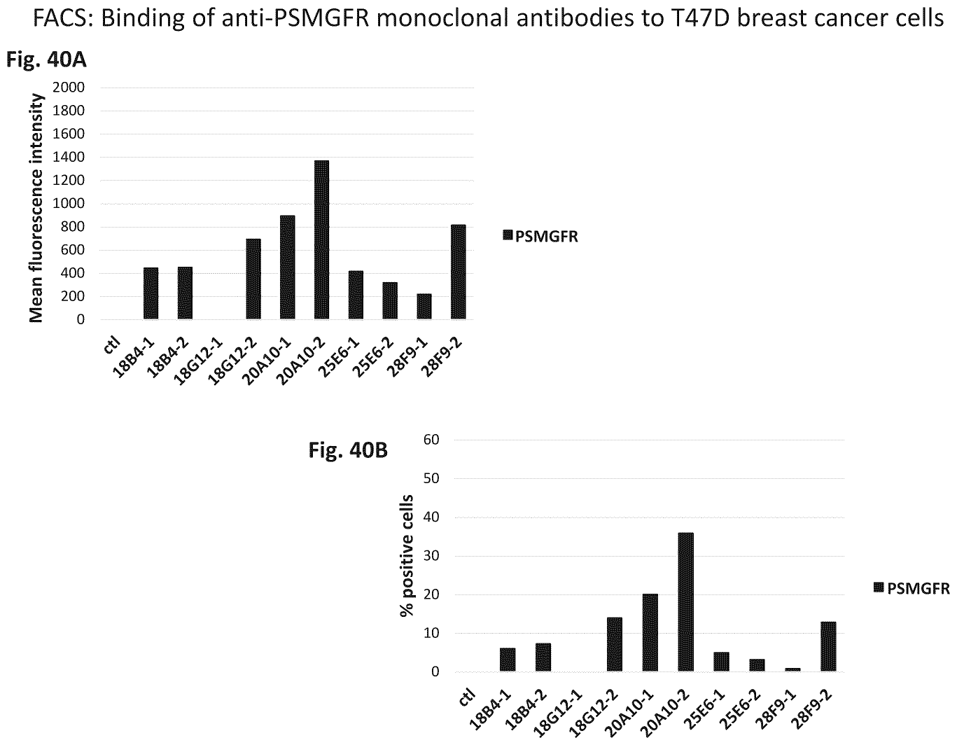

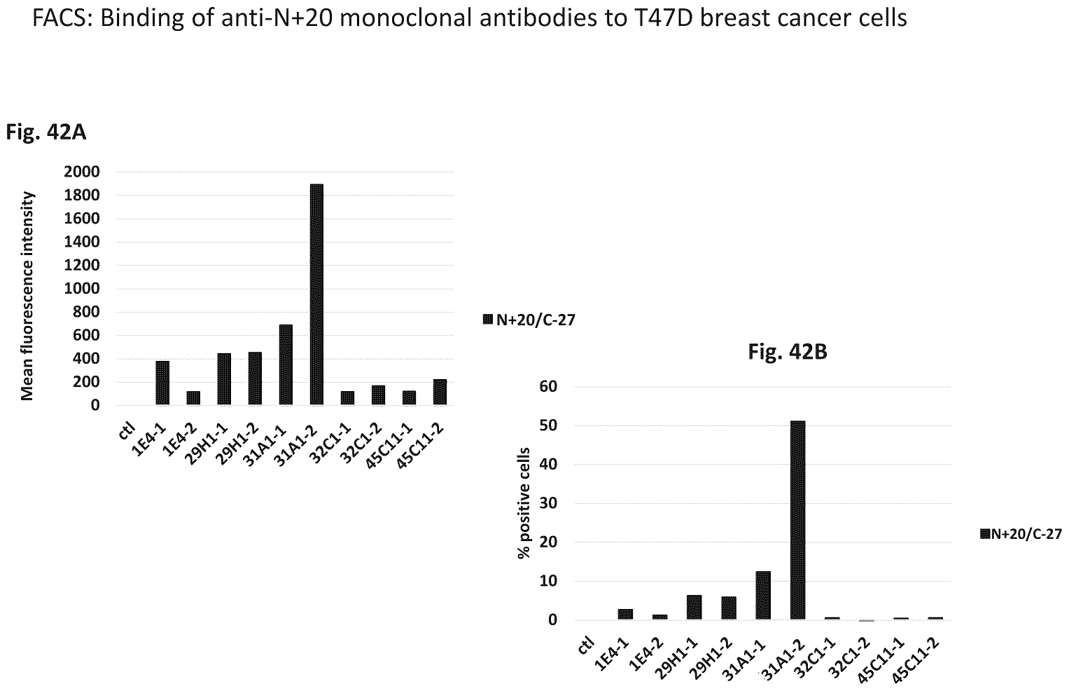

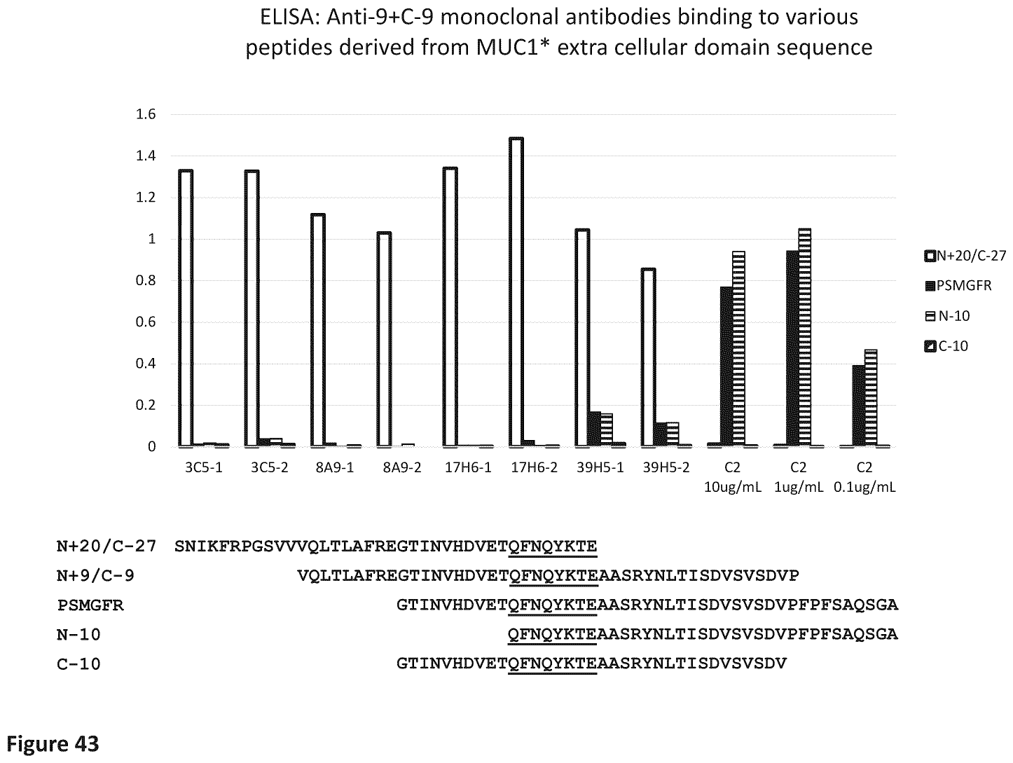

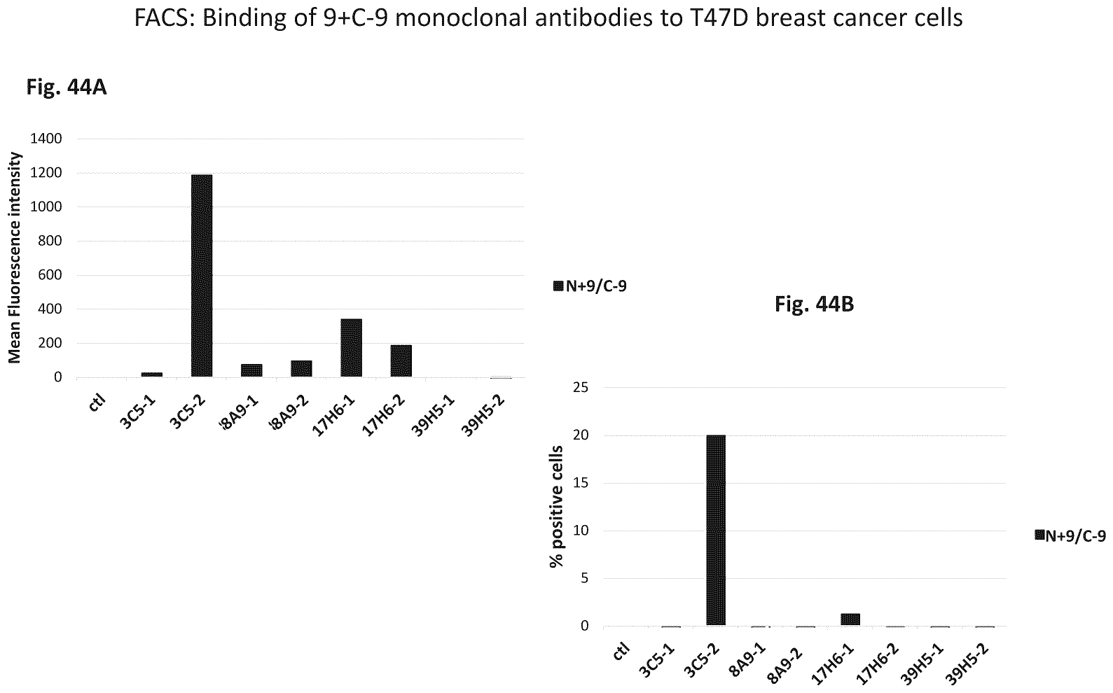

FIG. 39 through FIG. 44 show the binding characteristics of new anti-MUC1* antibodies. All antibodies were first selected by virtue of the fact that they bound to the immunizing peptide. For comparison to MNC2 and MNC3, new antibodies were tested for their ability to bind to PSMGFR, the N-10 peptide and the C-10 peptide. New anti-MUC1* antibodies were also tested by FACS to determine their ability to bind to the T47D breast cancer cell line. Because analysis of antibody binding to a single cell line that was generated from a patient decades ago, we expanded the analysis of the new antibodies to hundreds of tumor tissues across multiple cancer sub-types. The number of patients represented in each array varied. Normal tissues were also probed with the antibodies.

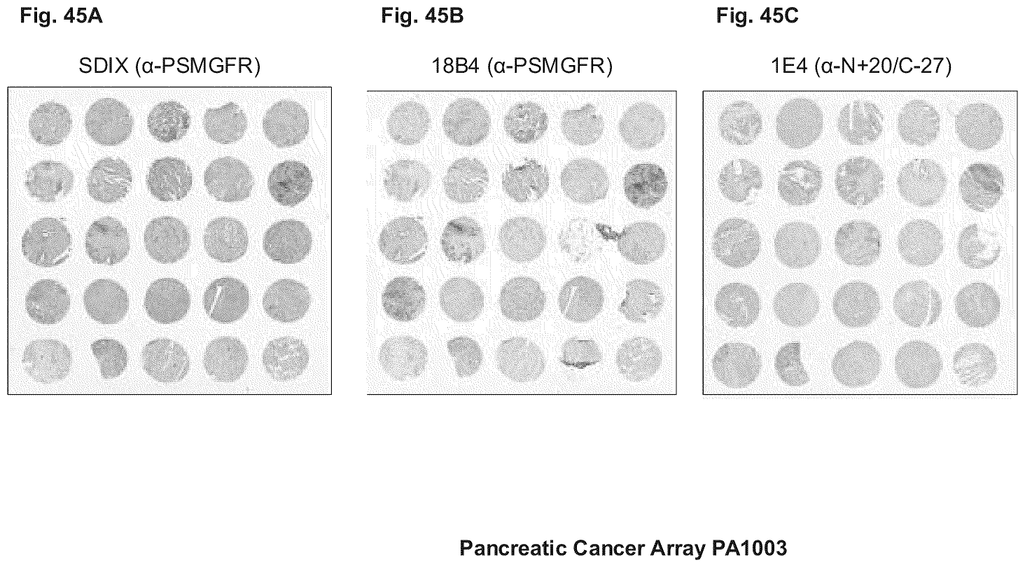

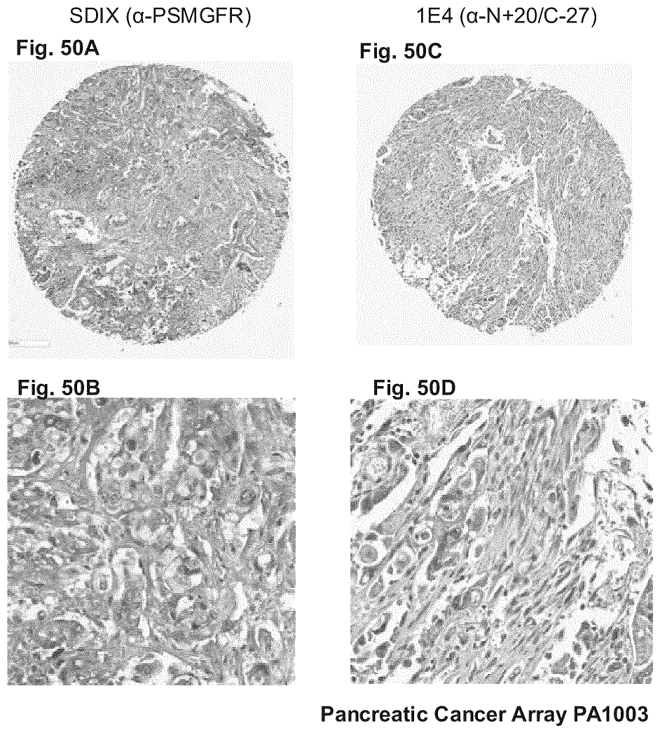

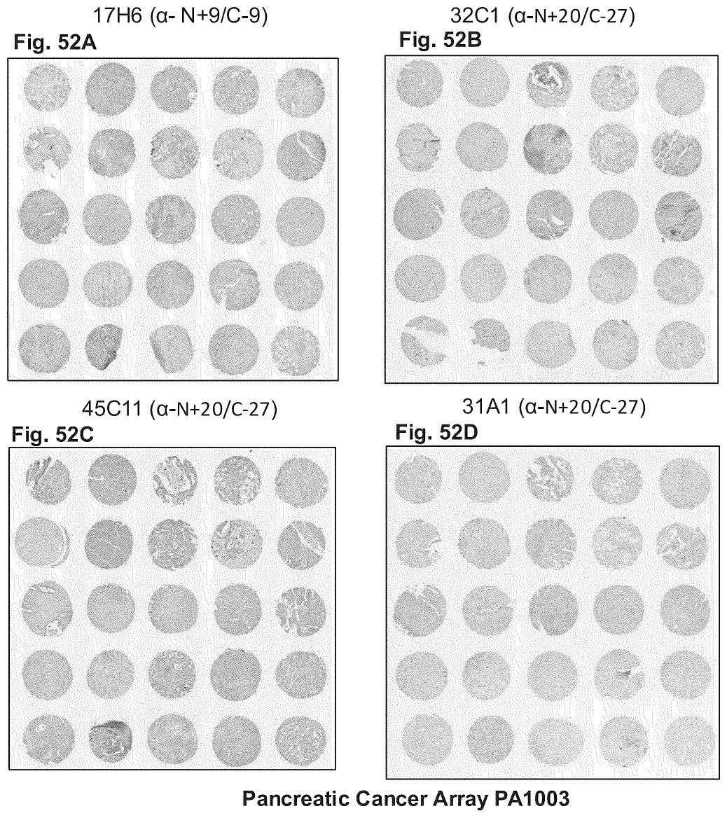

FIG. 45 through FIG. 52 compares the binding of the new anti-MUC1* antibodies to the SDIX polyclonal to investigate antibodies that bind to regions that are N-terminal to the PSMGFR sequence. We started with pancreatic cancer arrays because out previous work showed that although MNC2 recognized about 78% of pancreatic cancers, the binding was not so robust and some very nasty tumors were not recognized at all by MNC2 or the SDIX polyclonal.

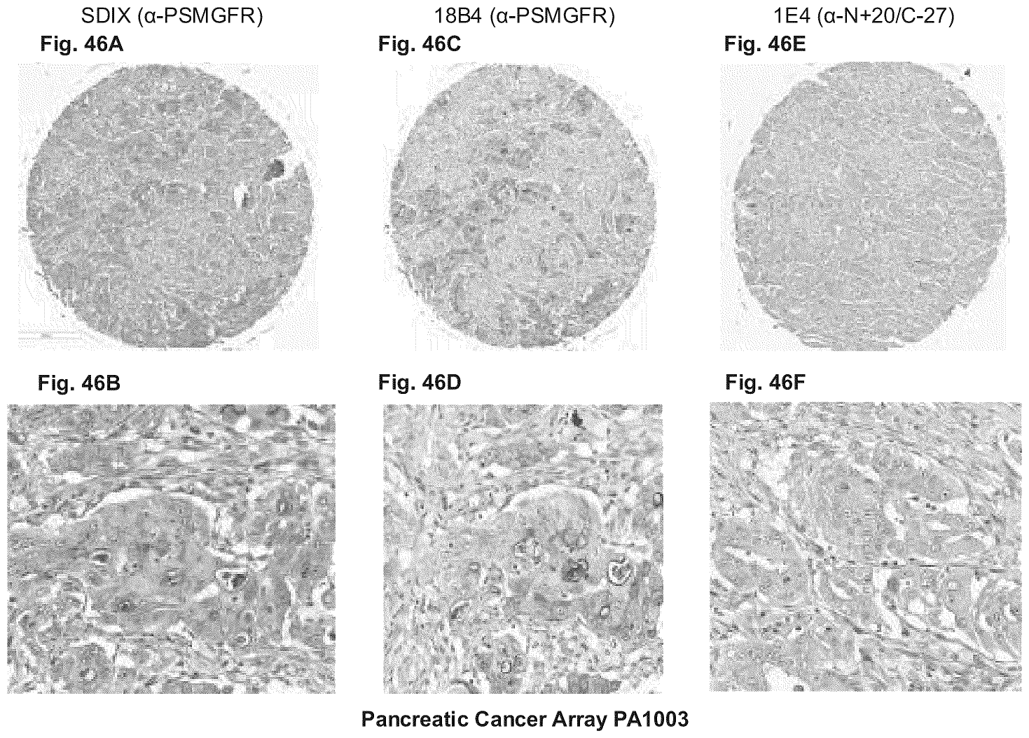

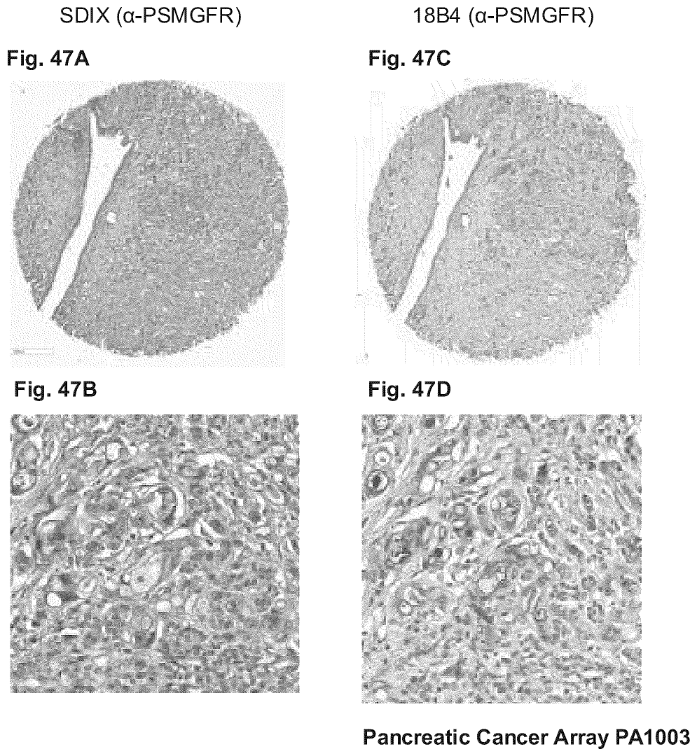

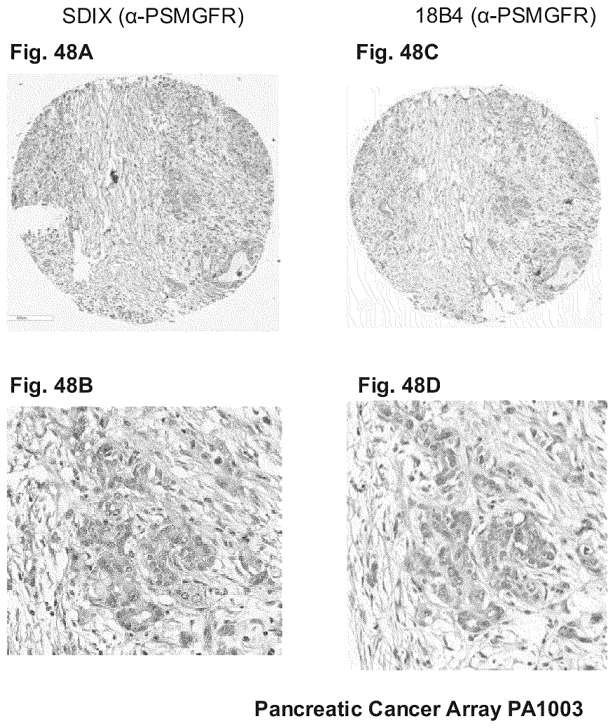

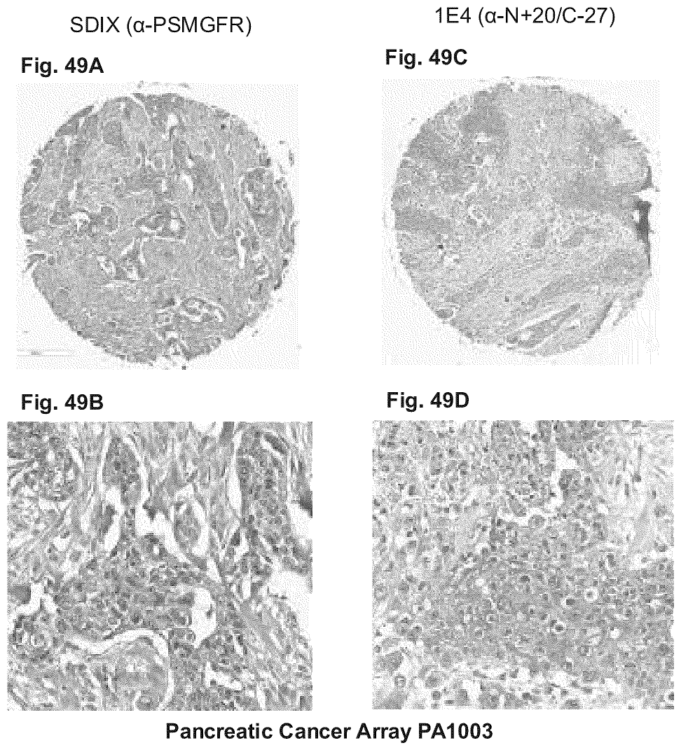

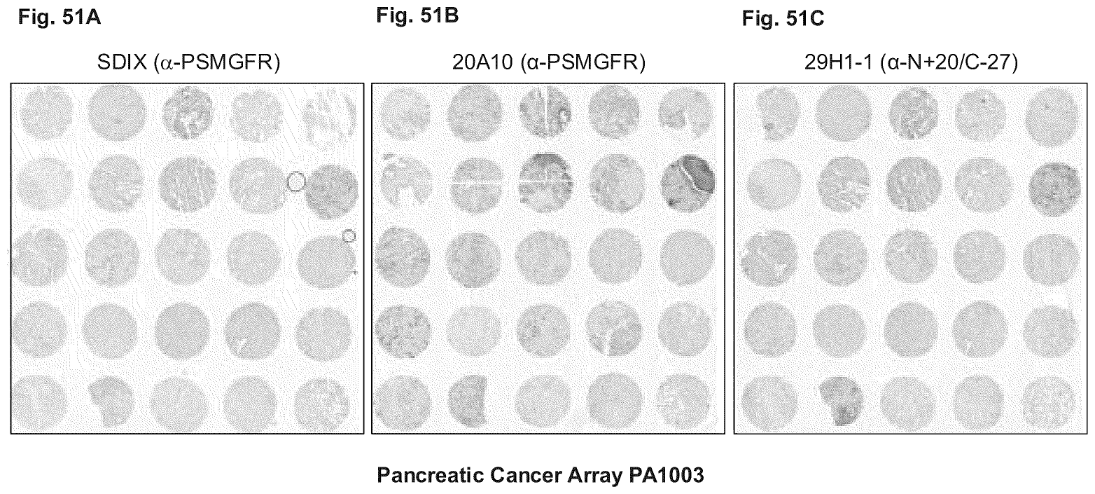

Some anti-PSMGFR antibodies, such as 18B4, appear to recognize the same pancreatic tumor tissues as the polyclonal anti-PSMGFR antibody SDIX (FIGS. 45A-45BC). In this small pancreatic cancer array, anti-PSMGFR N+20/C-27 antibody 1E4 appears to recognize the same tumors as SDIX and 18B4, however, the magnified view of these tumor specimens shows that antibody 1E4 recognizes a different population of cancer cells within the tumor than the anti-PSMGFR antibodies (FIGS. 46A-46F), Some of the tumors were not recognized well by SDIX but were recognized by monoclonal antibody 18B4 (FIGS. 47A-48D). Other pancreatic tumors were recognized better by anti-PSMGFR N+20/C-27 antibody 1E4 (FIGS. 49A-49D). Similarly, anti-PSMGFR N+20/C-27 antibody 29H1 recognizes some pancreatic tumors that are missed by anti-PSMGFR antibodies SDIX and 20A10 (FIGS. 51A-51C).

These studies showed that, in general, antibodies that bind to the MUC1* extra cellular domain that is extended beyond PSMGFR at the N-terminus recognize pancreatic cancers better than SDIX polyclonal. However, antibody specificity of pancreatic tumors appears to also be patient specific. Some patient specimens stained much better with the SDIX anti-PSMGFR antibody than the new antibodies that bind to PSMGFR N+20/C-27 or PSMGFR N+9/C-9. This supports the idea that patient tumors must be probed with a panel of MUC1* antibodies to determine which treatment is best suited for elimination of their tumor. In one aspect of the invention, the therapeutic agent incorporates some or all of the antibody that is the diagnostic agent or some or all of an antibody that is derived from the antibody that is the diagnostic antibody.

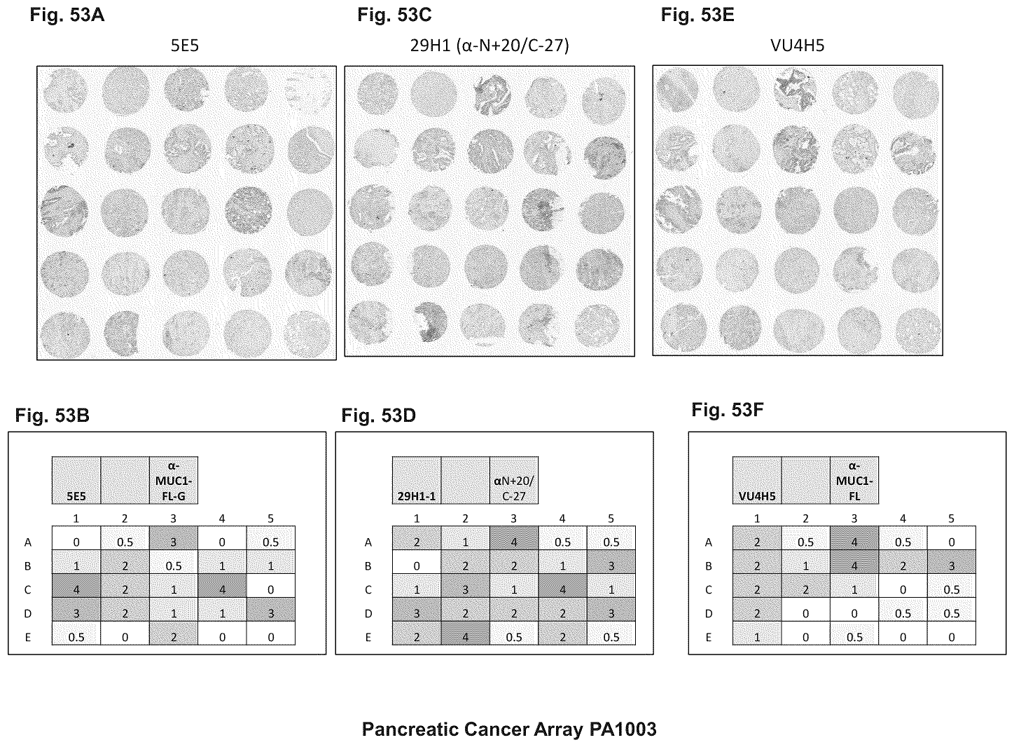

FIG. 53 demonstrates that these new antibodies that are extended at the N-terminus are recognize more pancreatic tumors than antibodies that bind to full-length MUC1. This figure compares the binding of 29H1 to a standard antibody, VU4H5, that binds to the tandem repeats of full-length MUC1, and to a new antibody, 5E5 that binds to a trapped O-linked glycan that is present on some cancer cells.

We next looked at esophageal tumors and prostate tumors. These studies were motivated by our previous findings that monoclonal antibody MNC2 as well as polyclonal antibody SDIX, which both bind to the PSMGFR peptide, showed poor recognition of esophageal tumors and prostate tumors. In fact, those tumors that showed some MNC2 reactivity in the well differentiated portions of a tumor specimen, lost that reactivity in the less well differentiated portion of the same specimen. These results argued that a cleavage enzyme other than MMP9 is dominant in most esophageal and prostate cancers. These studies support that idea.

The new anti-MUC1* antibodies, which bind to peptides PSMGFR N+20/C-27 and/or PSMGFR N+9/C-9, showed markedly better recognition of esophageal and prostate tumors when compared to MNC2, SDIX, and full-length MUC1 antibodies 5E5 and VU4H5.

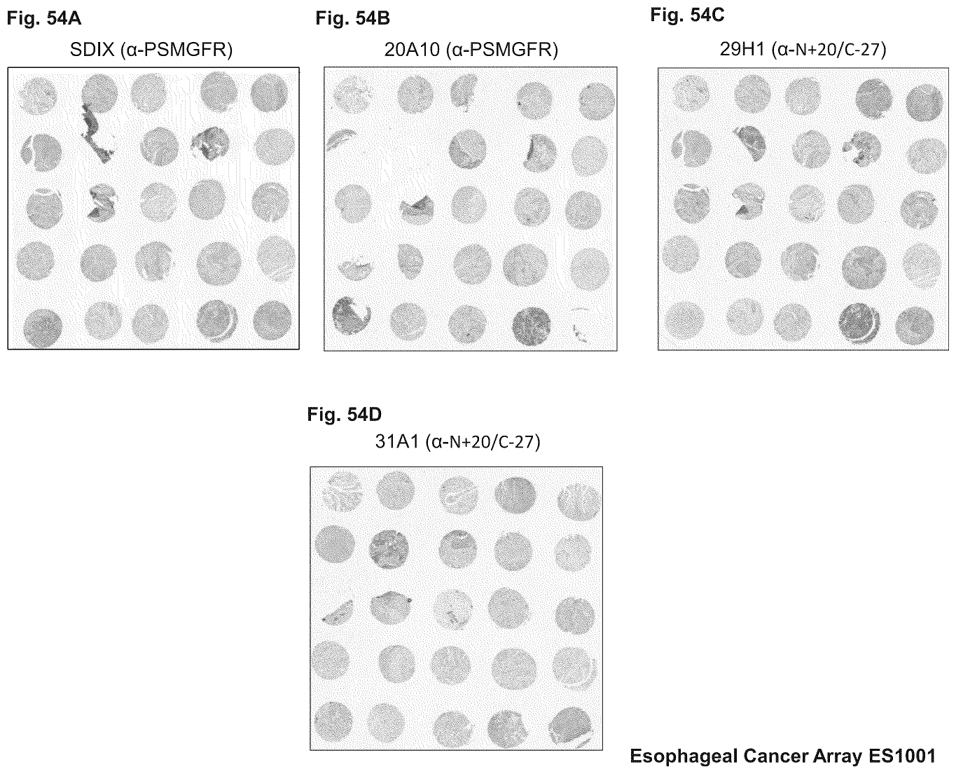

FIG. 54A-54C shows photographs of adjacent serial sections from an esophageal cancer array, which was stained by standard IHC methods with various anti-MUC1* antibodies. FIG. 54A shows the array stained with the SDIX polyclonal anti-MUC1* antibody, wherein the immunogen for the antibody was the PSMGFR peptide. FIG. 54B shows the array stained with the 20A10 monoclonal anti-MUC1* antibody, wherein the immunogen for the antibody was the PSMGFR peptide. FIG. 54C shows the array stained with the 29H1 monoclonal anti-MUC1* antibody, wherein the immunogen for the antibody was the PSMGFR N+20/C-27 peptide. FIG. 54D shows the array stained with the 31A1 monoclonal anti-MUC1* antibody, wherein the immunogen for the antibody was the PSMGFR N+20/C-27 peptide. This figure shows that antibodies SDIX and 20A10 that both bind to the PSMGFR peptide recognize the same tumor tissue specimens, albeit to differing degrees, while antibodies that bind to the PSMGFR N+20/C-27 peptide bind to more esophageal tumor specimens as well as most of those recognized by the anti-PSMGFR antibodies. These results are consistent with the idea that antibodies that bind to the PSMGFR N+20/C-27 peptide are general more specific for esophageal cancers than antibodies that bind to the PSMGFR peptide, but that certain patients may have an esophageal cancer that is better recognized by an anti-MUC1* antibody that binds to the PSMGFR peptide.

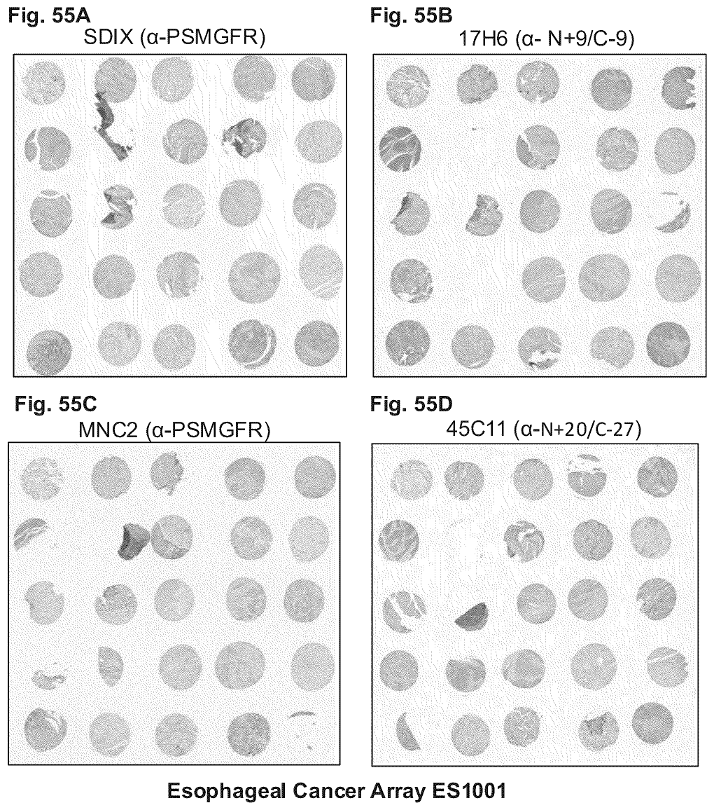

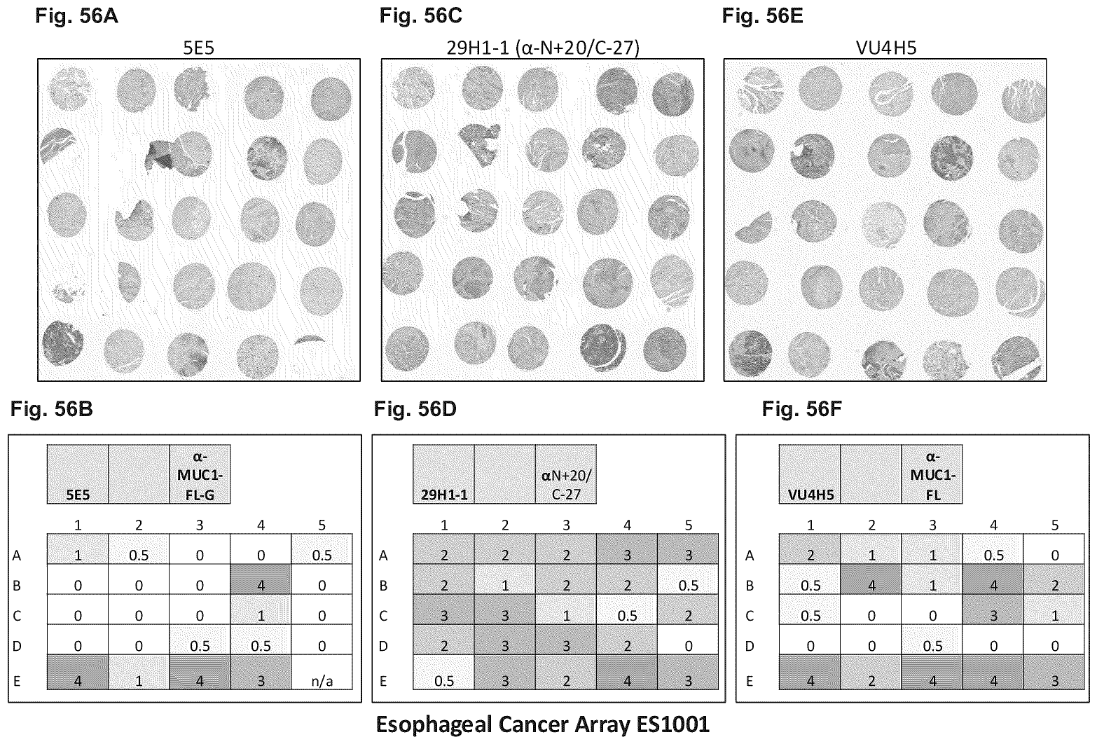

FIG. 55A-55C shows photographs of adjacent serial sections from an esophageal cancer array, which was stained by standard IHC methods with various anti-MUC1* antibodies. FIG. 55A shows the array stained with the SDIX polyclonal anti-MUC1* antibody, wherein the immunogen for the antibody was the PSMGFR peptide. FIG. 55B shows the array stained with the 17H6 monoclonal anti-MUC1* antibody, wherein the antibody binds to the PSMGFR N+9/C-9 peptide. FIG. 55C shows the array stained with the MNC2 monoclonal anti-MUC1* antibody, wherein the antibody binds to the PSMGFR peptide. FIG. 55D shows the array stained with the 45C11 monoclonal anti-MUC1* antibody, wherein the antibody binds to the PSMGFR N+20/C-27 peptide. These results are consistent with the idea that on most esophageal cancers, MUC1 is cleaved by an enzyme that exposes a cryptic epitope that is N-terminal to the PSMGFR sequence.

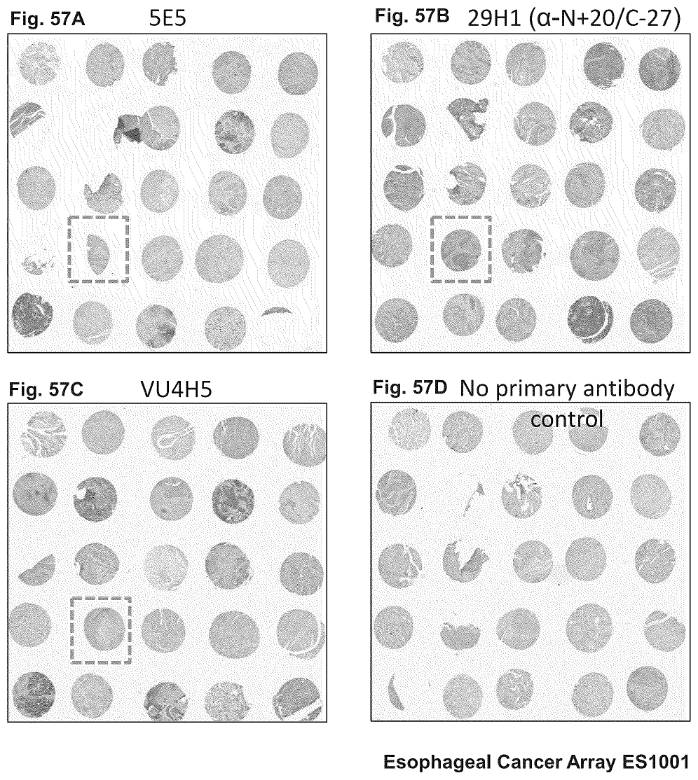

FIG. 56A-56F shows photographs and graphical representations of pathologist staining scores of adjacent serial sections from a esophageal cancer array, which was stained by standard IHC methods with either antibodies that recognize full-length MUC1 or an antibody that only recognizes MUC1*. FIG. 56A shows the esophageal cancer array stained with antibody 5E5, which is an antibody that binds to a trapped O-linked glycan in the tandem repeat domain of full-length MUC1. FIG. 56B shows the pathologist's score for each specimen in the array. FIG. 56C shows the esophageal cancer array stained with anti-MUC1* antibody 29H1, which is an antibody that binds to the PSMGFR N+20/C-27 peptide of MUC1*. FIG. 56D shows the pathologist's score for each specimen in the array. FIG. 56E shows the esophageal cancer array stained with antibody VU4H5, which is an antibody that binds to an epitope in the tandem repeat domain of full-length MUC1. FIG. 56F shows the pathologist's score for each specimen in the array. As can be seen if the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1, and that the antibodies that bind to the PSMGFR N+20/C-27 peptide recognize epitopes that are prevalent on esophageal cancers.

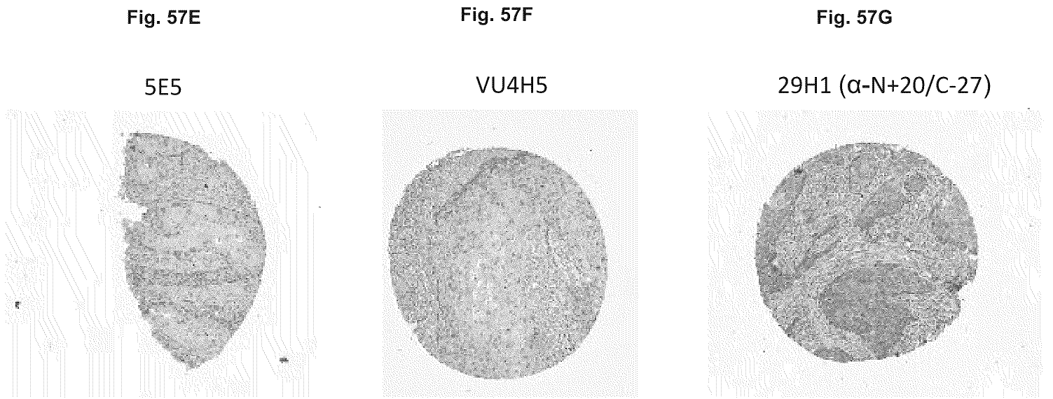

FIG. 57A-57G shows photographs of the prostate cancer array, which was stained with either antibody 5E5 or VU4H5, which both recognize full-length MUC1 or 29H1 that only recognizes MUC1* and binds to the PSMGFR N+20/C-27 peptide. FIG. 57A shows the esophageal cancer array stained with antibody 5E5. FIG. 57B shows the esophageal cancer array stained with antibody 29H1. FIG. 57B shows the esophageal cancer array stained with antibody 29H1. FIG. 57C shows the esophageal cancer array stained with antibody VU4H5. FIG. 57D shows the esophageal cancer array stained with the secondary antibody only, as a control. FIG. 57E shows the tissue marked by red box in FIG. 57A at greater magnification, wherein staining was done with 5E5. FIG. 57F shows the tissue marked by red box in FIG. 57B at greater magnification, wherein staining was done with 29H1. FIG. 57G shows the tissue marked by red box in FIG. 57C at greater magnification, wherein staining was done with VU4H5. The dashed red boxes indicate just one patient's specimen of many esophageal tumor specimens that stain negative for antibodies that recognize full-length MUC1, but highly positive when probed with anti-MUC1* antibodies, and particularly those antibodies that bind to the PSMGFR N+20/C-27 peptide.

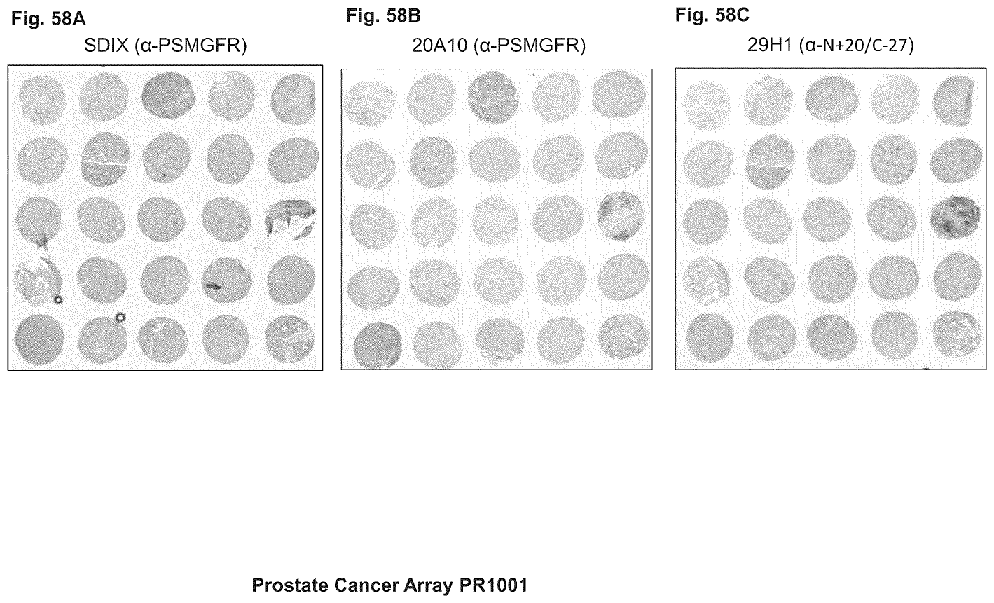

FIG. 58A-58C shows photographs of adjacent serial sections from a prostate cancer array, which was stained by standard IHC methods with various anti-MUC1* antibodies. FIG. 58A shows the array stained with the SDIX polyclonal anti-MUC1* antibody, wherein the immunogen for the antibody was the PSMGFR peptide. FIG. 58B shows the array stained with the 18B4 monoclonal anti-MUC1* antibody, wherein the antibody binds to the PSMGFR peptide. FIG. 58C shows the array stained with the 1E4 monoclonal anti-MUC1* antibody, wherein the antibody binds to the PSMGFR N+20/C-27 peptide.

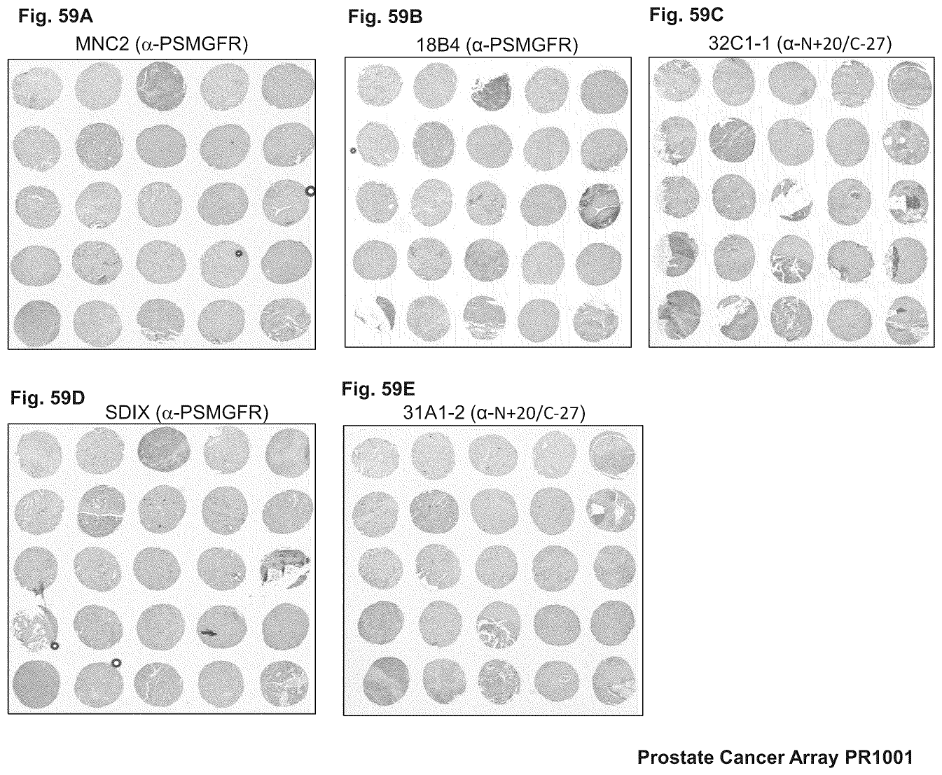

FIG. 59A-59E shows photographs of adjacent serial sections from a prostate cancer array, which was stained by standard IHC methods with various anti-MUC1* antibodies. FIG. 59A shows the array stained with the MNC2 monoclonal antibody that binds to the PSMGFR peptide but not the C-10 peptide. FIG. 59B shows the array stained with the 18B4 antibody that binds to the PSMGFR peptide. FIG. 59C shows the array stained with the 32C1 antibody that binds to the PSMGFR N+20/C-27 peptide. FIG. 59D shows the array stained with the SDIX polyclonal anti-MUC1* antibody, wherein the immunogen for the antibody was the PSMGFR peptide. FIG. 59E shows the array stained with the 31A1 monoclonal anti-MUC1* antibody that binds to the PSMGFR N+20/C-27 peptide.

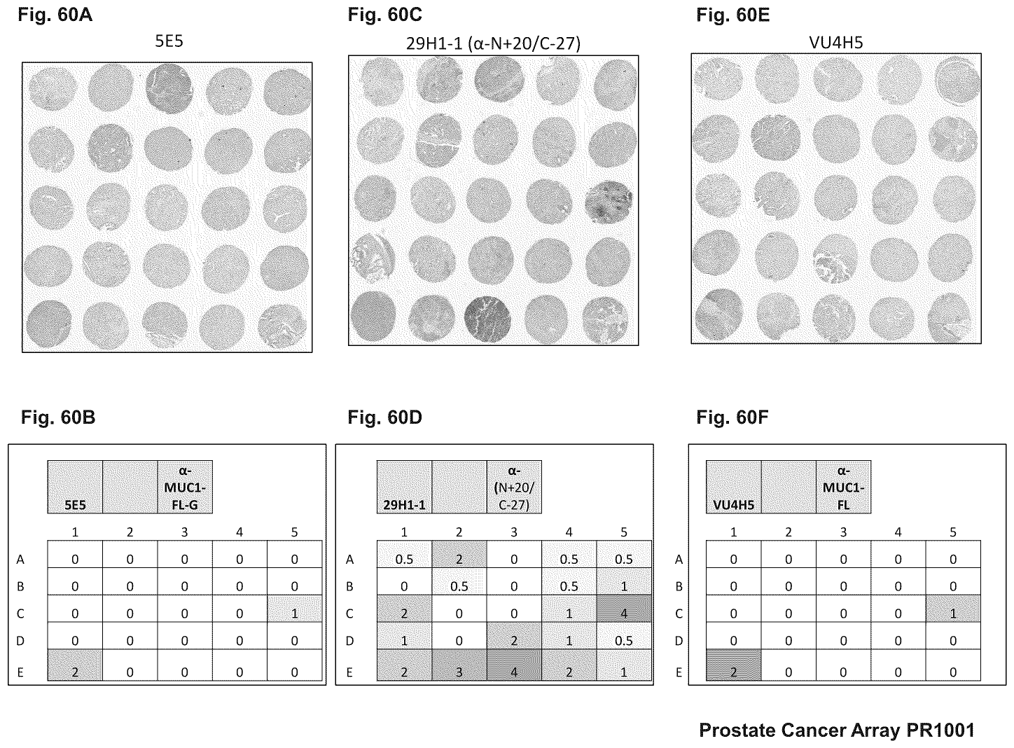

FIG. 60A-60F shows photographs and graphical representations of pathologist staining scores of adjacent serial sections from a prostate cancer array, which was stained by standard IHC methods with either antibodies that recognize full-length MUC1 or an antibody that only recognizes MUC1*. FIG. 60A shows the prostate cancer array stained with antibody 5E5, which is an antibody that binds to a trapped O-linked glycan in the tandem repeat domain of full-length MUC1. FIG. 60B shows the pathologist's score for each specimen in the array. FIG. 60C shows the prostate cancer array stained with anti-MUC1* antibody 29H1, which is an antibody that binds to the PSMGFR N+20/C-27 peptide of MUC1*. FIG. 60D shows the pathologist's score for each specimen in the array. FIG. 60E shows the prostate cancer array stained with antibody VU4H5, which is an antibody that binds to an epitope in the tandem repeat domain of full-length MUC1. FIG. 60F shows the pathologist's score for each specimen in the array. As can be seen if the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1, and that the antibodies that bind to the PSMGFR N+20/C-27 peptide recognize epitopes that are prevalent on prostate cancers.

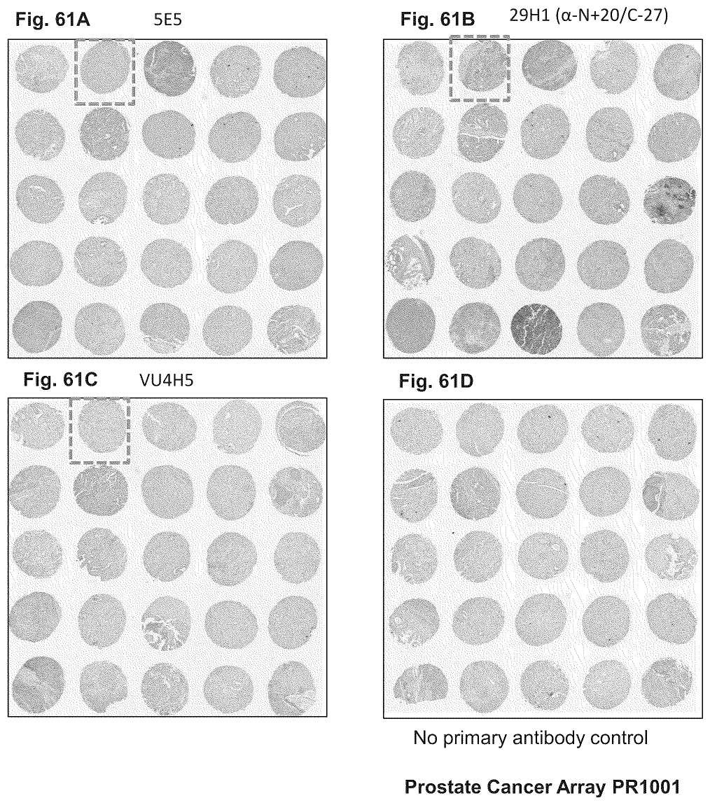

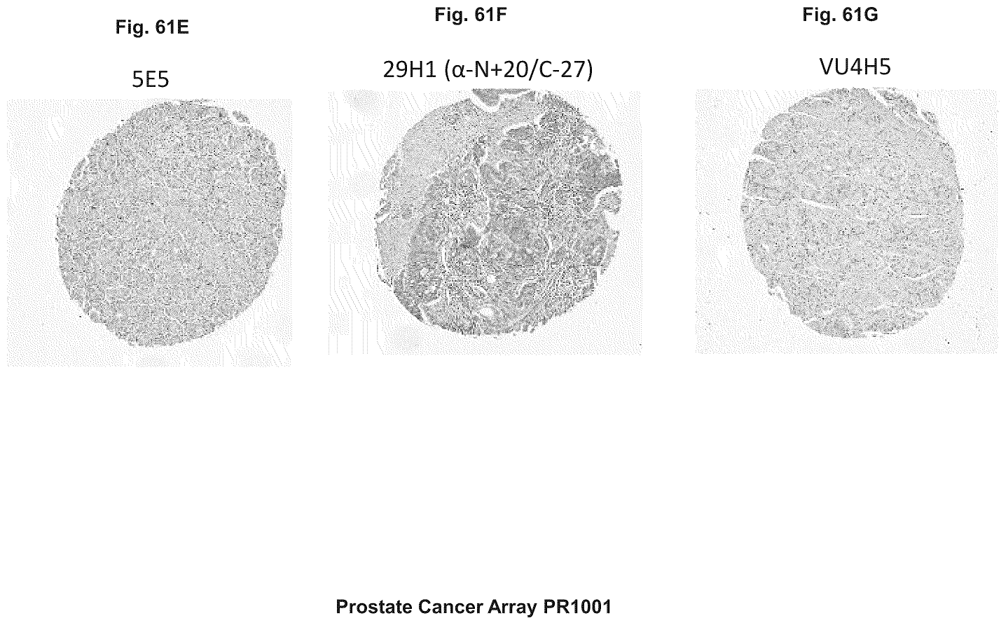

FIG. 61A-61G shows photographs of the prostate cancer array, which was stained with either antibody 5E5 or VU4H5, which both recognize full-length MUC1 or 29H1 that only recognizes MUC1* and binds to the PSMGFR N+20/C-27 peptide. FIG. 61A shows the prostate cancer array stained with antibody 5E5. FIG. 61B shows the prostate cancer array stained with antibody 29H1. FIG. 61B shows the prostate cancer array stained with antibody 29H1. FIG. 61C shows the prostate cancer array stained with antibody VU4H5. FIG. 61D shows the prostate cancer array stained with the secondary antibody only, as a control. FIG. 61E shows the tissue marked by red box in FIG. 61A at greater magnification, wherein staining was done with 5E5. FIG. 61F shows the tissue marked by red box in FIG. 61B at greater magnification, wherein staining was done with 29H1. FIG. 61G shows the tissue marked by red box in FIG. 61C at greater magnification, wherein staining was done with VU4H5. The dashed red boxes indicate just one patient's specimen of many prostate tumor specimens that stain negative for antibodies that recognize full-length MUC1, but highly positive when probed with anti-MUC1* antibodies, and particularly those antibodies that bind to the PSMGFR N+20/C-27 peptide.

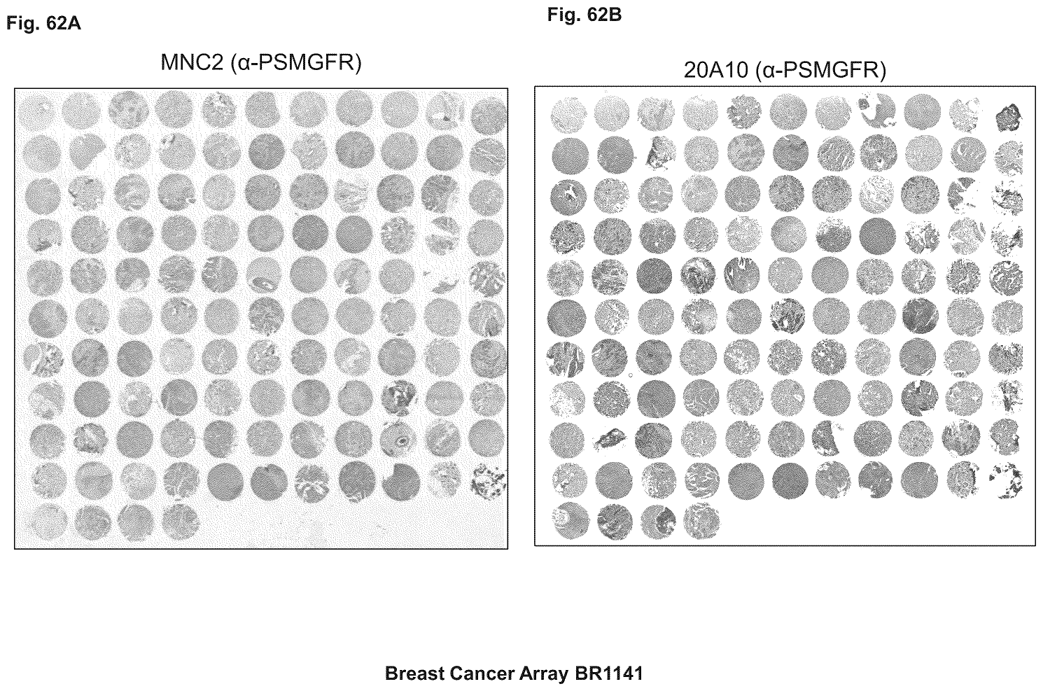

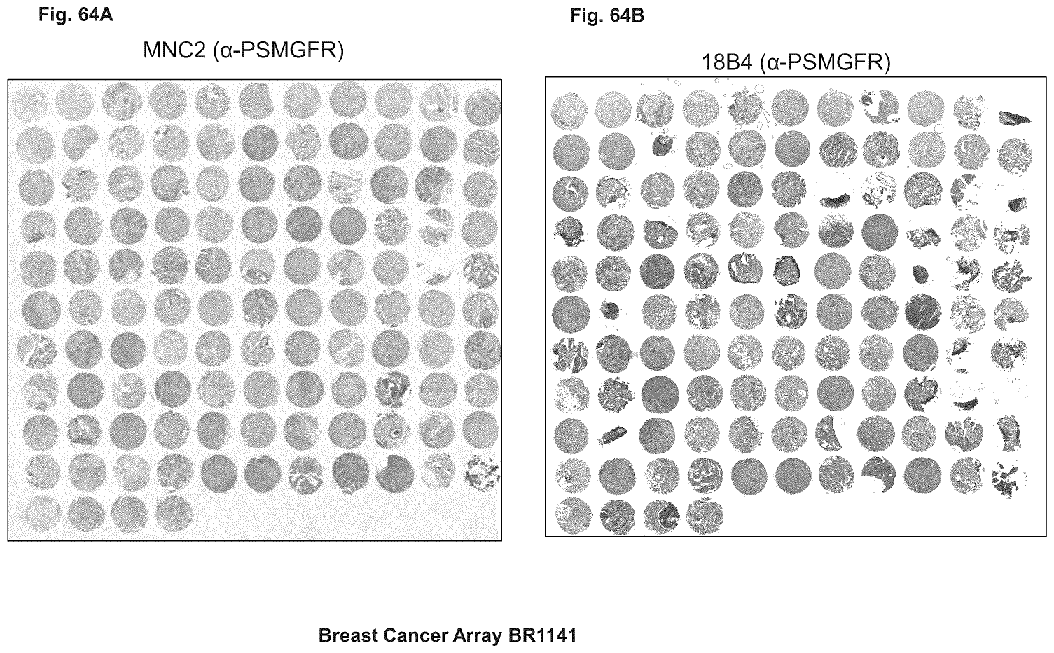

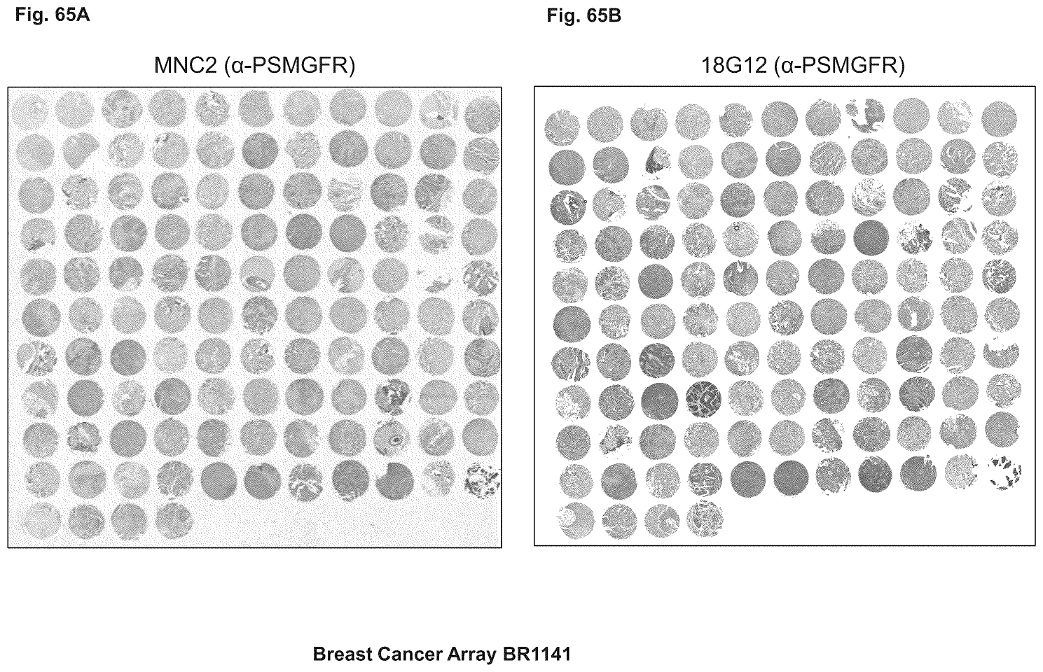

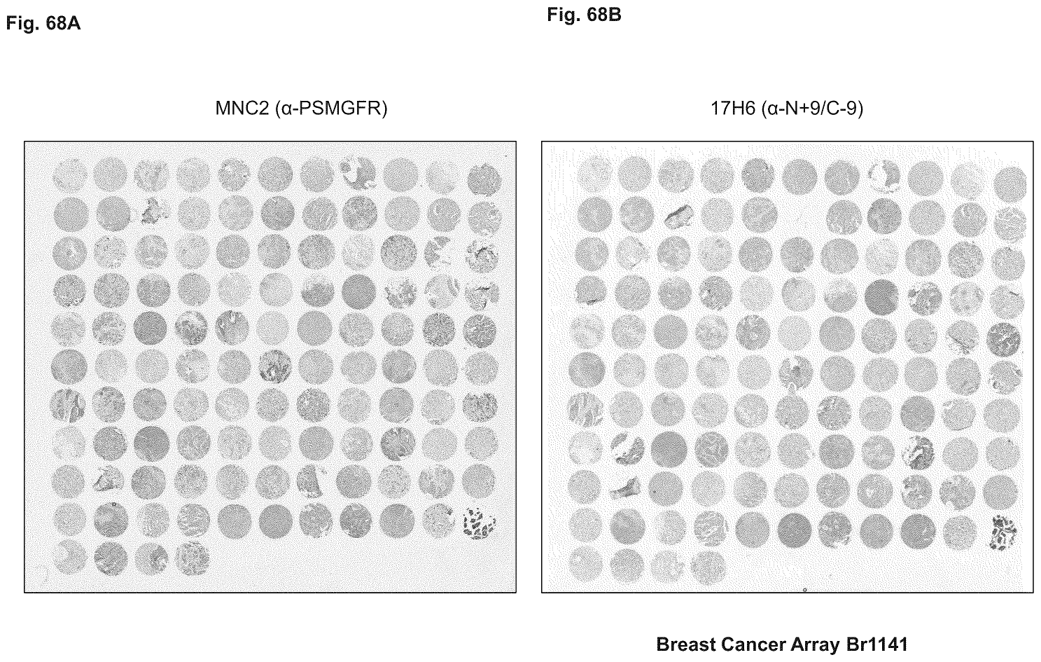

MNC2 recognizes a MUC1* that is present in large percentages of breast cancers. However, tumor heterogeneity and the potential of tumor escape by proliferating a population of cells in which MUC1*, the growth factor receptor, is cleaved by a different cleavage enzyme, and thereby recognized by a different anti-MUC1* antibody, suggests that treatment with more than one anti-MUC1* antibody would be beneficial. To this end, we compared more closely the recognition of new anti-MUC1* antibodies to MNC2 (FIG. 62-FIG. 73).

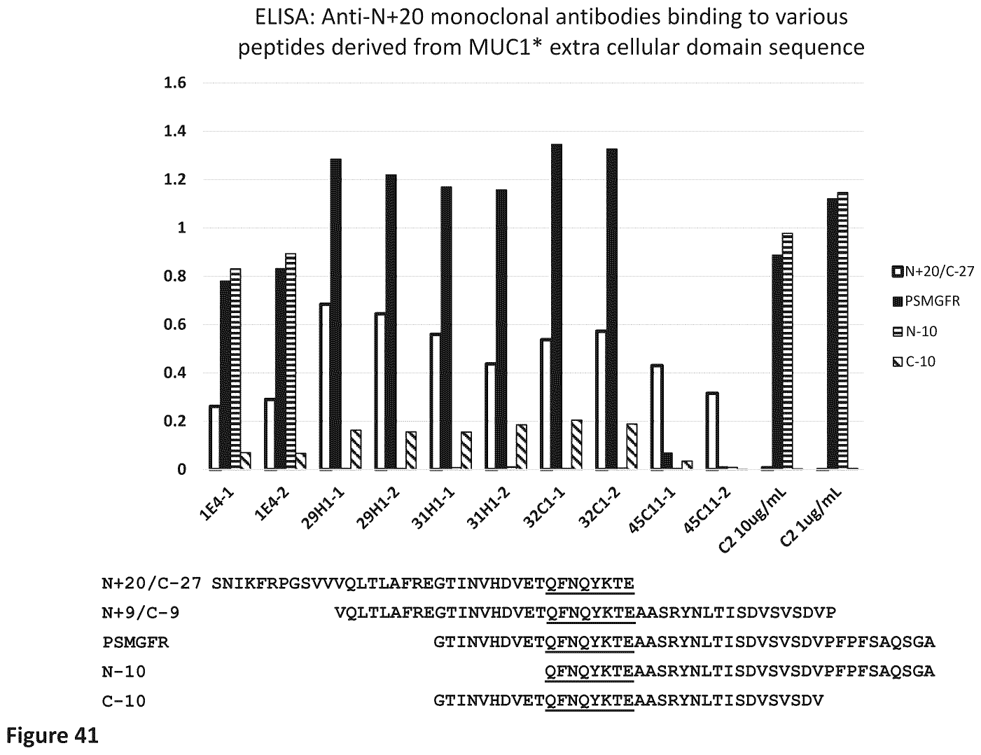

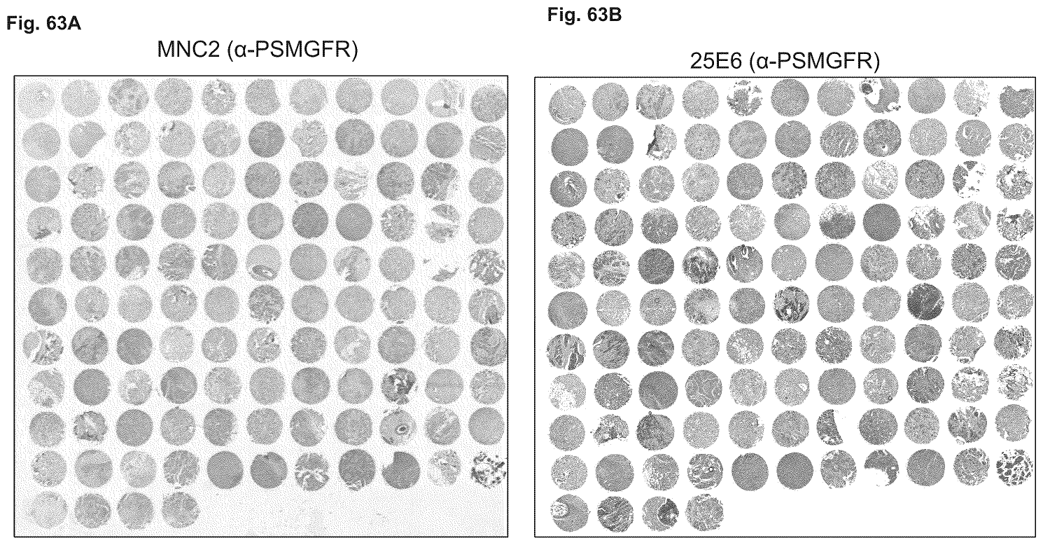

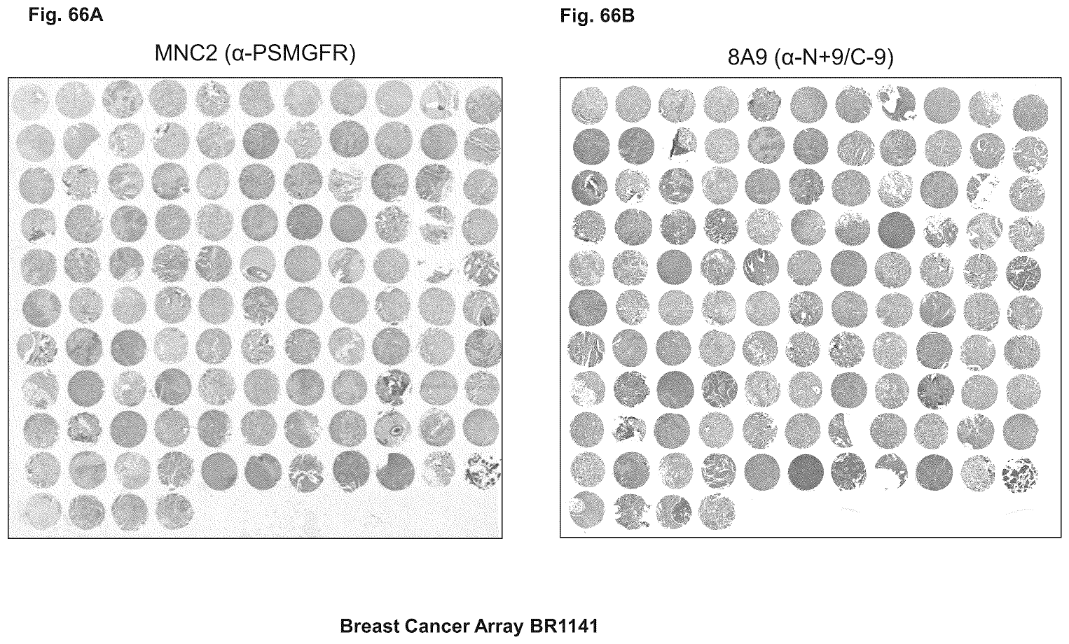

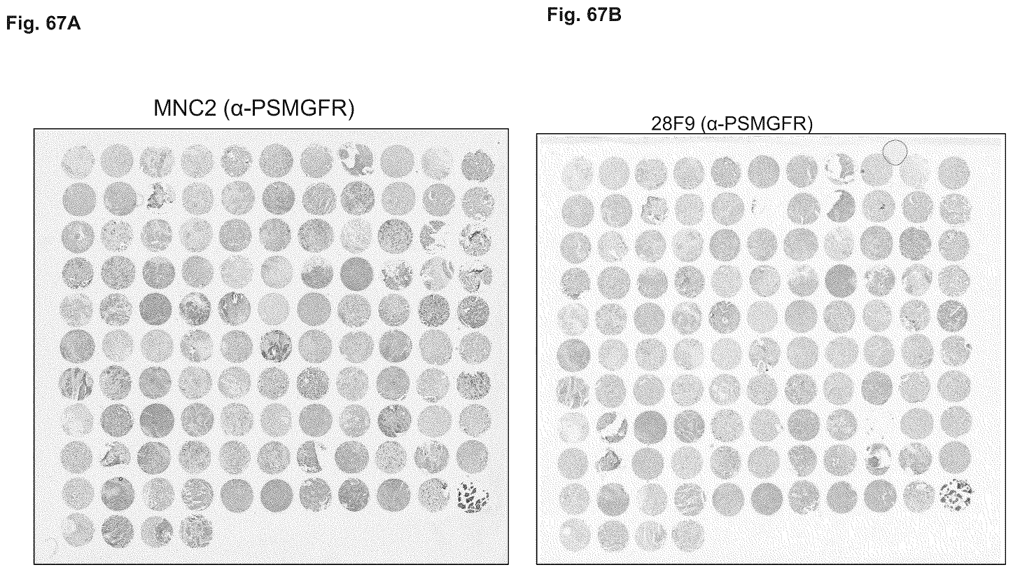

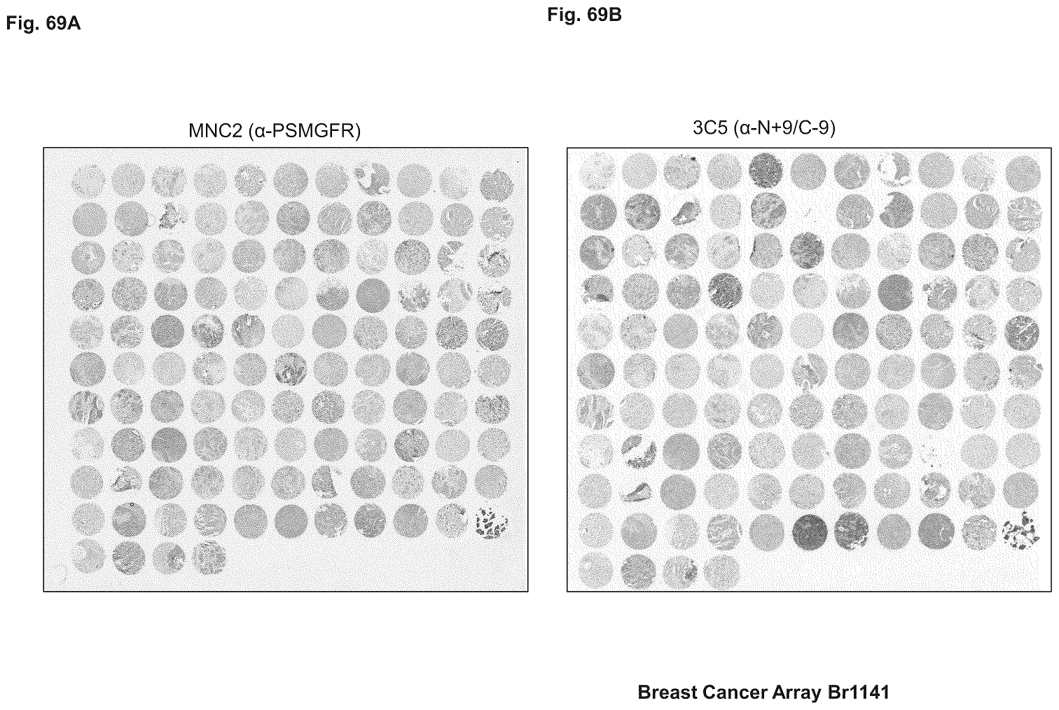

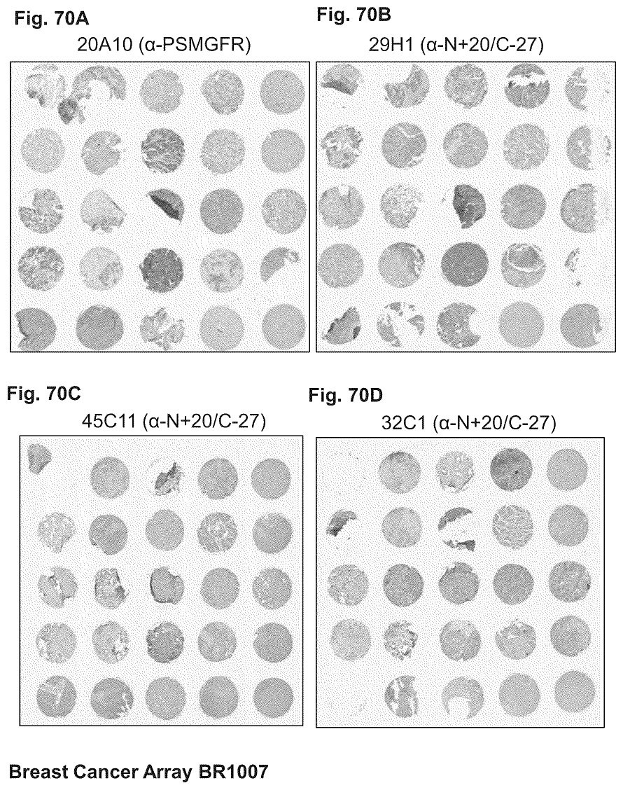

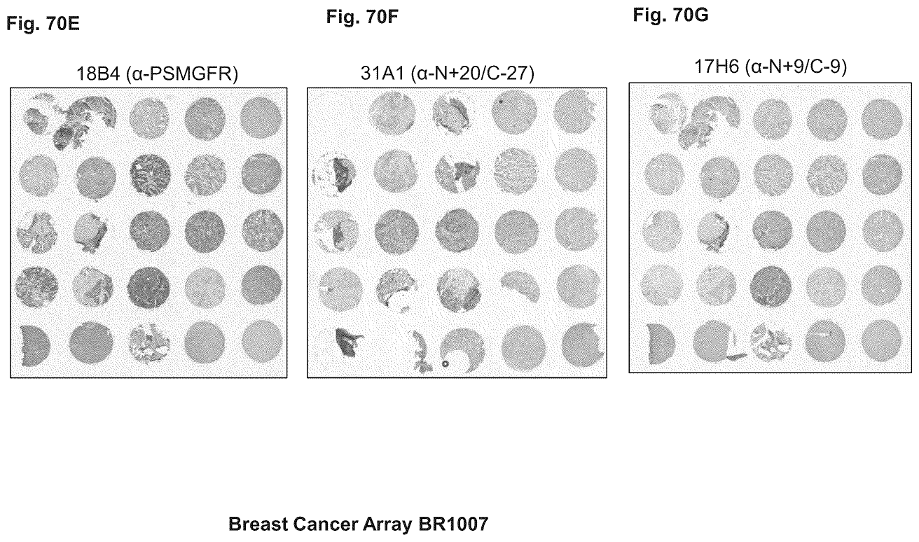

Breast cancer array BR1141 was stained with either MNC2 or 20A10, which both bind to PSMGFR peptide, N-10 peptide, but not the C-10 peptide. To a first order approximation, the two antibodies recognize the same or a very close epitope of a MUC1* that is expressed in breast cancers (FIGS. 62A-62B). FIGS. 63A-65B shows the same breast cancer array but MNC2 compared to 25E6, 18B4 and 18G12. Recall that unlike MNC2, this set of new anti-PSMGFR antibodies are able to bind to the C-10 peptide (FIG. 41). As can be seen in the figure, there are differences between the binding of MNC2 and these new anti-PSMGFR antibodies. Differences in recognition of breast cancer populations between patients, as well as within the same tumor, are more pronounced when MNC2 is compared to anti-PSMGFR N+9/C-9 antibody 8A9 (FIGS. 66A-66B) and anti-PSMGFR antibody 28F9 (FIGS. 67A-67B). Referring to FIG. 41, antibody 28F9 showed the highest degree of binding to the C-10 peptide whereas MNC2 does not bind the C-10 peptide, arguing that these antibodies bind to very different epitopes on the truncated extra cellular domain of MUC1*. Differences between the binding of anti-PSMGFR N+9/C-9 antibody 3C5 and MNC2 are clearly visible in FIGS. 69A-69B. Differences in breast cancer recognition between anti-PSMGFR antibodies 20A10 and 18B4 and other antibodies that bind to peptide PSMGFR N+20/C-27, such as 29H1, 45C11 and 32C1, 31A1 or antibodies that bind to the PSMGFR N+9/C-9 peptide, such as 17H6 are shown in FIGS. 70A-70G.

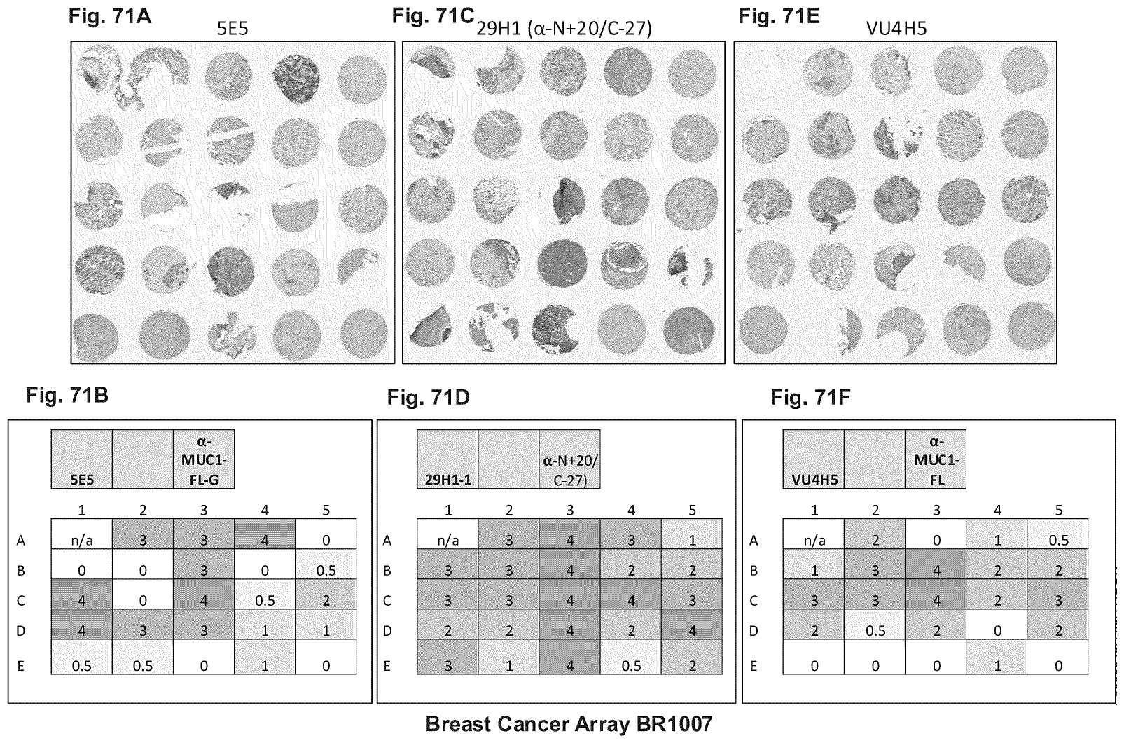

A smaller breast cancer array, BR1007, was probed with anti-MUC1* antibody 29H1 and compared to the recognition of the same array when probed with anti-full-length-MUC1 antibodies 5E5 and VU4H5 (FIG. 71A-71F). As can be seen in the figure, antibody 5E5 recognizes some specimens that VU4H5 does not recognize, however, anti-MUC1* antibody 29H1 recognizes specimens recognized by both antibodies that recognize full-length MUC1 plus other specimens that are not recognized by either anti-MUC1 antibody. These findings show that anti-MUC1* antibodies that bind to peptides that include amino acids that are N-terminally extended beyond PSMGFR sequence are not recognizing full-length MUC1.

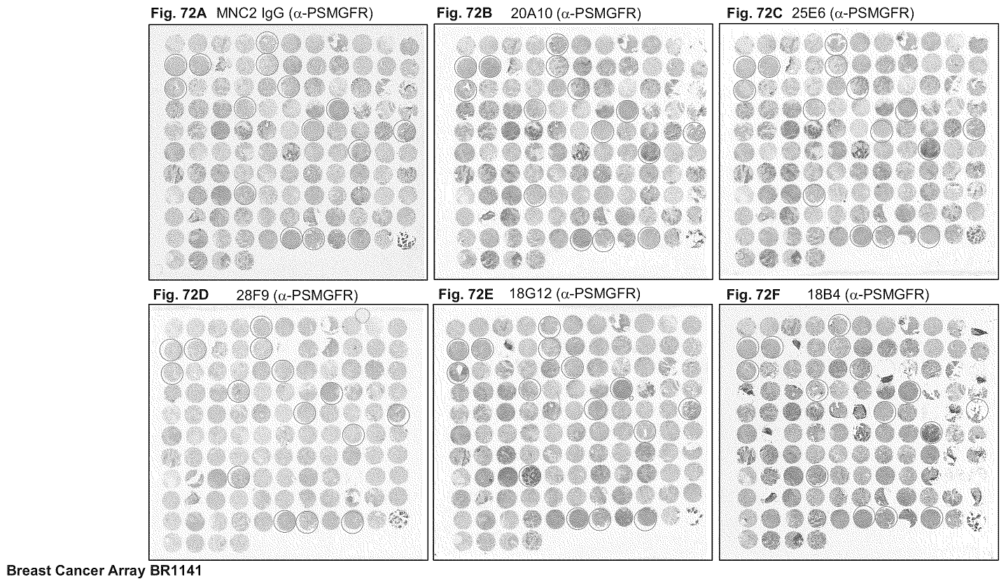

In FIG. 72A-72F, the binding of MNC2 to breast cancer array BR1141 was compared to a panel of anti-PSMGFR antibodies. All these antibodies bind to the PSMGFR peptide and roughly produce the same staining pattern of this breast cancer array. However, there are some differences in how these antibodies recognize individual specimens within the array, which could represent MUC1 to MUC1* cleavage by different enzymes. Referring to FIG. 39, MNC2 and 20A10 bind to the N-10 peptide but not to the C-10 peptide, indicating the 10 membrane proximal amino acids are important for their binding. Antibodies 18B4, 18G12 and 25E6 show some binding to the C-10 peptide and 28F9 shows even more binding to C-10 peptide. Notably, 18B4 does not bind to the N-10 peptide, indicating that it binds to an epitope that is more N-terminal within PSMGFR than the others. Albeit with the previously mentioned exceptions, the recognition of tumors within this array by anti-PSMGFR antibodies was very similar.

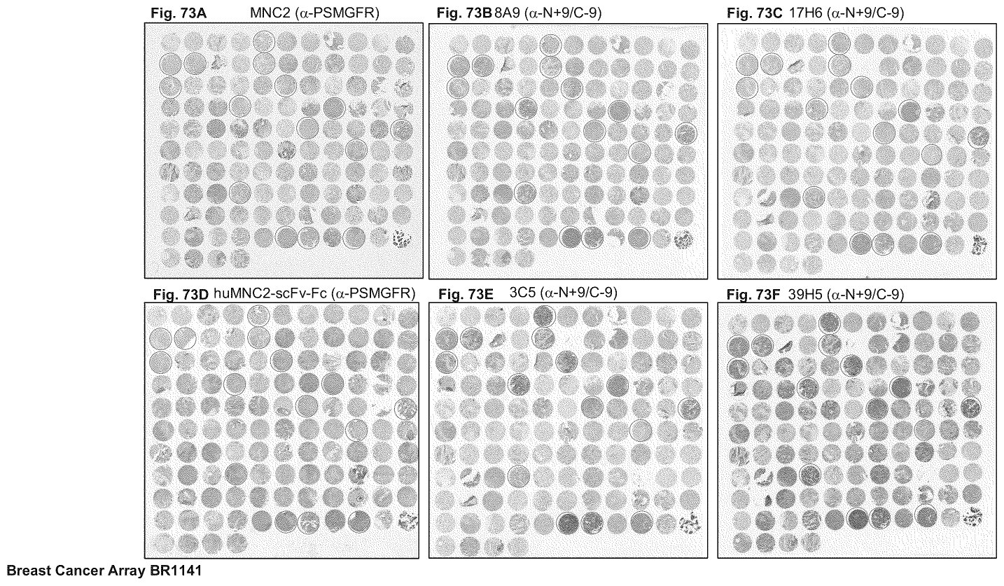

In contrast, antibodies that bind to the PSMGFR N+9/C-9 peptide robustly recognized a subset of tumors that was either not recognized by MNC2 or weakly recognized by MNC2 and other anti-PSMGFR antibodies (FIG. 73A-73F). The photographs shown are of adjacent serial sections of breast cancer tissue array BR1141 that have been stained with various anti-MUC1* monoclonal antibodies, wherein antibodies that bind to the PSMGFR N+9/C-9 peptide are compared to MNC2 and its humanized single chain form, huMNC2-scFv-Fc, which both bind to PSMGFR, N-10 but not to C-10 peptides. FIG. 73A shows breast cancer specimen that was stained with MNC2. FIG. 73B shows breast cancer specimen that was stained with 8A9. FIG. 73C shows breast cancer specimen that was stained with 17H6. FIG. 73D shows breast cancer specimen that was stained with huMNC2-scFv-Fc. FIG. 73E shows breast cancer specimen that was stained with 3C5. FIG. 73F shows breast cancer specimen that was stained with 39H5. Referring now to the patient specimens that are marked by red circles, it is plain to see that antibodies that bind to the PSMGFR N+9/C-9 peptide recognize a population of breast cancer cells that MNC2 anti-PSMGFR antibodies miss or bind weakly to. Anti-MUC1* antibodies 8A9, 17H6, 3C5, and 39H5 recognize a unique subset of cancer cells that are either not recognized or recognized to a lesser degree by anti-PSMGFR antibodies such as MNC2, 20A10, 25E6, 28F9, 18G12, or 18B4.

Collectively, these data show that: (i) diagnosis of MUC1 positive cancers, even within a cancer sub-type such as breast cancers, is more accurate when a tumor is probed with an anti-MUC1* antibody rather than an antibody that binds to full-length MUC1; (ii) diagnosis of MUC1 positive cancers, even within a cancer sub-type such as breast cancers, is more accurate when a tumor is probed with more than one anti-MUC1*; (iii) diagnosis of MUC1 positive cancers, even within a cancer sub-type such as breast cancers, is even more accurate when a tumor is probed with more than one anti-MUC1*, wherein the at least two different antibodies are chosen from among two different groups, wherein the groups are antibodies that bind to the PSMGFR peptide, antibodies that bind to the PSMGFR N+20/C-27 peptide, and antibodies that bind to the PSMGFR N+9/C-9 peptide.

Anti-MUC1* antibodies of the invention, which can be used for use in the diagnosis of cancers, include antibodies that bind to the PSMGFR peptide, the PSMGFR N+20/C-27 peptide, the PSMGFR N+9/C-9 peptide, or more specifically antibodies that bind to a peptide having at least 15 contiguous amino acids of the sequences below, with up to four amino acids substitutions;
(i) PSMGFR region of MUC1;
(ii) PSMGFR peptide as set forth in SEQ ID NO:4;
(iii) PSMGFR N+20/C-22, a peptide having amino acid sequence of SNIKFRPGSVVVQLTLAFREGTINVHDVETQFNQYKTEAASRY (SEQ ID NO:5);
(iv) PSMGFR N+12/C-22, a peptide having amino acid sequence of SVVVQLTLAFREGTINVHDVETQFNQYKTEAASRY (SEQ ID NO:6);
(v) PSMGFR N+9/C-30, a peptide having amino acid sequence of VQLTLAFREGTINVHDVETQFNQY (SEQ ID NO:7);
(vi) PSMGFR N+20/C-41, a peptide having amino acid sequence of SNIKFRPGSVVVQLTLAFREGTIN (SEQ ID NO:8)
(vii) PSMGFR N+20/C-27, a peptide having amino acid sequence of SNIKFRPGSVVVQLTLAFREGTINVHDVETQFNQYKTE (SEQ ID NO:9); or
(viii) PSMGFR N+9/C-9, a peptide having amino acid sequence of VQLTLAFREGTINVHDVETQFNQYKTEAASRYNLTISDVSVSDVP (SEQ ID NO:10). Specifically anti-PSMGFR antibodies MNC2, MNE6, 18B4, 18G12, 20A10, 25E6, anti-PSMGFR N+20/C-27 antibodies 1E4, 29H1, 31A1, 32C1, 45C11, and anti-PSMGFR N+9/C-9 antibodies 3C5, 8A9, 17H6, and 39H5 are antibodies that can be used to diagnose cancers. These antibodies may be human, humanized or non-human. They may be antibody intact antibodies or antibody fragments. Antibodies may be generated by immunizing animals with peptides of sequences (i)-(viii) above. The animal that is immunized with the MUC1* extra cellular domain peptides to produce the antibodies may be human, rabbit, mouse, goat, donkey, camelid, llama, alpaca or other non-human species.

An antibody of the invention can be used in a diagnostic assay wherein it may be derivatized with, or attached to an imaging agent, a dye, a fluorescent entity, a color producing reagent or any other entity that renders the antibody optically, visually, electrically or radioactively detectable. Antibodies of the invention can be used in a variety of diagnostic formats.

In another example, anti-MUC1* antibodies of the invention can be attached to an imaging agent for use in a live patient as a whole body diagnostic to determine if the patient has a MUC1* positive tumor or to determine if the patient would benefit from a therapeutic comprising all, or a fragment of, an anti-MUC1* antibody, which may be derived from or have similar binding characteristics as the antibody used in the diagnostic. The species of the diagnostic antibody and the therapeutic antibody do not need to be the same. Antibodies generated in camelid species are particularly useful for in vivo diagnostic assays because camelids generate small monovalent antibodies that have a short half-life in humans.

In yet another example, anti-MUC1* antibodies of the invention may be attached to an imaging agent and used intra-surgically to detect or mark cancerous tissues so they can be excised completely during the surgery.

In one aspect of the invention, a bodily fluid or tissue specimen from a patient diagnosed with cancer or suspected to be at risk of cancer is contacted with one or more anti-MUC1* antibodies of the invention; analysis of the binding of the antibody to the cells of the specimen indicate a level of binding or a pattern of binding that is indicative of cancer. A therapeutic agent for the treatment of cancer is then administered to the patient. In one aspect of the invention the therapeutic agent comprises all or a fragment of an anti-MUC1* antibody.

In one example, diagnostic assays employing anti-MUC1* antibodies or fragments thereof are used to screen patients to determine their potential benefit from a MUC1* targeting therapeutic. The anti-MUC1* antibody used in the diagnostic and the antibody or fragment thereof that is incorporated into the therapeutic may be derived from the same antibody. The species of the diagnostic antibody and the therapeutic antibody do not need to be the same. Diagnostic assays may encompass use of one or more anti-MUC1* antibodies. A patient specimen that reacts with one or more anti-MUC1* antibodies indicates that the patient may benefit from administration of therapeutics that contain the one or more reactive antibodies, or fragments thereof.

One example, includes the steps of: (i) a suspect cellular or tissue specimen, which may be a biopsy, from a patient diagnosed with cancer or suspected of developing cancer is contacted with an anti-MUC1* antibody; (ii) a normal cellular or tissue specimen from the patient or from a healthy donor is contacted with the same anti-MUC1* antibody, which may be an archived reference specimen; (iii) antibody binding is detected; (iv) the extent and pattern of antibody binding to the suspect specimen is compared to that of the normal specimen; (v) a determination that the suspect specimen overexpresses MUC1*, or expresses MUC1* in a uniform pattern as opposed to expression that is restricted to the apical border, indicates that the patient is suffering from a MUC1* positive cancer; (vi) a therapeutic agent for the treatment of cancer is then administered to the patient, which may be a therapeutic agent that incorporates an anti-MUC1* antibody, or fragment thereof.

In one aspect of the invention, a bodily fluid or tissue specimen from a patient diagnosed with or suspected of having cancer is contacted with an anti-MUC1* antibody of the invention and a higher than normal level of MUC1* is detected or an abnormal pattern of MUC1* is detected, indicating that the patient has a MUC1* positive cancer and a therapeutic agent is then administered to the patient, which incorporates an anti-MUC1* antibody or antibody fragment. In one case the therapeutic agent into which the antibody or antibody fragment is incorporated is an immuno-oncology agent, such as a CAR T cell, an engineered NK cell or a dendritic cell. In another case, the therapeutic agent into which the antibody or antibody fragment is incorporated is a huMNC2-CAR44 T cell. In yet another aspect of the invention the therapeutic agent into which the antibody or antibody fragment is incorporated is a bispecific antibody. In yet another aspect of the invention the therapeutic agent into which the antibody or antibody fragment is incorporated is an antibody drug conjugate (ADC). In yet another aspect of the invention the therapeutic agent into which the antibody or antibody fragment is incorporated is a bispecific T cell engager (BiTE).

In another example, the diagnostic assay may comprise an anti-MUC1* antibody and a second antibody, and the steps may comprise determining the ratio of the amount of a first antibody to a second antibody. The first antibody may bind to MUC1* extra cellular domain and the second antibody may bind to a portion of the MUC1 extra cellular domain that is N-terminal of the cleavage site, such as the tandem repeat sequences. In the case of contacting a tissue specimen, the higher the ratio of MUC1* to full-length MUC1, the more progressed is the cancer and the more likely it is that the patient would benefit from a MUC1* targeting therapeutic.

The invention includes antibodies as well as antibody-like proteins, including but not limited to polyclonal, monoclonal, chimeras, humanized, single chain, antibody fragments and the like. In addition, the invention includes the use of protein scaffolds for generating antibody mimics to obtain proteins that can be characterized by binding assays described herein and The invention further includes using methods set forth here to identify antibodies that recognize specific epitopes, within the MUC1* extra cellular domain, that are differentially expressed on cancer cells.

In one aspect, the present invention is directed to a human or humanized anti-MUC1* antibody or antibody fragment or antibody-like protein that binds to a region on extracellular domain of MUC1 isoform or cleavage product that is devoid of the tandem repeat domains. The human or humanized anti-MUC1* antibody or antibody fragment or antibody-like protein may specifically bind to
(i) PSMGFR region of MUC1;
(ii) PSMGFR peptide as set forth in SEQ ID NO:4;
(iii) PSMGFR N+20/C-22, a peptide having amino acid sequence of SNIKFRPGSVVVQLTLAFREGTINVHDVETQFNQYKTEAASRY (SEQ ID NO:5);
(iv) PSMGFR N+12/C-22, a peptide having amino acid sequence of SVVVQLTLAFREGTINVHDVETQFNQYKTEAASRY (SEQ ID NO:6);
(v) PSMGFR N+9/C-30, a peptide having amino acid sequence of VQLTLAFREGTINVHDVETQFNQY (SEQ ID NO:7);
(vi) PSMGFR N+20/C-41, a peptide having amino acid sequence of SNIKFRPGSVVVQLTLAFREGTIN (SEQ ID NO:8)
(vii) PSMGFR N+20/C-27, a peptide having amino acid sequence of SNIKFRPGSVVVQLTLAFREGTINVHDVETQFNQYKTE (SEQ ID NO:9); or
(viii) PSMGFR N+9/C-9, a peptide having amino acid sequence of VQLTLAFREGTINVHDVETQFNQYKTEAASRYNLTISDVSVSDVP (SEQ ID NO:10).

The human or humanized antibody may be IgG1, IgG2, IgG3, IgG4 or IgM. The human or humanized antibody fragment or antibody-like protein may be scFv or scFv-Fc.

The human or humanized antibody, antibody fragment or antibody-like protein as in above may comprise a heavy chain variable region and light chain variable region which is derived from mouse monoclonal MN-E6 antibody, and has at least 80%, 90% or 95% or 98% sequence identity to the mouse monoclonal MN-E6 antibody.

The human or humanized antibody, antibody fragment or antibody-like protein according to above may include complementarity determining regions (CDRs) in the heavy chain variable region and light chain variable region having at least 90% or 95% or 98% sequence identity to CDR1, CDR2 or CDR3 regions of the antibodies 1E4, 29H1, 31A1, 32C1, and 45C11 reactive with PSMGFR N+20/C-27; 17H6, 39H5, 3C5, 8A9 reactive with PSMGFR N+9/C-9; 18G12, 20A10, 25E6, 28F9, 18B4, MNC2, and MNE6 reactive with PSMGFR.

In another aspect, the invention is directed to a human or humanized anti-MUC1* antibody or antibody fragment or antibody-like protein according to above, which inhibits the binding of NME protein to MUC1*. The NME may be NME1, NME6, NME7AB, NME7 or NME8.

In still another aspect, the invention is directed to a chimeric antigen receptor (CAR) comprising a scFv or a humanized variable region that binds to the extracellular domain of a MUC1 that is devoid of tandem repeats, a linker molecule, a transmembrane domain and a cytoplasmic domain. The single chain antibody fragment may bind to
(i) PSMGFR region of MUC1;
(ii) PSMGFR peptide as set forth in SEQ ID NO:4;
(iii) PSMGFR N+20/C-22, a peptide having amino acid sequence of SNIKFRPGSVVVQLTLAFREGTINVHDVETQFNQYKTEAASRY (SEQ ID NO:5);
(iv) PSMGFR N+12/C-22, a peptide having amino acid sequence of SVVVQLTLAFREGTINVHDVETQFNQYKTEAASRY (SEQ ID NO:6);
(v) PSMGFR N+9/C-30, a peptide having amino acid sequence of VQLTLAFREGTINVHDVETQFNQY (SEQ ID NO:7);
(vi) PSMGFR N+20/C-41, a peptide having amino acid sequence of SNIKFRPGSVVVQLTLAFREGTIN (SEQ ID NO:8)
(vii) PSMGFR N+20/C-27, a peptide having amino acid sequence of SNIKFRPGSVVVQLTLAFREGTINVHDVETQFNQYKTE (SEQ ID NO:9); or
(viii) PSMGFR N+9/C-9, a peptide having amino acid sequence of VQLTLAFREGTINVHDVETQFNQYKTEAASRYNLTISDVSVSDVP (SEQ ID NO:10).

In this regard, a preferred embodiment is huMNC2-CAR44 set forth in SEQ ID NO: 236)

In one aspect, the invention is directed to a method for the treatment of a person diagnosed with, suspected of having or at risk of developing a MUC1 or MUC1* positive cancer involving administering to the person an effective amount of a cancer specific antibody such as MNC2 or MNE6, or fragment thereof, wherein the antibody may be human, humanized or of a non-human species. In a particular aspect of the invention, the MUC1* targeting therapeutic is an immune cell transduced with a chimeric antigen receptor, also known as CAR T, wherein the antibody fragment of the CAR is derived from a MUC1* cancer cell specific antibody. In one aspect it is derived from MNC2. In another case it is derived from MNE6.

In another aspect, the invention is directed to a diagnostic assay for the identification of persons who might benefit from treatment of a MUC1 or MUC1* positive cancer with a therapeutic that includes an antibody, or fragment thereof, selected from the group of 1E4, 29H1, 31A1, 32C1, 45C11, 17H6, 39H5, 3C5, 8A9, 18G12, 20A10, 25E6, 28F9, 18B4, MNC2, and MNE6 antibodies. In one aspect of the invention, the anti-MUC1* antibody or a fragment thereof comprises all or part of the therapeutic and may be derived from the antibody or fragment thereof that is used for the diagnostic, wherein the therapeutic and diagnostic need not be the same species. In another instance, the anti-MUC1* antibody or fragment thereof that comprises all or part of the therapeutic is not derived from the antibody or fragment thereof that is used for the diagnostic, wherein the therapeutic and diagnostic need not be the same species.

In one aspect of the invention, the therapeutic agent targets MUC1*. In another aspect of the invention, the therapeutic that comprises some or all of an anti-MUC1* antibody is a cancer immunotherapy composition, a CAR T, a BiTE, an antibody or an antibody drug conjugate, ADC.

In one aspect of the invention, the diagnostic is a companion diagnostic to determine eligibility for treatment with the therapeutic. In another aspect of the invention, the diagnostic is used to assess efficacy of the therapeutic treatment. In yet another aspect of the invention, the diagnostic together with results of clinical trials of the therapeutic are analyzed such that results of the diagnostic can be used to predict which patients will benefit from the treatment. In another aspect of the invention, the cancer cell antibody or fragment thereof is derivatized with an imaging agent, which composition is then administered to the patient to enable visualization of reactive tumors within the patient. In this way, the antibody plus imaging agent can be used to diagnose cancer, assess response of a therapeutic treatment or assess response to a therapeutic treatment wherein the therapeutic targets MUC1* and may comprise some or all of the cancer cell antibody used in the diagnostic. In one aspect of the invention, the antibody attached to the imaging agent is a camelid antibody, including but not limited to llama, alpaca, and camel.

The diagnostic assays described here can be used on samples that may be tissues, biopsy specimens, cells, or bodily fluids taken from the test subject, patient or a normal person as a control. The diagnostic assays can be performed in vitro or in vivo. The diagnostic assays can be used intraoperatively (e.g. tissue at a surgical site can be studied without removal of the tissue from the subject). In this way, the diagnostic assay guides the surgeon to remove all the MUC1* positive tissues that are detectable, whether or not the tissues appear to be part of the tumor. In either of these studies, a primary indicator of tumorigenesis or potential for tumorigenesis is the amount of MUC1* at a cell or tissue surface that is accessible to anti-PSMGFR antibodies or cancer cell antibodies. By extension, an exposed cancer cell antibody binding epitope means that the PSMGFR region of MUC1* is also accessible to growth factors that bind to and activate growth and survival functions mediated by the MUC1* growth factor receptor. In another technique, antibodies to the MUC1* region and to the tandem repeats, IBR or UR can be exposed to the sample and a determination made of the ratio of binding of MUC1* to MUC1 full-length. A healthy sample will exhibit little or no antibody binding to the MUC1* region. A sample indicating tumorigenesis will show a non-zero ratio of anti-MUC1* antibody to anti-tandem repeat antibody or anti-IBR antibody, wherein as cancer stage/grade increases, the ratio of MUC1* to MUC1 containing tandem repeats, IBR or UR increases.

In addition to detecting an amount of MUC1* or tandem repeat containing MUC1 on cells and tissues, portions of MUC1 that contain tandem repeats, which are shed from the tissues can be detected in bodily fluids such as blood, breast milk or secretions, urine, lung efflux and the like. In these cases, a level of MUC1 cleavage to transmembrane MUC1* is inferred by measuring an amount of shed MUC1 using antibodies, including but not limited to antibodies that bind to the tandem repeats, unique regions that are N-terminal to an IBR or the IBR itself.

Measuring or inferring an amount of MUC1* on cells or tissues, that is greater than normal tissues or a prior sample from the patient, is an indicator of potential for tumor formation, existence of a tumor, or progression of a tumor, and can thereby serve as a diagnostic and/or an evaluator of the efficacy of a treatment for the patient's cancer. In one aspect, an amount of MUC1* is measured by contacting a tissue specimen with an anti-MUC1* antibody and determining that the amount of MUC1* is greater than the amount expressed on normal tissues or in a healthy person.

Sequence Listing Free Text
In the antibody sequences below, underlined sequence refers to
CDR sequence and double underlined region refers to framework region.
Full-length MUC1 Receptor (Mucin 1 precursor, Genbank Accession
number: P15941
(SEQ ID NO: 1)
MTPGTQSPFF LLLLLTVLTV VTGSGHASST PGGEKETSAT QRSSVPSSTE KNAVSMTSSV
LSSHSPGSGS STTQGQDVTL APATEPASGS AATWGQDVTS VPVTRPALGS TTPPAHDVTS
APDNKPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS TAPPAHGVTS
APDTRPAPGS TAPPAHGVTS APDTRPAPGS TAPPAHGVTS APDNRPALGS TAPPVHNVTS
ASGSASGSAS TLVHNGTSAR ATTTPASKST PESIPSHHSD TPTTLASHST KTDASSTHHS
SVPPLTSSNH STSPQLSTGV SFFFLSFHIS
NLQFNSSLED PSTDYYQELQ RDISEMFLQI YKQGGFLGLS NIKFRPGSVV VQLTLAFREG
TINVHDVETQ FNQYKTEAAS RYNLTISDVS VSDVPFPFSA QSGAGVPGWG IALLVLVCVL
VALAIVYLIA LAVCQCRRKN YGQLDIFPAR DTYHPMSEYP TYHTHGRYVP PSSTDRSPYE
KVSAGNGGSS LSYTNPAVAA ASANL
A truncated MUC1 receptor isoform having nat-PSMGFR and PSIBR at its
N-terminus and including the transmembrane and cytoplasmic sequences
of a full-length MUC1 receptor which may be cleaved after translation
and prior to expression of the receptor on the cell surface:
(SEQ ID NO: 2)
GFLGLS NIKFRPGSVV VQLTLAFREG TINVHDVETQ FNQYKTEAAS RYNLTISDVS
VSDVPFPFSA QSGAGVPGWG IALLVLVCVL VALAIVYLIA LAVCQCRRKN YGQLDIFPAR
DTYHPMSEYP TYHTHGRYVP PSSTDRSPYE KVSAGNGGSS LSYTNPAVAA ASANL
A truncated MUC1 receptor isoform having nat-PSMGFR + PSIBR + Unique
Region at its N-terminus and including the transmembrane and
cytoplasmic sequences of a full-length MUC1 receptor:
(SEQ ID NO: 3)
ATTTPASKSTPFSIPSHHSDTPTTLASHSTKTDASSTHHSTVPPLTSSNHSTSPQLSTGVSFFFLSFHIS
NLQFNSSLEDPSTDYYQELQRDISEMFLQIYKQGGFLGLSNIKERPGSVVVQLTLAFREGTINVHDVETQ
FNQYKTEAASRYNLTISDVSVSDVPFPFSAQSGAGVPGWGIALLVLVCVLVALAIVYLIALAVCQCRRKN
YGQLDIFPARDTYHPMSEYPTYHTHGRYVPPSSTDRSPYEKVSAGNGGSSLSYTNPAVAAASANL
PSMGFR
(SEQ ID NO: 4)
GTINVHDVETQFNQYKTEAASRYNLTISDVSVSDVPFPFSAQSGA
PSMGFR N + 20/C − 22
(SEQ ID NO: 5)
SNIKFRPGSVVVQLTLAFREGT INVHDVETQFNQYKTEAASRY
PSMGFR N + 12/C − 22
(SEQ ID NO: 6)
SVVVQLTLAFREGTINVHDVETQFNQYKTEAASRY
PSMGFR N + 9/C − 30
(SEQ ID NO: 7)
VQLTLAFREGTINVHDVETQFNQY
PSMGFR N + 20/C − 41
(SEQ ID NO: 8)
SNIKFRPGSVVVQLTLAFREGTIN
PSMGFR N + 20/C − 27
(SEQ ID NO: 9)
SNIKFRPGSVVVQLTLAFREGTINVHDVETQFNQYKTE
PSMGFR N + 9/C-9
(SEQ ID NO: 10)
VQLTLAFREGTINVHDVETQFNQYKTEAASRYNLTISDVSVSDVP
Antibody 17H6 Heavy chain-Signal sequence-FR1-CDR1-FR2-CDR2-FR3-
CDR3-FR4
(SEQ ID NO: 11)
ATGAAGTTGTGGCTGAACTGGATTTTCCTTGTAACACTTTTAAATGGTATCCAGTGTGAGGTGAAGCTGG
TGGAGTCTGGAGGAGGCTTGGTACAGCCTGGGGGTTCTCTGAGACTCTCCTGTGCAACTTCTGGGTTCAC
CTTCACTGATTACTACATGAGCTGGGTCCGCCAGCCTCCAAGAAAGGCACTTGAGTGGTTGGGTTTTATT
AGAAACAAAGCTAATGGTTACACAGCAGAGTACAGTGCGTCTGTGAAGGGTCGGTTCACCATCTCCAGAG
ATGTTTCCCAAAACCTCCTCTATCTTCAAATGAACATCCTGAGAGCTGAGGACAGTGCCACTTATTACTG
TGCAAAAGATTACTACGGTAGTAACCCTGCCTGGTTTGCTTACTGGGGCCAAGGGACTCTGGTCACTGTC
TCTGCA
Antibody 17H6 Heavy chain-Signal peptide-FR1-CDR1-FR2-CDR2-FR3-CDR3-
FR4
(SEQ ID NO: 12)
MKLWLNWIFLVTLLNGIQCEVKLVESGGGLVQPGGSLRLSCATSGFTFTDYYMSWVRQPPRKALEWLGFI
RNKANGYTAEYSASVKGRETISRDVSQNLLYLQMNILRAEDSATYYCAKDYYGSNPAWFAYWGQGTLVTV
SA
Antibody 17H6 Light chain-Signal sequence-FR1-CDR1-FR2-CDR2-FR3-
CDR3-FR4
(SEQ ID NO: 13)
ATGAAGTTGCCTGTGAGGCTGTTGGTGCTGATGTTCTGGATTCCTGCTTCCAACAGTGATATTTTGATGA
CCCAGACTCCACTCTCCCTGCCTGTCAGTCTTGGAGATCAAGCCTCCATCTCTTGCAGATCTAGTCAGAG
CATTGTACATAGTAGTGGAAACACCTTTTTAGAATGGTACCTGCAGAAACCTGGCCAGTCTCCAAAGCTC
CTGATCTACAAAGTTTCCAACCGATTTTCTGGGGTCCCAGACAGGTTCAGTGGCAGTGGATCAGGGATAG
ATTTCACACTCAAGATCAGCAGAGTGGAGGCTGAGGATCTGGGAGTTTATTACTGCTTTCAAGGTTCACA
TGTTCCTTTCACGTTCGGCTCGGGGACAAAGTTGGAAATAAAA
Antibody 17H6 Light chain-Signal peptide-FR1-CDR1-FR2-CDR2-FR3-CDR3-
FR4
(SEQ ID NO: 14)
MKLPVRLLVLMFWIPASNSDILMTQTPLSLPVSLGDQASISCRSSQSIVHSSGNTFLEWYLQKPGQSPKL
LIYKVSNRFSGVPDRESGSGSGIDFTLKISRVEAEDLGVYYCFQGSHVPFTFGSGTKLEIK
Antibody 17H6 Heavy Chain CDR1
(SEQ ID NO: 15)
GATTACTACATGAGC
Antibody 17H6 Heavy Chain CDR1
(SEQ ID NO: 16)
DYYMS
Antibody 17H6 Heavy Chain CDR2
(SEQ ID NO: 17)
TTTATTAGAAACAAAGCTAATGGTTACACAGCAGAGTACAGTGCGTCTGTGAAGGGT
Antibody 17H6 Heavy Chain CDR2
(SEQ ID NO: 18)
FIRNKANGYTAEYSASVKG
Antibody 17H6 Heavy Chain CDR3
(SEQ ID NO: 19)
GATTACTACGGTAGTAACCCTGCCTGGTTTGCTTAC
Antibody 17H6 Heavy Chain CDR3
(SEQ ID NO: 20)
DYYGSNPAWFAY
Antibody 17H6 Light Chain CDR1
(SEQ ID NO: 21)
AGATCTAGTCAGAGCATTGTACATAGTAGTGGAAACACCTTTTTAGAA
Antibody 17H6 Light Chain CDR1
(SEQ ID NO: 22)
RSSQSIVHSSGNTFLE
Antibody 17H6 Light Chain CDR2
(SEQ ID NO: 23)
AAAGTTTCCAACCGATTTTCT
Antibody 17H6 Light Chain CDR2
(SEQ ID NO: 24)
KVSNRFS
Antibody 17H6 Light Chain CDR3
(SEQ ID NO: 25)
TTTCAAGGTTCACATGTTCCTTTCACG
Antibody 17H6 Light Chain CDR3
(SEQ. ID. NO: 26)
FQGSHVPFT
Antibody 39H5 Heavy chain-Signal sequence-FR1-CDR1-FR2-CDR2-FR3-
CDR3-FR4
(SEQ ID NO: 27)
ATGGCTTGGGTGTGGACCTTGCTATTCCTGATGGCAGCTGCCCAAAGTGCCCAAGCACAGATCCAGTTGG
TGCAGTCTGGACCTGAGCTGAAGAAGCCTGGAGAGACAGTCAAGATCTCCTGCAAGGCTTCTGGGTATAC
CTTCACAAACTATGGAATGAACTGGGTGAAGCAGGCTCCAGGAAAGGGTTTAAAGTGGATGGGCTGGATA
AACACCTACACTGGAGAGCCAACATATGTTGGTGACTTCAAGGGACGGTTTGCCTTCTCTTTGGAGACCT
CTGCCAGCACTGCCTATTTGCAGATCAACAACCTCAAAAATGAGGACACGGCTACATATTTTTGTGTTAG
AGGTATCCACGGCTACGTGGACTACTGGGGCCAAGGCACCACTCTCACAGTCTCCTCA
Antibody 39H5 Heavy chain-Signal sequence-FR1-CDR1-FR2-CDR2-FR3-
CDR3-FR4
(SEQ. ID. NO: 28)
MAWVWTLLFLMAAAQSAQAQIQLVQSGPELKKPGETVKISCKASGYTFTNYGMNWVKQAPGKGLKWMGWI
NTYTGEPTYVGDFKGRFAFSLETSASTAYLQINNLKNEDTATYFCVRGIHGYVDYWGQGTTLTVSS
Antibody 39H5 Light chain-Signal sequence-FR1-CDR1-FR2-CDR2-FR3-
CDR3-FR4
(SEQ ID NO: 29)
ATGAAGTTGCCTGTTAGGCTGTTGGTGCTGATGTTCTGGATTCCTGCTTCCAGCAGTGATGTTTTGATGA
CCCAAACTCCACTCTCCCTGCCTGTCAGTCTTGGAGATCAAGCCTCCATCTCTTGCAGATCTAGTCAGAG
CATTGTACATAGAAATGGAAACACCTATTTAGAATGGTACCTGCAGAAACCAGGCCAGTCTCCAAAGCTC
CTGATCTACAAAGTTTCCAACCGATTTTCTGGGGTCCCAGACAGGTTCAGTGGCAGTGGATCAGGGACAG
ATTTCACACTCAAGATCAGCAGAGTGGAGGCTGAGGATCTGGGAGTTTATTACTGCTTTCAAGGTTCACA
TCTTCCGTGGACGTTCGGTGGAGGCACCAAGCTGGAAATCAAA
Antibody 39H5 Light chain-Signal sequence-FR1-CDR1-FR2-CDR2-FR3-
CDR3-FR4
(SEQ ID NO: 30)
MKLPVRLLVLMFWIPASSSDVLMTQTPLSLPVSLGDQASISCRSSQSIVHRNGNTYLEWYLQKPGQSPKL
LIYKVSNRFSGVPDRESGSGSGTDFTLKISRVEAEDLGVYYCFQGSHLPWTFGGGTKLEIK
Antibody 39H5 Heavy Chain CDR1
(SE. ID NO: 31)
AACTATGGAATGAAC
Antibody 39H5 Heavy Chain CDR1
(SEQ ID NO: 32)
NYGMN
Antibody 39H5 Heavy Chain CDR2
(SEQ ID NO: 33)
TGGATAAACACCTACACTGGAGAGCCAACATATGTTGGTGACTTCAAGGGA
Antibody 39H5 Heavy Chain CDR2
(SEQ ID NO: 34)
WINTYTGEPTYVGDFKG
Antibody 39H5 Heavy Chain CDR3
(SEQ ID NO: 35)
GGTATCCACGGCTACGTGGACTAC
Antibody 39H5 Heavy Chain CDR3
(SEQ ID NO: 36)
GIHGYVDY
Antibody 39H5 Light Chain CDR1
(SEQ ID NO: 37)
AGATCTAGTCAGAGCATTGTACATAGAAATGGAAACACCTATTTAGAA
Antibody 39H5 Light Chain CDR1
(SEQ ID NO: 38)
RSSQSIVHRNGNTYLE
Antibody 39H5 Light Chain CDR2
(SEQ ID NO: 39)
AAAGTTTCCAACCGATTTTCT
Antibody 39H5 Light Chain CDR2
(SEQ ID NO: 40)
KVSNRFS
Antibody 39H5 Light Chain CDR3
(SEQ ID NO: 41)
TTTCAAGGTTCACATCTTCCGTGGACG
Antibody 39H5 Light Chain CDR3
(SEQ ID NO: 42)
FQGSHLPWT
Antibody 3C5 Heavy chain-Signal sequence-FR1-CDR1-FR2-CDR2-FR3-CDR3-
FR4
(SEQ ID NO: 43)
ATGGCTTGGGTGTGGACCTTGCTGTTCCTGATGGCAGCTGCCCAAAGTGCCCAAGCACAGATCCAGTTGG
TGCAGTCTGGACCTGAGCTGAAGAAGCCTGGAGAGACAGTCAAGATCTCCTGCAAGGCTTCTGGGTATAC
CTTCACAAACTATGGAATGAACTGGGTGAAGCAGGCTCCAGGAAAGGGTTTAAAGTGGATGGGCTGGATA
AACACCTACACTGGAAAGCCAACATATGCTGATGACTTCAAGGGACGGTTTGCCTTCTCTTTGGAGACCT
CTGCCAGCACTGCCTATTTGCAGATCAACAACCTCAAAAATGAGGACACGGCTACATATTTCTGTGCAAG
AGGGGGACTAGATGGTTACTACGGCTACTGGGGCCAAGGCACCACTCTCACAGTCTCCTCA
Antibody 3C5 Heavy chain-Signal sequence-FR1-CDR1-FR2-CDR2-FR3-CDR3-
FR4
(SEQ ID NO: 44)
MAWVWTLLFLMAAAQSAQAQIQLVQSGPELKKPGETVKISCKASGYTFTNYGMNWVKQAPGKGLKWMGWI
NTYTGKPTYADDFKGRFAFSLETSASTAYLQINNLKNEDTATYFCARGGLDGYYGYWGQGTTLTVSS
Antibody 3C5 Light chain-Signal sequence-FR1-CDR1-FR2-CDR2-FR3-CDR3-
FR4
(SEQ ID NO: 45)
ATGAGTCCTGCCCAGTTCCTGTTTCTGCTAGTGCTCTCGATTCAGGAAACCAACGGTGATGTTGTGATGG
CTCAGACCCCACTCACTTTGTCGGTTACCATTGGACAACCAGCCTCCATCTCTTGCAAATCAAGTCAGAG
CCTCTTACATAGTAAAGGAAAGACATATTTGAATTGGTTATTACAGAGGCCAGGCCAGTCTCCAAAGCTC
CTAATCTATCTGGTGTCTAAACTGGAATCTGGAGTCCCTGACAGGTTCAGTGGCAGTGGATCAGGGACAG
ATTTCACACTGAAAATCAGCAGAGTGGAGGCTGAAGATTTGGGAGTTTATTACTGCTTGCAAACTACACA
TTTTCCGTGGACGTTCGGTGGAGGCACCAAGCTGGAAATCAAA
Antibody 3C5 Light chain-Signal sequence-FR1-CDR1-FR2-CDR2-FR3-CDR3-
FR4
(SEQ ID NO: 46)
MSPAQFLFLLVLSIQETNGDVVMAQTPLTLSVTIGQPASISCKSSQSLLHSKGKTYLNWLLQRPGQSPKL
LIYLVSKLESGVPDRESGSGSGTDETLKISRVEAEDLGVYYCLQTTHEPWTFGGGTKLEIK
Antibody 3C5 Heavy Chain CDR1
(SEQ ID NO: 47)
AACTATGGAATGAAC
Antibody 3C5 Heavy Chain CDR1
(SEQ ID NO: 48)
NYGMN
Antibody 3C5 Heavy Chain CDR2
(SEQ ID NO: 49)
TGGATAAACACCTACACTGGAAAGCCAACATATGCTGATGACTTCAAGGGA
Antibody 3C5 Heavy Chain CDR2
(SEQ ID NO: 50)
WINTYTGKPTYADDEKG
Antibody 3C5 Heavy Chain CDR3
(SEQ ID NO: 51)
GGGGGACTAGATGGTTACTACGGCTAC
Antibody 3C5 Heavy Chain CDR3
(SEQ ID NO: 52)
GGLDGYYGY
Antibody 3C5 Light Chain CDR1
(SEQ ID NO: 53)
AAATCAAGTCAGAGCCTCTTACATAGTAAAGGAAAGACATATTTGAAT
Antibody 3C5 Light Chain CDR1
(SEQ ID NO: 54)
KSSQSLLHSKGKTYLN
Antibody 3C5 Light Chain CDR2
(SEQ ID NO: 55)
CTGGTGTCTAAACTGGAATCT
Antibody 3C5 Light Chain CDR2
(SEQ ID NO: 56)
LVSKLES
Antibody 3C5 Light Chain CDR3
(SEQ ID NO: 57)
TTGCAAACTACACATTTTCCGTGGACG
Antibody 3C5 Light Chain CDR3
(SEQ ID NO: 58)
LQTTHEPWT
Antibody 8A9 Heavy chain-Signal sequence-FR1-CDR1-FR2-CDR2-FR3-CDR3-
FR4
(SEQ ID NO: 59)
ATGAAGTTGTGGCTGAACTGGATTTTCCTTGTAACACTTTTAAATGGTATCCAGTGTGAGGTGGAGCTGG
TGGAGTCTGGAGGAGGCTTGGTACAGCCTGGGGGTTCTCTGAGACTCTCCTGTGCAACTTCTGGGTTCAC
CTTCACTGATCACTACATGAGCTGGGTCCGCCAGCCTCCAGGAAAGGCACTTGAGTGGTTGGGATTTATT
AGAAACAAAGCTAATGGTTACACAACAGAGTACAGTGCATCTGTGAAGGGTCGGTTCACCATCTCCAGAG
ATAATTCCCAAAGCATCCTCTATCTTCAAATGAAAACCCTGAGAACTGAGGACAGTGCCACTTATTACTG
TGCAAGACCTTCTGACTGGGACTCCTGGTTTGCTTACTGGGGCCAAGGGACTCTGGTCACTGTCTCTGCA
Antibody 8A9 Heavy chain-Signal sequence-FR1-CDR1-FR2-CDR2-FR3-CDR3-
FR4
(SEQ ID NO: 60)
MKLWLNWIFLVTLLNGIQCEVELVESGGGLVQPGGSLRLSCATSGFTFTDHYMSWVRQPPGKALEWLGFI
RNKANGYTTEYSASVKGRFTISRDNSQSILYLQMKTLRTEDSATYYCARPSDWDSWFAYWGQGTLVTVSA
Antibody 8A9 Light chain-Signal sequence-FR1-CDR1-FR2-CDR2-FR3-CDR3-
FR4
(SEQ ID NO: 61)
ATGAAGTTGCCTGTTAGGCTGTTGGTGCTGATGTTCTGGATTCCTGCTTCCAGCAGTGATGTTTTGATGA
CCCAAACTCCACTCTCCCTGCCTGTCAGTCTTGGTGATCAAGCCTCCATCTCTTGCAGATCTAGTCAGAG
CATTGTACATAGTAATGGCAACACCTATTTAGATTGGTACTTGCAGAAACCAGGCCAGTCTCCAAAGCTC
CTGATCTACAGAGTTTCCAACCGATTTTCTGGGGTCCCAGACAGGTTCAGTGGCAGTGGATCAGGGACAG
ATTTCACACTCAAGATCAGCAGAGTGGAGGCTGAGGATCTGGGACTTTATTACTGTTTTCAAGGTTCACA
TGTTCCGTGGGCGTTCGGTGGAGGCACCAAGCTGGAAATCAAA
Antibody 8A9 Light chain-Signal sequence-FR1-CDR1-FR2-CDR2-FR3-CDR3-
FR4
(SEQ ID NO: 62)
MKLPVRLLVLMFWIPASSSDVLMTQTPLSLPVSLGDQASISCRSSQSIVHSNGNTYLDWYLQKPGQSPKL
LIYRVSNRFSGVPDRESGSGSGTDETLKISRVEAEDLGLYYCFQGSHVPWAFGGGTKLEIK
Antibody 8A9 Heavy Chain CDR1
(SEQ ID NO: 63)
GATCACTACATGAGC
Antibody 8A9 Heavy Chain CDR1
(SEQ ID NO: 64)
DHYMS
Antibody 8A9 Heavy Chain CDR2
(SEQ ID NO: 65)
TTTATTAGAAACAAAGCTAATGGTTACACAACAGAGTACAGTGCATCTGTGAAGGGT
Antibody 8A9 Heavy Chain CDR2
(SEQ ID NO: 66)
FIRNKANGYTTEYSASVKG
Antibody 8A9 Heavy Chain CDR3
(SEQ ID NO: 67)
CCTTCTGACTGGGACTCCTGGTTTGCTTAC
Antibody 8A9 Heavy Chain CDR3
(SEQ ID NO: 68)
PSDWDSWFAY
Antibody 8A9 Light Chain CDR1
(SEQ ID NO: 69)
AGATCTAGTCAGAGCATTGTACATAGTAATGGCAACACCTATTTAGAT
Antibody 8A9 Light Chain CDR1
(SEQ ID NO: 70)
RSSQSIVHSNGNTYLD
Antibody 8A9 Light Chain CDR2
(SEQ ID NO: 71)
AGAGTTTCCAACCGATTTTCT
Antibody 8A9 Light Chain CDR2
(SEQ ID NO: 72)
RVSNRFS
Antibody 8A9 Light Chain CDR3
(SEQ ID NO: 73)
TTTCAAGGTTCACATGTTCCGTGGGCG
Antibody 8A9 Light Chain CDR3
(SEQ ID NO: 74)
FQGSHVPWA
Antibody 18G12 Heavy chain-Signal sequence-FR1-CDR1-FR2-CDR2-FR3-
CDR3-FR4
(SEQ ID NO: 75)
ATGGGATGGAGCTATATCATCCTCTTTTTGGTCGCAACAGCTACAGGTGTCCACTCCCAGGTCCAACTGC
AGCAGTCTGGGGCTGAACTGGTGAAGCCTGGGGCTTCAGTGAAGTTGTCCTGCAAGGCTTCTGGCTACAC
CTTCACCGGCTACTTTTTGTACTGGGTGAAGCAGAGGCCTGGACAAGGCCTTGAGTGGATTGGGGGGATT
AATCCTGACAATGGTGGTATTGACTTCAATGAGAAGTTCAGGAACAAGGCCACACTGACTGTAGACAAAT
CCTCCAGCACAGCCTACATGCAACTCAGCAGCCTGACATCTGAGGACTCTGCGGTCTATTATTGTACATT
ACTAATAGGGAACTATTGGGGCCAAGGCACCACTCTCACAGTCTCCTCA
Antibody 18G12 Heavy chain-Signal sequence-FR1-CDR1-FR2-CDR2-FR3-
CDR3-FR4
(SEQ ID NO: 76)
MGWSYIILFLVATATGVHSQVQLQQSGAELVKPGASVKLSCKASGYTFTGYFLYWVKQRPGQGLEWIGGI
NPDNGGIDENEKFRNKATLTVDKSSSTAYMQLSSLTSEDSAVYYCTLLIGNYWGQGTTLTVSS
Antibody 18G12 Light chain-Signal sequence-FR1-CDR1-FR2-CDR2-FR3-
CDR3-FR4
(SEQ ID NO: 77)
ATGAGTCCTGCCCAGTTCCTGTTTCTGTTAGTGCTCTGGATTCGGGAAACCAATGGTGATGTTGTGATGA
CCCAGACTCCACTCACTTTGTCGGTAACCATTGGACAGCCAGCCTCCATCTCTTGCAAGTCAAGTCAGAG
CCTCTTACATAGTGATGGAAAGACATATTTGATTTGGTTGTTACAGAGGCCAGGCCAGTCTCCAAAGCGC
CTAATCTATCTGGTGTCTAAACTGGACTCTGGAGTCCCTGACAGGTTCACTGGCAGTGGATCAGGGACAG
ATTTCACACTGAAAATCAGCAGAGTGGAGGCTGAGGATTTGGGAGTTTATTTTTGCTGTCAAGGTACACA
TTTTCCGTGGACGTTCGGTGGAGGCACCATGCTGGAAATCAAA
Antibody 18G12 Light chain-Signal sequence-FR1-CDR1-FR2-CDR2-FR3-
CDR3-FR4
(SEQ ID NO: 78)
MSPAQFLFLLVLWIRETNGDVVMTQTPLTLSVTIGQPASISCKSSQSLLHSDGKTYLIWLLQRPGQSPKR
LIYLVSKLDSGVPDRFTGSGSGTDETLKISRVEAEDLGVYFCCQGTHFPWTFGGGTMLEIK
Antibody 18G12 Heavy Chain CDR1
(SEQ ID NO: 79)
GGCTACTTTTTGTAC
Antibody 18G12 Heavy Chain CDR1
(SEQ ID NO: 80)
GYFLY
Antibody 18G12 Heavy Chain CDR2
(SEQ ID NO: 81)
GGGATTAATCCTGACAATGGTGGTATTGACTTCAATGAGAAGTTCAGGAAC
Antibody 18G12 Heavy Chain CDR2
(SEQ ID NO: 82)
GINPDNGGIDENEKERN
Antibody 18G12 Heavy Chain CDR3
(SEQ ID NO: 83)
CTAATAGGGAACTAT
Antibody 18G12 Heavy Chain CDR3
(SEQ ID NO: 84)
LIGNY
Antibody 18G12 Light Chain CDR1
(SEQ ID NO: 85)
AAGTCAAGTCAGAGCCTCTTACATAGTGATGGAAAGACATATTTGATT
Antibody 18G12 Light Chain CDR1
(SEQ ID NO: 86)
KSSQSLLHSDGKTYLI
Antibody 18G12 Light Chain CDR2
(SEQ ID NO: 87)
CTGGTGTCTAAACTGGACTCT
Antibody 18G12 Light Chain CDR2
(SEQ ID NO: 88)
LVSKLDS
Antibody 18G12 Light Chain CDR3
(SEQ ID NO: 89)
TGTCAAGGTACACATTTTCCGTGGACG
Antibody 18G12 Light Chain CDR3
(SEQ ID NO: 90)
CQGTHFPWT
Antibody 20A10 Heavy chain-Signal sequence-FR1-CDR1-FR2-CDR2-FR3-
CDR3-FR4
(SEQ ID NO: 91)
ATGAACTTCGGGTTCAGCTTGATTTTCCTTGTCCTTGTTTTAAAAGGTGTCCAGTGTGAAGTGATGCTGG
TGGAGTCTGGGGGAGGCTTAGTGAAGCCTGGAGGGTCCCTGAAACTCTCCTGTGCAGCCTCTGGATTCAC
TTTCAGTACCTATGCCATGTCTTGGATTCGCCAGACTCCAGAGAAGAGGCTGGAGTGGGTCGCATCCATT
GGTCGTGCTGGTTCCACCTACTATTCAGACAGTGTGAAGGGCCGATTCACCATCTCCAGAGATAATGTCC
GGAACATCCTGTACCTGCAAATGAGCAGTCTGAGGTCTGAGGACACGGCCATGTATTACTGTGCTAGAGG
CCCGATCTACAATGATTACGACGAGTTTGCTTACTGGGGCCAAGGGACTCTGGTCACTGTCTCTGCA
Antibody 20A10 Heavy chain-Signal sequence-FR1-CDR1-FR2-CDR2-FR3-
CDR3-FR4
(SEQ ID NO: 92)
MNFGFSLIFLVLVLKGVQCEVMLVESGGGLVKPGGSLKLSCAASGFTFSTYAMSWIRQTPEKRLEWVASI
GRAGSTYYSDSVKGRFTISRDNVRNILYLQMSSLRSEDTAMYYCARGPIYNDYDEFAYWGQGTLVTVSA
Antibody 20A10 Light chain-Signal sequence-FR1-CDR1-FR2-CDR2-FR3-
CDR3-FR4
(SEQ ID NO: 93)
ATGGAATCACAGACTCAGGTCTTCCTCTCCCTGCTGCTCTGGGTATCTGGTACCTGTGGGAACATTATGA
TGACACAGTCGCCATCATCTCTGGCTGTGTCTGCAGGAGAAAAGGTCACTATGAGCTGTAAGTCCAGTCA
AAGTGTTTTATACAGTTCAAATCAGAAGAACTATTTGGCCTGGTACCAGCAGAAACCAGGGCAGTCTCCT
AAACTGCTGATCTACTGGGCATCCACTAGGGAATCTGGTGTCCCTGATCGCTTCACAGGCAGTGGATCTG
GGACAGATTTTACTCTTACCATCAGCAGTGTACAAGCTGAAGACCTGGCAGTTTATTACTGTCATCAATA
CCTCTCCTCGCTCACGTTCGGTGCTGGGACCAAGCTGGAGCTGAAA
Antibody 20A10 Light chain-Signal sequence-FR1-CDR1-FR2-CDR2-FR3-
CDR3-FR4
(SEQ ID NO: 94)
MESQTQVFLSLLLWVSGTCGNIMMTQSPSSLAVSAGEKVTMSCKSSQSVLYSSNQKNYLAWYQQKPGQSP
KLLIYWASTRESGVPDRETGSGSGTDETLTISSVQAEDLAVYYCHQYLSSLTFGAGTKLELK
Antibody 20A10 Heavy Chain CDR1
(SEQ ID NO: 95)
ACCTATGCCATGTCT
Antibody 20A10 Heavy Chain CDR1
(SEQ ID NO: 96)
TYAMS
Antibody 20A10 Heavy Chain CDR2
(SEQ ID NO: 97)
TCCATTGGTCGTGCTGGTTCCACCTACTATTCAGACAGTGTGAAGGGC
Antibody 20A10 Heavy Chain CDR2
(SEQ ID NO: 98)
SIGRAGSTYYSDSVKG
Antibody 20A10 Heavy Chain CDR3
(SEQ ID NO: 99)
GGCCCGATCTACAATGATTACGACGAGTTTGCTTAC
Antibody 20A10 Heavy Chain CDR3
(SEQ ID NO: 100)
GPIYNDYDEFAY
Antibody 20A10 Light Chain CDR1
(SEQ ID NO: 101)
AAGTCCAGTCAAAGTGTTTTATACAGTTCAAATCAGAAGAACTATTTGGCC
Antibody 20A10 Light Chain CDR1
(SEQ ID NO: 102)
KSSQSVLYSSNQKNYLA
Antibody 20A10 Light Chain CDR2
(SEQ ID NO: 103)
TGGGCATCCACTAGGGAATCT
Antibody 20A10 Light Chain CDR2
(SEQ ID NO: 104)
WASTRES
Antibody 20A10 Light Chain CDR3
(SEQ ID NO: 105)
CATCAATACCTCTCCTCGCTCACG
Antibody 20A10 Light Chain CDR3
(SEQ ID NO: 106)
HQYLSSLT
Antibody 25E6 Heavy chain-Signal sequence-FR1-CDR1-FR2-CDR2-FR3-
CDR3-FR4
(SEQ ID NO: 107)
ATGAACTTCGGGCTCAGCTTGATTTTCCTTGCCCTCATTTTAAAAGGTGTCCAGTGTGAGGTGCAGCTGG
TGGAGTCTGGGGGAGACTTAGTGAAGCCTGGAGGGTCCCTGAAACTCTCCTGTGCAGCCTCTGGTTTCAC
TTTCAGTAGTTATGGAATGTCTTGGGTTCGCCAGACTCCAGACAAGAGGCTGGAGTGGGTCGCAACCATT
AGTAATGGTGGTAGACACACCTTCTATCCAGACAGTGTGAAGGGGCGATTCACCATCTCCAGAGACAATG
CCAAGAACACCCTGTATCTGCAAATGAGCAGTCTGAAGTCTGAGGACACAGCCATGTATTTATGTGTAAG
ACAGACTGGGACGGAGGGCTGGTTTGCTTACTGGGGCCAAGGGACTCTGGTCACTGTCTCTGCA
Antibody 25E6 Heavy chain-Signal sequence-FR1-CDR1-FR2-CDR2-FR3-
CDR3-FR4
(SEQ ID NO: 108)
MNFGLSLIFLALILKGVQCEVQLVESGGDLVKPGGSLKLSCAASGFTFSSYGMSWVRQTPDKRLEWVATI
SNGGRHTFYPDSVKGRFTISRDNAKNTLYLQMSSLKSEDTAMYLCVRQTGTEGWFAYWGQGTLVTVSA
Antibody 25E6 Light chain-Signal sequence-FR1-CDR1-FR2-CDR2-FR3-
CDR3-FR4
(SEQ ID NO: 109)
ATGAGTCCTGCCCAGTTCCTGTTTCTGTTAGTGCTCTGGATTCGGGAAACCAACGGTGATGTTGTGATGA
CCCAGACTCCACTCACTTTGTCGGTTACCATTGGACAACCAGCCTCCATCTCTTGCAAGTCAAGTCAGAG
CCTCTTAGATAGTGATGGAAAGACATATTTGAATTGGTTGTTACAGAGGCCAGGCCAGTCTCCAAAGCGC
CTAATCTATCTGGTGTCTAAACTGGACTCTGGAGTCCCTGACAGGTTCACTGGCAGTGGATCAGGGACAG
ATTTCACACTGAAAATCAGCAGAGTGGAGGCTGAGGATTTGGGAGTTTATTATTGCTGGCAAGGTACACA
TTTTCCTCAGACGTTCGGTGGAGGCACCAAGCTGGAAATCAAA
Antibody 25E6 Light chain-Signal sequence-FR1-CDR1-FR2-CDR2-FR3-
CDR3-FR4
(SEQ ID NO: 110)
MSPAQFLFLLVLWIRETNGDVVMTQTPLTLSVTIGQPASISCKSSQSLLDSDGKTYLNWLLQRPGQSPKR
LIYLVSKLDSGVPDRFTGSGSGTDFTLKISRVEAEDLGVYYCWQGTHEPQTFGGGTKLEIK
Antibody 25E6 Heavy Chain CDR1
(SEQ ID NO: 111)
AGTTATGGAATGTCT
Antibody 25E6 Heavy Chain CDR1
(SEQ ID NO: 112)
SYGMS
Antibody 25E6 Heavy Chain CDR2
(SEQ ID NO: 113)
ACCATTAGTAATGGTGGTAGACACACCTTCTATCCAGACAGTGTGAAGGGG
Antibody 25E6 Heavy Chain CDR2
(SEQ ID NO: 114)
TISNGGRHTFYPDSVKG
Antibody 25E6 Heavy Chain CDR3
(SEQ ID NO: 115)
CAGACTGGGACGGAGGGCTGGTTTGCTTAC
Antibody 25E6 Heavy Chain CDR3
(SEQ ID NO: 116)
QTGTEGWFAY
Antibody 25E6 Light Chain CDR1
(SEQ ID NO: 117)
AAGTCAAGTCAGAGCCTCTTAGATAGTGATGGAAAGACATATTTGAAT
Antibody 25E6 Light Chain CDR1
(SEQ ID NO: 118)
KSSQSLLDSDGKTYLN
Antibody 25E6 Light Chain CDR2
(SEQ ID NO: 119)
CTGGTGTCTAAACTGGACTCT
Antibody 25E6 Light Chain CDR2
(SEQ ID NO: 120)
LVSKLDS
Antibody 25E6 Light Chain CDR3
(SEQ ID NO: 121)
TGGCAAGGTACACATTTTCCTCAGACG
Antibody 25E6 Light Chain CDR3
(SEQ ID NO: 122)
WQGTHEPQT
Antibody 28F9 Heavy chain-Signal sequence-FR1-CDR1-FR2-CDR2-FR3-
CDR3-FR4
(SEQ ID NO: 123)
ATGGGATGGAGCTATATCATCCTCTTTTTGGTAGCAACAGCTACAGGTGTCCACTCCCAGGTCCAACTGC
AGCAGCCTGGGGCTGAACTGGTGCAGCCTGGGGCTTCAGTGAAGTTGTCCTGCAAGGCTTCTGGCTACAC
CTTCACCGGCTACTTTTTGTACTGGGTGAAGCAGAGGCCTGGACATGGCCTTGAGTGGATTGGGGGAATT
CATCCTAGCAATGGTGATACTGACTTCAATGAGAAGTTCAAGAACAAGGCCACACTGACTGTAGACATAT
CCTCCAGCACTGCCTACATGCAACTCAGCAGCCTGACATCTGAGGACTCTGCGGTCTATTATTGTACATT
ACTAATAGGGGTCTACTGGGGCCAAGGCACCACTCTCACAGTCTCCTCA
Antibody 28F9 Heavy chain-Signal sequence-FR1-CDR1-FR2-CDR2-FR3-
CDR3-FR4
(SEQ ID NO: 124)
MGWSYIILFLVATATGVHSQVQLQQPGAELVQPGASVKLSCKASGYTFTGYFLYWVKQRPGHGLEWIGGI
HPSNGDTDENEKFKNKATLTVDISSSTAYMQLSSLTSEDSAVYYCTLLIGVYWGQGTTLTVSS
Antibody 28F9 Light chain-Signal sequence-FR1-CDR1-FR2-CDR2-FR3-
CDR3-FR4
(SEQ ID NO: 125)
ATGAGTCCTGCCCAGTTCCTGTTTCTGTTAGTGCTCTGGATTCGGGAAACCAACGGTGATGTTGTGATGA
CCCAGACTCCACTCACTTTGTCGGTTACCATTGGACAACCAGCCTCCATCTCTTGCAAGTCAAGTCAGAG
CCTCTTACATAGTGATGGAAAGACATATTTGATTTGGTTGTTACAGAGGCCAGGCCAGTCTCCAAAGCGC
CTAATCTATCTGGTGTCTAAACTGGACTCTGGAGTCCCTGACAGGTTCACCGGCAGTGGATCAGGGACAG
ATTTCACACTGAAAATCAGCAGAGTGGAGGCTGAGGATTTGGGAGTTTATTTTTGCTGTCAAGGTACACA
TTTTCCGTGGACGTTCGGTGGAGGCACCATGCTGGAAATCAAA
Antibody 28F9 Light chain-Signal sequence-FR1-CDR1-FR2-CDR2-FR3-
CDR3-FR4
(SEQ ID NO: 126)
MSPAQFLFLLVLWIRETNGDVVMTQTPLTLSVTIGQPASISCKSSQSLLHSDGKTYLIWLLQRPGQSPKR
LIYLVSKLDSGVPDRFTGSGSGTDFTLKISRVEAEDLGVYFCCQGTHEPWTFGGGTMLEIK
Antibody 28F9 Heavy Chain CDR1
(SEQ ID NO: 127)
GGCTACTTTTTGTAC
Antibody 28F9 Heavy Chain CDR1
(SEQ ID NO: 128)
GYFLY
Antibody 28F9 Heavy Chain CDR2
(SEQ ID NO: 129)
GGAATTCATCCTAGCAATGGTGATACTGACTTCAATGAGAAGTTCAAGAAC
Antibody 28F9 Heavy Chain CDR2
(SEQ ID NO: 130)
GIHPSNGDTDENEKFKN
Antibody 28F9 Heavy Chain CDR3
(SEQ ID NO: 131)
CTAATAGGGGTCTAC
Antibody 28F9 Heavy Chain CDR3
(SEQ ID NO: 132)
LIGVY
Antibody 28F9 Light Chain CDR1
(SEQ ID NO: 133)
AAGTCAAGTCAGAGCCTCTTACATAGTGATGGAAAGACATATTTGATT
Antibody 28F9 Light Chain CDR1
(SEQ ID NO: 134)
KSSQSLLHSDGKTYLI
Antibody 28F9 Light Chain CDR2
(SEQ ID NO: 135)
CTGGTGTCTAAACTGGACTCT
Antibody 28F9 Light Chain CDR2
(SEQ ID NO: 136)
LVSKLDS
Antibody 28F9 Light Chain CDR3
(SEQ ID NO: 137)
TGTCAAGGTACACATTTTCCGTGGACG
Antibody 28F9 Light Chain CDR3
(SEQ ID NO: 138)
CQGTHEPWT
Antibody 18B4 Heavy chain-Signal sequence-FR1-CDR1-FR2-CDR2-FR3-
CDR3-FR4
(SEQ ID NO: 139)
ATGTACTTGGGACTGAACTATGTATTCATAGTTTTTCTCTTAAATGGTGTCCAGAGTGAAGTGAAACTTG
AGGAGTCTGGAGGAGGCTTGGTGCAACCTGGGGGATCCATGAAACTCTCTTGTGCTGCCTCTGGATTCAC
TTTTAATGACGCCTGGATGGACTGGGTCCGCCAGTCTCCAGAGAAGGGGCTTGAGTGGGTTGCTGAAATT
AGAAGCACAGCTAATATTCATACAACATACTATGCTGAGTCTGTCCAAGGGAGGTTCACCATCTCAAGAG
ATGATTCCAAAAGTAGTGTCTACCTGCAAATGAACAGCTTGAGAGCTGAAGACACTGGCATTTATTATTG
TACCCCATTACTCTACGGATTTGCTTACTGGGGCCAAGGGACTCTGGTCACTGTCTCTGCA
Antibody 18B4 Heavy chain-Signal sequence-FR1-CDR1-FR2-CDR2-FR3-
CDR3-FR4
(SEQ ID NO: 140)
MYLGLNYVFIVFLLNGVQSEVKLEESGGGLVQPGGSMKLSCAASGETENDAWMDWVRQSPEKGLEWVAEI
RSTANIHTTYYAESVQGRFTISRDDSKSSVYLQMNSLRAEDTGIYYCTPLLYGFAYWGQGTLVTVSA
Antibody 18B4 Light chain-Signal sequence-FR1-CDR1-FR2-CDR2-FR3-
CDR3-FR4
(SEQ ID NO: 141)
ATGAAGTTGCCTGTTAGGCTGTTGGTGCTGATGTTCTGGATTCCTGCTTCCAGCAGTGATGTTGTGATGA
CCCAAAGTCCACTCTCCCTGCCTGTCAGTCTTGGAGATCAAGCCTCCATCTCTTGCAGAACTAGTCAGAG
CCTTGTACACAGTAATGGAAACACCTATTTACATTGGCACCTGCAGAAGCCAGGCCAGTCTCCAAAGGTC
CTGATCTACAAAGTTTCCAGCCGATTTTCTGGGGTCCCAGACAGGTTCAGTGGCAGTGGATCGGGGACAG
ATTTCACACTCAAGATCAGCAGAGTGGAGGCTGAGGATCTGGGAGTTTATTTCTGCTCTCAAAATACACA
TGTTCCGTACACGTTCGGAGGGGGGACCAAGCTGGAAATAAAA
Antibody 18B4 Heavy chain-Signal sequence-FR1-CDR1-FR2-CDR2-FR3-
CDR3-FR4
(SEQ ID NO: 142)
MKLPVRLLVLMFWIPASSSDVVMTQSPLSLPVSLGDQASISCRTSQSLVHSNGNTYLHWHLQKPGQSPKV
LIYKVSSRFSGVPDRESGSGSGTDFTLKISRVEAEDLGVYFCSQNTHVPYTFGGGTKLEIK
Antibody 18B4 Heavy Chain CDR1
(SEQ ID NO: 143)
GACGCCTGGATGGAC
Antibody 18B4 Heavy Chain CDR1
(SEQ ID NO: 144)
DAWMD
Antibody 18B4 Heavy Chain CDR2
(SEQ ID NO: 145)
GAAATTAGAAGCACAGCTAATATTCATACAACATACTATGCTGAGTCTGTCCAAGGG
Antibody 18B4 Heavy Chain CDR2
(SEQ ID NO: 146)
EIRSTANIHTTYYAESVQG
Antibody 18B4 Heavy Chain CDR3
(SEQ ID NO: 147)
TTACTCTACGGATTTGCTTAC
Antibody 18B4 Heavy Chain CDR3
(SEQ ID NO: 148)
LLYGFAY
Antibody 18B4 Light Chain CDR1
(SEQ ID NO: 149)
AGAACTAGTCAGAGCCTTGTACACAGTAATGGAAACACCTATTTACAT
Antibody 18B4 Light Chain CDR1
(SEQ ID NO: 150)
RTSQSLVHSNGNTYLH
Antibody 18B4 Light Chain CDR2
(SEQ ID NO: 151)
AAAGTTTCCAGCCGATTTTCT
Antibody 18B4 Light Chain CDR2
(SEQ ID NO: 152)
KVSSRFS
Antibody 18B4 Light Chain CDR3
(SEQ ID NO: 153)
TCTCAAAATACACATGTTCCGTACACG
Antibody 18B4 Light Chain CDR3
(SEQ ID NO: 154)
SQNTHVPYT
Antibody 1E4 Heavy chain-Signal sequence-FR1-CDR1-FR2-CDR2-FR3-CDR3-
FR4
(SEQ ID NO: 155)
ATGGAATGGCCTTGTATCTTTCTCTTCCTCCTGTCAGTAACTGAAGGTGTCCACTCCCAGGTTCAGCTGC
AGCAGTCTGGGGCTGAGCTGGTGAGGCCTGGGTCCTCAGTGAAGATTTCCTGTAAGGCTTCTGGCTATGC
ATTCAGTACCTACTGGATGAACTGGGTGAAGCAGAGGCCTGGACAGGGTCTTGAGTGGATTGGACAGATT
TATCCTGGAGATAGTGATACTAACTACAATGGAAAGTTCAAGGGTAAAGCCACACTGACTGCAGACAAGT
CCTCCAACACAGCCTACATGCAGCTCAGCAGCCTAACATCTGAGGACTCTGCGGTCTTTTTCTGTGCAAG
AGGTAACCACGCCTCTATGGACTACTGGGGTCAAGGAACCTCAGTCACCGTCTCCTCA
Antibody 1E4 Heavy chain-Signal sequence-FR1-CDR1-FR2-CDR2-FR3-CDR3-
FR4
(SEQ ID NO: 156)
MEWPCIFLFLLSVTEGVHSQVQLQQSGAELVRPGSSVKISCKASGYAFSTYWMNWVKQRPGQGLEWIGQI
YPGDSDTNYNGKFKGKATLTADKSSNTAYMQLSSLTSEDSAVFFCARGNHASMDYWGQGTSVTVSS
Antibody 1E4 Light chain-Signal sequence-FR1-CDR1-FR2-CDR2-FR3-CDR3-
FR4
(SEQ ID NO: 157)
ATGAAGTTGCCTGTTAGGCTGTTGGTGCTGATGTTCTGGATTCCTGCTTCCAGCAGTGATGTTGTGATGA
CCCAAACTCCACTCTCCCTGCCTGTCAGTCTTGGAGATCAAGCCTCCATCTCTTGCAGATCTAGTCAGAG
CCTTGTACACAGTAATGGAAACACCTATTTACATTGGTACCTGCAGAAGCCAGGCCAGTCTCCAAAGCTC
CTGATCTACAAAGTTTCCAACCGATTTTCTGGGGTCCCAGACAGGTTCAGTGGCAGTGGATCAGGGACAG
ATTTCACACTCAAGATCAGCAGAGTGGAGGCTGAGGATCTGGGAGTTTATTTCTGCTCTCAAAAAACACA
TGTTCCGTGGACGTTCGGTGGAGGCACCAAGCTGGAAATCAAA
Antibody 1E4 Light chain-Signal sequence-FR1-CDR1-FR2-CDR2-FR3-CDR3-
FR4
(SEQ ID NO: 158)
MKLPVRLLVLMFWIPASSS DVVMTQTPLSLPVSLGDQASISCRSSQSLVHSNGNTYLHWYLQKPGQSPKL
LIYKVSNRFSGVPDRESGSGSGTDETLKISRVEAEDLGVYFCSQKTHVPWTFGGGTKLEIK
Antibody 1E4 Heavy Chain CDR1
(SEQ ID NO: 159)
ACCTACTGGATGAAC
Antibody 1E4 Heavy Chain CDR1
(SEQ ID NO: 160)
TYWMN
Antibody 1E4 Heavy Chain CDR2
(SEQ ID NO: 161)
CAGATTTATCCTGGAGATAGTGATACTAACTACAATGGAAAGTTCAAGGGT
Antibody 1E4 Heavy Chain CDR2
(SEQ ID NO: 162)
QIYPGDSDTNYNGKFKG
Antibody 1E4 Heavy Chain CDR3
(SEQ ID NO: 163)
GGTAACCACGCCTCTATGGACTAC
Antibody 1E4 Heavy Chain CDR3
(SEQ ID NO: 164)
GNHASMDY
Antibody 1E4 Light Chain CDR1
(SEQ ID NO: 165)
AGATCTAGTCAGAGCCTTGTACACAGTAATGGAAACACCTATTTACAT
Antibody 1E4 Light Chain CDR1
(SEQ ID NO: 166)
RSSQSLVHSNGNTYLH
Antibody 1E4 Light Chain CDR2
(SEQ ID NO: 167)
AAAGTTTCCAACCGATTTTCT
Antibody 1E4 Light Chain CDR2
(SEQ ID NO: 168)
KVSNRFS
Antibody 1E4 Light Chain CDR3
(SEQ ID NO: 169)
TCTCAAAAAACACATGTTCCGTGGACG
Antibody 1E4 Light Chain CDR3
(SEQ ID NO: 170)
SQKTHVPWT
Antibody 29H1 Heavy chain-Signal sequence-FR1-CDR1-FR2-CDR2-FR3-
CDR3-FR4
(SEQ ID NO: 171)
ATGTACTTGGGACTGAACTATGTATTCATAGTTTTTCTCTTAAATGGTGTCCAGAGTGAAGTGAAGCTTG
AGGAGTCTGGAGGAGGCTTGGTACAACCTGGAGGATCCATGAAACTCTCTTGTGCTGCCTCTGGATTCAC
TTTTAGTGACGCCTGGATGGACTGGGTCCGCCAGTCTCCAGAGAAGGGGCTTGAATGGGTTGCTGAAATT
AGAAGCAAAGCTACTAATCATGCAACATACTATGCTGAGTCTGTGAAAGGGAGGTTCACCATCTCAAGAG
ATGATTCCAAAAGTAGTGTCTACCTGCAAATGAACAGCTTAAGAGCTGAAGACACTGGCATTTATTACTG
TACCCCCCTACTTTACGGGTTTGCTTACTGGGGCCAAGGGACTCTGGTCACTGTCTCTGCA
Antibody 29H1 Heavy chain-Signal sequence-FR1-CDR1-FR2-CDR2-FR3-
CDR3-FR4
(SEQ ID NO: 172)
MYLGLNYVFIVFLLNGVQSEVKLEESGGGLVQPGGSMKLSCAASGFTFSDAWMDWVRQSPEKGLEWVAEI
RSKATNHATYYAESVKGRFTISRDDSKSSVYLQMNSLRAEDTGIYYCTPLLYGFAYWGQGTLVTVSA
Antibody 29H1 Light chain-Signal sequence-FR1-CDR1-FR2-CDR2-FR3-
CDR3-FR4
(SEQ ID NO: 173)
ATGAAGTTGCCTGTTAGGCTGTTGGTGCTGATGTTCTGGATTCCTGCTTCCAGCAGTGATGTTGTGATGA
CCCAAACTCCACTCTCCCTGCCTGTCAGTCTTGGAGATCAAGCCTCCATCTCTTGCAGATCTGGTCAGAG
CCTTGTACACAGTAATGGACACACCTATTTACATTGGTACCTGCAGAAGCCAGGCCAGTCTCCAAGGCTC
CTGATCTACAAAGTTTCCAACCGATTTTCTGGGGTCCCAGACAGGTTCAGTGGCAGTGGATCAAGGGCAG
ATTTCACACTCAAGATCAGCAGAGTGGAGGCTGAGGATCTGGGAGTTTATTTCTGCTCTCAAACTACACA
TGTTCCGTGGACGTTCGGTGGAGGCACCAAGCTGGAAATCAAA
Antibody 29H1 Light chain-Signal sequence-FR1-CDR1-FR2-CDR2-FR3-
CDR3-FR4
(SEQ ID NO: 174)
MKLPVRLLVLMFWIPASSSDVVMTQTPLSLPVSLGDQASISCRSGQSLVHSNGHTYLHWYLQKPGQSPRL
LIYKVSNRFSGVPDRESGSGSRADETLKISRVEAEDLGVYFCSQTTHVPWTFGGGTKLEIK
Antibody 29H1 Heavy Chain CDR1
(SEQ ID NO: 175)
GACGCCTGGATGGAC
Antibody 29H1 Heavy Chain CDR1
(SEQ ID NO: 176)
DAWMD
Antibody 29H1 Heavy Chain CDR2
(SEQ ID NO: 177)
GAAATTAGAAGCAAAGCTACTAATCATGCAACATACTATGCTGAGTCTGTGAAAGGG
Antibody 29H1 Heavy Chain CDR2
(SEQ ID NO: 178)
EIRSKATNHATYYAESVKG
Antibody 29H1 Heavy Chain CDR3
(SEQ ID NO: 179)
CTACTTTACGGGTTTGCTTAC
Antibody 29H1 Heavy Chain CDR3
(SEQ ID NO: 180)
LLYGFAY
Antibody 29H1 Light Chain CDR1
(SEQ ID NO: 181)
AGATCTGGTCAGAGCCTTGTACACAGTAATGGACACACCTATTTACAT
Antibody 29H1 Light Chain CDR1
(SEQ ID NO: 182)
RSGQSLVHSNGHTYLH
Antibody 29H1 Light Chain CDR2
(SEQ ID NO: 183)
AAAGTTTCCAACCGATTTTCT
Antibody 29H1 Light Chain CDR2
(SEQ ID NO: 184)
KVSNRFS
Antibody 29H1 Light Chain CDR3
(SEQ ID NO: 185)
TCTCAAACTACACATGTTCCGTGGACG
Antibody 29H1 Light Chain CDR3
(SEQ ID NO: 186)
SQTTHVPWT
Antibody 31A1 Heavy chain-Signal sequence-FR1-CDR1-FR2-CDR2-FR3-
CDR3-FR4
(SEQ ID NO: 187)
ATGGAAAGGCACTGGATCTTTCTCTTCCTGTTTTCAGTAACTGCAGGTGTCCACTCCCAGGTCCAGCTTC
AGCAGTCTGGGGCTGAACTGGCAAAACCTGGGGCCTCAGTGAAGATGTCCTGCAAGGCTTCTGGCTACAC
CTTTACTAGCTACTGGATGCACTGGGTAAAACAGAGGCCTGGACAGGGTCTGGAATGGATTGGATACATT
AATCCTAGCACTGGTTATACTGAGTACAATCAGAAGTTCAAGGACAAGGCCACATTGACTGCAGACAAAT
CCTCCAGCACAGCCTACATGCAACTGAGCAGCCTGACATCTGAGGACTCTGCAGTCTATTACTGTGCAAG
AGCCTACATTGACTACTGGGGCCAAGGCACCACTCTCACAGTCTCCTCA
Antibody 31A1 Heavy chain-Signal sequence-FR1-CDR1-FR2-CDR2-FR3-
CDR3-FR4
(SEQ ID NO: 188)
MERHWIFLFLFSVTAGVHSQVQLQQSGAELAKPGASVKMSCKASGYTFTSYWMHWVKQRPGQGLEWIGYI
NPSTGYTEYNQKFKDKATLTADKSSSTAYMQLSSLTSEDSAVYYCARAYIDYWGQGTTLTVSS
Antibody 31A1 Light chain-Signal sequence-FR1-CDR1-FR2-CDR2-FR3-
CDR3-FR4
(SEQ ID NO: 189)
ATGAAGTTGCCTGTTAGGCTGTTGGTGCTGATGTTCTGGATTCCTGCTTCCAGCAGTGATGTTTTGATGA
CCCAAACTCCACTCTCCCTGCCTGTCAGTCTTGGAGATCAAGCCTCCTTCTCTTGCAGATCTAGTCAGAG
CATTGTACATAGTAATGGAAACACCTATTTAGAA TGGTACCTGCAGAAACCAGGCCAGTCTCCAAAGCTC
CTGATCTACAAAGTTTCCAACCGATTTTCTGGGGTCCCAGACAGGTTCAGTGGCAGTGGATCAGGGACAG
ATTTCACACTCAAGATCAACAGAGTGGAGGCTGAGGATCTGGGAGTTTATTACTGCTTTCAAGTTTCACA
TTTTCCGTGGACGTTCGGTGGAGGCACCAAGCTGGAAATCAAA
Antibody 31A1 Light chain-Signal sequence-FR1-CDR1-FR2-CDR2-FR3-
CDR3-FR4
(SEQ ID NO: 190)
MKLPVRLLVLMFWIPASSSDVLMTQTPLSLPVSLGDQASFSCRSSQSIVHSNGNTYLEWYLQKPGQSPKL
LIYKVSNRFSGVPDRESGSGSGTDETLKINRVEAEDLGVYYCFQVSHFPWTFGGGTKLEIK
Antibody 31A1 Heavy Chain CDR1
(SEQ ID NO: 191)
AGCTACTGGATGCAC
Antibody 31A1 Heavy Chain CDR1
(SEQ ID NO: 192)
SYWMH
Antibody 31A1 Heavy Chain CDR2
(SEQ ID NO: 193)
TACATTAATCCTAGCACTGGTTATACTGAGTACAATCAGAAGTTCAAGGAC
Antibody 31A1 Heavy Chain CDR2
(SEQ ID NO: 194)
YINPSTGYTEYNQKEKD
Antibody 31A1 Heavy Chain CDR3
(SEQ ID NO: 195)
GCCTACATTGACTAC
Antibody 31A1 Heavy Chain CDR3
(SEQ ID NO: 196)
AYIDY
Antibody 31A1 Light Chain CDR1
(SEQ ID NO: 197)
AGATCTAGTCAGAGCATTGTACATAGTAATGGAAACACCTATTTAGAA
Antibody 31A1 Light Chain CDR1
(SEQ ID NO: 198)
RSSQSIVHSNGNTYLE
Antibody 31A1 Light Chain CDR2
(SEQ ID NO: 199)
AAAGTTTCCAACCGATTTTCT
Antibody 31A1 Light Chain CDR2
(SEQ ID NO: 200)
KVSNRFS
Antibody 31A1 Light Chain CDR3
(SEQ ID NO: 201)
TTTCAAGTTTCACATTTTCCGTGGACG
Antibody 31A1 Light Chain CDR3
(SEQ ID NO: 202)
FQVSHFPWT
Antibody 32C1 Heavy chain-Signal sequence-FR1-CDR1-FR2-CDR2-FR3-
CDR3-FR4
(SEQ ID NO: 203)
ATGTACTTGGGACTGAACTGTGTATTCATAGTTTTTCTCTTAAAAGGTGTCCAGAGTGAAGTGAAGCTT
AGGAGTCTGGAGGAGGCTTGGTGCAATCTGGAGGATCCATGAAACTCTCCTGTGTTGCCTCTGGATTCAC
TTTCAGTAATTACTGGATGAAC TGGGTCCGCCAGTCTCCAGAGAAGGGGCTTGAGTGGGTTGCTGAAATT
AGATTGAAATCTAATAATTATGCAATACATTATGCGGAGTCTGTGAAGGGGAGGTTCACCATCTCAAGAG
ATGATTCCAAAAGTAGTGTCTACCTGCAAATGAACAACTTAAGAGCTGAAGACACTGGCATTTATTACTG
TACCAGGGTCCCGGGACTGGATGCTTACTGGGGCCAAGGGACTCTGGTCACTGTCTCTGCA
Antibody 32C1 Heavy chain-Signal sequence-FR1-CDR1-FR2-CDR2-FR3-
CDR3-FR4
(SEQ ID NO: 204)
MYLGLNCVFIVFLLKGVQSEVEESGGGLVQSGGSMKLSCVASGFTFS NYWMNWVRQSPEKGLEWVAEI
RLKANNYATHYAESVKGRFTISRDDSKSSVYLQMNNLRAEDTGIYYCTRVPGLDAYWGQGTLVTVSA
Antibody 32C1 Light chain-Signal sequence-FR1-CDR1-FR2-CDR2-FR3-
CDR3-FR4
(SEQ ID NO: 205)
ATGAAGTTGCCTGTTAGGCTGTTGGTGCTGATGTTCTGGATTCCTGCTTCCAGCAGTGATGTTGTGATGA
CCCAAACTCCACTCTCCCTGCCTGTCAGTCTTGGAGATCAAGCCTCCATCTCTTGCAGATCTAGTCAGAG
CCTTGTACACAGTAATGGAAACACCTATTTACATTGGTACCTGCAGAAGCCAGGCCAGTCTCCAAAGCTC
CTGATCTACAAAGTTTCCAACCGATTTTCTGGGGTCCCAGACAGGTTCAGTGGCAGTGGATCAGGGACAG
ATTTCACACTCAAGATCAGCAGTGTGGAGGCTGAGGATCTGGGAGTTTATTTCTGCTCTCAAATTACACA
TGTTCCGTACACGTTCGGAGGGGGGACCAATCTGGAAATAAAA
Antibody 32C1 Light chain-Signal sequence-FR1-CDR1-FR2-CDR2-FR3-
CDR3-FR4
(SEQ ID NO: 206)
MKLPVRLLVLMFWIPASSSDVVMTQTPLSLPVSLGDQASISCRSSQSLVHSNGNTYLHWYLQKPGQSPKL
LIY KVSNRFSGVPDRESGSGSGTDETLKISSVEAEDLGVYFCSQITHVPYTFGGGTNLEIK
Antibody 32C1 Heavy Chain CDR1
(SEQ ID NO: 207)
AATTACTGGATGAAC
Antibody 32C1 Heavy Chain CDR1
(SEQ ID NO: 208)
NYWMN
Antibody 32C1 Heavy Chain CDR2
(SEQ ID NO: 209)
GAAATTAGATTGAAATCTAATAATTATGCAATACATTATGCGGAGTCTGTGAAGGGG
Antibody 32C1 Heavy Chain CDR2
(SEQ ID NO: 210)
EIRLKSNNYAIHYAESVKG
Antibody 32C1 Heavy Chain CDR3
(SEQ ID NO: 211)
GTCCCGGGACTGGATGCTTAC
Antibody 32C1 Heavy Chain CDR3
(SEQ ID NO: 212)
VPGLDAY
Antibody 32C1 Light Chain CDR1
(SEQ ID NO: 213)
AGATCTAGTCAGAGCCTTGTACACAGTAATGGAAACACCTATTTACAT
Antibody 32C1 Light Chain CDR1
(SEQ ID NO: 214)
RSSQSLVHSNGNTYLH
Antibody 32C1 Light Chain CDR2
(SEQ ID NO: 215)
AAAGTTTCCAACCGATTTTCT
Antibody 32C1 Light Chain CDR2
(SEQ ID NO: 216)
KVSNRFS
Antibody 32C1 Light Chain CDR3
(SEQ ID NO: 217)
TCTCAAATTACACATGTTCCGTACACG
Antibody 32C1 Light Chain CDR3
(SEQ ID NO: 218)
SQITHVPYT
Antibody 45C11 Heavy chain-Signal sequence-FR1-CDR1-FR2-CDR2-FR3-
CDR3-FR4
(SEQ ID NO: 219)
ATGAAATGCAGCTGGGTTATCTTCTTCCTGATGGCAGTGGTTACAGGGGTCAATTCAGAGGTTCAGCTGC
AGCAGTCTGGGGCAGACCTTGTGAAGCCAGGGGCCTCAGTCAAGTTGTCCTGCACAGCTTCTGGCTTCAA
CATTAAAGACACCTTTATGCACTGGGTGAAGCAGAGGCCTGAACAGGGCCTGGAGTGGATTGGAAGGATT
GATCCTGCGAATGGTAATACTAAATATGACCCGAAATTCCAGGGCAAGGCCACTATAACAGCAGACACAT
CCTCCAACACAGCCTACCTGCAGCTCAGCAGCCTGACATCTGAGGACACTGCCGTCTATTACTGTGCTAA
ACCGTATGGTAACTACGGCTATTACTATGCTTTGGACTACTGGGGTCAAGGAACCTCAGTCACCGTCTCC
TCA
Antibody 45C11 Heavy chain-Signal sequence-FR1-CDR1-FR2-CDR2-FR3-
CDR3-FR4
(SEQ ID NO: 220)
MKCSWVIFFLMAVVTGVNSEVQLQQSGADLVKPGASVKLSCTASGENIKDTFMHWVKQRPEQGLEWIGRI
DPANGNTKYDPKFQGKATITADTSSNTAYLQLSSLTSEDTAVYYCAKPYGNYGYYYALDYWGQGTSVTVS
S
Antibody 45C11 Light chain-Signal sequence-FR1-CDR1-FR2-CDR2-FR3-
CDR3-FR4
(SEQ ID NO: 221)
ATGAGGTTCCAGGTTCAGGTTCTGGGGCTCCTTCTGCTCTGGATATCAGGTGCCCAGTGTGATGTCCAGA
TAACCCAGTCTCCATCTTATCTTGCTGCATCTCCTGGAGAAACCATTACTATTAATTGCAGGGCAAGTAA
GAGCATTAGCAAATATTTAGCCTGGTATCAAGAGAAACCTGGGAAAACTAATAAGCTTCTTATCTACTCT
GGATCCACTTTGCAATCTGGAATTCCATCAAGGTTCAGTGGCAGTGGATCTGGTACAGATTTCACTCTCA
CCATCAGTAGCCTGGAGCCTGAAGATTTTGCAATGTATTACTGTCAACAGCATAATGAATTCCCGTGGAC
GTTCGGTGGAGGCACCAAGCTGGAAATCAAA
Antibody 45C11 Light chain-Signal sequence-FR1-CDR1-FR2-CDR2-FR3-
CDR3-FR4
(SEQ ID NO: 222)
MRFQVQVLGLLLLWISGAQCDVQITQSPSYLAASPGETITINCRASKSISKYLAWYQEKPGKINKLLIYS
GSTLQSGIPSRESGSGSGTDFTLTISSLEPEDFAMYYCQQHNEFPWTFGGGTKLEIK
Antibody 45C11 Heavy Chain CDR1
(SEQ ID NO: 223)
GACACCTTTATGCAC
Antibody 45C11 Heavy Chain CDR1
(SEQ ID NO: 224)
DTFMH
Antibody 45C11 Heavy Chain CDR2
(SEQ ID NO: 225)
AGGATTGATCCTGCGAATGGTAATACTAAATATGACCCGAAATTCCAGGGC
Antibody 45C11 Heavy Chain CDR2
(SEQ ID NO: 226)
RIDPANGNTKYDPKFQG
Antibody 45C11 Heavy Chain CDR3
(SEQ ID NO: 227)
CCGTATGGTAACTACGGCTATTACTATGCTTTGGACTAC
Antibody 45C11 Heavy Chain CDR3
(SEQ ID NO: 228)
PYGNYGYYYALDY
Antibody 45C11 Light Chain CDR1
(SEQ ID NO: 229)
AGGGCAAGTAAGAGCATTAGCAAATATTTAGCC
Antibody 45C11 Light Chain CDR1
(SEQ ID NO: 230)
RASKSISKYLA
Antibody 45C11 Light Chain CDR2
(SEQ ID NO: 231)
TCTGGATCCACTTTGCAATCT
Antibody 45C11 Light Chain CDR2
(SEQ ID NO: 232)
SGSTLQS
Antibody 45C11 Light Chain CDR3
(SEQ ID NO: 233)
CAACAGCATAATGAATTCCCGTGGACG
Antibody 45C11 Light Chain CDR3
(SEQ ID NO: 234)
QQHNEFPWT
Tandem repeat domain peptide
(SEQ ID NO: 235)
PDTRPAPGSTAPPAHGVTSA
CAR44: CD8/HUMNC2/CD8/4-1BB/CD3
(SEQ ID NO: 236)
MALPVTALLLPLALLLHAARPEVQLVESGGGLVKPGGSLRLSCAASGFTFSGYAMSWVRQAPGKGLEWVS
TISSGGTYIYYPDSVKGRFTISRDNAKNSLYLQMNSLRAEDTAVYYCARLGGDNYYEYFDVWGKGTTVTV
SSGGGGSGGGGSGGGGSDIVLTQSPASLAVSPGQRATITCRASKSVSTSGYSYMHWYQQKPGQPPKLLIY
LASNLESGVPARFSGSGSGTDFTLTINPVEANDTANYYCQHSRELPFTFGGGTKVEIKRTTTTPAPRPPT
PAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKESRSADAPAYKQGQNQLYNELNLGRREEYDVLDKRR
GRDPEMGGKPRRKNPQEGLYNELQKDKMAEAYSEIGMKGERRRGKGHDGLYQGLSTATKDTYDALHMQAL
PPR
MIN-A2-1 LIGHT CHAIN VARIABLE REGION AMINO ACID SEQUENCE
(SEQ ID NO: 237)
DIVLTQSTEIMSASPGEKVTITCSASSSISYIHWFQQKPGTSPKLWIFGTSNLAS
GVPARFSGSGSGTSYSLTVSRMEAEDTATYYCQQRSNYPFTFGSGTKLQIKRADAAPTVS
MIN-A2-2 LIGHT CHAIN VARIABLE REGION AMINO ACID SEQUENCE
(SEQ ID NO: 238)
DIVMTQSPAIMSASPGEKVTMTCSASSSVSYMHWFQQKPGTSPKLWIYSTSNLAS
GAPARFSGSGSGTSYSLTVSRMESEDAATYYCQQRSSYPSTFGGGTKLEIKRADAAPTVS
MIN-C9-1 LIGHT CHAIN VARIABLE REGION AMINO ACID SEQUENCE
(SEQ ID NO: 239)
DIVLTQTTAIMSASPGEKVTITCSASSSVSYMYWFQQKPGTSPKLWIYSTSNLAS
GVPARFSGSGSGTSYSLTISRMEAEDAATYYCQQRSSYPSTFGGGTKLEIKRADAAPTVS
MIN-C9-2 LIGHT CHAIN VARIABLE REGION AMINO ACID SEQUENCE
(SEQ ID NO: 240)
DIVITQSTAIMSASPGEKVTITCSASSSVSYTYWFQQKPGTSPKLWIYSTSNLAS
GVPARFSGSGSGTSYSLTISRMEAEDAATYYCQQRSSYPSTEGGGTKLEIKRADAAPTVS
MIN-D7-1 LIGHT CHAIN VARIABLE REGION AMINO ACID SEQUENCE
(SEQ ID NO: 241)
DIVITQTPAIMSASPGEKVTMTCSASSSVSYMHWFQQKPGTSPKLWIYSTSNLAS
GVPARFSGSGSGTSYSLTVSRMESEDAATYYCQQRSSYPSTFGGGTKLEIKRADA
APTVS
MIN-D7-2 LIGHT CHAIN VARIABLE REGION AMINO ACID SEQUENCE
(SEQ ID NO: 242)
DIVLTQSTAIMSASPGEKVTMTCSASSSVSYMHWFQQKPGTSPKLWIYSTSNLAS
GVPARFSGSGSGTSYSLTVSRMESEDAATYYCQQRSSYPSTFGGGTKLEIKRADA
APTVS
MIN-F2-1 LIGHT CHAIN VARIABLE REGION AMINO ACID SEQUENCE
(SEQ ID NO: 243)
DIVMTQSPEIMSASPGEKVTITCSASSSISYIHWFQQKPGTSPKLWIFGTSNLAS
GVPARFSGSGSGTSYSLTVSRMEAEDTATYYCQQRSNYPFTFGSGTKLQIKRADA
APTVS
MIN-F2-2 LIGHT CHAIN VARIABLE REGION AMINO ACID SEQUENCE
(SEQ ID NO: 244)
DIVITQSTEIMSASPGEKVTITCSASSSISYIHWFQQKPGTSPKLWIFGTSNLAS
GVPARFSGSGSGTSYSLTVSRMEAEDTATYYCQQRSNYPFTFGSGTKLQIKRADA
APTVS
MIN-A2-1 HEAVY CHAIN VARIABLE REGION AMINO ACID SEQUENCE
(SEQ ID NO: 245)
EVKLQESGPELKKPGETVEISCKASGYTFTNYGMNWVKQAPGKGLKWMGWINTYTGEPTYAGDEKGRFAF
SLETSASTAYLQINTLKNEDTATYFCARSGDGYWYYAMDYWGQGTSVTVSSAKTTPPSVY
MIN-A2-2 HEAVY CHAIN VARIABLE REGION AMINO ACID SEQUENCE
(SEQ ID NO: 246)
EVQLQQSGPELKKPGETVKISCKASGYTFTNYGMNWVKQAPGKGLKWMGWINTYTGEPTYAGDFKGRFAF
SLETSASTAYLQINTLKNEDTATYFCARSGDGYWYYAMDYWGQGTSVTVSSAKTTPPSVY
MIN-C9-1 HEAVY CHAIN VARIABLE REGION AMINO ACID SEQUENCE
(SEQ ID NO: 247)
QVQLQESGPELKQPGETVKISCKASGYTFTNNGMNWVKQAPGKGLKWMGWINTYTGEPTYADDFKGRFAF
SLDTSASTAYLQINNLKNEDMATYFCARTGTARAFYAMDYWGQGTSVTVSSTKTTAPSVY
MIN-C9-2 HEAVY CHAIN VARIABLE REGION AMINO ACID SEQUENCE
(SEQ ID NO: 248)
QVQLQQSGPELKQPGETVKISCKASGYTFTNNGMNWVKQAPGKGLKWMGWINTYTGEPTYADDEKGREAF
SLGTSASTAYLQINNLKNEDMATYFCARTGTARAFYAMDYWGQGTSVTVSSTKTTAPSVY
MIN-D7-1 HEAVY CHAIN VARIABLE REGION AMINO ACID SEQUENCE
(SEQ ID NO: 249)
EVQLEQSGPELKKPGETVKISCKASGYTFINYGMNWVKQAPGKGLKWMGWINTYTGEPTYVDDFKGRFAF
SLETSARTAYLQINNLKNEDMATYFCARTGTTAILNGMDYWGQGTSVTVSSAKTTPPSVY
MIN-D7-2 HEAVY CHAIN VARIABLE REGION AMINO ACID SEQUENCE
(SEQ ID NO: 250)
EVQLQQSGPELKKPGETVKISCKASGYTFTNYGMNWVKQAPGKGLKWMGWINTYTGEPTYAGDFKGRFAF
SLETSASTAYLQINTLKNEDTATYFCARSGDGYWYYAMDYWGQGTSVTVSSAKTTPPSVY
MIN-F2-1 HEAVY CHAIN VARIABLE REGION AMINO ACID SEQUENCE
(SEQ ID NO: 251)
EVKLEESGPELKKPGETVKISCKASGYTFINYGMNWVKQAPGKGLKWMGWINTYTGEPTYVDDFKGRFAF
SLETSARTAYLQINNLKNEDMATYFCARTGTTAILNGMDYWGQGTSVTVSSAKTTPPSVY
MIN-F2-2 HEAVY CHAIN VARIABLE REGION AMINO ACID SEQUENCE
(SEQ ID NO: 252)
EVQLEQSGAELVRPGASVKLSCKALGYTFTDYEMHWVKQTPVHGLEWIGAIHPGSGGTAYNQKFKGKATL
TADKSSSTAYMELSSLTSEDSAVYYCTNYGSFAYWGQGTLVTVSAAKTTPPSVY
MIN-F2-3 HEAVY CHAIN VARIABLE REGION AMINO ACID SEQUENCE
(SEQ ID NO: 253)
RCRLQQSGPELKKPGETVKISCKASGYTFINYGMNWVKQAPGKGLKWMGWINTYTGEPTYVDDFKGRFAF
SLETSARTAYLQINNLKNEDMATYFCARTGTTAILNGMDYWGQGTSVTVSSAKTTPPSCL
MIN-F2-4 HEAVY CHAIN VARIABLE REGION AMINO ACID SEQUENCE
(SEQ ID NO: 254)
EVQLEQSGPELKKPGETVKISCKASGYTFINYGMNWVKQAPGKGLKWMGWINTYTGEPTYVDDFKGRFAF
SLETSARTAYLQINNLKNEDMATYFCARTGTTAILNGMDYWGQGTSVTVSSAKTTPPSVY
MIN-14 LIGHT CHAIN VARIABLE REGION AMINO ACID SEQUENCE
(SEQ ID NO: 255)
DIQMTQSPSSLSASLGERVSLTCRASQDIGSSLNWLQQEPDGTIKRLIYATSSLDSGVPKRFSGSRSGSD
YSLTISSLESEDFVDYYCLQYASSPHVRCWDQAGAETGCCTNC
MIN-17-1 LIGHT CHAIN VARIABLE REGION AMINO ACID SEQUENCE
(SEQ ID NO: 256)
DIVLTQSPASLAVSLGQRATISYRASKSVSTSGYSYMHWNQQKPGQPPRLLIYLVSNLESGVPARESGSG
SGTDFTLNIHPVEEEDAATYYCQHIRELTRSEGGPSW
MIN-17-2 LIGHT CHAIN VARIABLE REGION AMINO ACID SEQUENCE
(SEQ ID NO: 257)
DIQMTQSPASQSASLGESVTITCLASQTIGTWLAWYQQKPGKSPQLLIYAATSLADGVPSRESGSGSGTK
FSFKISSLQAEDFVSYYCQQLYSTPWTFGGGTKLEIKRADAAPTV
MIN-29 LIGHT CHAIN VARIABLE REGION AMINO ACID SEQUENCE
(SEQ ID NO: 258)
DIVLTQSPASLAVSLGQRATISYRASKSVSTSGYSYMHWNQQKPGQPPRLLIYLVSNLESGVPARESGSG
SGTDFTLNIHPVEEEDAATYYCQHIRELTRSEGGPSW
MIN-34 LIGHT CHAIN VARIABLE REGION AMINO ACID SEQUENCE
(SEQ ID NO: 259)
DIVLTQSPASLAVSLGQRATISYRASKSVSTSGYSYMHWNQQKPGQPPRLLIYLVSNLESGVPARESGSG
SGTDFTLNIHPVEEEDAATYYCQHIRELTRSEGGPSW
MIN-42 LIGHT CHAIN VARIABLE REGION AMINO ACID SEQUENCE
(SEQ ID NO: 260)
DIVMTQSQKFMSTSVGDRVSVTCKASQNVGTNVGWYQQKPGQSPKALIYSASYRYSGVPDRFTGSGSGTD
FTLTISNVQSEDLAEYFCQQYNNYPYTFGGGTKLEIKRADAAPTV
MIN-45 LIGHT CHAIN VARIABLE REGION AMINO ACID SEQUENCE
(SEQ ID NO: 261)
DIQMTQPPASLSASVGETVTITCRASGNIHNFLAWYQQKQGKSPQLLVYNAKTLADGVPSRFSGSGSGTQ
YSLKINSLQPEDFGSYYCQHFWSTPWTFGGGTKLEIKRADAAPTV
MIN-14 HEAVY CHAIN VARIABLE REGION AMINO ACID SEQUENCE
(SEQ ID NO: 262)
QVQLQQPGAELVKPGASVKLSCKASGYTFTSYWMHWVKQRPGQGLEWIGEINPSNGRTNYNEKFKSKATL
TVDKSSSTAYMQLSSLTSEDSAVYYCATYGNYWYF
MIN-17-2 HEAVY CHAIN VARIABLE REGION AMINO ACID SEQUENCE
(SEQ ID NO: 263)
QITLKESGPGIVQPSQPFRLTCTFSGFSLSTSGIGVTWIRQPSGKGLEWLATIWWDDDNRYNPSLKSRLT
VSKDTS NNQAFLNIITVETADTAIYYCAQSTMVTA
MIN-17-1 HEAVY CHAIN VARIABLE REGION AMINO ACID SEQUENCE
(SEQ ID NO: 264)
QVQLQQPGAELVKPGASVKLSCKASGYTFTSYWMHWVKQRPGQGLEWIGEINPSNGRTNYNEK-
FKSKATLTVDKS SSTAYMQLSSLTSEDSAVYYCATYGNYWYF
MIN-29 HEAVY CHAIN VARIABLE REGION AMINO ACID SEQUENCE
(SEQ ID NO: 265)
DVKLVESGGDLXKLTEGEDIWEGLTLCRDSDQSPLAPVSKPGRVVRPQ
RSCTVIQGCVLRLQTAHLQVQGVLGIVSGDGESALHSVWIVGATTITINGC
DQLQPLLWSLANPRHVIATESESRGCTG
MIN-34 HEAVY CHAIN VARIABLE REGION AMINO ACID SEQUENCE
(SEQ ID NO: 266)
QVQLKQSGPGLVQPSQSLSITCTVSGFSLTSYGVHWVRQSPGKGLEWLGVIWGGGSTDYNAAFISRLSIS
KDNS KSQVFFKMNSLQANDTAIYYCARNDYPAWF
MIN-42 HEAVY CHAIN VARIABLE REGION AMINO ACID SEQUENCE
(SEQ ID NO: 267)
EVQLVESGGDLVKPGRSLKLSCAASGFTESSFGMSWVRQTPDKRLEWVATISSGGTYTYYPDSVKGRFTI
SRDNAKNTLYLQMSSLKSEDTAMYYCSRRFYYDYD
MIN-45 HEAVY CHAIN VARIABLE REGION AMINO ACID SEQUENCE
(SEQ ID NO: 268)
EVQLQQSGPELVKPGASVKISCKASGYSFTGYFMSWVMQSHGKSLEWIGRINPYNGDTFYNQKEKGKATL
TVDKSSTTAHIELRSLASEDSAVYYCARKGLYG
(SEQ ID NO: 269)
SASSSISYIH
DESCRIBES MIN-A2-1 LIGHT CHAIN VARIABLE
COMPLEMENTARITY DETERMINING REGION 1 (CDR1) AMINO ACID SEQUENCE.
(SEQ ID NO: 270)
SASSSVSYMH
DESCRIBES MIN-A2-2 LIGHT CHAIN VARIABLE
COMPLEMENTARITY DETERMINING REGION 1 (CDR1) AMINO ACID SEQUENCE.
(SEQ ID NO: 271)
SASSSVSYMY
DESCRIBES MIN-C9-1 LIGHT CHAIN VARIABLE
COMPLEMENTARITY DETERMINING REGION 1 (CDR1) AMINO ACID SEQUENCE.
(SEQ ID NO: 272)
SASSSVSYTY
DESCRIBES MIN-C9-2 LIGHT CHAIN VARIABLE
COMPLEMENTARITY DETERMINING REGION 1 (CDR1) AMINO ACID SEQUENCE.
(SEQ ID NO: 273)
SASSSVSYMH
DESCRIBES MIN-D7-1 LIGHT CHAIN VARIABLE
COMPLEMENTARITY DETERMINING REGION 1 (CDR1) AMINO ACID SEQUENCE.
(SEQ ID NO: 274)
SASSSVSYMH
DESCRIBES MIN-D7-2 LIGHT CHAIN VARIABLE
COMPLEMENTARITY DETERMINING REGION 1 (CDR1) AMINO ACID SEQUENCE.
(SEQ ID NO: 275)
SASSSISYIH
DESCRIBES MIN-F2-1 LIGHT CHAIN VARIABLE
COMPLEMENTARITY DETERMINING REGION 1 (CDR1) AMINO ACID SEQUENCE.
(SEQ ID NO: 276)
SASSSISYIH
DESCRIBES MIN-F2-2 LIGHT CHAIN VARIABLE
COMPLEMENTARITY DETERMINING REGION 1 (CDR1) AMINO ACID SEQUENCE.
(SEQ ID NO: 277)
GTSNLAS
DESCRIBES MIN-A2-1 LIGHT CHAIN VARIABLE
COMPLEMENTARITY DETERMINING REGION 2 (CDR2) AMINO ACID SEQUENCE.
(SEQ ID NO: 278)
STSNLAS
DESCRIBES MIN-A2-2 LIGHT CHAIN VARIABLE
COMPLEMENTARITY DETERMINING REGION 2 (CDR2) AMINO ACID SEQUENCE.
(SEQ ID NO: 279)
STSNLAS
DESCRIBES MIN-C9-1 LIGHT CHAIN VARIABLE
COMPLEMENTARITY DETERMINING REGION 2 (CDR2) AMINO ACID SEQUENCE.
(SEQ ID NO: 280)
STSNLAS
DESCRIBES MIN-C9-2 LIGHT CHAIN VARIABLE
COMPLEMENTARITY DETERMINING REGION 2 (CDR2) AMINO ACID SEQUENCE.
(SEQ ID NO: 281)
STSNLAS
DESCRIBES MIN-D7-1 LIGHT CHAIN VARIABLE
COMPLEMENTARITY DETERMINING REGION 2 (CDR2) AMINO ACID SEQUENCE.
(SEQ ID NO: 282)
STSNLAS
DESCRIBES MIN-D7-2 LIGHT CHAIN VARIABLE
COMPLEMENTARITY DETERMINING REGION 2 (CDR2) AMINO ACID SEQUENCE.
(SEQ ID NO: 283)
GTSNLAS
DESCRIBES MIN-F2-1 LIGHT CHAIN VARIABLE
COMPLEMENTARITY DETERMINING REGION 2 (CDR2) AMINO ACID SEQUENCE.
(SEQ ID NO: 284)
GTSNLAS
DESCRIBES MIN-F2-2 LIGHT CHAIN VARIABLE
COMPLEMENTARITY DETERMINING REGION 2 (CDR2) AMINO ACID SEQUENCE.
(SEQ ID NO: 285)
QQRSNYPFT
DESCRIBES MIN-A2-1 LIGHT CHAIN VARIABLE
COMPLEMENTARITY DETERMINING REGION 3 (CDR3) AMINO ACID SEQUENCE.
(SEQ ID NO: 286)
QQRSSYPST
DESCRIBES MIN-A2-2 LIGHT CHAIN VARIABLE
COMPLEMENTARITY DETERMINING REGION 3 (CDR3) AMINO ACID SEQUENCE.
(SEQ ID NO: 287)
QQRSSYPST
DESCRIBES MIN-C9-1 LIGHT CHAIN VARIABLE
COMPLEMENTARITY DETERMINING REGION 3 (CDR3) AMINO ACID SEQUENCE.
(SEQ ID NO: 288)
QQRSSYPST
DESCRIBES MIN-C9-2 LIGHT CHAIN VARIABLE
COMPLEMENTARITY DETERMINING REGION 3 (CDR3) AMINO ACID SEQUENCE.
(SEQ ID NO: 289)
QQRSSYPST
DESCRIBES MIN-D7-1 LIGHT CHAIN VARIABLE
COMPLEMENTARITY DETERMINING REGION 3 (CDR3) AMINO ACID SEQUENCE.
(SEQ ID NO: 290)
QQRSSYPST
DESCRIBES MIN-D7-2 LIGHT CHAIN VARIABLE
COMPLEMENTARITY DETERMINING REGION 3 (CDR3) AMINO ACID SEQUENCE.
(SEQ ID NO: 291)
QQRSNYPFT
DESCRIBES MIN-F2-1 LIGHT CHAIN VARIABLE
COMPLEMENTARITY DETERMINING REGION 3 (CDR3) AMINO ACID SEQUENCE.
(SEQ ID NO: 292)
QQRSNYPFT
DESCRIBES MIN-F2-2 LIGHT CHAIN VARIABLE
COMPLEMENTARITY DETERMINING REGION 3 (CDR3) AMINO ACID SEQUENCE.
(SEQ ID NO: 293)
NYGMN
DESCRIBES MIN-A2-1 HEAVY CHAIN VARIABLE
COMPLEMENTARITY DETERMINING REGION 1 (CDR1) AMINO ACID SEQUENCE.
(SEQ ID NO: 294)
NYGMN
DESCRIBES MIN-A2-2 HEAVY CHAIN VARIABLE
COMPLEMENTARITY DETERMINING REGION 1 (CDR1) AMINO ACID SEQUENCE.
(SEQ ID NO: 295)
NNGMN
DESCRIBES MIN-C9-1 HEAVY CHAIN VARIABLE
COMPLEMENTARITY DETERMINING REGION 1 (CDR1) AMINO ACID SEQUENCE.
(SEQ ID NO: 296)
NNGMN
DESCRIBES MIN-C9-2 HEAVY CHAIN VARIABLE
COMPLEMENTARITY DETERMINING REGION 1 (CDR1) AMINO ACID SEQUENCE.
(SEQ ID NO: 297)
NYGMN
DESCRIBES MIN-D7-1 HEAVY CHAIN VARIABLE
COMPLEMENTARITY DETERMINING REGION 1 (CDR1) AMINO ACID SEQUENCE.
(SEQ ID NO: 298)
NYGMN
DESCRIBES MIN-D7-2 HEAVY CHAIN VARIABLE
COMPLEMENTARITY DETERMINING REGION 1 (CDR1) AMINO ACID SEQUENCE.
(SEQ ID NO: 299)
NYGMN
DESCRIBES MIN-F2-1 HEAVY CHAIN VARIABLE
COMPLEMENTARITY DETERMINING REGION 1 (CDR1) AMINO ACID SEQUENCE.
(SEQ ID NO: 300)
DYEMH
DESCRIBES MIN-F2-2 HEAVY CHAIN VARIABLE
COMPLEMENTARITY DETERMINING REGION 1 (CDR1) AMINO ACID SEQUENCE.
(SEQ ID NO: 301)
NYGMN
DESCRIBES MIN-F2-3 HEAVY CHAIN VARIABLE
COMPLEMENTARITY DETERMINING REGION 1 (CDR1) AMINO ACID SEQUENCE.
(SEQ ID NO: 302)
NYGMN
DESCRIBES MIN-F2-4 HEAVY CHAIN VARIABLE
COMPLEMENTARITY DETERMINING REGION 1 (CDR1) AMINO ACID SEQUENCE.
(SEQ ID NO: 303)
WINTYTGEPTYAGDEKG
DESCRIBES MIN-A2-1 HEAVY CHAIN
VARIABLE COMPLEMENTARITY DETERMINING REGION 2 (CDR2) AMINO ACID
SEQUENCE.
(SEQ ID NO: 304)
WINTYTGEPTYAGDEKG
DESCRIBES MIN-A2-2 HEAVY CHAIN
VARIABLE COMPLEMENTARITY DETERMINING REGION 2 (CDR2) AMINO ACID
SEQUENCE.
(SEQ ID NO: 305)
WINTYTGEPTYADDFKG
DESCRIBES MIN-C9-1 HEAVY CHAIN
VARIABLE COMPLEMENTARITY DETERMINING REGION 2 (CDR2) AMINO ACID
SEQUENCE.
(SEQ ID NO: 306)
WINTYTGEPTYADDEKG
DESCRIBES MIN-C9-2 HEAVY CHAIN
VARIABLE COMPLEMENTARITY DETERMINING REGION 2 (CDR2) AMINO ACID
SEQUENCE.
(SEQ ID NO: 307)
WINTYTGEPTYVDDEKG
DESCRIBES MIN-D7-1 HEAVY CHAIN
VARIABLE COMPLEMENTARITY DETERMINING REGION 2 (CDR2) AMINO ACID
SEQUENCE.
(SEQ ID NO: 308)
WINTYTGEPTYAGDFKG
DESCRIBES MIN-D7-2 HEAVY CHAIN
VARIABLE COMPLEMENTARITY DETERMINING REGION 2 (CDR2) AMINO ACID SEQUENCE.
(SEQ ID NO: 309)
WINTYTGEPTYVDDFKG
DESCRIBES MIN-F2-1 HEAVY CHAIN
VARIABLE COMPLEMENTARITY DETERMINING REGION 2 (CDR2) AMINO ACID SEQUENCE.
(SEQ ID NO: 310)
AIHPGSGGTAYNQKFKG
DESCRIBES MIN-F2-2 HEAVY CHAIN
VARIABLE COMPLEMENTARITY DETERMINING REGION 2 (CDR2) AMINO ACID SEQUENCE.
(SEQ ID NO: 311)
WINTYTGEPTYVDDFKG
DESCRIBES MIN-F2-3 HEAVY CHAIN
VARIABLE COMPLEMENTARITY DETERMINING REGION 2 (CDR2) AMINO ACID SEQUENCE.
(SEQ ID NO: 312)
WINTYTGEPTYVDDFKG
DESCRIBES MIN-F2-4 HEAVY CHAIN
VARIABLE COMPLEMENTARITY DETERMINING REGION 2 (CDR2) AMINO ACID SEQUENCE.
(SEQ ID NO: 313)
SGDGYWYYA
DESCRIBES MIN-A2-1 HEAVY CHAIN VARIABLE
COMPLEMENTARITY DETERMINING REGION 3 (CDR3) AMINO ACID SEQUENCE.
(SEQ ID NO: 314)
SGDGYWYYA
DESCRIBES MIN-A2-2 HEAVY CHAIN VARIABLE
COMPLEMENTARITY DETERMINING REGION 3 (CDR3) AMINO ACID SEQUENCE.
(SEQ ID NO: 315)
TGTARAFYA
DESCRIBES MIN-C9-1 HEAVY CHAIN VARIABLE
COMPLEMENTARITY DETERMINING REGION 3 (CDR3) AMINO ACID SEQUENCE.
(SEQ ID NO: 316)
TGTARAFYA
DESCRIBES MIN-C9-2 HEAVY CHAIN VARIABLE
COMPLEMENTARITY DETERMINING REGION 3 (CDR3) AMINO ACID SEQUENCE.
(SEQ ID NO: 317)
TGTTAILNG
DESCRIBES MIN-D7-1 HEAVY CHAIN VARIABLE
COMPLEMENTARITY DETERMINING REGION 3 (CDR3) AMINO ACID SEQUENCE.
(SEQ ID NO: 318)
SGDGYWYYA
DESCRIBES MIN-D7-2 HEAVY CHAIN VARIABLE
COMPLEMENTARITY DETERMINING REGION3 (CDR3) AMINO ACID SEQUENCE.
(SEQ ID NO: 319)
TGTTAILNG
DESCRIBES MIN-F2-1 HEAVY CHAIN VARIABLE
COMPLEMENTARITY DETERMINING REGION 3 (CDR3) AMINO ACID SEQUENCE.
(SEQ ID NO: 320)
YGSFA
DESCRIBES MIN-F2-2 HEAVY CHAIN VARIABLE
COMPLEMENTARITY DETERMINING REGION 3 (CDR3) AMINO ACID SEQUENCE.
(SEQ ID NO: 321)
TGTTAILNG
DESCRIBES MIN-F2-3 HEAVY CHAIN VARIABLE
COMPLEMENTARITY DETERMINING REGION 3 (CDR3) AMINO ACID SEQUENCE.
(SEQ ID NO: 322)
TGTTAILNG
DESCRIBES MIN-F2-4 HEAVY CHAIN VARIABLE
COMPLEMENTARITY DETERMINING REGION 3 (CDR3) AMINO ACID SEQUENCE.
(SEQ ID NO: 323)
RASQDIGSSLN
DESCRIBES MIN-14 LIGHT CHAIN VARIABLE
COMPLEMENTARITY DETERMINING REGION 1 (CDR1) AMINO ACID SEQUENCE.
(SEQ ID NO: 324)
RASKSVSTSGYSYMH
DESCRIBES MIN-17-1 LIGHT CHAIN
VARIABLE COMPLEMENTARITY DETERMINING REGION 1 (CDR1) AMINO ACID SEQUENCE.
(SEQ ID NO: 325)
LASQTIGTWLA
DESCRIBES MIN-17-2 LIGHT CHAIN VARIABLE
COMPLEMENTARITY DETERMINING REGION 1 (CDR1) AMINO ACID SEQUENCE.
(SEQ ID NO: 326)
RASKSVSTSGYSYMH
DESCRIBES MIN-29 LIGHT CHAIN VARIABLE
COMPLEMENTARITY DETERMINING REGION 1 (CDR1) AMINO ACID SEQUENCE.
(SEQ ID NO: 327)
RASKSVSTSGYSYMH
DESCRIBES MIN-34 LIGHT CHAIN VARIABLE
COMPLEMENTARITY DETERMINING REGION 1 (CDR1) AMINO ACID SEQUENCE.
(SEQ ID NO: 328)
KASQNVGTNVG
DESCRIBES MIN-42 LIGHT CHAIN VARIABLE
COMPLEMENTARITY DETERMINING REGION 1 (CDR1) AMINO ACID SEQUENCE.
(SEQ ID NO: 329)
RASGNIHNFLA
DESCRIBES MIN-45 LIGHT CHAIN VARIABLE
COMPLEMENTARITY DETERMINING REGION 1 (CDR1) AMINO ACID SEQUENCE.
(SEQ ID NO: 330)
ATSSLDS
DESCRIBES MIN-14 LIGHT CHAIN VARIABLE
COMPLEMENTARITY DETERMINING REGION 2 (CDR2) AMINO ACID SEQUENCE.
(SEQ ID NO: 331)
LVSNLES
DESCRIBES MIN-17-1 LIGHT CHAIN VARIABLE
COMPLEMENTARITY DETERMINING REGION 2 (CDR2) AMINO ACID SEQUENCE.
(SEQ ID NO: 332)
AATSLAD
DESCRIBES MIN-17-2 LIGHT CHAIN VARIABLE
COMPLEMENTARITY DETERMINING REGION 2 (CDR2) AMINO ACID SEQUENCE.
(SEQ ID NO: 333)
LVSNLES
DESCRIBES MIN-29 LIGHT CHAIN VARIABLE
COMPLEMENTARITY DETERMINING REGION 2 (CDR2) AMINO ACID SEQUENCE.
(SEQ ID NO: 334)
LVSNLES
DESCRIBES MIN-34 LIGHT CHAIN VARIABLE
COMPLEMENTARITY DETERMINING REGION 2 (CDR2) AMINO ACID SEQUENCE.
(SEQ ID NO: 335)
SASYRYS
DESCRIBES MIN-42 LIGHT CHAIN VARIABLE
COMPLEMENTARITY DETERMINING REGION 2 (CDR2) AMINO ACID SEQUENCE.
(SEQ ID NO: 336)
NAKTLAD
DESCRIBES MIN-45 LIGHT CHAIN VARIABLE
COMPLEMENTARITY DETERMINING REGION 2 (CDR2) AMINO ACID SEQUENCE.
(SEQ ID NO: 337)
SYWMH
DESCRIBES MIN-14 HEAVY CHAIN COMPLEMENTARITY
DETERMINING REGION 1 (CDR1) AMINO ACID SEQUENCE.
(SEQ ID NO: 338)
SYWMH
DESCRIBES MIN-17-1 HEAVY CHAIN COMPLEMENTARITY
DETERMINING REGION 1 (CDR1) AMINO ACID SEQUENCE.
(SEQ ID NO: 339)
GIGVT
DESCRIBES MIN-17-2 HEAVY CHAIN COMPLEMENTARITY
DETERMINING REGION 1 (CDR1) AMINO ACID SEQUENCE.
(SEQ ID NO: 340)
SYGVH
DESCRIBES MIN-34 HEAVY CHAIN COMPLEMENTARITY
DETERMINING REGION 1 (CDR1) AMINO ACID SEQUENCE.
(SEQ ID NO: 341)
SFGMS
DESCRIBES MIN-42 HEAVY CHAIN COMPLEMENTARITY
DETERMINING REGION 1 (CDR1) AMINO ACID SEQUENCE.
(SEQ ID NO: 342)
GYFMS
DESCRIBES MIN-45 HEAVY CHAIN COMPLEMENTARITY
DETERMINING REGION 1 (CDR1) AMINO ACID SEQUENCE.
(SEQ ID NO: 343)
EINPSNGRTNYNEKFKS
DESCRIBES MIN-14 HEAVY CHAIN
COMPLEMENTARITY DETERMINING REGION 2 (CDR2) AMINO ACID SEQUENCE.
(SEQ ID NO: 344)
EINPSNGRTNYNEKFKS
DESCRIBES MIN-17-1 HEAVY CHAIN
COMPLEMENTARITY DETERMINING REGION 2 (CDR2) AMINO ACID SEQUENCE.
(SEQ ID NO: 345)
TIWWDDDNRYNPSLKS
DESCRIBES MIN-17-2 HEAVY CHAIN
COMPLEMENTARITY DETERMINING REGION 2 (CDR2) AMINO ACID SEQUENCE.
(SEQ ID NO: 346)
GIVSGDGESALHSVWIVG
DESCRIBES MIN-29 HEAVY CHAIN
COMPLEMENTARITY DETERMINING REGION 2 (CDR2) AMINO ACID SEQUENCE.
(SEQ ID NO: 347)
VIWGGGSTDYNAAFIS
DESCRIBES MIN-34 HEAVY CHAIN
COMPLEMENTARITY DETERMINING REGION 2 (CDR2) AMINO ACID SEQUENCE.
(SEQ ID NO: 348)
TISSGGTYTYYPDSVKG
DESCRIBES MIN-42 HEAVY CHAIN
COMPLEMENTARITY DETERMINING REGION 2 (CDR2) AMINO ACID SEQUENCE.
(SEQ ID NO: 349)
RINPYNGDTFYNQKFKG
DESCRIBES MIN-45 HEAVY CHAIN
COMPLEMENTARITY DETERMINING REGION 2 (CDR2) AMINO ACID SEQUENCE.
HUMANIZED E6 HEAVY CHAIN VARIABLE REGION SEQUENCE:
(SEQ ID NO: 350)
EVQLVESGGGLVKPGGSLRLSCAASGFTFSRYGMSWVRQAPGKRLEWVSTISGGGTYIYYPDSVKGRFTI
SRDNAKNTLYLQMNSLRAEDTAVYYCTRDNYGRNYDYGMDYWGQGTLVTVSS
HUMANIZED E6 HEAVY CHAIN VARIABLE COMPLEMENTARITY DETERMINING REGIONS
1 (CDR1) SEQUENCE:
(SEQ ID NO: 351)
RYGMS
HUMANIZED E6 HEAVY CHAIN VARIABLE COMPLEMENTARITY DETERMINING REGIONS
2 (CDR2) SEQUENCE:
(SEQ ID NO: 352)
TISGGGTYIYYPDSVKG
HUMANIZED E6 HEAVY CHAIN VARIABLE COMPLEMENTARITY DETERMINING REGIONS
3 (CDR3) SEQUENCE:
(SEQ ID NO: 353)
DNYGRNYDYGMDY
HUMANIZED E6 LIGHT CHAIN VARIABLE REGION SEQUENCE:
(SEQ ID NO: 354)
EIVLTQSPATLSLSPGERATLTCSATSSVSYIHWYQQRPGQSPRLLIYSTSNLASGIPARFSGSGSGSDY
TLTISSLEPEDFAVYYCQQRSSSPFTFGSGTKVEIK
HUMANIZED E6 LIGHT CHAIN VARIABLE COMPLEMENTARITY DETERMINING REGIONS
1 (CDR1) SEQUENCE:
(SEQ ID NO: 355)
SATSSVSYIH
HUMANIZED E6 LIGHT CHAIN VARIABLE COMPLEMENTARITY DETERMINING REGIONS
2 (CDR2) SEQUENCE:
(SEQ ID NO: 356)
STSNLAS
HUMANIZED E6 LIGHT CHAIN VARIABLE COMPLEMENTARITY DETERMINING REGIONS
3 (CDR3) SEQUENCE:
(SEQ ID NO: 357)
QQRSSSPFT
HUMANIZED C2 HEAVY CHAIN VARIABLE REGION SEQUENCE:
(SEQ ID NO: 358)
EVQLVESGGGLVKPGGSLRLSCAASGFTFSGYAMSWVRQAPGKGLEWVSTISSGGTYIYYPDSVKGRFTI
SRDNAKNSLYLQMNSLRAEDTAVYYCARLGGDNYYEYFDVWGKGTTVTVSS
HUMANIZED C2 HEAVY CHAIN VARIABLE COMPLEMENTARITY DETERMINING REGIONS
1 (CDR1) SEQUENCE:
(SEQ ID NO: 359)
GYAMS
HUMANIZED C2 HEAVY CHAIN VARIABLE COMPLEMENTARITY DETERMINING REGIONS
2 (CDR2) SEQUENCE:
(SEQ ID NO: 360)
TISSGGTYIYYPDSVKG
HUMANIZED C2 HEAVY CHAIN VARIABLE COMPLEMENTARITY DETERMINING REGIONS
3 (CDR3) SEQUENCE:
(SEQ ID NO: 361)
LGGDNYYEYFDV
HUMANIZED C2 LIGHT CHAIN VARIABLE REGION SEQUENCE:
(SEQ ID NO: 362)
DIVLTQSPASLAVSPGQRATITCRASKSVSTSGYSYMHWYQQKPGQPPKLLIYLASNLESGVPARESGSG
SGTDFTLTINPVEANDTANYYCQHSRELPFTFGGGTKVEIKRT
HUMANIZED C2 LIGHT CHAIN VARIABLE COMPLEMENTARITY DETERMINING REGIONS
1 (CDR1) SEQUENCE:
(SEQ ID NO: 363)
RASKSVSTSGYSYMH
HUMANIZED C2 LIGHT CHAIN VARIABLE COMPLEMENTARITY DETERMINING REGIONS
2 (CDR2) SEQUENCE:
(SEQ ID NO: 364)
LASNLES
HUMANIZED C2 LIGHT CHAIN VARIABLE COMPLEMENTARITY DETERMINING REGIONS
3 (CDR3) SEQUENCE:
(SEQ ID NO: 365)
QHSRELPFT
HUMANIZED C2 LIGHT CHAIN VARIABLE COMPLEMENTARITY DETERMINING REGIONS
3 (CDR3) SEQUENCE:
(SEQ ID NO: 366)
LQSKNFPPT
PSECTAG2 C2 SCFV-FC
(SEQ ID NO: 367)
METDTLLLWVLLLWVPGSTGDAAQPAEVQLVESGGGLVKPGGSLRLSCAASGFTFSGYAMSWVRQAPGKG
LEWVSTISSGGTYIYYPDSVKGRFTISRDNAKNSLYLQMNSLRAEDTAVYYCARLGGDNYYEYEDVWGKG
TTVTVSSGGGGSGGGGSGGGGSDIVLTQSPASLAVSPGQRATITCRASKSVSTSGYSYMHWYQQKPGQPP
KLLIYLASNLESGVPARFSGSGSGTDFTLTINPVEANDTANYYCQHSRELPFTFGGGTKVEIKRTEPKSC
DKTHTCPPCPAPELLGGPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHE
ALHNHYTQKSLSLSPGK**
PSECTAG2 E6 SCFV-FC
(SEQ ID NO: 368)
METDTLLLWVLLLWVPGSTGDAAQPAEVQLVESGGGLVKPGGSLRLSCAASGFTFSRYGMSWVRQAPGKR
LEWVSTISGGGTYIYYPDSVKGRFTISRDNAKNTLYLQMNSLRAEDTAVYYCTRDNYGRNYDYGMDYWGQ
GTLVTVSSGGGGSGGGGSGGGGSEIVLTQSPATLSLSPGERATLTCSATSSVSYIHWYQQRPGQSPRLLI
YSTSNLASGIPARFSGSGSGSDYTLTISSLEPEDFAVYYCQQRSSSPFTFGSGTKVEIKEPKSCDKTHTC
PPCPAPELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVESCSVMHEALHNHY
TQKSLSLSPGK**
HUMANIZED C2 SCFV (VH-VL) SEQUENCE:
(SEQ ID NO: 369)
EVQLVESGGGLVKPGGSLRLSCAASGFTFSGYAMSWVRQAPGKGLEWVSTISSGGTYIYYPDSVKGRETI
SRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTD
FTLTINPVEANDTANYYCQHSRELPFTFGGGTKVEIKRT
HUMANIZED E6 SCFV (VL-VH) SEQUENCE:
(SEQ ID NO: 370)
DIVLTQSPASLAVSPGQRATITCRASKSVSTSGYSYMHWYQQKPGQPPKLLIYLASNLESGVPARFSGSG
SGTDFTLTINPVEANDTANYYCQHSRELPFTFGGGTKVEIKRTGGGGSGGGGSGGGGSEVQLVESGGGLV
KPGGSLRLSCAASGFTFSGYAMSWVRQAPGKGLEWVSTISSGGTYIYYPDSVKGRFTISRDNAKNSLYLQ
MNSLRAEDTAVYYCARLGGDNYYEYFDVWGKGTTVTVSS
HUMANIZED C3 HEAVY CHAIN VARIABLE REGION SEQUENCE:
(SEQ ID NO: 371)
QVQLVQSGAEVKKPGASVKVSCKASGYTFTDYAMNWVRQAPGQGLEWMGVISTFSGNTNFNQKFKGRVTM
TTDTSTSTAYMELRSLRSDDTAVYYCARSDYYGPYFDYWGQGTTLTVSS
HUMANIZED C3 HEAVY CHAIN VARIABLE COMPLEMENTARITY DETERMINING REGIONS
1 (CDR1) SEQUENCE:
(SEQ ID NO: 372)
DYAMN
HUMANIZED C3 HEAVY CHAIN VARIABLE COMPLEMENTARITY DETERMINING REGIONS
2 (CDR2) SEQUENCE:
(SEQ ID NO: 373)
VISTFSGNTNFNQKEKG
HUMANIZED C3 HEAVY CHAIN VARIABLE COMPLEMENTARITY DETERMINING REGIONS
3 (CDR3) SEQUENCE:
(SEQ ID NO: 374)
SDYYGPYFDY
HUMANIZED C3 LIGHT CHAIN VARIABLE REGION SEQUENCE:
DIVMTQTPLSLSVTPGQPASISCRSSQTIVHSNGNTYLEWYLQKPGQSPQLLIYKVSNRFSGVPDRESGS
(SEQ ID NO: 375)
GSGTDFTLKISRVEAEDVGVYYCFQGSHVPFTFGGGTKVEIKRT
HUMANIZED C3 LIGHT CHAIN VARIABLE COMPLEMENTARITY DETERMINING REGIONS
1 (CDR1) SEQUENCE:
(SEQ ID NO: 376)
RSSQTIVHSNGNTYLE
HUMANIZED C3 LIGHT CHAIN VARIABLE COMPLEMENTARITY DETERMINING REGIONS
2 (CDR2) SEQUENCE:
(SEQ ID NO: 377)
KVSNRFS
HUMANIZED C3 LIGHT CHAIN VARIABLE COMPLEMENTARITY DETERMINING REGIONS
3 (CDR3) SEQUENCE:
(SEQ ID NO: 378)
FQGSHVPFT
HUMANIZED C8 HEAVY CHAIN VARIABLE REGION SEQUENCE:
(SEQ ID NO: 379)
EVQLVESGGGLVKPGGSLRLSCAASGFTFSGYAMSWVRQAPGKGLEWVSTISSGGTYIYYPDSVKGRFTI
SRDNAKNSLYLQMNSLRAEDTAVYYCARLGGDNYYEYWGKGTTVTVSS
HUMANIZED C8 HEAVY CHAIN VARIABLE COMPLEMENTARITY DETERMINING REGION 1
(CDR1) SEQUENCE:
(SEQ ID NO: 380)
GYAMS
HUMANIZED C8 HEAVY CHAIN VARIABLE COMPLEMENTARITY DETERMINING REGION 2
(CDR2) SEQUENCE:
(SEQ ID NO: 381)
TISSGGTYIYYPDSVKG
HUMANIZED C8 HEAVY CHAIN VARIABLE COMPLEMENTARITY DETERMINING REGION 3
(CDR3) SEQUENCE:
(SEQ ID NO: 382)
LGGDNYYEY
HUMANIZED C8 LIGHT CHAIN VARIABLE REGION SEQUENCE
(SEQ ID NO: 383)
DIVMTQSPDSLAVSLGERATINCRASKSVSTSGYSYMHWYQQKPGQPPKLLIYLVSNLESGVPDRESGSG
SGTDETLTISSLQAEDVAVYYCQHIRELTRSEFGGGTKVEIKRT
HUMANIZED C8 LIGHT CHAIN VARIABLE COMPLEMENTARITY DETERMINING REGION 1
(CDR1) SEQUENCE:
(SEQ ID NO: 384)
RASKSVSTSGYSYM
HUMANIZED C8 LIGHT CHAIN VARIABLE COMPLEMENTARITY DETERMINING REGION 2
(CDR2) SEQUENCE:
(SEQ ID NO: 385)
LVSNLES
HUMANIZED C8 LIGHT CHAIN VARIABLE COMPLEMENTARITY DETERMINING REGION 3
(CDR3) SEQUENCE:
(SEQ ID NO: 386)
QHIRELTRSE

All of the references cited herein are incorporated by reference in their entirety.

Those skilled in the art will recognize, or be able to ascertain using no more than routine experimentation, many equivalents to the specific embodiments of the invention specifically described herein.

What is claimed is:

1. An anti-MUC1* antibody or antibody fragment comprising six complementarity determining regions (CDRs) of:
18B4 (SEQ ID NOs: 144, 146, 148, 150, 152, and 154),
18G12 (SEQ ID NOs: 80, 82, 84, 86, 88, and 90),
25E6 (SEQ ID NOs: 112, 114, 116, 118, 120, and 122),
28F9 (SEQ ID NOs: 128, 130, 132, 134, 136, and 138),
1E4 (SEQ ID NOs: 160, 162, 164, 166, 168, and 170),
29H1 (SEQ ID NOs: 176, 178, 180, 182, 184, and 186),
31A1 (SEQ ID NOs: 192, 194, 196, 198, 200, and 202),
32C1 (SEQ ID NOs: 208, 210, 212, 214, 216, and 218),
45C11 (SEQ ID NOs: 224, 226, 228, 230, 232, and 234),
3C5 (SEQ ID NOs: 48, 50, 52, 54, 56, and 58),
8A9 (SEQ ID NOs: 64, 66, 68, 70, 72, and 74),
17H6 (SEQ ID NOs: 16, 18, 20, 22, 24, and 26), or
39H5 (SEQ ID NOs: 32, 34, 36, 38, 40, and 42);
wherein the six CDRs of each antibody are heavy chain CDR1, heavy chain CDR2, heavy chain CDR3, light chain CDR1, light chain CDR2, and light chain CDR3, respectively.

2. The anti-MUC1* antibody or antibody fragment of claim 1, wherein the antibody or antibody fragment comprises six CDRs of 18B4 (SEQ ID NOs: 144, 146, 148, 150, 152, and 154).

3. The anti-MUC1* antibody or antibody fragment of claim 1, wherein the antibody or antibody fragment comprises six CDRs of 18G12 (SEQ ID NOs: 80, 82, 84, 86, 88, and 90).

4. The anti-MUC1* antibody or antibody fragment of claim 1, wherein the antibody or antibody fragment comprises six CDRs of 25E6 (SEQ ID NOs: 112, 114, 116, 118, 120, and 122).

5. The anti-MUC1* antibody or antibody fragment of claim 1, wherein the antibody or antibody fragment is human or humanized.

6. A conjugate comprising the anti-MUC1* antibody or antibody fragment of claim 1 attached to an imaging agent, a dye, a fluorescent entity, a color producing reagent or any other entity that renders the antibody or antibody fragment optically, visually, electrically or radioactively detectable.

7. A method of diagnosing cancer in a subject comprising contacting a cell or tissue of the subject with the conjugate of claim 6.

8. The method of claim 7, wherein the contacting is carried out in vitro.

9. The method of claim 7, wherein the contacting is carried out in vivo.

10. The method of claim 7, wherein the diagnosing further comprises:
a) determining that:
i. an amount of the antibody or antibody fragment binding to the cell or tissue is greater than an amount of the antibody or antibody fragment bound to a normal cell or normal tissue; or
ii. a pattern of the antibody or antibody fragment binding to the cell or tissue is not restricted to an apical border of the cell or tissue; and
b) concluding that the subject is suffering from a MUC1* positive cancer.

11. A method of determining suitability of treating a patient suffering from cancer or metastasis of cancer with a MUC1* targeting therapeutic agent, the method comprising contacting a cell or tissue from the patient with the conjugate of claim 6.

12. The method of claim 11, further comprising:
a) determining that the conjugate binds specifically to the cell or tissue; and
b) concluding that the patient is suitable for treatment with the MUC1* targeting therapeutic agent.

13. The method of 11, wherein the MUC1* targeting therapeutic agent comprises the 6 CDRs of the antibody or antibody fragment.

14. A method of treating a MUC1* positive cancer in a subject, comprising:
a) determining that the conjugate of claim 6 specifically binds to a cell or tissue of the MUC1* positive cancer; and
b) administering a MUC1* targeting therapeutic agent to the subject.

15. The method of claim 14, wherein the MUC1* targeting therapeutic agent comprises a cancer immunotherapy.

16. The method of claim 14, wherein the MUC1* targeting therapeutic agent comprises a chimeric antigen receptor (CAR) T cell.

17. The method of claim 14, wherein the MUC1* targeting therapeutic agent comprises a bispecific T cell engager.

18. The method of claim 14, wherein the MUC1* targeting therapeutic agent comprises an antibody drug conjugate.

19. The method of claim 14, wherein the MUC1* targeting therapeutic agent comprises the 6 CDRs of the antibody or antibody fragment.

20. The method of claim 14, wherein the MUC1* targeting therapeutic agent comprises the antibody or antibody fragment.


SEQUENCE LISTING (386 sequences)

SEQ ID NO: 1 - protein, 1255 aa, synthetic construct
  REGION 1..1255: note = Full-length MUC1 Receptor (Mucin 1 precursor, Genbank Accession number: P15941
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 171 aa, synthetic construct
  REGION 1..171: note = A truncated MUC1 receptor isoform having nat-PSMGFR and PSIBR at its N-terminus and including the transmembrane and cytoplasmic sequences of a full-length MUC1 receptor which may be cleaved after translation and prior to expression of the receptor on the
GFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 3 - protein, 275 aa, synthetic construct
  REGION 1..275: note = A truncated MUC1 receptor isoform having nat-PSMGFR + PSIBR + Unique Region at its N-terminus and including the transmembrane and cytoplasmic sequences of a full-length MUC1 receptor
ATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 4 - protein, 45 aa, synthetic construct
  REGION 1..45: note = PSMGFR
GTINVHDVETQFNQYKTEAASRYNLTISDVSVSDVPFPFSAQSGA

SEQ ID NO: 5 - protein, 43 aa, synthetic construct
  REGION 1..43: note = PSMGFR N+20/C-22
SNIKFRPGSVVVQLTLAFREGTINVHDVETQFNQYKTEAASRY

SEQ ID NO: 6 - protein, 35 aa, synthetic construct
  REGION 1..35: note = PSMGFR N+12/C-22
SVVVQLTLAFREGTINVHDVETQFNQYKTEAASRY

SEQ ID NO: 7 - protein, 24 aa, synthetic construct
  REGION 1..24: note = PSMGFR N+9/C-30
VQLTLAFREGTINVHDVETQFNQY

SEQ ID NO: 8 - protein, 24 aa, synthetic construct
  REGION 1..24: note = PSMGFR N+20/C-41
SNIKFRPGSVVVQLTLAFREGTIN

SEQ ID NO: 9 - protein, 38 aa, synthetic construct
  REGION 1..38: note = PSMGFR N+20/C-27
SNIKFRPGSVVVQLTLAFREGTINVHDVETQFNQYKTE

SEQ ID NO: 10 - protein, 45 aa, synthetic construct
  REGION 1..45: note = PSMGFR N+9/C-9
VQLTLAFREGTINVHDVETQFNQYKTEAASRYNLTISDVSVSDVP

SEQ ID NO: 11 - DNA, 426 bp, synthetic construct
  misc_feature 1..426: note = Antibody 17H6 Heavy chain - Signal sequence-FR1-CDR1-FR2-CDR2-FR3-CDR3-FR4
  source 1..426: mol_type = other DNA
atgaagttgtggctgaactggattttccttgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaagaaaggcacttgagtggttgggttttattagaaacaaagctaatggttacacagcagagtacagtgcgtctgtgaagggtcggttcaccatctccagagatgtttcccaaaacctcctctatcttcaaatgaacatcctgagagctgaggacagtgccacttattactgtgcaaaagattactacggtagtaaccctgcctggtttgcttactggggccaagggactctggtcactgtctctgca

SEQ ID NO: 12 - protein, 142 aa, synthetic construct
  REGION 1..142: note = Antibody 17H6 Heavy chain - Signal peptide-FR1-CDR1-FR2-CDR2-FR3-CDR3-FR4
MKLWLNWIFLVTLLNGIQCEVKLVESGGGLVQPGGSLRLSCATSGFTFTDYYMSWVRQPPRKALEWLGFIRNKANGYTAEYSASVKGRFTISRDVSQNLLYLQMNILRAEDSATYYCAKDYYGSNPAWFAYWGQGTLVTVSA

SEQ ID NO: 13 - DNA, 393 bp, synthetic construct
  misc_feature 1..393: note = Antibody 17H6 Light chain - Signal sequence-FR1-CDR1-FR2-CDR2-FR3-CDR3-FR4
  source 1..393: mol_type = other DNA
atgaagttgcctgtgaggctgttggtgctgatgttctggattcctgcttccaacagtgatattttgatgacccagactccactctccctgcctgtcagtcttggagatcaagcctccatctcttgcagatctagtcagagcattgtacatagtagtggaaacacctttttagaatggtacctgcagaaacctggccagtctccaaagctcctgatctacaaagtttccaaccgattttctggggtcccagacaggttcagtggcagtggatcagggatagatttcacactcaagatcagcagagtggaggctgaggatctgggagtttattactgctttcaaggttcacatgttcctttcacgttcggctcggggacaaagttggaaataaaa

SEQ ID NO: 14 - protein, 131 aa, synthetic construct
  REGION 1..131: note = Antibody 17H6 Light chain - Signal peptide-FR1-CDR1-FR2-CDR2-FR3-CDR3-FR4
MKLPVRLLVLMFWIPASNSDILMTQTPLSLPVSLGDQASISCRSSQSIVHSSGNTFLEWYLQKPGQSPKLLIYKVSNRFSGVPDRFSGSGSGIDFTLKISRVEAEDLGVYYCFQGSHVPFTFGSGTKLEIK

SEQ ID NO: 15 - DNA, 15 bp, synthetic construct
  misc_feature 1..15: note = Antibody 17H6 Heavy Chain CDR1
  source 1..15: mol_type = other DNA
gattactacatgagc

SEQ ID NO: 16 - protein, 5 aa, synthetic construct
  REGION 1..5: note = Antibody 17H6 Heavy Chain CDR1
DYYMS

SEQ ID NO: 17 - DNA, 57 bp, synthetic construct
  misc_feature 1..57: note = Antibody 17H6 Heavy Chain CDR2
  source 1..57: mol_type = other DNA
tttattagaaacaaagctaatggttacacagcagagtacagtgcgtctgtgaagggt

SEQ ID NO: 18 - protein, 19 aa, synthetic construct
  REGION 1..19: note = Antibody 17H6 Heavy Chain CDR
FIRNKANGYTAEYSASVKG

SEQ ID NO: 19 - DNA, 36 bp, synthetic construct
  misc_feature 1..36: note = Antibody 17H6 Heavy Chain CDR3
  source 1..36: mol_type = other DNA
gattactacggtagtaaccctgcctggtttgcttac

SEQ ID NO: 20 - protein, 12 aa, synthetic construct
  REGION 1..12: note = Antibody 17H6 Heavy Chain CDR3
DYYGSNPAWFAY

SEQ ID NO: 21 - DNA, 48 bp, synthetic construct
  misc_feature 1..48: note = Antibody 17H6 Light Chain CDR1
  source 1..48: mol_type = other DNA
agatctagtcagagcattgtacatagtagtggaaacacctttttagaa

SEQ ID NO: 22 - protein, 16 aa, synthetic construct
  REGION 1..16: note = Antibody 17H6 Light Chain CDR1
RSSQSIVHSSGNTFLE

SEQ ID NO: 23 - DNA, 21 bp, synthetic construct
  misc_feature 1..21: note = Antibody 17H6 Light Chain CDR2
  source 1..21: mol_type = other DNA
aaagtttccaaccgattttct

SEQ ID NO: 24 - protein, 7 aa, synthetic construct
  REGION 1..7: note = Antibody 17H6 Light Chain CDR2
KVSNRFS

SEQ ID NO: 25 - DNA, 27 bp, synthetic construct
  misc_feature 1..27: note = Antibody 17H6 Light Chain CDR3
  source 1..27: mol_type = other DNA
tttcaaggttcacatgttcctttcacg

SEQ ID NO: 26 - protein, 9 aa, synthetic construct
  REGION 1..9: note = Antibody 17H6 Light Chain CDR3
FQGSHVPFT

SEQ ID NO: 27 - DNA, 408 bp, synthetic construct
  misc_feature 1..408: note = Antibody 39H5 Heavy chain - Signal sequence-FR1-CDR1-FR2-CDR2-FR3-CDR3-FR4
  source 1..408: mol_type = other DNA
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttggtgacttcaagggacggtttgccttctctttggagacctctgccagcactgcctatttgcagatcaacaacctcaaaaatgaggacacggctacatatttttgtgttagaggtatccacggctacgtggactactggggccaaggcaccactctcacagtctcctca

SEQ ID NO: 28 - protein, 136 aa, synthetic construct
  REGION 1..136: note = Antibody 39H5 Heavy chain - Signal sequence-FR1-CDR1-FR2-CDR2-FR3-CDR3-FR4
MAWVWTLLFLMAAAQSAQAQIQLVQSGPELKKPGETVKISCKASGYTFTNYGMNWVKQAPGKGLKWMGWINTYTGEPTYVGDFKGRFAFSLETSASTAYLQINNLKNEDTATYFCVRGIHGYVDYWGQGTTLTVSS

SEQ ID NO: 29 - DNA, 393 bp, synthetic construct
  misc_feature 1..393: note = Antibody 39H5 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagaaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatcttccgtggacgttcggtggaggcaccaagctggaaatcaaa

SEQ ID NO: 30 - protein, 131 aa, synthetic construct
  REGION 1..131: note = Antibody 39H5 Light chain - Signal sequence-FR1-CDR1-FR2-CDR2-FR3-CDR3-FR4
MKLPVRLLVLMFWIPASSSDVLMTQTPLSLPVSLGDQASISCRSSQSIVHRNGNTYLEWYLQKPGQSPKLLIYKVSNRFSGVPDRFSGSGSGTDFTLKISRVEAEDLGVYYCFQGSHLPWTFGGGTKLEIK

SEQ ID NO: 31 - DNA, 15 bp, synthetic construct
  misc_feature 1..15: note = Antibody 39H5 Heavy Chain CDR1
  source 1..15: mol_type = other DNA
aactatggaatgaac

SEQ ID NO: 32 - protein, 5 aa, synthetic construct
  REGION 1..5: note = Antibody 39H5 Heavy Chain CDR1
NYGMN

SEQ ID NO: 33 - DNA, 51 bp, synthetic construct
  misc_feature 1..51: note = Antibody 39H5 Heavy Chain CDR2
  source 1..51: mol_type = other DNA
tggataaacacctacactggagagccaacatatgttggtgacttcaaggga

SEQ ID NO: 34 - protein, 17 aa, synthetic construct
  REGION 1..17: note = Antibody 39H5 Heavy Chain CDR2
WINTYTGEPTYVGDFKG

SEQ ID NO: 35 - DNA, 24 bp, synthetic construct
  misc_feature 1..24: note = Antibody 39H5 Heavy Chain CDR3
  source 1..24: mol_type = other DNA
ggtatccacggctacgtggactac

SEQ ID NO: 36 - protein, 8 aa, synthetic construct
  REGION 1..8: note = Antibody 39H5 Heavy Chain CDR3
GIHGYVDY

SEQ ID NO: 37 - DNA, 48 bp, synthetic construct
  misc_feature 1..48: note = Antibody 39H5 Light Chain CDR1
  source 1..48: mol_type = other DNA
agatctagtcagagcattgtacatagaaatggaaacacctatttagaa

SEQ ID NO: 38 - protein, 16 aa, synthetic construct
  REGION 1..16: note = Antibody 39H5 Light Chain CDR1
RSSQSIVHRNGNTYLE

SEQ ID NO: 39 - DNA, 21 bp, synthetic construct
  misc_feature 1..21: note = Antibody 39H5 Light Chain CDR2
  source 1..21: mol_type = other DNA
aaagtttccaaccgattttct

SEQ ID NO: 40 - protein, 7 aa, synthetic construct
  REGION 1..7: note = Antibody 39H5 Light Chain CDR2
KVSNRFS

SEQ ID NO: 41 - DNA, 27 bp, synthetic construct
  misc_feature 1..27: note = Antibody 39H5 Light Chain CDR3
  source 1..27: mol_type = other DNA
tttcaaggttcacatcttccgtggacg

SEQ ID NO: 42 - protein, 9 aa, synthetic construct
  REGION 1..9: note = Antibody 39H5 Light Chain CDR3
FQGSHLPWT

SEQ ID NO: 43 - DNA, 411 bp, synthetic construct
  misc_feature 1..411: note = Antibody 3C5 Heavy chain - Signal sequence-FR1-CDR1-FR2-CDR2-FR3-CDR3-FR4
  source 1..411: mol_type = other DNA
atggcttgggtgtggaccttgctgttcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggaaagccaacatatgctgatgacttcaagggacggtttgccttctctttggagacctctgccagcactgcctatttgcagatcaacaacctcaaaaatgaggacacggctacatatttctgtgcaagagggggactagatggttactacggctactggggccaaggcaccactctcacagtctcctca

SEQ ID NO: 44 - protein, 137 aa, synthetic construct
  REGION 1..137: note = Antibody 3C5 Heavy chain - Signal sequence-FR1-CDR1-FR2-CDR2-FR3-CDR3-FR4
MAWVWTLLFLMAAAQSAQAQIQLVQSGPELKKPGETVKISCKASGYTFTNYGMNWVKQAPGKGLKWMGWINTYTGKPTYADDFKGRFAFSLETSASTAYLQINNLKNEDTATYFCARGGLDGYYGYWGQGTTLTVSS

SEQ ID NO: 45 - DNA, 393 bp, synthetic construct
  misc_feature 1..393: note = Antibody 3C5 Light chain - Signal sequence-FR1-CDR1-FR2-CDR2-FR3-CDR3-FR4
  source 1..393: mol_type = other DNA
atgagtcctgcccagttcctgtttctgctagtgctctcgattcaggaaaccaacggtgatgttgtgatggctcagaccccactcactttgtcggttaccattggacaaccagcctccatctcttgcaaatcaagtcagagcctcttacatagtaaaggaaagacatatttgaattggttattacagaggccaggccagtctccaaagctcctaatctatctggtgtctaaactggaatctggagtccctgacaggttcagtggcagtggatcagggacagatttcacactgaaaatcagcagagtggaggctgaagatttgggagtttattactgcttgcaaactacacattttccgtggacgttcggtggaggcaccaagctggaaatcaaa

SEQ ID NO: 46 - protein, 131 aa, synthetic construct
  REGION 1..131: note = Antibody 3C5 Light chain - Signal sequence-FR1-CDR1-FR2-CDR2-FR3-CDR3-FR4
MSPAQFLFLLVLSIQETNGDVVMAQTPLTLSVTIGQPASISCKSSQSLLHSKGKTYLNWLLQRPGQSPKLLIYLVSKLESGVPDRFSGSGSGTDFTLKISRVEAEDLGVYYCLQTTHFPWTFGGGTKLEIK

SEQ ID NO: 47 - DNA, 15 bp, synthetic construct
  misc_feature 1..15: note = Antibody 3C5 Heavy Chain CDR1
  source 1..15: mol_type = other DNA
aactatggaatgaac

SEQ ID NO: 48 - protein, 5 aa, synthetic construct
  REGION 1..5: note = Antibody 3C5 Heavy Chain CDR1
NYGMN

SEQ ID NO: 49 - DNA, 51 bp, synthetic construct
  misc_feature 1..51: note = Antibody 3C5 Heavy Chain CDR2
  source 1..51: mol_type = other DNA
tggataaacacctacactggaaagccaacatatgctgatgacttcaaggga

SEQ ID NO: 50 - protein, 17 aa, synthetic construct
  REGION 1..17: note = Antibody 3C5 Heavy Chain CDR2
WINTYTGKPTYADDFKG

SEQ ID NO: 51 - DNA, 27 bp, synthetic construct
  misc_feature 1..27: note = Antibody 3C5 Heavy Chain CDR3
  source 1..27: mol_type = other DNA
gggggactagatggttactacggctac

SEQ ID NO: 52 - protein, 9 aa, synthetic construct
  REGION 1..9: note = Antibody 3C5 Heavy Chain CDR3
GGLDGYYGY

SEQ ID NO: 53 - DNA, 48 bp, synthetic construct
  misc_feature 1..48: note = Antibody 3C5 Light Chain CDR1
  source 1..48: mol_type = other DNA
aaatcaagtcagagcctcttacatagtaaaggaaagacatatttgaat

SEQ ID NO: 54 - protein, 16 aa, synthetic construct
  REGION 1..16: note = Antibody 3C5 Light Chain CDR1
KSSQSLLHSKGKTYLN

SEQ ID NO: 55 - DNA, 21 bp, synthetic construct
  misc_feature 1..21: note = Antibody 3C5 Light Chain CDR2
  source 1..21: mol_type = other DNA
ctggtgtctaaactggaatct

SEQ ID NO: 56 - protein, 7 aa, synthetic construct
  REGION 1..7: note = Antibody 3C5 Light Chain CDR2
LVSKLES

SEQ ID NO: 57 - DNA, 27 bp, synthetic construct
  misc_feature 1..27: note = Antibody 3C5 Light Chain CDR3
  source 1..27: mol_type = other DNA
ttgcaaactacacattttccgtggacg

SEQ ID NO: 58 - protein, 9 aa, synthetic construct
  REGION 1..9: note = Antibody 3C5 Light Chain CDR3
LQTTHFPWT

SEQ ID NO: 59 - DNA, 420 bp, synthetic construct
  misc_feature 1..420: note = Antibody 8A9 Heavy chain - Signal sequence-FR1-CDR1-FR2-CDR2-FR3-CDR3-FR4
  source 1..420: mol_type = other DNA
atgaagttgtggctgaactggattttccttgtaacacttttaaatggtatccagtgtgaggtggagctggtggagtctggaggaggcttggtacagcctgggggttctctgagactctcctgtgcaacttctgggttcaccttcactgatcactacatgagctgggtccgccagcctccaggaaaggcacttgagtggttgggatttattagaaacaaagctaatggttacacaacagagtacagtgcatctgtgaagggtcggttcaccatctccagagataattcccaaagcatcctctatcttcaaatgaaaaccctgagaactgaggacagtgccacttattactgtgcaagaccttctgactgggactcctggtttgcttactggggccaagggactctggtcactgtctctgca

SEQ ID NO: 60 - protein, 140 aa, synthetic construct
  REGION 1..140: note = Antibody 8A9 Heavy chain - Signal sequence-FR1-CDR1-FR2-CDR2-FR3-CDR3-FR4
MKLWLNWIFLVTLLNGIQCEVELVESGGGLVQPGGSLRLSCATSGFTFTDHYMSWVRQPPGKALEWLGFIRNKANGYTTEYSASVKGRFTISRDNSQSILYLQMKTLRTEDSATYYCARPSDWDSWFAYWGQGTLVTVSA

SEQ ID NO: 61 - DNA, 393 bp, synthetic construct
  misc_feature 1..393: note = Antibody 8A9 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttttgatgacccaaactccactctccctgcctgtcagtcttggtgatcaagcctccatctcttgcagatctagtcagagcattgtacatagtaatggcaacacctatttagattggtacttgcagaaaccaggccagtctccaaagctcctgatctacagagtttccaaccgattttctggggtcccagacaggttcagtggcagtggatcagggacagatttcacactcaagatcagcagagtggaggctgaggatctgggactttattactgttttcaaggttcacatgttccgtgggcgttcggtggaggcaccaagctggaaatcaaa

SEQ ID NO: 62 - protein, 131 aa, synthetic construct
  REGION 1..131: note = Antibody 8A9 Light chain - Signal sequence-FR1-CDR1-FR2-CDR2-FR3-CDR3-FR4
MKLPVRLLVLMFWIPASSSDVLMTQTPLSLPVSLGDQASISCRSSQSIVHSNGNTYLDWYLQKPGQSPKLLIYRVSNRFSGVPDRFSGSGSGTDFTLKISRVEAEDLGLYYCFQGSHVPWAFGGGTKLEIK

SEQ ID NO: 63 - DNA, 15 bp, synthetic construct
  misc_feature 1..15: note = Antibody 8A9 Heavy Chain CDR1
  source 1..15: mol_type = other DNA
gatcactacatgagc

SEQ ID NO: 64 - protein, 5 aa, synthetic construct
  REGION 1..5: note = Antibody 8A9 Heavy Chain CDR1
DHYMS

SEQ ID NO: 65 - DNA, 57 bp, synthetic construct
  misc_feature 1..57: note = Antibody 8A9 Heavy Chain CDR2
  source 1..57: mol_type = other DNA
tttattagaaacaaagctaatggttacacaacagagtacagtgcatctgtgaagggt

SEQ ID NO: 66 - protein, 19 aa, synthetic construct
  REGION 1..19: note = Antibody 8A9 Heavy Chain CDR2
FIRNKANGYTTEYSASVKG

SEQ ID NO: 67 - DNA, 30 bp, synthetic construct
  misc_feature 1..30: note = Antibody 8A9 Heavy Chain CDR3
  source 1..30: mol_type = other DNA
ccttctgactgggactcctggtttgcttac

SEQ ID NO: 68 - protein, 10 aa, synthetic construct
  REGION 1..10: note = Antibody 8A9 Heavy Chain CDR3
PSDWDSWFAY

SEQ ID NO: 69 - DNA, 48 bp, synthetic construct
  misc_feature 1..48: note = Antibody 8A9 Light Chain CDR1
  source 1..48: mol_type = other DNA
agatctagtcagagcattgtacatagtaatggcaacacctatttagat

SEQ ID NO: 70 - protein, 16 aa, synthetic construct
  REGION 1..16: note = Antibody 8A9 Light Chain CDR1
RSSQSIVHSNGNTYLD

SEQ ID NO: 71 - DNA, 21 bp, synthetic construct
  misc_feature 1..21: note = Antibody 8A9 Light Chain CDR2
  source 1..21: mol_type = other DNA
agagtttccaaccgattttct

SEQ ID NO: 72 - protein, 7 aa, synthetic construct
  REGION 1..7: note = Antibody 8A9 Light Chain CDR2
RVSNRFS

SEQ ID NO: 73 - DNA, 27 bp, synthetic construct
  misc_feature 1..27: note = Antibody 8A9 Light Chain CDR3
  source 1..27: mol_type = other DNA
tttcaaggttcacatgttccgtgggcg

SEQ ID NO: 74 - protein, 9 aa, synthetic construct
  REGION 1..9: note = Antibody 8A9 Light Chain CDR3
FQGSHVPWA

SEQ ID NO: 75 - DNA, 399 bp, synthetic construct
  misc_feature 1..399: note = Antibody 18G12 Heavy chain - Signal sequence-FR1-CDR1-FR2-CDR2-FR3-CDR3-FR4
  source 1..399: mol_type = other DNA
atgggatggagctatatcatcctctttttggtcgcaacagctacaggtgtccactcccaggtccaactgcagcagtctggggctgaactggtgaagcctggggcttcagtgaagttgtcctgcaaggcttctggctacaccttcaccggctactttttgtactgggtgaagcagaggcctggacaaggccttgagtggattggggggattaatcctgacaatggtggtattgacttcaatgagaagttcaggaacaaggccacactgactgtagacaaatcctccagcacagcctacatgcaactcagcagcctgacatctgaggactctgcggtctattattgtacattactaatagggaactattggggccaaggcaccactctcacagtctcctca

SEQ ID NO: 76 - protein, 133 aa, synthetic construct
  REGION 1..133: note = Antibody 18G12 Heavy chain - Signal sequence-FR1-CDR1-FR2-CDR2-FR3-CDR3-FR4
MGWSYIILFLVATATGVHSQVQLQQSGAELVKPGASVKLSCKASGYTFTGYFLYWVKQRPGQGLEWIGGINPDNGGIDFNEKFRNKATLTVDKSSSTAYMQLSSLTSEDSAVYYCTLLIGNYWGQGTTLTVSS

SEQ ID NO: 77 - DNA, 393 bp, synthetic construct
  misc_feature 1..393: note = Antibody 18G12 Light chain - Signal sequence-FR1-CDR1-FR2-CDR2-FR3-CDR3-FR4
  source 1..393: mol_type = other DNA
atgagtcctgcccagttcctgtttctgttagtgctctggattcgggaaaccaatggtgatgttgtgatgacccagactccactcactttgtcggtaaccattggacagccagcctccatctcttgcaagtcaagtcagagcctcttacatagtgatggaaagacatatttgatttggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttatttttgctgtcaaggtacacattttccgtggacgttcggtggaggcaccatgctggaaatcaaa

SEQ ID NO: 78 - protein, 131 aa, synthetic construct
  REGION 1..131: note = Antibody 18G12 Light chain - Signal sequence-FR1-CDR1-FR2-CDR2-FR3-CDR3-FR4
MSPAQFLFLLVLWIRETNGDVVMTQTPLTLSVTIGQPASISCKSSQSLLHSDGKTYLIWLLQRPGQSPKRLIYLVSKLDSGVPDRFTGSGSGTDFTLKISRVEAEDLGVYFCCQGTHFPWTFGGGTMLEIK

SEQ ID NO: 79 - DNA, 15 bp, synthetic construct
  misc_feature 1..15: note = Antibody 18G12 Heavy Chain CDR1
  source 1..15: mol_type = other DNA
ggctactttttgtac

SEQ ID NO: 80 - protein, 5 aa, synthetic construct
  REGION 1..5: note = Antibody 18G12 Heavy Chain CDR1
GYFLY

SEQ ID NO: 81 - DNA, 51 bp, synthetic construct
  misc_feature 1..51: note = Antibody 18G12 Heavy Chain CDR2
  source 1..51: mol_type = other DNA
gggattaatcctgacaatggtggtattgacttcaatgagaagttcaggaac

SEQ ID NO: 82 - protein, 17 aa, synthetic construct
  REGION 1..17: note = Antibody 18G12 Heavy Chain CDR2
GINPDNGGIDFNEKFRN

SEQ ID NO: 83 - DNA, 15 bp, synthetic construct
  misc_feature 1..15: note = Antibody 18G12 Heavy Chain CDR3
  source 1..15: mol_type = other DNA
ctaatagggaactat

SEQ ID NO: 84 - protein, 5 aa, synthetic construct
  REGION 1..5: note = Antibody 18G12 Heavy Chain CDR3
LIGNY

SEQ ID NO: 85 - DNA, 48 bp, synthetic construct
  misc_feature 1..48: note = Antibody 18G12 Light Chain CDR1
  source 1..48: mol_type = other DNA
aagtcaagtcagagcctcttacatagtgatggaaagacatatttgatt

SEQ ID NO: 86 - protein, 16 aa, synthetic construct
  REGION 1..16: note = Antibody 18G12 Light Chain CDR1
KSSQSLLHSDGKTYLI

SEQ ID NO: 87 - DNA, 21 bp, synthetic construct
  misc_feature 1..21: note = Antibody 18G12 Light Chain CDR2
  source 1..21: mol_type = other DNA
ctggtgtctaaactggactct

SEQ ID NO: 88 - protein, 7 aa, synthetic construct
  REGION 1..7: note = Antibody 18G12 Light Chain CDR2
LVSKLDS

SEQ ID NO: 89 - DNA, 27 bp, synthetic construct
  misc_feature 1..27: note = Antibody 18G12 Light Chain CDR3
  source 1..27: mol_type = other DNA
tgtcaaggtacacattttccgtggacg

SEQ ID NO: 90 - protein, 9 aa, synthetic construct
  REGION 1..9: note = Antibody 18G12 Light Chain CDR3
CQGTHFPWT

SEQ ID NO: 91 - DNA, 417 bp, synthetic construct
  misc_feature 1..417: note = Antibody 20A10 Heavy chain - Signal sequence-FR1-CDR1-FR2-CDR2-FR3-CDR3-FR4
  source 1..417: mol_type = other DNA
atgaacttcgggttcagcttgattttccttgtccttgttttaaaaggtgtccagtgtgaagtgatgctggtggagtctgggggaggcttagtgaagcctggagggtccctgaaactctcctgtgcagcctctggattcactttcagtacctatgccatgtcttggattcgccagactccagagaagaggctggagtgggtcgcatccattggtcgtgctggttccacctactattcagacagtgtgaagggccgattcaccatctccagagataatgtccggaacatcctgtacctgcaaatgagcagtctgaggtctgaggacacggccatgtattactgtgctagaggcccgatctacaatgattacgacgagtttgcttactggggccaagggactctggtcactgtctctgca

SEQ ID NO: 92 - protein, 139 aa, synthetic construct
  REGION 1..139: note = Antibody 20A10 Heavy chain - Signal sequence-FR1-CDR1-FR2-CDR2-FR3-CDR3-FR4
MNFGFSLIFLVLVLKGVQCEVMLVESGGGLVKPGGSLKLSCAASGFTFSTYAMSWIRQTPEKRLEWVASIGRAGSTYYSDSVKGRFTISRDNVRNILYLQMSSLRSEDTAMYYCARGPIYNDYDEFAYWGQGTLVTVSA

SEQ ID NO: 93 - DNA, 396 bp, synthetic construct
  misc_feature 1..396: note = Antibody 20A10 Light chain - Signal sequence-FR1-CDR1-FR2-CDR2-FR3-CDR3-FR4
  source 1..396: mol_type = other DNA
atggaatcacagactcaggtcttcctctccctgctgctctgggtatctggtacctgtgggaacattatgatgacacagtcgccatcatctctggctgtgtctgcaggagaaaaggtcactatgagctgtaagtccagtcaaagtgttttatacagttcaaatcagaagaactatttggcctggtaccagcagaaaccagggcagtctcctaaactgctgatctactgggcatccactagggaatctggtgtccctgatcgcttcacaggcagtggatctgggacagattttactcttaccatcagcagtgtacaagctgaagacctggcagtttattactgtcatcaatacctctcctcgctcacgttcggtgctgggaccaagctggagctgaaa

SEQ ID NO: 94 - protein, 132 aa, synthetic construct
  REGION 1..132: note = Antibody 20A10 Light chain - Signal sequence-FR1-CDR1-FR2-CDR2-FR3-CDR3-FR4
MESQTQVFLSLLLWVSGTCGNIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELK

SEQ ID NO: 95 - DNA, 15 bp, synthetic construct
  misc_feature 1..15: note = Antibody 20A10 Heavy Chain CDR1
  source 1..15: mol_type = other DNA
acctatgccatgtct

SEQ ID NO: 96 - protein, 5 aa, synthetic construct
  REGION 1..5: note = Antibody 20A10 Heavy Chain CDR1
TYAMS

SEQ ID NO: 97 - DNA, 48 bp, synthetic construct
  misc_feature 1..48: note = Antibody 20A10 Heavy Chain CDR2
  source 1..48: mol_type = other DNA
tccattggtcgtgctggttccacctactattcagacagtgtgaagggc

SEQ ID NO: 98 - protein, 16 aa, synthetic construct
  REGION 1..16: note = Antibody 20A10 Heavy Chain CDR2
SIGRAGSTYYSDSVKG

SEQ ID NO: 99 - DNA, 36 bp, synthetic construct
  misc_feature 1..36: note = Antibody 20A10 Heavy Chain CDR3
  source 1..36: mol_type = other DNA
ggcccgatctacaatgattacgacgagtttgcttac

SEQ ID NO: 100 - protein, 12 aa, synthetic construct
  REGION 1..12: note = Antibody 20A10 Heavy Chain CDR3
GPIYNDYDEFAY

SEQ ID NO: 101 - DNA, 51 bp, synthetic construct
  misc_feature 1..51: note = Antibody 20A10 Light Chain CDR1
  source 1..51: mol_type = other DNA
aagtccagtcaaagtgttttatacagttcaaatcagaagaactatttggcc

SEQ ID NO: 102 - protein, 17 aa, synthetic construct
  REGION 1..17: note = Antibody 20A10 Light Chain CDR1
KSSQSVLYSSNQKNYLA

SEQ ID NO: 103 - DNA, 21 bp, synthetic construct
  misc_feature 1..21: note = Antibody 20A10 Light Chain CDR2
  source 1..21: mol_type = other DNA
tgggcatccactagggaatct

SEQ ID NO: 104 - protein, 7 aa, synthetic construct
  REGION 1..7: note = Antibody 20A10 Light Chain CDR2
WASTRES

SEQ ID NO: 105 - DNA, 24 bp, synthetic construct
  misc_feature 1..24: note = Antibody 20A10 Light Chain CDR3
  source 1..24: mol_type = other DNA
catcaatacctctcctcgctcacg

SEQ ID NO: 106 - protein, 8 aa, synthetic construct
  REGION 1..8: note = Antibody 20A10 Light Chain CDR3
HQYLSSLT

SEQ ID NO: 107 - DNA, 414 bp, synthetic construct
  misc_feature 1..414: note = Antibody 25E6 Heavy chain - Signal sequence-FR1-CDR1-FR2-CDR2-FR3-CDR3-FR4
  source 1..414: mol_type = other DNA
atgaacttcgggctcagcttgattttccttgccctcattttaaaaggtgtccagtgtgaggtgcagctggtggagtctgggggagacttagtgaagcctggagggtccctgaaactctcctgtgcagcctctggtttcactttcagtagttatggaatgtcttgggttcgccagactccagacaagaggctggagtgggtcgcaaccattagtaatggtggtagacacaccttctatccagacagtgtgaaggggcgattcaccatctccagagacaatgccaagaacaccctgtatctgcaaatgagcagtctgaagtctgaggacacagccatgtatttatgtgtaagacagactgggacggagggctggtttgcttactggggccaagggactctggtcactgtctctgca

SEQ ID NO: 108 - protein, 138 aa, synthetic construct
  REGION 1..138: note = Antibody 25E6 Heavy chain - Signal sequence-FR1-CDR1-FR2-CDR2-FR3-CDR3-FR4
MNFGLSLIFLALILKGVQCEVQLVESGGDLVKPGGSLKLSCAASGFTFSSYGMSWVRQTPDKRLEWVATISNGGRHTFYPDSVKGRFTISRDNAKNTLYLQMSSLKSEDTAMYLCVRQTGTEGWFAYWGQGTLVTVSA

SEQ ID NO: 109 - DNA, 393 bp, synthetic construct
  misc_feature 1..393: note = Antibody 25E6 Light chain - Signal sequence-FR1-CDR1-FR2-CDR2-FR3-CDR3-FR4
  source 1..393: mol_type = other DNA
atgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 110 - protein, 131 aa, synthetic construct
  REGION 1..131: note = Antibody 25E6 Light chain - Signal sequence-FR1-CDR1-FR2-CDR2-FR3-CDR3-FR4
MSPAQFLFLLVLWIRETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 111 - DNA, 15 bp, synthetic construct
  misc_feature 1..15: note = Antibody 25E6 Heavy Chain CDR1
  source 1..15: mol_type = other DNA
agttatggaatgtct

SEQ ID NO: 112 - protein, 5 aa, synthetic construct
  REGION 1..5: note = Antibody 25E6 Heavy Chain CDR1
SYGMS

SEQ ID NO: 113 - DNA, 51 bp, synthetic construct
  misc_feature 1..51: note = Antibody 25E6 Heavy Chain CDR2
  source 1..51: mol_type = other DNA
accattagtaatggtggtagacacaccttctatccagacagtgtgaagggg

SEQ ID NO: 114 - protein, 17 aa, synthetic construct
  REGION 1..17: note = Antibody 25E6 Heavy Chain CDR2
TISNGGRHTFYPDSVKG

SEQ ID NO: 115 - DNA, 30 bp, synthetic construct
  misc_feature 1..30: note = Antibody 25E6 Heavy Chain CDR3
  source 1..30: mol_type = other DNA
cagactgggacggagggctggtttgcttac

SEQ ID NO: 116 - protein, 10 aa, synthetic construct
  REGION 1..10: note = Antibody 25E6 Heavy Chain CDR3
QTGTEGWFAY

SEQ ID NO: 117 - DNA, 48 bp, synthetic construct
  misc_feature 1..48: note = Antibody 25E6 Light Chain CDR1
  source 1..48: mol_type = other DNA
aagtcaagtcagagcctcttagatagtgatggaaagacatatttgaat

SEQ ID NO: 118 - protein, 16 aa, synthetic construct
  REGION 1..16: note = Antibody 25E6 Light Chain CDR1
KSSQSLLDSDGKTYLN

SEQ ID NO: 119 - DNA, 21 bp, synthetic construct
  misc_feature 1..21: note = Antibody 25E6 Light Chain CDR2
  source 1..21: mol_type = other DNA
ctggtgtctaaactggactct

SEQ ID NO: 120 - protein, 7 aa, synthetic construct
  REGION 1..7: note = Antibody 25E6 Light Chain CDR2
LVSKLDS

SEQ ID NO: 121 - DNA, 27 bp, synthetic construct
  misc_feature 1..27: note = Antibody 25E6 Light Chain CDR3
  source 1..27: mol_type = other DNA
tggcaaggtacacattttcctcagacg

SEQ ID NO: 122 - protein, 9 aa, synthetic construct
  REGION 1..9: note = Antibody 25E6 Light Chain CDR3
WQGTHFPQT

SEQ ID NO: 123 - DNA, 399 bp, synthetic construct
  misc_feature 1..399: note = Antibody 28F9 Heavy chain - Signal sequence-FR1-CDR1-FR2-CDR2-FR3-CDR3-FR4
  source 1..399: mol_type = other DNA
atgggatggagctatatcatcctctttttggtagcaacagctacaggtgtccactcccaggtccaactgcagcagcctggggctgaactggtgcagcctggggcttcagtgaagttgtcctgcaaggcttctggctacaccttcaccggctactttttgtactgggtgaagcagaggcctggacatggccttgagtggattgggggaattcatcctagcaatggtgatactgacttcaatgagaagttcaagaacaaggccacactgactgtagacatatcctccagcactgcctacatgcaactcagcagcctgacatctgaggactctgcggtctattattgtacattactaataggggtctactggggccaaggcaccactctcacagtctcctca

SEQ ID NO: 124 - protein, 133 aa, synthetic construct
  REGION 1..133: note = Antibody 28F9 Heavy chain - Signal sequence-FR1-CDR1-FR2-CDR2-FR3-CDR3-FR4
MGWSYIILFLVATATGVHSQVQLQQPGAELVQPGASVKLSCKASGYTFTGYFLYWVKQRPGHGLEWIGGIHPSNGDTDFNEKFKNKATLTVDISSSTAYMQLSSLTSEDSAVYYCTLLIGVYWGQGTTLTVSS

SEQ ID NO: 125 - DNA, 393 bp, synthetic construct
  misc_feature 1..393: note = Antibody 28F9 Light chain - Signal sequence-FR1-CDR1-FR2-CDR2-FR3-CDR3-FR4
  source 1..393: mol_type = other DNA
atgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttacatagtgatggaaagacatatttgatttggttgttacagaggccaggccagtctccaaagcgcctaatctatctggtgtctaaactggactctggagtccctgacaggttcaccggcagtggatcagggacagatttcacactgaaaatcagcagagtggaggctgaggatttgggagtttatttttgctgtcaaggtacacattttccgtggacgttcggtggaggcaccatgctggaaatcaaa

SEQ ID NO: 126 - protein, 131 aa, synthetic construct; identical to SEQ ID NO: 78
  REGION 1..131: note = Antibody 28F9 Light chain - Signal sequence-FR1-CDR1-FR2-CDR2-FR3-CDR3-FR4

SEQ ID NO: 127 - DNA, 15 bp, synthetic construct
  misc_feature 1..15: note = Antibody 28F9 Heavy Chain CDR1
  source 1..15: mol_type = other DNA
ggctactttttgtac

SEQ ID NO: 128 - protein, 5 aa, synthetic construct
  REGION 1..5: note = Antibody 28F9 Heavy Chain CDR1
GYFLY

SEQ ID NO: 129 - DNA, 51 bp, synthetic construct
  misc_feature 1..51: note = Antibody 28F9 Heavy Chain CDR2
  source 1..51: mol_type = other DNA
ggaattcatcctagcaatggtgatactgacttcaatgagaagttcaagaac

SEQ ID NO: 130 - protein, 17 aa, synthetic construct
  REGION 1..17: note = Antibody 28F9 Heavy Chain CDR2
GIHPSNGDTDFNEKFKN

SEQ ID NO: 131 - DNA, 15 bp, synthetic construct
  misc_feature 1..15: note = Antibody 28F9 Heavy Chain CDR3
  source 1..15: mol_type = other DNA
ctaataggggtctac

SEQ ID NO: 132 - protein, 5 aa, synthetic construct
  REGION 1..5: note = Antibody 28F9 Heavy Chain CDR3
LIGVY

SEQ ID NO: 133 - DNA, 48 bp, synthetic construct; identical to SEQ ID NO: 85
  misc_feature 1..48: note = Antibody 28F9 Light Chain CDR1
  source 1..48: mol_type = other DNA

SEQ ID NO: 134 - protein, 16 aa, synthetic construct
  REGION 1..16: note = Antibody 28F9 Light Chain CDR1
KSSQSLLHSDGKTYLI

SEQ ID NO: 135 - DNA, 21 bp, synthetic construct
  misc_feature 1..21: note = Antibody 28F9 Light Chain CDR2
  source 1..21: mol_type = other DNA
ctggtgtctaaactggactct

SEQ ID NO: 136 - protein, 7 aa, synthetic construct
  REGION 1..7: note = Antibody 28F9 Light Chain CDR2
LVSKLDS

SEQ ID NO: 137 - DNA, 27 bp, synthetic construct
  misc_feature 1..27: note = Antibody 28F9 Light Chain CDR3
  source 1..27: mol_type = other DNA
tgtcaaggtacacattttccgtggacg

SEQ ID NO: 138 - protein, 9 aa, synthetic construct
  REGION 1..9: note = Antibody 28F9 Light Chain CDR3
CQGTHFPWT

SEQ ID NO: 139 - DNA, 411 bp, synthetic construct
  misc_feature 1..411: note = Antibody 18B4 Heavy chain - Signal sequence-FR1-CDR1-FR2-CDR2-FR3-CDR3-FR4
  source 1..411: mol_type = other DNA
atgtacttgggactgaactatgtattcatagtttttctcttaaatggtgtccagagtgaagtgaaacttgaggagtctggaggaggcttggtgcaacctgggggatccatgaaactctcttgtgctgcctctggattcacttttaatgacgcctggatggactgggtccgccagtctccagagaaggggcttgagtgggttgctgaaattagaagcacagctaatattcatacaacatactatgctgagtctgtccaagggaggttcaccatctcaagagatgattccaaaagtagtgtctacctgcaaatgaacagcttgagagctgaagacactggcatttattattgtaccccattactctacggatttgcttactggggccaagggactctggtcactgtctctgca

SEQ ID NO: 140 - protein, 137 aa, synthetic construct
  REGION 1..137: note = Antibody 18B4 Heavy chain - Signal sequence-FR1-CDR1-FR2-CDR2-FR3-CDR3-FR4
MYLGLNYVFIVFLLNGVQSEVKLEESGGGLVQPGGSMKLSCAASGFTFNDAWMDWVRQSPEKGLEWVAEIRSTANIHTTYYAESVQGRFTISRDDSKSSVYLQMNSLRAEDTGIYYCTPLLYGFAYWGQGTLVTVSA

SEQ ID NO: 141 - DNA, 393 bp, synthetic construct
  misc_feature 1..393: note = Antibody 18B4 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttgtgatgacccaaagtccactctccctgcctgtcagtcttggagatcaagcctccatctcttgcagaactagtcagagccttgtacacagtaatggaaacacctatttacattggcacctgcagaagccaggccagtctccaaaggtcctgatctacaaagtttccagccgattttctggggtcccagacaggttcagtggcagtggatcggggacagatttcacactcaagatcagcagagtggaggctgaggatctgggagtttatttctgctctcaaaatacacatgttccgtacacgttcggaggggggaccaagctggaaataaaa

SEQ ID NO: 142 - protein, 131 aa, synthetic construct
  REGION 1..131: note = Antibody 18B4 Heavy chain - Signal sequence-FR1-CDR1-FR2-CDR2-FR3-CDR3-FR4
MKLPVRLLVLMFWIPASSSDVVMTQSPLSLPVSLGDQASISCRTSQSLVHSNGNTYLHWHLQKPGQSPKVLIYKVSSRFSGVPDRFSGSGSGTDFTLKISRVEAEDLGVYFCSQNTHVPYTFGGGTKLEIK

SEQ ID NO: 143 - DNA, 15 bp, synthetic construct
  misc_feature 1..15: note = Antibody 18B4 Heavy Chain CDR1
  source 1..15: mol_type = other DNA
gacgcctggatggac

SEQ ID NO: 144 - protein, 5 aa, synthetic construct
  REGION 1..5: note = Antibody 18B4 Heavy Chain CDR1
DAWMD

SEQ ID NO: 145 - DNA, 57 bp, synthetic construct
  misc_feature 1..57: note = Antibody 18B4 Heavy Chain CDR2
  source 1..57: mol_type = other DNA
gaaattagaagcacagctaatattcatacaacatactatgctgagtctgtccaaggg

SEQ ID NO: 146 - protein, 19 aa, synthetic construct
  REGION 1..19: note = Antibody 18B4 Heavy Chain CDR2
EIRSTANIHTTYYAESVQG

SEQ ID NO: 147 - DNA, 21 bp, synthetic construct
  misc_feature 1..21: note = Antibody 18B4 Heavy Chain CDR3
  source 1..21: mol_type = other DNA
ttactctacggatttgcttac

SEQ ID NO: 148 - protein, 7 aa, synthetic construct
  REGION 1..7: note = Antibody 18B4 Heavy Chain CDR3
LLYGFAY

SEQ ID NO: 149 - DNA, 48 bp, synthetic construct
  misc_feature 1..48: note = Antibody 18B4 Light Chain CDR1
  source 1..48: mol_type = other DNA
agaactagtcagagccttgtacacagtaatggaaacacctatttacat

SEQ ID NO: 150 - protein, 16 aa, synthetic construct
  REGION 1..16: note = Antibody 18B4 Light Chain CDR1
RTSQSLVHSNGNTYLH

SEQ ID NO: 151 - DNA, 21 bp, synthetic construct
  misc_feature 1..21: note = Antibody 18B4 Light Chain CDR2
  source 1..21: mol_type = other DNA
aaagtttccagccgattttct

SEQ ID NO: 152 - protein, 7 aa, synthetic construct
  REGION 1..7: note = Antibody 18B4 Light Chain CDR2
KVSSRFS

SEQ ID NO: 153 - DNA, 27 bp, synthetic construct
  misc_feature 1..27: note = Antibody 18B4 Light Chain CDR3
  source 1..27: mol_type = other DNA
tctcaaaatacacatgttccgtacacg

SEQ ID NO: 154 - protein, 9 aa, synthetic construct
  REGION 1..9: note = Antibody 18B4 Light Chain CDR3
SQNTHVPYT

SEQ ID NO: 155 - DNA, 408 bp, synthetic construct
  misc_feature 1..408: note = Antibody 1E4 Heavy chain - Signal sequence-FR1-CDR1-FR2-CDR2-FR3-CDR3-FR4
  source 1..408: mol_type = other DNA
atggaatggccttgtatctttctcttcctcctgtcagtaactgaaggtgtccactcccaggttcagctgcagcagtctggggctgagctggtgaggcctgggtcctcagtgaagatttcctgtaaggcttctggctatgcattcagtacctactggatgaactgggtgaagcagaggcctggacagggtcttgagtggattggacagatttatcctggagatagtgatactaactacaatggaaagttcaagggtaaagccacactgactgcagacaagtcctccaacacagcctacatgcagctcagcagcctaacatctgaggactctgcggtctttttctgtgcaagaggtaaccacgcctctatggactactggggtcaaggaacctcagtcaccgtctcctca

SEQ ID NO: 156 - protein, 136 aa, synthetic construct
  REGION 1..136: note = Antibody 1E4 Heavy chain - Signal sequence-FR1-CDR1-FR2-CDR2-FR3-CDR3-FR4
MEWPCIFLFLLSVTEGVHSQVQLQQSGAELVRPGSSVKISCKASGYAFSTYWMNWVKQRPGQGLEWIGQIYPGDSDTNYNGKFKGKATLTADKSSNTAYMQLSSLTSEDSAVFFCARGNHASMDYWGQGTSVTVSS

SEQ ID NO: 157 - DNA, 393 bp, synthetic construct
  misc_feature 1..393: note = Antibody 1E4 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaaaaacacatgttccgtggacgttcggtggaggcaccaagctggaaatcaaa

SEQ ID NO: 158 - protein, 131 aa, synthetic construct
  REGION 1..131: note = Antibody 1E4 Light chain - Signal sequence-FR1-CDR1-FR2-CDR2-FR3-CDR3-FR4
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQKTHVPWTFGGGTKLEIK

SEQ ID NO: 159 - DNA, 15 bp, synthetic construct
  misc_feature 1..15: note = Antibody 1E4 Heavy Chain CDR1
  source 1..15: mol_type = other DNA
acctactggatgaac

SEQ ID NO: 160 - protein, 5 aa, synthetic construct
  REGION 1..5: note = Antibody 1E4 Heavy Chain CDR1
TYWMN

SEQ ID NO: 161 - DNA, 51 bp, synthetic construct
  misc_feature 1..51: note = Antibody 1E4 Heavy Chain CDR2
  source 1..51: mol_type = other DNA
cagatttatcctggagatagtgatactaactacaatggaaagttcaagggt

SEQ ID NO: 162 - protein, 17 aa, synthetic construct
  REGION 1..17: note = Antibody 1E4 Heavy Chain CDR2
QIYPGDSDTNYNGKFKG

SEQ ID NO: 163 - DNA, 24 bp, synthetic construct
  misc_feature 1..24: note = Antibody 1E4 Heavy Chain CDR3
  source 1..24: mol_type = other DNA
ggtaaccacgcctctatggactac

SEQ ID NO: 164 - protein, 8 aa, synthetic construct
  REGION 1..8: note = Antibody 1E4 Heavy Chain CDR3
GNHASMDY

SEQ ID NO: 165 - DNA, 48 bp, synthetic construct
  misc_feature 1..48: note = Antibody 1E4 Light Chain CDR1
  source 1..48: mol_type = other DNA
agatctagtcagagccttgtacacagtaatggaaacacctatttacat

SEQ ID NO: 166 - protein, 16 aa, synthetic construct
  REGION 1..16: note = Antibody 1E4 Light Chain CDR1
RSSQSLVHSNGNTYLH

SEQ ID NO: 167 - DNA, 21 bp, synthetic construct
  misc_feature 1..21: note = Antibody 1E4 Light Chain CDR2
  source 1..21: mol_type = other DNA
aaagtttccaaccgattttct

SEQ ID NO: 168 - protein, 7 aa, synthetic construct
  REGION 1..7: note = Antibody 1E4 Light Chain CDR2
KVSNRFS

SEQ ID NO: 169 - DNA, 27 bp, synthetic construct
  misc_feature 1..27: note = Antibody 1E4 Light Chain CDR3
  source 1..27: mol_type = other DNA
tctcaaaaaacacatgttccgtggacg

SEQ ID NO: 170 - protein, 9 aa, synthetic construct
  REGION 1..9: note = Antibody 1E4 Light Chain CDR3
SQKTHVPWT

SEQ ID NO: 171 - DNA, 411 bp, synthetic construct
  misc_feature 1..411: note = Antibody 29H1 Heavy chain - Signal sequence-FR1-CDR1-FR2-CDR2-FR3-CDR3-FR4
  source 1..411: mol_type = other DNA
atgtacttgggactgaactatgtattcatagtttttctcttaaatggtgtccagagtgaagtgaagcttgaggagtctggaggaggcttggtacaacctggaggatccatgaaactctcttgtgctgcctctggattcacttttagtgacgcctggatggactgggtccgccagtctccagagaaggggcttgaatgggttgctgaaattagaagcaaagctactaatcatgcaacatactatgctgagtctgtgaaagggaggttcaccatctcaagagatgattccaaaagtagtgtctacctgcaaatgaacagcttaagagctgaagacactggcatttattactgtacccccctactttacgggtttgcttactggggccaagggactctggtcactgtctctgca

SEQ ID NO: 172 - protein, 137 aa, synthetic construct
  REGION 1..137: note = Antibody 29H1 Heavy chain - Signal sequence-FR1-CDR1-FR2-CDR2-FR3-CDR3-FR4
MYLGLNYVFIVFLLNGVQSEVKLEESGGGLVQPGGSMKLSCAASGFTFSDAWMDWVRQSPEKGLEWVAEIRSKATNHATYYAESVKGRFTISRDDSKSSVYLQMNSLRAEDTGIYYCTPLLYGFAYWGQGTLVTVSA

SEQ ID NO: 173 - DNA, 393 bp, synthetic construct
  misc_feature 1..393: note = Antibody 29H1 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctggtcagagccttgtacacagtaatggacacacctatttacattggtacctgcagaagccaggccagtctccaaggctcctgatctacaaagtttccaaccgattttctggggtcccagacaggttcagtggcagtggatcaagggcagatttcacactcaagatcagcagagtggaggctgaggatctgggagtttatttctgctctcaaactacacatgttccgtggacgttcggtggaggcaccaagctggaaatcaaa

SEQ ID NO: 174 - protein, 131 aa, synthetic construct
  REGION 1..131: note = Antibody 29H1 Light chain - Signal sequence-FR1-CDR1-FR2-CDR2-FR3-CDR3-FR4
MKLPVRLLVLMFWIPASSSDVVMTQTPLSLPVSLGDQASISCRSGQSLVHSNGHTYLHWYLQKPGQSPRLLIYKVSNRFSGVPDRFSGSGSRADFTLKISRVEAEDLGVYFCSQTTHVPWTFGGGTKLEIK

SEQ ID NO: 175 - DNA, 15 bp, synthetic construct
  misc_feature 1..15: note = Antibody 29H1 Heavy Chain CDR1
  source 1..15: mol_type = other DNA
gacgcctggatggac

SEQ ID NO: 176 - protein, 5 aa, synthetic construct
  REGION 1..5: note = Antibody 29H1 Heavy Chain CDR1
DAWMD

SEQ ID NO: 177 - DNA, 57 bp, synthetic construct
  misc_feature 1..57: note = Antibody 29H1 Heavy Chain CDR2
  source 1..57: mol_type = other DNA
gaaattagaagcaaagctactaatcatgcaacatactatgctgagtctgtgaaaggg

SEQ ID NO: 178 - protein, 19 aa, synthetic construct
  REGION 1..19: note = Antibody 29H1 Heavy Chain CDR2
EIRSKATNHATYYAESVKG

SEQ ID NO: 179 - DNA, 21 bp, synthetic construct
  misc_feature 1..21: note = Antibody 29H1 Heavy Chain CDR3
  source 1..21: mol_type = other DNA
ctactttacgggtttgcttac

SEQ ID NO: 180 - protein, 7 aa, synthetic construct
  REGION 1..7: note = Antibody 29H1 Heavy Chain CDR3
LLYGFAY

SEQ ID NO: 181 - DNA, 48 bp, synthetic construct
  misc_feature 1..48: note = Antibody 29H1 Light Chain CDR1
  source 1..48: mol_type = other DNA
agatctggtcagagccttgtacacagtaatggacacacctatttacat

SEQ ID NO: 182 - protein, 16 aa, synthetic construct
  REGION 1..16: note = Antibody 29H1 Light Chain CDR1
RSGQSLVHSNGHTYLH

SEQ ID NO: 183 - DNA, 21 bp, synthetic construct
  misc_feature 1..21: note = Antibody 29H1 Light Chain CDR2
  source 1..21: mol_type = other DNA
aaagtttccaaccgattttct

SEQ ID NO: 184 - protein, 7 aa, synthetic construct
  REGION 1..7: note = Antibody 29H1 Light Chain CDR2
KVSNRFS

SEQ ID NO: 185 - DNA, 27 bp, synthetic construct
  misc_feature 1..27: note = Antibody 29H1 Light Chain CDR3
  source 1..27: mol_type = other DNA
tctcaaactacacatgttccgtggacg

SEQ ID NO: 186 - protein, 9 aa, synthetic construct
  REGION 1..9: note = Antibody 29H1 Light Chain CDR3
SQTTHVPWT

SEQ ID NO: 187 - DNA, 399 bp, synthetic construct
  misc_feature 1..399: note = Antibody 31A1 Heavy chain - Signal sequence-FR1-CDR1-FR2-CDR2-FR3-CDR3-FR4
  source 1..399: mol_type = other DNA
atggaaaggcactggatctttctcttcctgttttcagtaactgcaggtgtccactcccaggtccagcttcagcagtctggggctgaactggcaaaacctggggcctcagtgaagatgtcctgcaaggcttctggctacacctttactagctactggatgcactgggtaaaacagaggcctggacagggtctggaatggattggatacattaatcctagcactggttatactgagtacaatcagaagttcaaggacaaggccacattgactgcagacaaatcctccagcacagcctacatgcaactgagcagcctgacatctgaggactctgcagtctattactgtgcaagagcctacattgactactggggccaaggcaccactctcacagtctcctca

SEQ ID NO: 188 - protein, 133 aa, synthetic construct
  REGION 1..133: note = Antibody 31A1 Heavy chain - Signal sequence-FR1-CDR1-FR2-CDR2-FR3-CDR3-FR4
MERHWIFLFLFSVTAGVHSQVQLQQSGAELAKPGASVKMSCKASGYTFTSYWMHWVKQRPGQGLEWIGYINPSTGYTEYNQKFKDKATLTADKSSSTAYMQLSSLTSEDSAVYYCARAYIDYWGQGTTLTVSS

SEQ ID NO: 189 - DNA, 393 bp, synthetic construct
  misc_feature 1..393: note = Antibody 31A1 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttttgatgacccaaactccactctccctgcctgtcagtcttggagatcaagcctccttctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaagtttcacattttccgtggacgttcggtggaggcaccaagctggaaatcaaa

SEQ ID NO: 190 - protein, 131 aa, synthetic construct
  REGION 1..131: note = Antibody 31A1 Light chain - Signal sequence-FR1-CDR1-FR2-CDR2-FR3-CDR3-FR4
MKLPVRLLVLMFWIPASSSDVLMTQTPLSLPVSLGDQASFSCRSSQSIVHSNGNTYLEWYLQKPGQSPKLLIYKVSNRFSGVPDRFSGSGSGTDFTLKINRVEAEDLGVYYCFQVSHFPWTFGGGTKLEIK

SEQ ID NO: 191 - DNA, 15 bp, synthetic construct
  misc_feature 1..15: note = Antibody 31A1 Heavy Chain CDR1
  source 1..15: mol_type = other DNA
agctactggatgcac

SEQ ID NO: 192 - protein, 5 aa, synthetic construct
  REGION 1..5: note = Antibody 31A1 Heavy Chain CDR1
SYWMH

SEQ ID NO: 193 - DNA, 51 bp, synthetic construct
  misc_feature 1..51: note = Antibody 31A1 Heavy Chain CDR2
  source 1..51: mol_type = other DNA
tacattaatcctagcactggttatactgagtacaatcagaagttcaaggac

SEQ ID NO: 194 - protein, 17 aa, synthetic construct
  REGION 1..17: note = Antibody 31A1 Heavy Chain CDR2
YINPSTGYTEYNQKFKD

SEQ ID NO: 195 - DNA, 15 bp, synthetic construct
  misc_feature 1..15: note = Antibody 31A1 Heavy Chain CDR3
  source 1..15: mol_type = other DNA
gcctacattgactac

SEQ ID NO: 196 - protein, 5 aa, synthetic construct
  REGION 1..5: note = Antibody 31A1 Heavy Chain CDR3
AYIDY

SEQ ID NO: 197 - DNA, 48 bp, synthetic construct
  misc_feature 1..48: note = Antibody 31A1 Light Chain CDR1
  source 1..48: mol_type = other DNA
agatctagtcagagcattgtacatagtaatggaaacacctatttagaa

SEQ ID NO: 198 - protein, 16 aa, synthetic construct
  REGION 1..16: note = Antibody 31A1 Light Chain CDR1
RSSQSIVHSNGNTYLE

SEQ ID NO: 199 - DNA, 21 bp, synthetic construct
  misc_feature 1..21: note = Antibody 31A1 Light Chain CDR2
  source 1..21: mol_type = other DNA
aaagtttccaaccgattttct

SEQ ID NO: 200 - protein, 7 aa, synthetic construct
  REGION 1..7: note = Antibody 31A1 Light Chain CDR2
KVSNRFS

SEQ ID NO: 201 - DNA, 27 bp, synthetic construct
  misc_feature 1..27: note = Antibody 31A1 Light Chain CDR3
  source 1..27: mol_type = other DNA
tttcaagtttcacattttccgtggacg

SEQ ID NO: 202 - protein, 9 aa, synthetic construct
  REGION 1..9: note = Antibody 31A1 Light Chain CDR3
FQVSHFPWT

SEQ ID NO: 203 - DNA, 411 bp, synthetic construct
  misc_feature 1..411: note = Antibody 32C1 Heavy chain - Signal sequence-FR1-CDR1-FR2-CDR2-FR3-CDR3-FR4
  source 1..411: mol_type = other DNA
atgtacttgggactgaactgtgtattcatagtttttctcttaaaaggtgtccagagtgaagtgaagcttgaggagtctggaggaggcttggtgcaatctggaggatccatgaaactctcctgtgttgcctctggattcactttcagtaattactggatgaactgggtccgccagtctccagagaaggggcttgagtgggttgctgaaattagattgaaatctaataattatgcaatacattatgcggagtctgtgaaggggaggttcaccatctcaagagatgattccaaaagtagtgtctacctgcaaatgaacaacttaagagctgaagacactggcatttattactgtaccagggtcccgggactggatgcttactggggccaagggactctggtcactgtctctgca

SEQ ID NO: 204 - protein, 137 aa, synthetic construct
  REGION 1..137: note = Antibody 32C1 Heavy chain - Signal sequence-FR1-CDR1-FR2-CDR2-FR3-CDR3-FR4
MYLGLNCVFIVFLLKGVQSEVKLEESGGGLVQSGGSMKLSCVASGFTFSNYWMNWVRQSPEKGLEWVAEIRLKSNNYAIHYAESVKGRFTISRDDSKSSVYLQMNNLRAEDTGIYYCTRVPGLDAYWGQGTLVTVSA

SEQ ID NO: 205 - DNA, 393 bp, synthetic construct
  misc_feature 1..393: note = Antibody 32C1 Light chain - Signal sequence-FR1-CDR1-FR2-CDR2-FR3-CDR3-FR4
  source 1..393: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagtgtggaggctgaggatctgggagtttatttctgctctcaaattacacatgttccgtacacgttcggaggggggaccaatctggaaataaaa

SEQ ID NO: 206 - protein, 131 aa, synthetic construct
  REGION 1..131: note = Antibody 32C1 Light chain - Signal sequence-FR1-CDR1-FR2-CDR2-FR3-CDR3-FR4
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISSVEAEDLGVYFCSQITHVPYTFGGGTNLEIK

SEQ ID NO: 207 - DNA, 15 bp, synthetic construct
  misc_feature 1..15: note = Antibody 32C1 Heavy Chain CDR1
  source 1..15: mol_type = other DNA
aattactggatgaac

SEQ ID NO: 208 - protein, 5 aa, synthetic construct
  REGION 1..5: note = Antibody 32C1 Heavy Chain CDR1
NYWMN

SEQ ID NO: 209 - DNA, 57 bp, synthetic construct
  misc_feature 1..57: note = Antibody 32C1 Heavy Chain CDR2
  source 1..57: mol_type = other DNA
gaaattagattgaaatctaataattatgcaatacattatgcggagtctgtgaagggg

SEQ ID NO: 210 - protein, 19 aa, synthetic construct
  REGION 1..19: note = Antibody 32C1 Heavy Chain CDR2
EIRLKSNNYAIHYAESVKG

SEQ ID NO: 211 - DNA, 21 bp, synthetic construct
  misc_feature 1..21: note = Antibody 32C1 Heavy Chain CDR3
  source 1..21: mol_type = other DNA
gtcccgggactggatgcttac

SEQ ID NO: 212 - protein, 7 aa, synthetic construct
  REGION 1..7: note = Antibody 32C1 Heavy Chain CDR3
VPGLDAY

SEQ ID NO: 213 - DNA, 48 bp, synthetic construct; identical to SEQ ID NO: 165
  misc_feature 1..48: note = Antibody 32C1 Light Chain CDR1
  source 1..48: mol_type = other DNA

SEQ ID NO: 214 - protein, 16 aa, synthetic construct
  REGION 1..16: note = Antibody 32C1 Light Chain CDR1
RSSQSLVHSNGNTYLH

SEQ ID NO: 215 - DNA, 21 bp, synthetic construct
  misc_feature 1..21: note = Antibody 32C1 Light Chain CDR2
  source 1..21: mol_type = other DNA
aaagtttccaaccgattttct

SEQ ID NO: 216 - protein, 7 aa, synthetic construct
  REGION 1..7: note = Antibody 32C1 Light Chain CDR2
KVSNRFS

SEQ ID NO: 217 - DNA, 27 bp, synthetic construct
  misc_feature 1..27: note = Antibody 32C1 Light Chain CDR3
  source 1..27: mol_type = other DNA
tctcaaattacacatgttccgtacacg

SEQ ID NO: 218 - protein, 9 aa, synthetic construct
  REGION 1..9: note = Antibody 32C1 Light Chain CDR3
SQITHVPYT

SEQ ID NO: 219 - DNA, 423 bp, synthetic construct
  misc_feature 1..423: note = Antibody 45C11 Heavy chain - Signal sequence-FR1-CDR1-FR2-CDR2-FR3-CDR3-FR4
  source 1..423: mol_type = other DNA
atgaaatgcagctgggttatcttcttcctgatggcagtggttacaggggtcaattcagaggttcagctgcagcagtctggggcagaccttgtgaagccaggggcctcagtcaagttgtcctgcacagcttctggcttcaacattaaagacacctttatgcactgggtgaagcagaggcctgaacagggcctggagtggattggaaggattgatcctgcgaatggtaatactaaatatgacccgaaattccagggcaaggccactataacagcagacacatcctccaacacagcctacctgcagctcagcagcctgacatctgaggacactgccgtctattactgtgctaaaccgtatggtaactacggctattactatgctttggactactggggtcaaggaacctcagtcaccgtctcctca

SEQ ID NO: 220 - protein, 141 aa, synthetic construct
  REGION 1..141: note = Antibody 45C11 Heavy chain - Signal sequence-FR1-CDR1-FR2-CDR2-FR3-CDR3-FR4
MKCSWVIFFLMAVVTGVNSEVQLQQSGADLVKPGASVKLSCTASGFNIKDTFMHWVKQRPEQGLEWIGRIDPANGNTKYDPKFQGKATITADTSSNTAYLQLSSLTSEDTAVYYCAKPYGNYGYYYALDYWGQGTSVTVSS

SEQ ID NO: 221 - DNA, 381 bp, synthetic construct
  misc_feature 1..381: note = Antibody 45C11 Light chain - Signal sequence-FR1-CDR1-FR2-CDR2-FR3-CDR3-FR4
  source 1..381: mol_type = other DNA
atgaggttccaggttcaggttctggggctccttctgctctggatatcaggtgcccagtgtgatgtccagataacccagtctccatcttatcttgctgcatctcctggagaaaccattactattaattgcagggcaagtaagagcattagcaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacagcataatgaattcccgtggacgttcggtggaggcaccaagctggaaatcaaa

SEQ ID NO: 222 - protein, 127 aa, synthetic construct
  REGION 1..127: note = Antibody 45C11 Light chain - Signal sequence-FR1-CDR1-FR2-CDR2-FR3-CDR3-FR4
MRFQVQVLGLLLLWISGAQCDVQITQSPSYLAASPGETITINCRASKSISKYLAWYQEKPGKTNKLLIYSGSTLQSGIPSRFSGSGSGTDFTLTISSLEPEDFAMYYCQQHNEFPWTFGGGTKLEIK

SEQ ID NO: 223 - DNA, 15 bp, synthetic construct
  misc_feature 1..15: note = Antibody 45C11 Heavy Chain CDR1
  source 1..15: mol_type = other DNA
gacacctttatgcac

SEQ ID NO: 224 - protein, 5 aa, synthetic construct
  REGION 1..5: note = Antibody 45C11 Heavy Chain CDR1
DTFMH

SEQ ID NO: 225 - DNA, 51 bp, synthetic construct
  misc_feature 1..51: note = Antibody 45C11 Heavy Chain CDR2
  source 1..51: mol_type = other DNA
aggattgatcctgcgaatggtaatactaaatatgacccgaaattccagggc

SEQ ID NO: 226 - protein, 17 aa, synthetic construct
  REGION 1..17: note = Antibody 45C11 Heavy Chain CDR2
RIDPANGNTKYDPKFQG

SEQ ID NO: 227 - DNA, 39 bp, synthetic construct
  misc_feature 1..39: note = Antibody 45C11 Heavy Chain CDR3
  source 1..39: mol_type = other DNA
ccgtatggtaactacggctattactatgctttggactac

SEQ ID NO: 228 - protein, 13 aa, synthetic construct
  REGION 1..13: note = Antibody 45C11 Heavy Chain CDR3
PYGNYGYYYALDY

SEQ ID NO: 229 - DNA, 33 bp, synthetic construct
  misc_feature 1..33: note = Antibody 45C11 Light Chain CDR1
  source 1..33: mol_type = other DNA
agggcaagtaagagcattagcaaatatttagcc

SEQ ID NO: 230 - protein, 11 aa, synthetic construct
  REGION 1..11: note = Antibody 45C11 Light Chain CDR1
RASKSISKYLA

SEQ ID NO: 231 - DNA, 21 bp, synthetic construct
  misc_feature 1..21: note = Antibody 45C11 Light Chain CDR2
  source 1..21: mol_type = other DNA
tctggatccactttgcaatct

SEQ ID NO: 232 - protein, 7 aa, synthetic construct
  REGION 1..7: note = Antibody 45C11 Light Chain CDR2
SGSTLQS

SEQ ID NO: 233 - DNA, 27 bp, synthetic construct
  misc_feature 1..27: note = Antibody 45C11 Light Chain CDR3
  source 1..27: mol_type = other DNA
caacagcataatgaattcccgtggacg

SEQ ID NO: 234 - protein, 9 aa, synthetic construct
  REGION 1..9: note = Antibody 45C11 Light Chain CDR3
QQHNEFPWT

SEQ ID NO: 235 - protein, 20 aa, synthetic construct
  REGION 1..20: note = Tandem repeat domain peptide
PDTRPAPGSTAPPAHGVTSA

SEQ ID NO: 236 - protein, 493 aa, synthetic construct
  REGION 1..493: note = CAR44: CD8/HUMNC2/CD8/4-1BB/CD3
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 237 - protein, 115 aa, synthetic construct
  REGION 1..115: note = MIN- A2-1 light chain variable region amino acid sequence
DIVLTQSTEIMSASPGEKVTITCSASSSISYIHWFQQKPGTSPKLWIFGTSNLASGVPARFSGSGSGTSYSLTVSRMEAEDTATYYCQQRSNYPFTFGSGTKLQIKRADAAPTVS

SEQ ID NO: 238 - protein, 115 aa, synthetic construct
  REGION 1..115: note = MIN-A2-2 light chain variable region amino acid sequence
DIVMTQSPAIMSASPGEKVTMTCSASSSVSYMHWFQQKPGTSPKLWIYSTSNLASGAPARFSGSGSGTSYSLTVSRMESEDAATYYCQQRSSYPSTFGGGTKLEIKRADAAPTVS

SEQ ID NO: 239 - protein, 115 aa, synthetic construct
  REGION 1..115: note = MIN-C9-1 light chain variable region amino acid sequence
DIVLTQTTAIMSASPGEKVTITCSASSSVSYMYWFQQKPGTSPKLWIYSTSNLASGVPARFSGSGSGTSYSLTISRMEAEDAATYYCQQRSSYPSTFGGGTKLEIKRADAAPTVS

SEQ ID NO: 240 - protein, 115 aa, synthetic construct
  REGION 1..115: note = MIN-C9-2 light chain variable region amino acid sequence
DIVITQSTAIMSASPGEKVTITCSASSSVSYTYWFQQKPGTSPKLWIYSTSNLASGVPARFSGSGSGTSYSLTISRMEAEDAATYYCQQRSSYPSTFGGGTKLEIKRADAAPTVS

SEQ ID NO: 241 - protein, 115 aa, synthetic construct
  REGION 1..115: note = MIN-D7-1 light chain variable region amino acid sequence
DIVITQTPAIMSASPGEKVTMTCSASSSVSYMHWFQQKPGTSPKLWIYSTSNLASGVPARFSGSGSGTSYSLTVSRMESEDAATYYCQQRSSYPSTFGGGTKLEIKRADAAPTVS

SEQ ID NO: 242 - protein, 115 aa, synthetic construct
  REGION 1..115: note = MIN-D7-2 light chain variable region amino acid sequence
DIVLTQSTAIMSASPGEKVTMTCSASSSVSYMHWFQQKPGTSPKLWIYSTSNLASGVPARFSGSGSGTSYSLTVSRMESEDAATYYCQQRSSYPSTFGGGTKLEIKRADAAPTVS

SEQ ID NO: 243 - protein, 115 aa, synthetic construct
  REGION 1..115: note = MIN-F2-1 light chain variable region amino acid sequence
DIVMTQSPEIMSASPGEKVTITCSASSSISYIHWFQQKPGTSPKLWIFGTSNLASGVPARFSGSGSGTSYSLTVSRMEAEDTATYYCQQRSNYPFTFGSGTKLQIKRADAAPTVS

SEQ ID NO: 244 - protein, 115 aa, synthetic construct
  REGION 1..115: note = MIN-F2-2 light chain variable region amino acid sequence
DIVITQSTEIMSASPGEKVTITCSASSSISYIHWFQQKPGTSPKLWIFGTSNLASGVPARFSGSGSGTSYSLTVSRMEAEDTATYYCQQRSNYPFTFGSGTKLQIKRADAAPTVS

SEQ ID NO: 245 - protein, 130 aa, synthetic construct
  REGION 1..130: note = MIN-A2-1 heavy chain variable region amino acid sequence
EVKLQESGPELKKPGETVEISCKASGYTFTNYGMNWVKQAPGKGLKWMGWINTYTGEPTYAGDFKGRFAFSLETSASTAYLQINTLKNEDTATYFCARSGDGYWYYAMDYWGQGTSVTVSSAKTTPPSVY

SEQ ID NO: 246 - protein, 130 aa, synthetic construct
  REGION 1..130: note = MIN-A2-2 heavy chain variable region amino acid sequence
EVQLQQSGPELKKPGETVKISCKASGYTFTNYGMNWVKQAPGKGLKWMGWINTYTGEPTYAGDFKGRFAFSLETSASTAYLQINTLKNEDTATYFCARSGDGYWYYAMDYWGQGTSVTVSSAKTTPPSVY

SEQ ID NO: 247 - protein, 130 aa, synthetic construct
  REGION 1..130: note = MIN-C9-1 heavy chain variable region amino acid sequence
QVQLQESGPELKQPGETVKISCKASGYTFTNNGMNWVKQAPGKGLKWMGWINTYTGEPTYADDFKGRFAFSLDTSASTAYLQINNLKNEDMATYFCARTGTARAFYAMDYWGQGTSVTVSSTKTTAPSVY

SEQ ID NO: 248 - protein, 130 aa, synthetic construct
  REGION 1..130: note = MIN-C9-2 heavy chain variable region amino acid sequence
QVQLQQSGPELKQPGETVKISCKASGYTFTNNGMNWVKQAPGKGLKWMGWINTYTGEPTYADDFKGRFAFSLGTSASTAYLQINNLKNEDMATYFCARTGTARAFYAMDYWGQGTSVTVSSTKTTAPSVY

SEQ ID NO: 249 - protein, 130 aa, synthetic construct
  REGION 1..130: note = MIN-D7-1 heavy chain variable region amino acid sequence
EVQLEQSGPELKKPGETVKISCKASGYTFINYGMNWVKQAPGKGLKWMGWINTYTGEPTYVDDFKGRFAFSLETSARTAYLQINNLKNEDMATYFCARTGTTAILNGMDYWGQGTSVTVSSAKTTPPSVY

SEQ ID NO: 250 - protein, 130 aa, synthetic construct; identical to SEQ ID NO: 246
  REGION 1..130: note = MIN-D7-2 heavy chain variable region amino acid sequence

SEQ ID NO: 251 - protein, 130 aa, synthetic construct
  REGION 1..130: note = MIN-F2-1 heavy chain variable region amino acid sequence
EVKLEESGPELKKPGETVKISCKASGYTFINYGMNWVKQAPGKGLKWMGWINTYTGEPTYVDDFKGRFAFSLETSARTAYLQINNLKNEDMATYFCARTGTTAILNGMDYWGQGTSVTVSSAKTTPPSVY

SEQ ID NO: 252 - protein, 124 aa, synthetic construct
  REGION 1..124: note = MIN-F2-2 heavy chain variable region amino acid sequence
EVQLEQSGAELVRPGASVKLSCKALGYTFTDYEMHWVKQTPVHGLEWIGAIHPGSGGTAYNQKFKGKATLTADKSSSTAYMELSSLTSEDSAVYYCTNYGSFAYWGQGTLVTVSAAKTTPPSVY

SEQ ID NO: 253 - protein, 130 aa, synthetic construct
  REGION 1..130: note = MIN-F2-3 heavy chain variable region amino acid sequence
RCRLQQSGPELKKPGETVKISCKASGYTFINYGMNWVKQAPGKGLKWMGWINTYTGEPTYVDDFKGRFAFSLETSARTAYLQINNLKNEDMATYFCARTGTTAILNGMDYWGQGTSVTVSSAKTTPPSCL

SEQ ID NO: 254 - protein, 130 aa, synthetic construct; identical to SEQ ID NO: 249
  REGION 1..130: note = MIN-F2-4 heavy chain variable region amino acid sequence

SEQ ID NO: 255 - protein, 113 aa, synthetic construct
  REGION 1..113: note = MIN-14 light chain variable region amino acid sequence
DIQMTQSPSSLSASLGERVSLTCRASQDIGSSLNWLQQEPDGTIKRLIYATSSLDSGVPKRFSGSRSGSDYSLTISSLESEDFVDYYCLQYASSPHVRCWDQAGAETGCCTNC

SEQ ID NO: 256 - protein, 107 aa, synthetic construct
  REGION 1..107: note = MIN-17-1 light chain variable region amino acid sequence
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSW

SEQ ID NO: 257 - protein, 115 aa, synthetic construct
  REGION 1..115: note = MIN-17-2 light chain variable region amino acid sequence
DIQMTQSPASQSASLGESVTITCLASQTIGTWLAWYQQKPGKSPQLLIYAATSLADGVPSRFSGSGSGTKFSFKISSLQAEDFVSYYCQQLYSTPWTFGGGTKLEIKRADAAPTV

SEQ ID NO: 258 - protein, 107 aa, synthetic construct; identical to SEQ ID NO: 256
  REGION 1..107: note = MIN-29 light chain variable region amino acid sequence

SEQ ID NO: 259 - protein, 107 aa, synthetic construct; identical to SEQ ID NO: 256
  REGION 1..107: note = MIN-34 light chain variable region amino acid sequence

SEQ ID NO: 260 - protein, 115 aa, synthetic construct
  REGION 1..115: note = MIN-42 light chain variable region amino acid sequence
DIVMTQSQKFMSTSVGDRVSVTCKASQNVGTNVGWYQQKPGQSPKALIYSASYRYSGVPDRFTGSGSGTDFTLTISNVQSEDLAEYFCQQYNNYPYTFGGGTKLEIKRADAAPTV

SEQ ID NO: 261 - protein, 115 aa, synthetic construct
  REGION 1..115: note = MIN-45 light chain variable region amino acid sequence
DIQMTQPPASLSASVGETVTITCRASGNIHNFLAWYQQKQGKSPQLLVYNAKTLADGVPSRFSGSGSGTQYSLKINSLQPEDFGSYYCQHFWSTPWTFGGGTKLEIKRADAAPTV

SEQ ID NO: 262 - protein, 105 aa, synthetic construct
  REGION 1..105: note = MIN-14 heavy chain variable region amino acid sequence
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCATYGNYWYF

SEQ ID NO: 263 - protein, 105 aa, synthetic construct
  REGION 1..105: note = MIN-17-2 heavy chain variable region amino acid sequence
QITLKESGPGIVQPSQPFRLTCTFSGFSLSTSGIGVTWIRQPSGKGLEWLATIWWDDDNRYNPSLKSRLTVSKDTSNNQAFLNIITVETADTAIYYCAQSTMVTA

SEQ ID NO: 264 - protein, 105 aa, synthetic construct; identical to SEQ ID NO: 262
  REGION 1..105: note = MIN-17-1 heavy chain variable region amino acid sequence

SEQ ID NO: 265 - protein, 127 aa, synthetic construct
  REGION 1..127: note = MIN-29 heavy chain variable region amino acid sequence
  SITE 12: note = misc_feature - Xaa can be any naturally occurring amino acid
DVKLVESGGDLXKLTEGEDIWEGLTLCRDSDQSPLAPVSKPGRVVRPQRSCTVIQGCVLRLQTAHLQVQGVLGIVSGDGESALHSVWIVGATTITINGCDQLQPLLWSLANPRHVIATESESRGCTG

SEQ ID NO: 266 - protein, 104 aa, synthetic construct
  REGION 1..104: note = MIN-34 heavy chain variable region amino acid sequence
QVQLKQSGPGLVQPSQSLSITCTVSGFSLTSYGVHWVRQSPGKGLEWLGVIWGGGSTDYNAAFISRLSISKDNSKSQVFFKMNSLQANDTAIYYCARNDYPAWF

SEQ ID NO: 267 - protein, 105 aa, synthetic construct
  REGION 1..105: note = MIN-42 heavy chain variable region amino acid sequence
EVQLVESGGDLVKPGRSLKLSCAASGFTFSSFGMSWVRQTPDKRLEWVATISSGGTYTYYPDSVKGRFTISRDNAKNTLYLQMSSLKSEDTAMYYCSRRFYYDYD

SEQ ID NO: 268 - protein, 103 aa, synthetic construct
  REGION 1..103: note = MIN-45 heavy chain variable region amino acid sequence
EVQLQQSGPELVKPGASVKISCKASGYSFTGYFMSWVMQSHGKSLEWIGRINPYNGDTFYNQKFKGKATLTVDKSSTTAHIELRSLASEDSAVYYCARKGLYG

SEQ ID NO: 269 - protein, 10 aa, synthetic construct
  REGION 1..10: note = MIN-A2-1 light chain variable complementarity determining region 1 (CDR1) amino acid sequence
SASSSISYIH

SEQ ID NO: 270 - protein, 10 aa, synthetic construct
  REGION 1..10: note = MIN-A2-2 light chain variable complementarity determining region 1 (CDR1) amino acid sequence
SASSSVSYMH

SEQ ID NO: 271 - protein, 10 aa, synthetic construct
  REGION 1..10: note = MIN-C9-1 light chain variable complementarity determining region 1 (CDR1) amino acid sequence
SASSSVSYMY

SEQ ID NO: 272 - protein, 10 aa, synthetic construct
  REGION 1..10: note = MIN-C9-2 light chain variable complementarity determining region 1 (CDR1) amino acid sequence
SASSSVSYTY

SEQ ID NO: 273 - protein, 10 aa, synthetic construct
  REGION 1..10: note = MIN-D7-1 light chain variable complementarity determining region 1 (CDR1) amino acid sequence
SASSSVSYMH

SEQ ID NO: 274 - protein, 10 aa, synthetic construct
  REGION 1..10: note = MIN-D7-2 light chain variable complementarity determining region 1 (CDR1) amino acid sequence
SASSSVSYMH

SEQ ID NO: 275 - protein, 10 aa, synthetic construct
  REGION 1..10: note = MIN-F2-1 light chain variable complementarity determining region 1 (CDR1) amino acid sequence
SASSSISYIH

SEQ ID NO: 276 - protein, 10 aa, synthetic construct
  REGION 1..10: note = MIN-F2-2 light chain variable complementarity determining region 1 (CDR1) amino acid sequence
SASSSISYIH

SEQ ID NO: 277 - protein, 7 aa, synthetic construct
  REGION 1..7: note = MIN-A2-1 light chain variable complementarity determining region 2 (CDR2) amino acid sequence
GTSNLAS

SEQ ID NO: 278 - protein, 7 aa, synthetic construct
  REGION 1..7: note = MIN-A2-2 light chain variable complementarity determining region 2 (CDR2) amino acid sequence
STSNLAS

SEQ ID NO: 279 - protein, 7 aa, synthetic construct
  REGION 1..7: note = MIN-C9-1 light chain variable complementarity determining region 2 (CDR2) amino acid sequence
STSNLAS

SEQ ID NO: 280 - protein, 7 aa, synthetic construct
  REGION 1..7: note = MIN-C9-2 light chain variable complementarity determining region 2 (CDR2) amino acid sequence
STSNLAS

SEQ ID NO: 281 - protein, 7 aa, synthetic construct
  REGION 1..7: note = MIN-D7-1 light chain variable complementarity determining region 2 (CDR2) amino acid sequence
STSNLAS

SEQ ID NO: 282 - protein, 7 aa, synthetic construct
  REGION 1..7: note = MIN-D7-2 light chain variable complementarity determining region 2 (CDR2) amino acid sequence
STSNLAS

SEQ ID NO: 283 - protein, 7 aa, synthetic construct
  REGION 1..7: note = MIN-F2-1 light chain variable complementarity determining region 2 (CDR2) amino acid sequence
GTSNLAS

SEQ ID NO: 284 - protein, 7 aa, synthetic construct
  REGION 1..7: note = MIN-F2-2 light chain variable complementarity determining region 2 (CDR2) amino acid sequence
GTSNLAS

SEQ ID NO: 285 - protein, 9 aa, synthetic construct
  REGION 1..9: note = MIN-A2-1 light chain variable complementarity determining region 3 (CDR3) amino acid sequence
QQRSNYPFT

SEQ ID NO: 286 - protein, 9 aa, synthetic construct
  REGION 1..9: note = MIN-A2-2 light chain variable complementarity determining region 3 (CDR3) amino acid sequence
QQRSSYPST

SEQ ID NO: 287 - protein, 9 aa, synthetic construct
  REGION 1..9: note = MIN-C9-1 light chain variable complementarity determining region 3 (CDR3) amino acid sequence
QQRSSYPST

SEQ ID NO: 288 - protein, 9 aa, synthetic construct
  REGION 1..9: note = MIN-C9-2 light chain variable complementarity determining region 3 (CDR3) amino acid sequence
QQRSSYPST

SEQ ID NO: 289 - protein, 9 aa, synthetic construct
  REGION 1..9: note = MIN-D7-1 light chain variable complementarity determining region 3 (CDR3) amino acid sequence
QQRSSYPST

SEQ ID NO: 290 - protein, 9 aa, synthetic construct
  REGION 1..9: note = MIN-D7-2 light chain variable complementarity determining region 3 (CDR3) amino acid sequence
QQRSSYPST

SEQ ID NO: 291 - protein, 9 aa, synthetic construct
  REGION 1..9: note = MIN-F2-1 light chain variable complementarity determining region 3 (CDR3) amino acid sequence
QQRSNYPFT

SEQ ID NO: 292 - protein, 9 aa, synthetic construct
  REGION 1..9: note = MIN-F2-2 light chain variable complementarity determining region 3 (CDR3) amino acid sequence
QQRSNYPFT

SEQ ID NO: 293 - protein, 5 aa, synthetic construct
  REGION 1..5: note = MIN-A2-1 heavy chain variable complementarity determining region 1 (CDR1) amino acid sequence
NYGMN

SEQ ID NO: 294 - protein, 5 aa, synthetic construct
  REGION 1..5: note = MIN-A2-2 heavy chain variable complementarity determining region 1 (CDR1) amino acid sequence
NYGMN

SEQ ID NO: 295 - protein, 5 aa, synthetic construct
  REGION 1..5: note = MIN-C9-1 heavy chain variable complementarity determining region 1 (CDR1) amino acid sequence
NNGMN

SEQ ID NO: 296 - protein, 5 aa, synthetic construct
  REGION 1..5: note = MIN-C9-2 heavy chain variable complementarity determining region 1 (CDR1) amino acid sequence
NNGMN

SEQ ID NO: 297 - protein, 5 aa, synthetic construct
  REGION 1..5: note = MIN-D7-1 heavy chain variable complementarity determining region 1 (CDR1) amino acid sequence
NYGMN

SEQ ID NO: 298 - protein, 5 aa, synthetic construct
  REGION 1..5: note = MIN-D7-2 heavy chain variable complementarity determining region 1 (CDR1) amino acid sequence
NYGMN

SEQ ID NO: 299 - protein, 5 aa, synthetic construct
  REGION 1..5: note = MIN-F2-1 heavy chain variable complementarity determining region 1 (CDR1) amino acid sequence
NYGMN

SEQ ID NO: 300 - protein, 5 aa, synthetic construct
  REGION 1..5: note = MIN-F2-2 heavy chain variable complementarity determining region 1 (CDR1) amino acid sequence
DYEMH

SEQ ID NO: 301 - protein, 5 aa, synthetic construct
  REGION 1..5: note = MIN-F2-3 heavy chain variable complementarity determining region 1 (CDR1) amino acid sequence
NYGMN

SEQ ID NO: 302 - protein, 5 aa, synthetic construct
  REGION 1..5: note = MIN-F2-4 heavy chain variable complementarity determining region 1 (CDR1) amino acid sequence
NYGMN

SEQ ID NO: 303 - protein, 17 aa, synthetic construct
  REGION 1..17: note = MIN-A2-1 heavy chain variable complementarity determining region 2 (CDR2) amino acid sequence
WINTYTGEPTYAGDFKG

SEQ ID NO: 304 - protein, 17 aa, synthetic construct
  REGION 1..17: note = MIN-A2-2 heavy chain variable complementarity determining region 2 (CDR2) amino acid sequence
WINTYTGEPTYAGDFKG

SEQ ID NO: 305 - protein, 17 aa, synthetic construct
  REGION 1..17: note = MIN-C9-1 heavy chain variable complementarity determining region 2 (CDR2) amino acid sequence
WINTYTGEPTYADDFKG

SEQ ID NO: 306 - protein, 17 aa, synthetic construct
  REGION 1..17: note = MIN-C9-2 heavy chain variable complementarity determining region 2 (CDR2) amino acid sequence
WINTYTGEPTYADDFKG

SEQ ID NO: 307 - protein, 17 aa, synthetic construct
  REGION 1..17: note = MIN-D7-1 heavy chain variable complementarity determining region 2 (CDR2) amino acid sequence
WINTYTGEPTYVDDFKG

SEQ ID NO: 308 - protein, 17 aa, synthetic construct
  REGION 1..17: note = MIN-D7-2 heavy chain variable complementarity determining region 2 (CDR2) amino acid sequence
WINTYTGEPTYAGDFKG

SEQ ID NO: 309 - protein, 17 aa, synthetic construct
  REGION 1..17: note = MIN-F2-1 heavy chain variable complementarity determining region 2 (CDR2) amino acid sequence
WINTYTGEPTYVDDFKG

SEQ ID NO: 310 - protein, 17 aa, synthetic construct
  REGION 1..17: note = MIN-F2-2 heavy chain variable complementarity determining region 2 (CDR2) amino acid sequence
AIHPGSGGTAYNQKFKG

SEQ ID NO: 311 - protein, 17 aa, synthetic construct
  REGION 1..17: note = MIN-F2-3 heavy chain variable complementarity determining region 2 (CDR2) amino acid sequence
WINTYTGEPTYVDDFKG

SEQ ID NO: 312 - protein, 17 aa, synthetic construct
  REGION 1..17: note = MIN-F2-4 heavy chain variable complementarity determining region 2 (CDR2) amino acid sequence
WINTYTGEPTYVDDFKG

SEQ ID NO: 313 - protein, 9 aa, synthetic construct
  REGION 1..9: note = MIN-A2-1 heavy chain variable complementarity determining region 3 (CDR3) amino acid sequence
SGDGYWYYA

SEQ ID NO: 314 - protein, 9 aa, synthetic construct
  REGION 1..9: note = MIN-A2-2 heavy chain variable complementarity determining region 3 (CDR3) amino acid sequence
SGDGYWYYA

SEQ ID NO: 315 - protein, 9 aa, synthetic construct
  REGION 1..9: note = MIN-C9-1 heavy chain variable complementarity determining region 3 (CDR3) amino acid sequence
TGTARAFYA

SEQ ID NO: 316 - protein, 9 aa, synthetic construct
  REGION 1..9: note = MIN-C9-2 heavy chain variable complementarity determining region 3 (CDR3) amino acid sequence
TGTARAFYA

SEQ ID NO: 317 - protein, 9 aa, synthetic construct
  REGION 1..9: note = MIN-D7-1 heavy chain variable complementarity determining region 3(CDR3) amino acid sequence
TGTTAILNG

SEQ ID NO: 318 - protein, 9 aa, synthetic construct
  REGION 1..9: note = MIN-D7-2 heavy chain variable complementarity determining region3 (CDR3) amino acid sequence
SGDGYWYYA

SEQ ID NO: 319 - protein, 9 aa, synthetic construct
  REGION 1..9: note = MIN-F2-1 heavy chain variable complementarity determining region 3 (CDR3) amino acid sequence
TGTTAILNG

SEQ ID NO: 320 - protein, 5 aa, synthetic construct
  REGION 1..5: note = MIN-F2-2 heavy chain variable complementarity determining region 3(CDR3) amino acid sequence
YGSFA

SEQ ID NO: 321 - protein, 9 aa, synthetic construct
  REGION 1..9: note = MIN-F2-3 heavy chain variable complementarity determining region 3 (CDR3) amino acid sequence
TGTTAILNG

SEQ ID NO: 322 - protein, 9 aa, synthetic construct
  REGION 1..9: note = MIN-F2-4 heavy chain variable complementarity determining region 3(CDR3) amino acid sequence
TGTTAILNG

SEQ ID NO: 323 - protein, 11 aa, synthetic construct
  REGION 1..11: note = MIN-14 light chain variable complementarity determining region 1 (CDR1) amino acid sequence
RASQDIGSSLN

SEQ ID NO: 324 - protein, 15 aa, synthetic construct
  REGION 1..15: note = MIN-17-1 light chain variable complementarity determining region 1 (CDR1) amino acid sequence
RASKSVSTSGYSYMH

SEQ ID NO: 325 - protein, 11 aa, synthetic construct
  REGION 1..11: note = MIN-17-2 light chain variable complementarity determining region 1 (CDR1) amino acid sequence
LASQTIGTWLA

SEQ ID NO: 326 - protein, 15 aa, synthetic construct
  REGION 1..15: note = MIN-29 light chain variable complementarity determining region 1 (CDR1) amino acid sequence
RASKSVSTSGYSYMH

SEQ ID NO: 327 - protein, 15 aa, synthetic construct
  REGION 1..15: note = MIN-34 light chain variable complementarity determining region 1 (CDR1) amino acid sequence
RASKSVSTSGYSYMH

SEQ ID NO: 328 - protein, 11 aa, synthetic construct
  REGION 1..11: note = MIN-42 light chain variable complementarity determining region 1 (CDR1) amino acid sequence
KASQNVGTNVG

SEQ ID NO: 329 - protein, 11 aa, synthetic construct
  REGION 1..11: note = MIN-45 light chain variable complementarity determining region 1 (CDR1) amino acid sequence
RASGNIHNFLA

SEQ ID NO: 330 - protein, 7 aa, synthetic construct
  REGION 1..7: note = MIN-14 light chain variable complementarity determining region 2 (CDR2) amino acid sequence
ATSSLDS

SEQ ID NO: 331 - protein, 7 aa, synthetic construct
  REGION 1..7: note = MIN-17-1 light chain variable complementarity determining region 2 (CDR2) amino acid sequence
LVSNLES

SEQ ID NO: 332 - protein, 7 aa, synthetic construct
  REGION 1..7: note = MIN-17-2 light chain variable complementarity determining region 2 (CDR2) amino acid sequence
AATSLAD

SEQ ID NO: 333 - protein, 7 aa, synthetic construct
  REGION 1..7: note = MIN-29 light chain variable complementarity determining region 2 (CDR2) amino acid sequence
LVSNLES

SEQ ID NO: 334 - protein, 7 aa, synthetic construct
  REGION 1..7: note = MIN-34 light chain variable complementarity determining region 2 (CDR2) amino acid sequence
LVSNLES

SEQ ID NO: 335 - protein, 7 aa, synthetic construct
  REGION 1..7: note = MIN-42 light chain variable complementarity determining region 2 (CDR2) amino acid sequence
SASYRYS

SEQ ID NO: 336 - protein, 7 aa, synthetic construct
  REGION 1..7: note = MIN-45 light chain variable complementarity determining region 2 (CDR2) amino acid sequence
NAKTLAD

SEQ ID NO: 337 - protein, 5 aa, synthetic construct
  REGION 1..5: note = MIN-14 heavy chain complementarity determining region 1 (CDR1) amino acid sequence
SYWMH

SEQ ID NO: 338 - protein, 5 aa, synthetic construct
  REGION 1..5: note = MIN-17-1 heavy chain complementarity determining region 1 (CDR1) amino acid sequence
SYWMH

SEQ ID NO: 339 - protein, 5 aa, synthetic construct
  REGION 1..5: note = MIN-17-2 heavy chain complementarity determining region 1 (CDR1) amino acid sequence
GIGVT

SEQ ID NO: 340 - protein, 5 aa, synthetic construct
  REGION 1..5: note = MIN-34 heavy chain complementarity determining region 1 (CDR1) amino acid sequence
SYGVH

SEQ ID NO: 341 - protein, 5 aa, synthetic construct
  REGION 1..5: note = MIN-42 heavy chain complementarity determining region 1 (CDR1) amino acid sequence
SFGMS

SEQ ID NO: 342 - protein, 5 aa, synthetic construct
  REGION 1..5: note = MIN-45 heavy chain complementarity determining region 1 (CDR1) amino acid sequence
GYFMS

SEQ ID NO: 343 - protein, 17 aa, synthetic construct
  REGION 1..17: note = MIN-14 heavy chain complementarity determining region 2 (CDR2) amino acid sequence
EINPSNGRTNYNEKFKS

SEQ ID NO: 344 - protein, 17 aa, synthetic construct
  REGION 1..17: note = MIN-17-1 heavy chain complementarity determining region 2 (CDR2) amino acid sequence
EINPSNGRTNYNEKFKS

SEQ ID NO: 345 - protein, 16 aa, synthetic construct
  REGION 1..16: note = MIN-17-2 heavy chain complementarity determining region 2 (CDR2) amino acid sequence
TIWWDDDNRYNPSLKS

SEQ ID NO: 346 - protein, 18 aa, synthetic construct
  REGION 1..18: note = MIN-29 heavy chain complementarity determining region 2 (CDR2) amino acid sequence
GIVSGDGESALHSVWIVG

SEQ ID NO: 347 - protein, 16 aa, synthetic construct
  REGION 1..16: note = MIN-34 heavy chain complementarity determining region 2 (CDR2) amino acid sequence
VIWGGGSTDYNAAFIS

SEQ ID NO: 348 - protein, 17 aa, synthetic construct
  REGION 1..17: note = MIN-42 heavy chain complementarity determining region 2 (CDR2) amino acid sequence
TISSGGTYTYYPDSVKG

SEQ ID NO: 349 - protein, 17 aa, synthetic construct
  REGION 1..17: note = MIN-45 heavy chain complementarity determining region 2 (CDR2) amino acid sequence
RINPYNGDTFYNQKFKG

SEQ ID NO: 350 - protein, 122 aa, synthetic construct
  REGION 1..122: note = Humanized E6 heavy chain variable region sequence
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 351 - protein, 5 aa, synthetic construct
  REGION 1..5: note = Humanized E6 heavy chain variable complementarity determining regions 1 (CDR1) sequence
RYGMS

SEQ ID NO: 352 - protein, 17 aa, synthetic construct
  REGION 1..17: note = Humanized E6 heavy chain variable complementarity determining regions 2 (CDR2) sequence
TISGGGTYIYYPDSVKG

SEQ ID NO: 353 - protein, 13 aa, synthetic construct
  REGION 1..13: note = Humanized E6 heavy chain variable complementarity determining regions 3 (CDR3) sequence
DNYGRNYDYGMDY

SEQ ID NO: 354 - protein, 106 aa, synthetic construct
  REGION 1..106: note = Humanized E6 light chain variable region sequence
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 355 - protein, 10 aa, synthetic construct
  REGION 1..10: note = Humanized E6 light chain variable complementarity determining regions 1 (CDR1) sequence
SATSSVSYIH

SEQ ID NO: 356 - protein, 7 aa, synthetic construct
  REGION 1..7: note = Humanized E6 light chain variable complementarity determining regions 2 (CDR2) sequence
STSNLAS

SEQ ID NO: 357 - protein, 9 aa, synthetic construct
  REGION 1..9: note = Humanized E6 light chain variable complementarity determining regions 3 (CDR3) sequence
QQRSSSPFT

SEQ ID NO: 358 - protein, 121 aa, synthetic construct
  REGION 1..121: note = Humanized C2 heavy chain variable region sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 359 - protein, 5 aa, synthetic construct
  REGION 1..5: note = Humanized C2 heavy chain variable complementarity determining regions 1 (CDR1) sequence
GYAMS

SEQ ID NO: 360 - protein, 17 aa, synthetic construct
  REGION 1..17: note = Humanized C2 heavy chain variable complementarity determining regions 2 (CDR2) sequence
TISSGGTYIYYPDSVKG

SEQ ID NO: 361 - protein, 12 aa, synthetic construct
  REGION 1..12: note = Humanized C2 heavy chain variable complementarity determining regions 3 (CDR3) sequence
LGGDNYYEYFDV

SEQ ID NO: 362 - protein, 113 aa, synthetic construct
  REGION 1..113: note = Humanized C2 light chain variable region sequence
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 363 - protein, 15 aa, synthetic construct
  REGION 1..15: note = Humanized C2 light chain variable complementarity determining regions 1 (CDR1) sequence
RASKSVSTSGYSYMH

SEQ ID NO: 364 - protein, 7 aa, synthetic construct
  REGION 1..7: note = Humanized C2 light chain variable complementarity determining regions 2 (CDR2) sequence
LASNLES

SEQ ID NO: 365 - protein, 9 aa, synthetic construct
  REGION 1..9: note = Humanized C2 light chain variable complementarity determining regions 3 (CDR3) sequence
QHSRELPFT

SEQ ID NO: 366 - protein, 9 aa, synthetic construct
  REGION 1..9: note = Humanized C2 light chain variable complementarity determining regions 3 (CDR3) sequence
LQSKNFPPT

SEQ ID NO: 367 - protein, 507 aa, synthetic construct
  REGION 1..507: note = pSECTag2 C2 scFV-FC
METDTLLLWVLLLWVPGSTGDAAQPAEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 501 aa, synthetic construct
  REGION 1..501: note = pSECTag2 E6 scFV-FC
METDTLLLWVLLLWVPGSTGDAAQPAEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 249 aa, synthetic construct
  REGION 1..249: note = Humanized C2 scFV (VH-VL) sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 370 - protein, 249 aa, synthetic construct
  REGION 1..249: note = Humanized E6 scFV (VL-VH) sequence
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTGGGGSGGGGSGGGGSEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 371 - protein, 119 aa, synthetic construct
  REGION 1..119: note = Humanized C3 heavy chain variable region sequence
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSS

SEQ ID NO: 372 - protein, 5 aa, synthetic construct
  REGION 1..5: note = Humanized C3 heavy chain variable complementarity determining regions 1 (CDR1) sequence
DYAMN

SEQ ID NO: 373 - protein, 17 aa, synthetic construct
  REGION 1..17: note = Humanized C3 heavy chain variable complementarity determining regions 2 (CDR2) sequence
VISTFSGNTNFNQKFKG

SEQ ID NO: 374 - protein, 10 aa, synthetic construct
  REGION 1..10: note = Humanized C3 heavy chain variable complementarity determining regions 3 (CDR3) sequence
SDYYGPYFDY

SEQ ID NO: 375 - protein, 114 aa, synthetic construct
  REGION 1..114: note = Humanized C3 light chain variable region sequence
DIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 376 - protein, 16 aa, synthetic construct
  REGION 1..16: note = Humanized C3 light chain variable complementarity determining regions 1 (CDR1) sequence
RSSQTIVHSNGNTYLE

SEQ ID NO: 377 - protein, 7 aa, synthetic construct
  REGION 1..7: note = Humanized C3 light chain variable complementarity determining regions 2 (CDR2) sequence
KVSNRFS

SEQ ID NO: 378 - protein, 9 aa, synthetic construct
  REGION 1..9: note = Humanized C3 light chain variable complementarity determining regions 3 (CDR3) sequence
FQGSHVPFT

SEQ ID NO: 379 - protein, 118 aa, synthetic construct
  REGION 1..118: note = Humanized C8 heavy chain variable region sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSS

SEQ ID NO: 380 - protein, 5 aa, synthetic construct
  REGION 1..5: note = Humanized C8 heavy chain variable complementarity determining region 1 (CDR1) sequence
GYAMS

SEQ ID NO: 381 - protein, 17 aa, synthetic construct
  REGION 1..17: note = Humanized C8 heavy chain variable complementarity determining region 2 (CDR2) sequence
TISSGGTYIYYPDSVKG

SEQ ID NO: 382 - protein, 9 aa, synthetic construct
  REGION 1..9: note = Humanized C8 heavy chain variable complementarity determining region 3 (CDR3) sequence
LGGDNYYEY

SEQ ID NO: 383 - protein, 114 aa, synthetic construct
  REGION 1..114: note = Humanized C8 light chain variable region sequence
DIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 384 - protein, 14 aa, synthetic construct
  REGION 1..14: note = Humanized C8 light chain variable complementarity determining region 1 (CDR1) sequence
RASKSVSTSGYSYM

SEQ ID NO: 385 - protein, 7 aa, synthetic construct
  REGION 1..7: note = Humanized C8 light chain variable complementarity determining region 2 (CDR2) sequence
LVSNLES

SEQ ID NO: 386 - protein, 45 aa, synthetic construct
  REGION 1..45: note = Humanized C8 light chain variable complementarity determining region 3 (CDR3) sequence
GLNHISILEARGGLULEUTHRARGSERGLUSEQUENCELISTING